SEQUENCE LISTING (1 sequence)

SEQ ID NO: 1 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

(19) United States
(12) Patent Application Publication
(10) Pub. No.: US 2005/0004126 A1
(43) Pub. Date: Jan. 6, 2005

(54) Method of determining potential allosterically-binding matrix metalloproteinase inhibitors

(76) Inventors: Charles Andrianjara, Fresnes (FR); Daniel Fred Ortwine, Saline, MI (US); Alexander Gregory Pavlovsky, Ann Arbor, MI (US); William Howard Roark, Ann Arbor, MI (US)

Correspondence Address: WARNER-LAMBERT COMPANY, 2800 PLYMOUTH RD, ANN ARBOR, MI, 48105, US

(21) Appl. No.: 10/835,619

(22) Filed: Apr. 29, 2004

Related U.S. Application Data
(62) Division of application No. 10/075,069, filed on Feb. 13, 2002, now abandoned.
(60) Provisional application No. 60/268,821, filed on Feb. 14, 2001.

(51) Int. Cl. A61K 31/535; A61K 31/50; A61K 31/517; A61K 31/47

(52) U.S. Cl. 514/248; 514/266.1; 514/249; 514/310; 514/314

(57) ABSTRACT

Compounds are provided that bind allosterically to the catalytic domain of MMP-13 and comprise a hydrophobic group, first and second hydrogen bond acceptors and at least one, and preferably both, of a third hydrogen bond acceptor and a second hydrophobic group. Cartesian coordinates for centroids of the above features are defined in the specification. When the ligand binds to MMP-13, the first, second and third (when present) hydrogen bond acceptors bond respectively with Thr245, Thr 247 and Met 253, the first hydrophobic group locates within the S1′ channel of MMP-13 and the second hydrophobic group (when present) is relatively open to solvent. The compounds specifically inhibit the matrix metalloproteinase-13 enzyme and thus are useful for treating diseases resulting from tissue breakdown, such as heart disease, multiple sclerosis, arthritis, atherosclerosis, and osteoporosis.

### FIELD OF THE INVENTION

This invention relates to compounds that inhibit matrix metalloproteinase enzymes and thus are useful for treating diseases resulting from tissue breakdown, such as heart disease, multiple sclerosis, arthritis, atherosclerosis, and osteoporosis.

### BACKGROUND OF THE INVENTION

Matrix metalloproteinases (sometimes referred to as MMPs) are naturally-occurring enzymes found in most mammals. Over-expression and activation of MMPs or an imbalance between MMPs and inhibitors of MMPs have been suggested as factors in the pathogenesis of diseases characterized by the breakdown of extracellular matrix or connective tissues.

Stromelysin-1 and gelatinase A are members of the matrix metalloproteinase (MMP) family. Other members include fibroblast collagenase (MMP-1), neutrophil collagenase (MMP-8), gelatinase B (92 kDa gelatinase) (MMP-9), stromelysin-2 (MMP-10), stromelysin-3 (MMP-11), matrilysin (MMP-7), collagenase 3 (MMP-13), and other newly discovered membrane-associated matrix metalloproteinases (Sato H., Takino T., Okada Y., Cao J., Shinagawa A., Yamamoto E., and Seiki M., Nature, 1994, 370, 61-65). These enzymes have been implicated with a number of diseases that result from breakdown of connective tissue, including such diseases as rheumatoid arthritis, osteoarthritis, osteoporosis, periodontitis, multiple sclerosis, gingivitis, corneal epidermal and gastric ulceration, atherosclerosis, neointimal proliferation which leads to restenosis and ischemic heart failure, and tumor metastasis. A method for preventing and treating these and other diseases is now recognized to be by inhibiting metalloproteinase enzymes, thereby curtailing and/or eliminating the breakdown of connective tissues that results in the disease states.

The catalytic zinc in matrix metalloproteinases is typically the focal point for inhibitor design. The modification of substrates by introducing zinc chelating groups has generated potent inhibitors such as peptide hydroxamates and thiol-containing peptides. Peptide hydroxamates and the natural endogenous inhibitors of MMPs (Tissue Inhibitors of Metalloproteinases (TIMPs)) have been used successfully to treat animal models of cancer and inflammation. MMP inhibitors have also been proposed to prevent and treat congestive heart failure and other cardiovascular diseases. See for example U.S. Pat. No. 5,948,780.

A major limitation on the use of currently known MMP inhibitors is their lack of specificity for any particular enzyme. Recent data has established that specific MMP enzymes are associated with some diseases, with no effect on others. The MMPs are generally categorized based on their substrate specificity, and indeed the collagenase subfamily of MMP-1, MMP-8, and MMP-13 selectively cleave native interstitial collagens, and thus are associated only with diseases linked to such interstitial collagen tissue. This is evidenced by the recent discovery that MMP-13 alone is overexpressed in breast carcinoma, while MMP-1 alone is overexpressed in papillary carcinoma (see Chen et al., J. Am. Chem. Soc., 2000, 122(40), 9648-9654).

There appears to be only one selective inhibitor of MMP-13, namely WAY-170523, as reported by Chen et al., supra. Therefore the need remains to find new low molecular weight compounds that are potent and selective MMP inhibitors, and that have an acceptable therapeutic index of toxicity/potency to make them amenable for use clinically in the prevention and treatment of the associated disease states.

NMR and X-ray structures of inhibited MMP-13 have been reported by Lovejoy et al., Nat. Struct. Biol., 1999, 6(3), 217-221 and Moy F. J. et al., J. Mol. Biol., 2000, 302, 673-691. The existence has been disclosed of a deep S1′ pocket within the MMP-13 protein that extends from the catalytic zinc in the active site. Chen et al., J. Am Chem. Soc., 2000, 122, 9648-9654 disclose that there are differences in size and shape within the S1′ pocket of different MMP enzymes and suggest that this difference across the MMP family of enzymes provides a possible approach for designing specificity into potent MMP inhibitors by designing compounds that appropriately fill the available space in the S1′ pocket while taking advantage of sequence differences between various MMPs. They also describe the S1′ site of MMP-13 as being unusually large and providing features that can be exploited in the design of potentially selective MMP-13 inhibitors. As a result of high throughput screening, the authors found a compound of the formula I below which exhibited weak inhibition against MMP-13 but was inactive against other MMP enzymes. 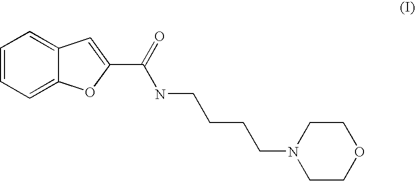
An NMR spectrum of the complex that forms between the compound of formula (I) and the catalytic domain of MMP-13 [MMP-13 CD] confirmed that the compound sits in the S1′ pocket but does not bind to zinc. Further compounds were tested that combined a first portion containing functionality designed to form a direct complex with the catalytic zinc atom in the active site, and a second portion of the molecule which is intended to sit in the S1′ pocket. The best compound reported had an IC₅₀ for MMP-13 of 17 nM and showed 5800-fold and 56-fold specificity against MMP-1 and MMP-9 respectively. Other compounds that combine a first portion containing a functionality that forms a direct complex with the catalytic zinc atom in the active site of a matrix metalloproteinase and a second portion that is intended to sit in the S1′ pocket are described in WO 01/05389 (Stallings et. al., G. D. Searle). This approach may not lead to compounds of practical utility since complex formation is via an N-hydroxy group or a group closely related thereto located adjacent to an aryl ring, and such compounds have been reported to be carcinogenic or mutagenic, see Weisburger, J. H. et al., “Biochemical formation and pharmacological, toxicological and pathological properties of hydroxylamines and hydroxamic acids”, Pharmacol. Rev., 1973, 25(1), 1-66.

### SUMMARY OF THE INVENTION

The invention provides compounds that bind allosterically into the S1′ site and S1″ site of MMP 13. The S1′ channel is a specific part of the S1′ site and is formed largely by Leu218, Val219, His222 and by residues from Leu239 to Tyr244. The S1″ binding site has been newly discovered and is defined by residues from Tyr246 to Pro255. Without wishing to be bound by any particular theory, the inventors believe that this site could be a recognition site for triple helix collagen, the natural substrate for MMP-13. The S1″ site contains at least two hydrogen bond donors and aromatic groups which interact with the compound of the invention. It is possible that the conformation of the S1″ site is modified only when an appropriate compound binds to MMP-13, thereby interfering with the collagen recognition process. This pattern of binding offers the possibility of greater selectivity than is achieved with known ligands that bind to the catalytic zinc atom at the active site and/or into the S1′ pocket.

The invention provides compounds that bind allosterically to and inhibit MMP-13 and that have a pharmacophore comprising at least a first hydrophobic group and at least first and second hydrogen bond acceptors. The compound will normally have a second hydrophobic group, a third hydrogen bond acceptor or both a second hydrophobic group and a third hydrogen bond acceptor.

The pharmacophore of a compound means the minimum functionality that a compound has to contain in order to exhibit activity and is commonly defined in terms of centres that interact with a receptor. One way of defining the pharmacophore is by the combination of active centers and their relative positions in space.

In one aspect, the invention provides a compound that binds allosterically to MMP-13 and that comprises first and second hydrophobic groups and first and second hydrogen bond acceptors, wherein:
(a) the relative positions of centroids of the above features are defined by the following Cartesian coordinates in Å:
   (i) first hydrogen bond acceptor, 0.00, 0.00, 0.00;
   (ii) second hydrogen bond acceptor, 5.08, 2.23, 0.0;
   (iii) first hydrophobic group, −1.52, −3.06, −0.23;
   (iv) second hydrophobic group, 9.07, 0.00, 0.00; and
(b) tolerances in the positions of the hydrophobic groups and the hydrogen bond acceptors are ±1.0 Å and ±1.5 Å respectively.

The invention also provides a compound that binds allosterically to MMP-13 and that comprises a hydrophobic group and first, second and third hydrogen bond acceptors, wherein:
(a) the relative positions of centroids of the above features are defined by the following Cartesian coordinates in Å:
   (i) first hydrogen bond acceptor, 0.00, 0.00, 0.00;
   (ii) second hydrogen bond acceptor, 5.08, 2.23, 0.0;
   (iii) third hydrogen bond acceptor, 7.15, 0.80, 0.00;
   (iv) first hydrophobic group, −1.52, −3.06, −0.23; and
(b) tolerances in the positions of the hydrophobic group and the hydrogen bond acceptors are ±1.0 Å and ±1.5 Å respectively.

The invention further provides a compound that binds allosterically to MMP-13 and that comprises first and second hydrophobic groups and first, second and third hydrogen bond acceptors, wherein:
(a) the relative positions of centroids of the above features are defined by the following Cartesian coordinates in Å:
   (i) first hydrogen bond acceptor, 0.00, 0.00, 0.00;
   (ii) second hydrogen bond acceptor, 5.08, 2.23, 0.0;
   (iii) third hydrogen bond acceptor, 7.15, 0.80, 0.00;
   (iv) first hydrophobic group, −1.52, −3.06, −0.23;
   (v) second hydrophobic group, 9.07, 0.00, 0.00; and
(b) tolerances in the positions of the hydrophobic groups and the hydrogen bond acceptors are ±1.0 Å and ±1.5 Å respectively.

A further way of defining the pharmacophore is in terms of the centers present and the sites on the receptor with which they interact.

Thus there may further be provided a ligand that binds allosterically to MMP-13 and that comprises a scaffold, first and second hydrogen bond acceptors and first and second hydrophobic groups connected by side chains to the scaffold, a cyclic structure forming part of the scaffold being located between the first and second hydrogen bond acceptors, and the hydrogen bond acceptors and hydrophobic groups being arranged so that when the ligand binds to MMP-13:
the first and second hydrogen bond acceptors interact respectively with the backbone NH's of Thr245 and Thr 247;
the first hydrophobic group locates within the S1′ channel; and
the second hydrophobic group is open to solvent.

There may yet further be provided a ligand that binds allosterically to MMP-13 and that comprises a scaffold, first, second and third hydrogen bond acceptors, and a hydrophobic group connected by a side chain to the scaffold, a cyclic structure forming part of the scaffold being located between the first and second hydrogen bond acceptors, and the hydrogen bond acceptors and hydrophobic group being arranged so that when the ligand binds to MMP-13:
the first, second and third hydrogen bond acceptors bond respectively with backbone NH's of Thr245, Thr 247 and Met 253; and
the first hydrophobic group locates within the S1′ channel.

Preferred is a ligand that binds allosterically to MMP-13 and that comprises a scaffold, first, second and third hydrogen bond acceptors, and first and second hydrophobic groups connected by side chains to the scaffold, a cyclic structure forming part of the scaffold being located between the first and second hydrogen bond acceptors, and the hydrogen bond acceptors and hydrophobic groups being arranged so that when the ligand binds to MMP-13:
the first, second and third hydrogen bond acceptors bond respectively with the backbone NH's of Thr245, Thr 247 and Met 253;
the first hydrophobic group locates within the S1′ channel; and
the second hydrophobic group is open to solvent.

In some compounds the third hydrogen bond acceptor may additionally form a hydrogen bond via a bridging water molecule with the backbone carbonyl of His251.

The existence and properties of the pharmacophore described above are supported by:
(i) crystal structure determinations of MMP-13 CD having ligands according to the invention bonded thereto, which structure determinations have provided detailed information concerning the sites which are important for allosteric binding between a ligand and MMP-13 CD; and
(ii) structure-activity relationships that have been determined by the present applicants for compounds within seven series that have been prepared by them and which are described in their co-pending six WO applications which claim respectively the U.S. priority applications Nos. U.S. 60/268,780, U.S. 60/268,736, U.S. 60/268,756, U.S. 60/268,661, U.S. 60/268,757, and U.S. 60/268,782, filed on Feb. 14, 2001 and in their co-pending WO application PCT/EP01/11824 filed on Oct. 12, 2001, the disclosures of which are incorporated herein by reference. Structure-activity relationships for compounds disclosed in the co-pending applications are given below, and the synthesis of a number of the compounds is for convenience of reference additionally described in this application.

In a further aspect, the invention relates to the use of a compound as aforesaid for the preparation of a medicament for the treatment of a disease by inhibition of MMP-13.

In another aspect the invention relates to the use of a compound as aforesaid for the manufacture of a medicament for the treatment of any of arthritis, rheumatoid arthritis, osteoarthritis, osteoporosis, peridontal disease, inflammatory bowel disease, psoriasis, multiple sclerosis, cardiac insufficiency, atherosclerosis, asthma, chronic obstructive pulmonary disease (COPD), age-related macular degeneration or cancer.

Further, the invention provides a method of treatment of any of arthritis, rheumatoid arthritis, osteoarthritis, osteoporosis, peridontal disease, inflammatory bowel disease, psoriasis, multiple sclerosis, cardiac insufficiency, atherosclerosis, asthma, chronic obstructive pulmonary disease (COPD), age-related macular degeneration or cancer which comprises administering to a patient an effective amount of a compound as aforesaid.

### DESCRIPTION OF PREFERRED FEATURES

Preferred Features of the Pharmacophore

As mentioned previously, the main features of the pharmacophore may broadly comprise a first and optionally a second hydrophobic group and a first, a second and optionally a third hydrogen bond acceptor connected by side chains to a scaffold. These main features will now be described in more detail in relation to particularly preferred embodiments of the invention.

The various positions outlined below are determined by counting the atoms in a clockwise fashion when the first hydrophobic group is located on the left hand side of the compound, and the first and second hydrogen bond acceptors are located on the upper side of the compound, as exemplified for instance in FIGS. 4 to 8.

Turning first to preferred embodiments of the pharmacophore defined with relation to the scaffold itself, a first preferred embodiment comprises a first 5 or 6-membered scaffold ring which may optionally contain one or more heteroatoms, preferably one heteroatom selected from nitrogen, oxygen or sulfur. In a second embodiment of the pharmacophore of the present invention, the scaffold comprises a first scaffold ring as defined above to which is fused a second 5 or 6-membered scaffold ring, preferably a 6-membered aromatic scaffold ring. The second scaffold ring is defined as above for the first scaffold ring. Yet another and third embodiment of the pharmacophore comprises a first scaffold ring, a second scaffold ring fused to said first scaffold ring and a third 5 or 6-membered scaffold ring, which is as defined above for the first scaffold ring, and which is fused to the second scaffold ring.

The hydrophobic group, or when two such groups are present the first hydrophobic group, may be an n-alkyl. n-alkenyl or n-alkynyl group having between 4 and 10 carbon atoms, optionally containing embedded oxygen or sulfur atoms, a bicyclic ring system containing between 8 and 10 atoms and which may contain one or several heteroatoms, or a 5- or 6-membered monocyclic group, preferably aromatic which may contain one or more heteroatoms, e.g. morpholine or piperidine, and which may be 4-substituted or 3,4-disubstituted, but which is of width (including substituents) less than 4.0 Å e.g. phenyl. For best activity, the π-system of the aromatic ring is electron rich by reason of a hetero atom e.g. 3-pyridyl or 4-pyridyl or because the ring has electron-donating groups. Electron-withdrawing groups, e.g. —CO₂, —NO₂, —SO₂NH₂ or —F are disfavoured.

The hydrophobic group, or where there are two such groups the first hydrophobic group, is preferably linked by a first linker chain, which is three atoms long, to a first 5 or 6-membered ring of the scaffold. The first linker chain atom adjacent to said first scaffold ring forms part of the first hydrogen bond acceptor (e.g. sulfonyl, ester, unsubstituted amide, or alkynyl). Preferably the first linker chain has a methylene group located adjacent to the hydrophobic group.

The second hydrophobic group when present can contribute significantly to selectivity because it has been found to stabilize and interact with the S1″ site of the protein. It is preferably a 5 or 6-membered ring, preferably aromatic, which may contain one or several heteroatoms, a bicyclic ring system containing between 8 and 10 atoms and which may also contain one or several heteroatoms or a planar saturated or unsaturated system e.g. cyclohexylmethyl. Optimally, it is an aromatic system that is capable of pi-orbital overlap with aromatic residues in the protein. The ring may have a wide range of substituents in the meta- or para-positions.

The second hydrophobic group it is preferably linked to the scaffold by a second linker chain which is three atoms long when the scaffold comprises only a first scaffold ring. In this situation, the second linker chain atom adjacent to the first scaffold ring preferably forms part of the second hydrogen bond acceptor. When the scaffold contains more than one ring, the second hydrophobic group is preferably linked to the second scaffold ring by a third linker chain preferably comprising an unsubstituted methylene linking group.

Turning now in more detail to the first preferred embodiment of the pharmacophore which comprises a first scaffold ring, it comprises a first hydrophobic group as defined above which is linked to the first scaffold ring through a first linker chain. It also comprises a second hydrophobic group linked to the first scaffold ring through a second linker chained as defined above. The junctions of the first and second linker chains with the first scaffold ring are on different atoms of this ring and are separated by one atom or more, preferably by one atom. Also, the first and second linker chain atoms adjacent to the ring respectively form part of the first and second hydrogen bond acceptors. Furthermore, the scaffold ring preferably contains a substituent (preferably methyl or methoxy) located opposite to the junction of the first linker chain with the ring.

With regard to the second preferred embodiment of the pharmacophore of the present invention, which can be used for increased potency, the scaffold comprises a second scaffold ring fused to the first scaffold ring at locations two and three ring atoms distant from the junction between the first scaffold ring and the first linker chain. The atom of the second scaffold ring adjacent to the atom of the first scaffold ring that is two positions distant from said junction forms part of the second hydrogen bond acceptor. Because of size limitations in bicyclic structures, the positions of the first scaffold ring to either side of the junction of the first ring with the first linker chain have only hydrogen atoms or ring heteroatoms. In order to provide a limited region of additional volume and to give an enhancement in activity, the atom of the second scaffold ring adjacent to the atom of the first scaffold ring that is three positions distant from said junction has a substituent which is a single atom or is a methyl group. The second scaffold ring is preferably 6-membered, and the atom of the second scaffold ring that is two positions distant from the atom that forms part of the second hydrogen bond acceptor preferably forms part of the third hydrogen bond acceptor.

As for the third embodiment of the pharmacophore, in which the second scaffold ring is preferably 6-membered, a third scaffold ring is fused to the second scaffold ring at those atoms of the second scaffold ring which are two and three positions distant from the atom that forms part of the second hydrogen bond acceptor. An atom of the third scaffold ring forms part of the third hydrogen bond acceptor.

Forms of the Present Compounds

The present compounds can exist in unsolvated forms as well as solvated forms, including hydrated forms. In general, the solvated forms, including hydrated forms, are equivalent to unsolvated forms and are intended to be encompassed within the scope of the present invention. The compounds are capable of further forming both pharmaceutically acceptable salts, including but not limited to acid addition and/or base salts.

Pharmaceutically acceptable acid addition salts of the compounds of Formula I include salts derived form inorganic acids such as hydrochloric, nitric, phosphoric, sulfuric, hydrobromic, hydriodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are the salts of amino acids such as arginate, gluconate, galacturonate, and the like; see, for example, Berge, et al., “Pharmaceutical Salts,” J. of Pharmaceutical Science, 1977; 66:1-19.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine; see, for example, Berge, et al., supra.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

Compositions, Uses and Methods of Treatment

This invention also provides pharmaceutical compositions comprising a compound as defined above together with a pharmaceutically acceptable carrier, diluent, or excipient therefor. All of these forms can be used in the method of the present invention.

The compounds of the present invention can be formulated and administered in a wide variety of oral and parenteral dosage forms, including transdermal and rectal administration. All that is required is that an MMP inhibitor be administered to a mammal suffering from a disease in an effective amount, which is that amount required to cause an improvement in the disease and/or the symptoms associated with such disease. It will be recognized to those skilled in the art that the following dosage forms may comprise as the active component, either a compound as defined above or a corresponding pharmaceutically acceptable salt or solvate of a compound as defined above.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally. It will be obvious to those skilled in the art that the following dosage forms may comprise as the active component, either a compound as defined above or a corresponding pharmaceutically acceptable salt of a compound as defined above. The active compound generally is present in a concentration of about 5% to about 95% by weight of the formulation.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 1 mg to 1000 mg, preferably 10 mg to 100 mg according to the particular application and the potency of the active component. The composition can, if desired, also contain other compatible therapeutic agents.

In therapeutic use as agents to inhibit a matrix metalloproteinase enzyme for the treatment of atherosclerotic plaque rupture, aortic aneurism, heart failure, restenosis, periodontal disease, corneal ulceration, cancer metastasis, tumor angiogenesis, arthritis, or other autoimmune or inflammatory disorders dependent upon breakdown of connective tissue, the compounds utilized in the pharmaceutical method of this invention are administered at a dose that is effective to inhibit the hydrolytic activity of matrix metalloproteinase 13. The initial dosage of about 1 mg to about 100 mg per kilogram daily will be effective. A daily dose range of about 25 mg to about 75 mg per kilogram is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages that are less than the optimum dose of the compound. Thereafter, the dosage is increased by small increments until the optimum effect under the circumstance is reached. For convenience, the total daily dosage may be divided and administered in portions during the day if desired. Typical dosages will be from about 0.1 to about 500 mg/kg, and ideally about 25 to about 250 mg/kg, such that it will be an amount that is effective to treat the particular disease being prevented or controlled.

### BRIEF DESCRIPTION OF THE DRAWINGS

How the invention may be put into effect will now be described with reference to the accompanying drawings, in which:

FIG. 1 is a sequence listing for MMP-13;

FIG. 2 is a partly cut-away view of the MMP-13 molecule showing the catalytic domain and the S1′ and S1″ binding sites;

FIG. 3 is a view of the catalytic domain of MMP-13 with a compound according to the invention bound into the S1′ and S1″ sites; and

FIGS. 4-8 are diagrams showing how a representative compound of each of the five series of compounds discussed below binds into S1′ and S1″ binding sites.

FIG. 9 is a diagram of the pharmacophore showing the location of first and second hydrophobic groups and first, second and third hydrogen bond acceptors, their respective coordonates, and angles and distances between them.

### DETAILED DESCRIPTION OF PREFERRED EMBODIMENTS

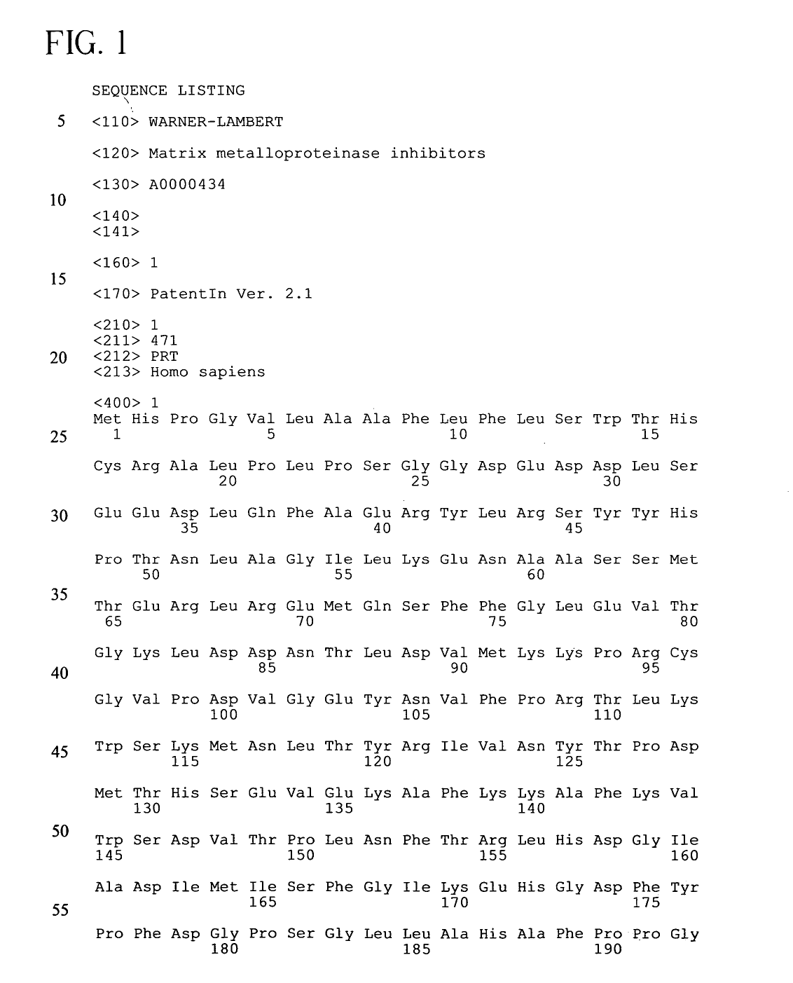

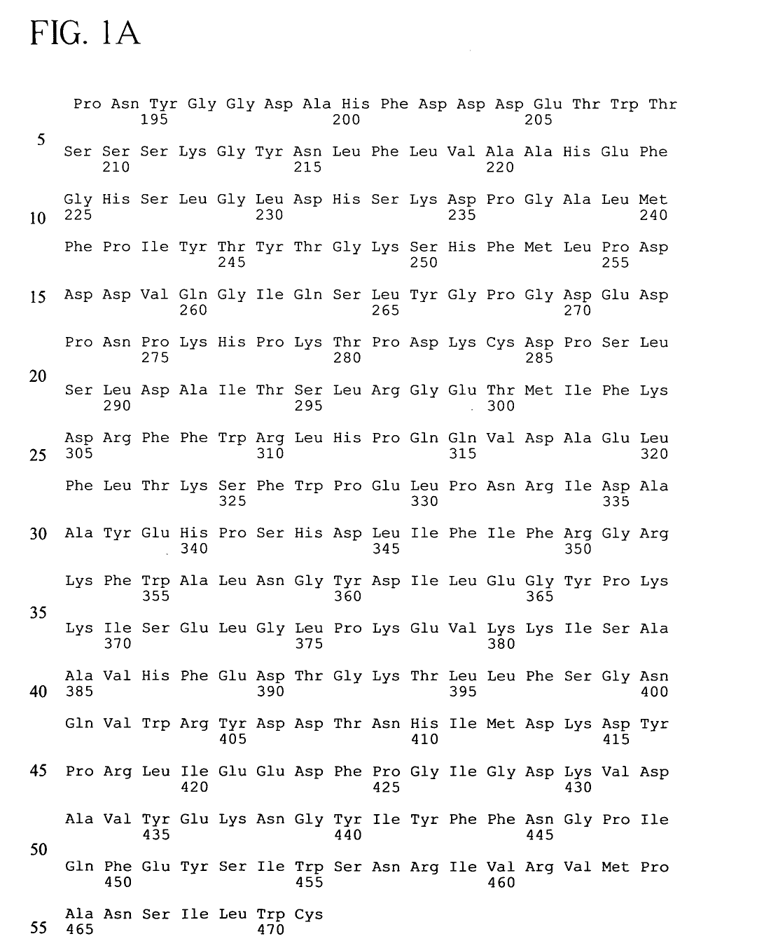

As previously discussed, the crystal structure of MMP-13 is known. The sequence listing of FIG. 1 is in accordance with that entered into the SWISS-PROT database under the sequence No P45452. In other publications of the MMP-13 sequence the numbering of the amino acid residues may differ, but a skilled person will readily identify any differences and the particular amino acid residues which are mentioned herein.

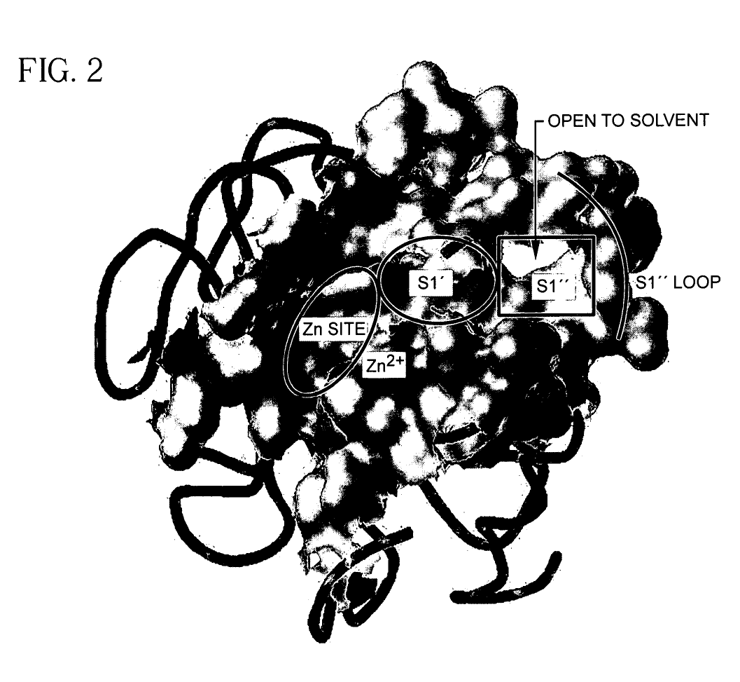

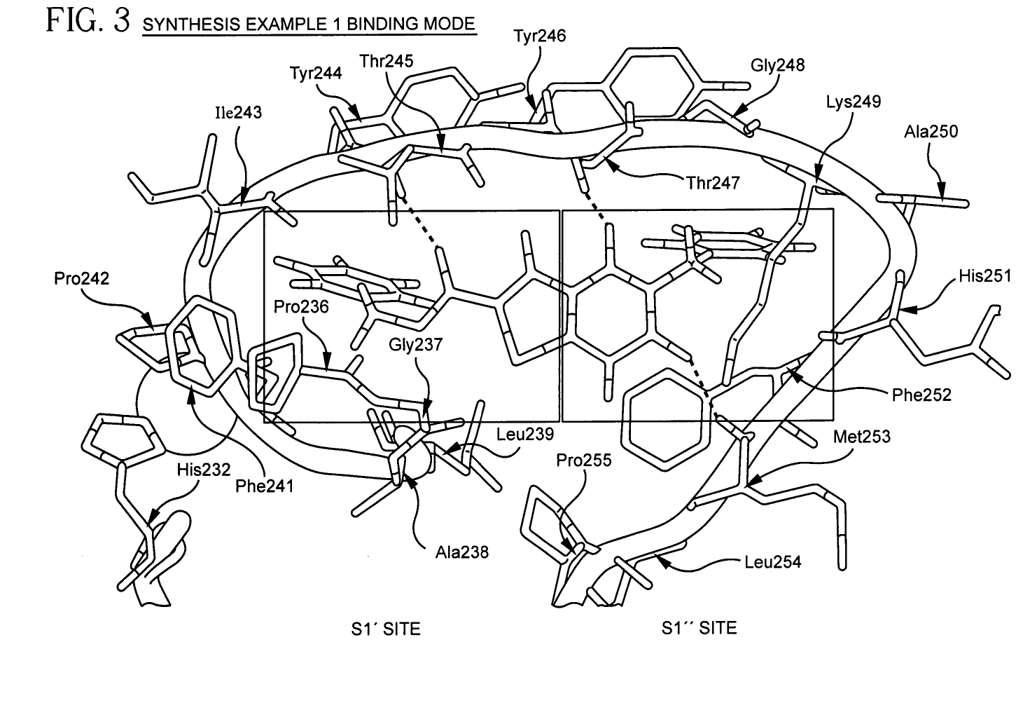

FIG. 2 is a view of the MMP-13 molecule partly cut away to reveal the binding sites. The active center of the enzyme contains a zinc atom. Ligands bind to this site by chelation to the zinc atom, and additionally locate in a pocket S1′ as discussed by Lovejoy et al.; supra. The present ligands bind at a newly discovered site S1″ which is, as shown, at a greater distance from the zinc atom. They do not bind by chelation at the zinc in the active site. Note the presence of an open space within the S1″ pocket through which the second hydrophobic group can be located in order to be open to solvent. The term “open to solvent” therefore refers to a position of the second hydrophobic group (when present) which is probably partially outside the MMP-13 protein through this open space and this in turn appears to expose this substituent to the intracellular medium in which MMP-13 is normally located.

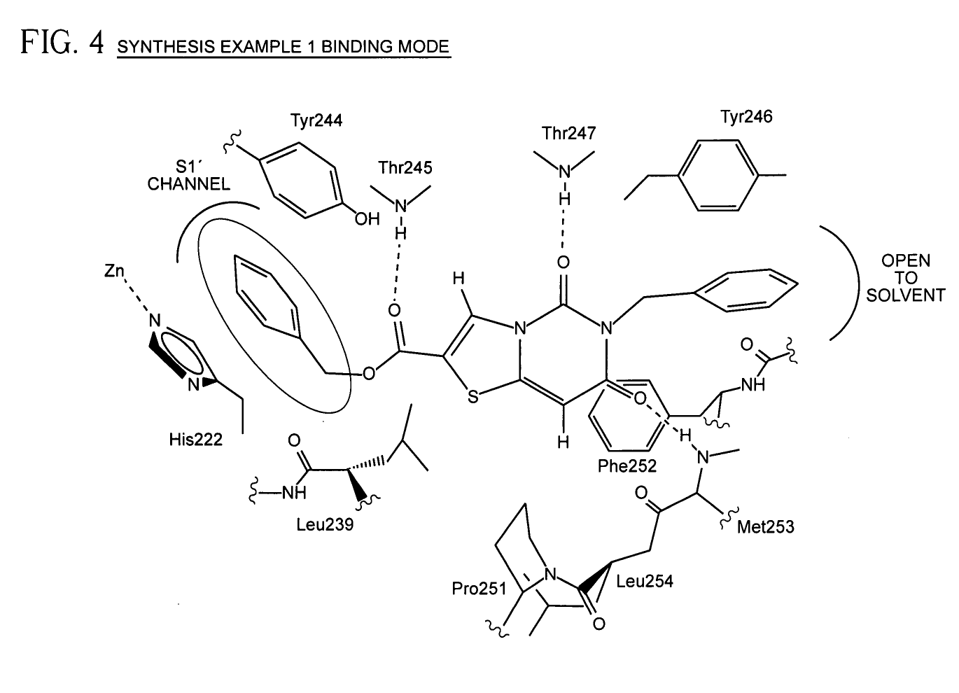

FIGS. 4-8 are discussed in relation to the particular series of compounds to which they relate.

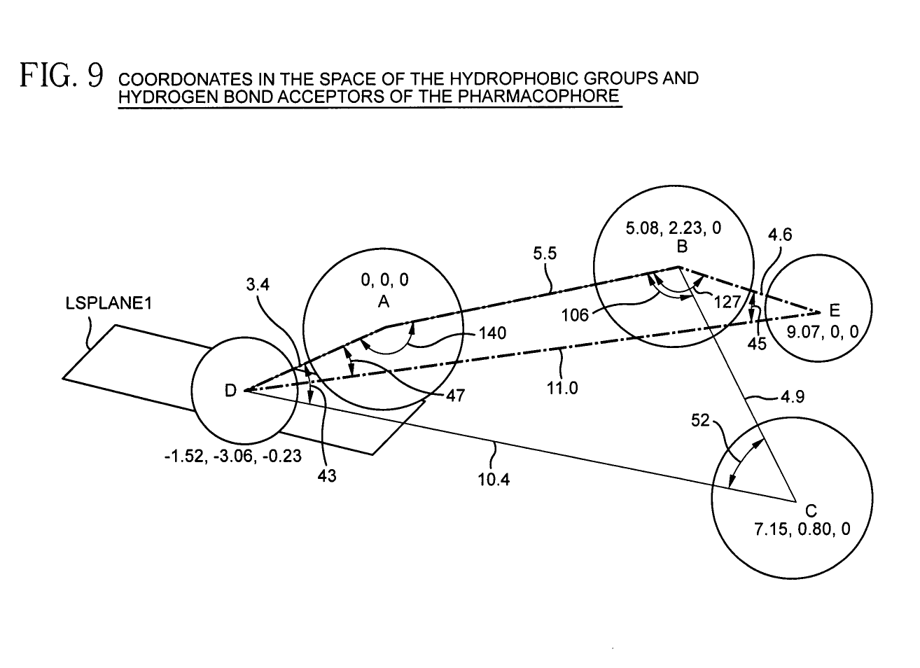

FIG. 9 is a view of the pharmacophore wherein is represented the location of the first and the second hydrophobic group (respectively the site D and E), and the first, second and third hydrogen bond acceptor (respectively the site A, B and C). Each site is characterized by its coordinates in the space, the distances and the angles between the others sites.

Thiazolopyrimidinediones

We have made a first group of compounds which are thiazolopyrimidinediones and are inhibitors of matrix metalloproteinase enzymes, and especially MMP-13. Preferred compounds that we have made, and their ability to inhibit the activity of various matrix metalloproteinase enzymes are summarized in Tables 1a and 1b below:

TABLE 1a

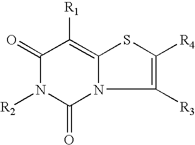

| | MMLP01 | MMP02 | MMP03 | MMP07 | MMP09 | MMP13 | MMP14 |
|---|---|---|---|---|---|---|---|
| | (FL) | (FL) | (CD) | (FL) | (FL) | (CD) | (CD) |
| Synthesis | IC₅₀ | IC₅₀ | IC₅₀ | IC₅₀ | IC₅₀ | IC₅₀ | IC₅₀ |
| Example | μM | μM | μM | μM | μM | μM | μM |
| 1 | >100 | >100 | >100 | >100 | >100 | 0.0230 | >100 |
| 2 | >100 | 81 | >100 | >100 | >100 | 0.51 | >100 |
| 3 | >100 | >100 | >30 | >100 | >100 | 0.0056 | >100 |
| 4 | 100 | >100 | 30 | 100 | 100 | 0.0054 | 100 |
| 5 | >100 | >100 | 29 | >100 | >100 | 0.0015 | >100 |
| 6 | >100 | >100 | >100 | >100 | >100 | 0.0057 | >100 |
| 7 | >100 | >100 | >100 | >100 | >100 | 0.0235 | >100 |
| 8 | >100 | >100 | >100 | >100 | >100 | 0.0840 | >100 |

TABLE 1b

| | | | MMP13 |
|---|---|---|---|
| | MMP01 | MMP03 | IC50 |
| Compound | IC50 (μM) | IC50 (μM) | (μM) |
| 6-Benzyl-5,7-dioxo-6,7-dihydro-5H- | >100 | 68 | 0.490 |
| thiazolo[3,2-c]pyrimidine-2-carboxylic acid (pyridin- | | | |
| 4-ylmethyl)-amide hydrochloride | | | |
| 6-Benzyl-8-methyl-5,7-dioxo-6,7-dihydro-5H- | >30 | >30 | 0.120 |
| thiazolo[3,2-c]pyrimidine-2-carboxylic acid 3-fluoro- | | | |
| benzylamide | | | |
| 6-Benzoyl-5,7-dioxo-6,7-dihydro-5H- | >30 | >30 | 6.700 |
| thiazolo[3,2-c]pyrimidine-2-carboxylic acid | | | |
| benzylamide | | | |
| 6-(3,4-Dichlorobenzyl)-5,7-dioxo-6,7-dihydro-5H- | >30 | >30 | 0.293 |
| thiazolo[3,2-c]pyrimidine-2-carboxylic acid | | | |
| benzylamide | | | |
| 6-(4-Chlorobenzyl)-5,7-dioxo-6,7-dihydro-5H- | >30 | 43 | 0.415 |
| thiazolo[3,2-c]pyrimidine-2-carboxylic acid | | | |
| benzylamide | | | |
| 6-(4-Chlorobenzyl)-5,7-dioxo-6,7-dihydro-5H- | >30 | >100 | 4.300 |
| thiazolo[3,2-c]pyrimidine-2-carboxylic acid | | | |
| 3,4-dichlorobenzylamide | | | |
| 6-(4-Pyridylmethyl)-5,7-dioxo-6,7-dihydro-5H- | >100 | >100 | 1.800 |
| thiazolo[3,2-c]pyrimidine-2-carboxylic acid | | | |
| benzylamide hydrochloride | | | |
| 6-Benzyl-8-methyl-5,7-dioxo-6,7-dihydro-5H- | >100 | 51 | 0.094 |
| thiazolo[3,2-c]pyrimidine-2-carboxylic acid | | | |
| benzylamide | | | |
| 6-Benzyl-8-methyl-5,7-dioxo-6,7-dihydro-5H- | >30 | 16 | 0.0217 |
| thiazolo[3,2-c]pyrimidine-2-carboxylic acid | | | |
| 4-methoxybenzylamide | | | |
| 6-Benzyl-8-methyl-5,7-dioxo-6,7-dihydro-5H- | >30 | 20 | 0.265 |
| thiazolo[3,2-c]pyrimidine-2-carboxylic acid | | | |
| 3,4-dichlorobenzylamide | | | |
| 6-Benzyl-5,7-dioxo-2,3,6,7-tetrahydro-5H- | >100 | >100 | 6.500 |
| thiazolo[3,2-c]pyrimidine-2-carboxylic acid pyridin- | | | |
| 4-ylmethyl ester hydrochloride | | | |
| 6-Benzyl-5,7-dioxo-2,3,6,7-tetrahydro-5H- | >100 | >100 | 0.590 |
| thiazolo[3,2-c]pyrimidine-2-carboxylic acid (pyridin- | | | |
| 4-ylmethyl)-amide | | | |
| 6-Benzyl-1,5,7-trioxo-1,2,3,5,6,7-hexahydro- | >100 | 23 | 0.072 |
| 1λ⁴-thiazolo[3,2-c]pyrimidine-3-carboxylic acid benzyl | | | |
| ester | | | |
| 6-Benzyl-8-methyl-5,7-dioxo-6,7-dihydro-5H- | >100 | 19 | 0.025 |
| thiazolo[3,2-c]pyrimidine-2-carboxylic acid 3-methyl- | | | |
| benzylamide | | | |
| 6-Benzyl-8-methyl-5,7-dioxo-6,7-dihydro-5H- | >30 | >100 | 0.0245 |
| thiazolo[3,2-c]pyrimidine-2-carboxylic acid 4-fluoro- | | | |
| benzylamide | | | |
| 6-Benzyl-8-formyl-5,7-dioxo-6,7-dihydro-5H- | >100 | 11 | 0.0224 |
| thiazolo[3,2-c]pyrimidine-2-carboxylic acid | | | |
| 4-methoxy-benzylamide | | | |
| 6-Benzyl-8-methyl-5,7-dioxo-6,7-dihydro-5H- | >30 | >30 | 0.0487 |
| thiazolo[3,2-c]pyrimidine-2-carboxylic acid (pyridin- | | | |
| 4-ylmethyl)-amide hydrochloride | | | |
| 6-Benzyl-8-methyl-5,7-dioxo-6,7-dihydro-5H- | >30 | >10 | 0.0175 |
| thiazolo[3,2-c]pyrimidine-2-carboxylic acid | | | |
| (6-methoxy-pyridin-3-ylmethyl)-amide | | | |
| 6-Benzyl-8-methyl-5,7-dioxo-6,7-dihydro-5H- | >30 | 20.5 | 0.0208 |
| thiazolo[3,2-c]pyrimidine-2-carboxylic acid | | | |
| (6-methoxy-pyridin-3-ylmethyl)-amide hydrochloride | | | |
| 6-Benzyl-8-methyl-5,7-dioxo-6,7-dihydro-5H- | >30 | 10 | 0.0046 |
| thiazolo[3,2-c]pyrimidine-2-carboxylic acid (2,1,3- | | | |
| benzothiadiazol-5-ylmethyl)-amide | | | |
| 6-Benzyl-8-methyl-5,7-dioxo-6,7-dihydro-5H- | >100 | >30 | 0.029 |
| thiazolo[3,2-c]pyrimidine-2-carboxylic acid | | | |
| 3,4-difluoro-benzylamide | | | |
| 6-benzyl-8-methyl-5,7-dioxo-6,7-dihydro-5H- | >100 | >30 | 0.225 |
| thiazolo[3,2-c]pyrimidine-2-carboxylic acid (pyridin- | | | |
| 3-ylmethyl)-amide | | | |
| 6-benzyl-8-methyl-5,7-dioxo-6,7-dihydro-5H- | >30 | >30 | 0.260 |
| thiazolo[3,2-c]pyrimidine-2-carboxylic acid (pyridin- | | | |
| 3-ylmethyl)-amide hydrochloride | | | |
| 6-Benzyl-8-methyl-5,7-dioxo-6,7-dihydro-5H- | >100 | 12 | 0.025 |
| thiazolo[3,2-c]pyrimidine-2-carboxylic acid 3-fluoro- | | | |
| 4-methoxy-benzylamide | | | |
| 6-Benzyl-8-methyl-5,7-dioxo-6,7-dihydro-5H- | >30 | 19 | 0.225 |
| thiazolo[3,2-c]pyrimidine-2-carboxylic acid 4-methyl- | | | |
| benzylamide | | | |
| 6-Benzyl-8-methyl-5,7-dioxo-6,7-dihydro-5H- | >30 | >100 | 2.21 |
| thiazolo[3,2-c]pyrimidine-2-carboxylic acid | | | |
| 4-trifluoromethyl-benzylamide | | | |
| 6-Benzyl-8-methyl-5,7-dioxo-6,7-dihydro-5H- | >30 | 23 | 0.0869 |
| thiazolo[3,2-c]pyrimidine-2-carboxylic acid 4-chloro- | | | |
| benzylamide | | | |
| 6-Benzyl-8-methyl-5,7-dioxo-6,7-dihydro-5H- | >30 | >100 | 0.815 |
| thiazolo[3,2-c]pyrimidine-2-carboxylic acid | | | |
| 4-trifluoromethoxy-benzylamide | | | |
| 4-[2-(4-Methoxy-benzylcarbamoyl)-8-methyl- | >100 | 36 | 0.00175 |
| 5,7-dioxo-7H-thiazolo[3,2-c]pyrimidin-6-ylmethyl]- | | | |
| benzoic acid sodium salt | | | |
| 4-[2-(4-Methoxy-benzylcarbamoyl)-8-methyl- | >30 | >100 | 0.0455 |
| 5,7-dioxo-7H-thiazolo[3,2-c]pyrimidin-6-ylmethyl]- | | | |
| benzoic acid 2-dimethylamino-ethyl ester | | | |
| hydrochloride | | | |
| 4-[2-(4-fluoro-benzylcarbamoyl)-8-methyl-5,7-dioxo- | >100 | 68 | 0.0022 |
| 7H-thiazolo[3,2-c]pyrimidin-6-ylmethyl]-benzoic acid | | | |
| 4-[2-(4-Fluoro-benzylcarbamoyl)-8-methyl-5,7-dioxo- | >100 | 55.5 | 0.0020 |
| 7H-thiazolo[3,2-c]pyrimidin-6-ylmethyl]-benzoic acid | | | |
| Sodium Salt | | | |
| 4-[2-(4-Fluoro-benzylcarbamoyl)-8-methyl-5,7-dioxo- | >100 | >30 | 0.094 |
| 7H-thiazolo[3,2-c]pyrimidin-6-ylmethyl]-benzoic acid | | | |
| 2-dimethylamino-ethyl ester | | | |
| 4-[2-(4-Fluoro-benzylcarbamoyl)-8-methyl-5,7-dioxo- | >30 | >100 | 0.071 |
| 7H-thiazolo[3,2-c]pyrimidin-6-ylmethyl]-benzoic acid | | | |
| 2-dimethylamino-ethyl ester hydrochloride | | | |
| 4-{8-Methyl-5,7-dioxo-2-[(pyridin-4-ylmethyl)- | >100 | >100 | 0.235 |
| carbamoyl]-7H-thiazolo[3,2-c]pyrimidin-6-ylmethyl}- | | | |
| benzoic acid 2-dimethylamino-ethyl ester | | | |
| dihydrochloride | | | |
| 8-Methyl-6-(2-methyl-thiazol-4-ylmethyl)-5,7-dioxo- | >100 | >30 | 0.770 |
| 6,7-dihydro-5H-thiazolo[3,2-c]pyrimidine-2-carboxylic | | | |
| acid 4-fluoro-benzylamide | | | |
| 2-Chloro-4-[2-(4-fluoro-benzylcarbamoyl)-8-methyl- | >30 | >30 | 0.240 |
| 5,7-dioxo-7H-thiazolo[3,2-c]pyrimidin-6-ylmethyl]- | | | |
| benzoic acid methyl ester | | | |
| 8-Methyl-5,7-dioxo-6-thiazol-2-ylmethyl-6,7-dihydro- | >100 | >30 | 0.530 |
| 5H-thiazolo[3,2-c]pyrimidine-2-carboxylic acid | | | |
| 4-fluoro-benzylamide hydrochloride | | | |
| 4-[2-(4-Fluoro-benzylcarbamoyl)-8-methyl-5,7-dioxo- | >100 | 18 | 0.018 |
| 7H-thiazolo[3,2-c]pyrimidin-6-ylmethyl]-2-methyl- | | | |
| benzoic acid methyl ester | | | |
| 4-[2-(4-Fluoro-benzylcarbamoyl)-8-methyl-5,7-dioxo- | >30 | 10 | 0.099 |
| 7H-thiazolo[3,2-c]pyrimidin-6-ylmethyl]-2-methoxy- | | | |
| benzoic acid methyl ester | | | |
| 6-(4-Fluoro-benzyl)-8-methyl-5,7-dioxo-6,7-dihydro- | >30 | >30 | 0.0605 |
| 5H-thiazolo[3,2-c]pyrimidine-2-carboxylic acid | | | |
| (pyridin-4-ylmethyl)-amide hydrochloride | | | |
| 6-(4-Bromo-benzyl)-8-methyl-5,7-dioxo-6,7-dihydro- | >30 | >30 | 0.0365 |
| 5H-thiazolo[3,2-c]pyrimidine-2-carboxylic acid | | | |
| (pyridin-4-ylmethyl)-amide hydrochloride | | | |
| 6-(4-Chloro-benzyl)-8-methyl-5,7-dioxo-6,7-dihydro- | >30 | >30 | 0.0530 |
| 5H-thiazolo[3,2-c]pyrimidine-2-carboxylic acid | | | |
| (pyridin-4-ylmethyl)-amide | | | |
| 8-Methyl-6-[4-(morpholine-4-carbonyl)-benzyl]- | >100 | >100 | 0.0370 |
| 5,7-dioxo-6,7-dihydro-5H-thiazolo[3,2-c]pyrimidine- | | | |
| 2-carboxylic acid (pyridin-4-ylmethyl)-amide | | | |
| hydrochloride | | | |
| {5-[2-(4-Fluoro-benzylcarbamoyl)-8-methyl-5,7-dioxo- | >30 | >30 | 0.2050 |
| 7H-thiazolo[3,2-c]pyrimidin-6-ylmethyl]-isoxazol- | | | |
| 3-yl}-carbamic acid methyl ester | | | |
| 8-Methyl-5,7-dioxo-6-[4-(2H-tetrazol-5-yl)-benzyl]- | >100 | 16 | 0.0009 |
| 6,7-dihydro-5H-thiazolo[3,2-c]pyrimidine-2-carboxylic | | | |
| acid 4-fluoro-benzylamide | | | |
| 8-Methyl-6-[4-(morpholine-4-carbonyl)-benzyl]- | >30 | >30 | 0.0110 |
| 5,7-dioxo-6,7-dihydro-5H-thiazolo[3,2-c]pyrimidine- | | | |
| 2-carboxylic acid 4-fluoro-benzylamide | | | |
| 6-(6-Fluoro-quinolin-2-ylmethyl)-8-methyl-5,7-dioxo- | >30 | 18 | 0.0860 |
| 6,7-dihydro-5H-thiazolo[3,2-c]pyrimidine-2-carboxylic | | | |
| acid 4-fluoro-benzylamide | | | |
| 2-[2-(4-Fluoro-benzylcarbamoyl)-8-methyl-5,7-dioxo- | >30 | >30 | 1.850 |
| 7H-thizolo[3,2-c]pyrimidin-6-ylmethyl]-5-methoxy- | | | |
| pyrimidine-4-carboxylic acid methyl ester | | | |
| 6-But-2-ynyl-8-methyl-5,7-dioxo-6,7-dihydro-5H- | >100 | >30 | 0.3150 |
| thiazolo[3,2-c]pyrimidine-2-carboxylic acid 4-fluoro- | | | |
| benzylamide | | | |
| 8-Methyl-5,7-dioxo-6-(2-oxo-2H-1-benzopyran- | >30 | >30 | 0.0120 |
| 6-ylmethyl)-6,7-dihydro-5H-thiazolo[3,2-c]pyrimidine- | | | |
| 2-carboxylic acid 4-fluoro-benzylamide | | | |
| 6-(3-Cyano-benzyl)-8-methyl-5,7-dioxo-6,7-dihydro- | >30 | >30 | 0.1733 |
| 5H-thiazolo[3,2-c]pyrimidine-2-carboxylic acid | | | |
| (pyridin-4-ylmethyl)-amide hydrochloride | | | |
| 8-Methyl-5,7-dioxo-6-(4-sulfamoyl-benzyl)- | >30 | >100 | 0.0463 |
| 6,7-dihydro-5H-thiazolo[3,2-c]pyrimidine-2-carboxylic | | | |
| acid (pyridin-4-ylmethyl)-amide hydrochloride | | | |
| 6-(4-Cyano-benzyl)-8-methyl-5,7-dioxo-6,7-dihydro- | >30 | >30 | 0.0547 |
| 5H-thiazolo[3,2-c]pyrimidine-2-carboxylic acid | | | |
| (pyridin-4-ylmethyl)-amide hydrochloride | | | |
| 8-Methyl-5,7-dioxo-6-(2-phenylmethanesulfonyl- | nt | nt | 0.560 |
| ethyl)-6,7-dihydro-5H-thiazolo[3,2-c]pyrimidine- | | | |
| 2-carboxylic acid 4-fluoro-benzylamide | | | |
| 6-(E)-But-2-enyl-8-methyl-5,7-dioxo-6,7-dihydro-5H- | nt | nt | 0.530 |
| thiazolo[3,2-c]pyrimidine-2-carboxylic acid 4-fluoro- | | | |
| benzylamide | | | |
| 8-Methyl-5,7-dioxo-6-(E)-pent-2-enyl-6,7-dihydro-5H- | nt | nt | 0.160 |
| thiazolo[3,2-c]pyrimidine-2-carboxylic acid 4-fluoro- | | | |
| benzylamide | | | |
| 6-sec-Butyl-8-methyl-5,7-dioxo-6,7-dihydro-5H- | nt | nt | 0.400 |
| thiazolo[3,2-c]pyrimidine-2-carboxylic acid 4-fluoro- | | | |
| benzylamide | | | |
| 8-Methyl-5,7-dioxo-6-pent-2-ynyl-6,7-dihydro-5H- | nt | nt | 0.0920 |
| thiazolo[3,2-c]pyrimidine-2-carboxylic acid 4-fluoro- | | | |
| benzylamide | | | |
| 8-Methyl-6-(3-methyl-but-2-enyl)-5,7-dioxo- | nt | nt | 0.820 |
| 6,7-dihydro-5H-thiazolo[3,2-c]pyrimidine-2-carboxylic | | | |
| acid 4-fluoro-benzylamide | | | |
| 6-[2-(4-Fluoro-benzenesulfonyl)-ethyl]-8-methyl- | nt | nt | 0.6450 |
| 5,7-dioxo-6,7-dihydro-5H-thiazolo[3,2-c]pyrimidine- | | | |
| 2-carboxylic acid 4-fluoro-benzylamide | | | |
| 6-[3-(4-Fluoro-phenyl)-3-oxo-propyl]-8-methyl- | nt | nt | 0.510 |
| 5,7-dioxo-6,7-dihydro-5H-thiazolo[3,2-c]pyrimidine- | | | |
| 2-carboxylic acid 4-fluoro-benzylamide | | | |
| 8-Methyl-5,7-dioxo-6-(2-phenoxy-ethyl)-6,7-dihydro- | nt | nt | 0.0580 |
| 5H-thiazolo[3,2-c]pyrimidine-2-carboxylic acid | | | |
| 4-fluoro-benzylamide | | | |
| 6-(3,4-Dichloro-benzyl)-5,7-dioxo-6,7-dihydro-5H- | >100 | >100 | 0.0840 |
| thiazolo[3,2-c]pyrimidine-2-carboxylic acid | | | |
| 4-methoxy-benzylamide | | | |
| 4-[2-(4-Methoxy-benzylcarbamoyl)-8-methyl- | >100 | 65 | 0.0106 |
| 5,7-dioxo-7H-thiazolo[3,2-c]pyrimidin-6-ylmethyl]- | | | |
| benzoic acid methyl ester | | | |
| 4-[2-(3-Fluoro-benzylcarbamoyl)-8-methyl-5,7-dioxo- | >30 | 19 | 0.0715 |
| 7H-thiazolo[3,2-c]pyrimidin-6-ylmethyl]-benzoic acid | | | |
| methyl ester | | | |
| 4-[2-(4-Fluoro-benzylcarbamoyl)-8-methyl-5,7-dioxo- | >100 | 17 | 0.0180 |
| 7H-thiazolo[3,2-c]pyrimidin-6-ylmethyl]-benzoic acid | | | |
| methyl ester | | | |
| 6-(4-Cyano-benzyl)-8-methyl-5,7-dioxo-6,7-dihydro- | >100 | 25 | 0.023 |
| 5H-thiazolo[3,2-c]pyrimidine-2-carboxylic acid | | | |
| 4-fluoro-benzylamide | | | |
| 6-Benzyl-8-methyl-5,7-dioxo-6,7-dihydro-5H- | >30 | 22 | 0.0060 |
| thiazolo[3,2-c]pyrimidine-2-carboxylic acid | | | |
| 3-methoxy-benzylamide | | | |

nt: not tested

The assays used to evaluate the biological activity of the above compounds are well-known and routinely used by those skilled in the study of MMP inhibitors and their use to treat clinical conditions. They measure the amount by which a test compound reduces the hydrolysis of a thiopeptolide substrate caused by a matrix metalloproteinase enzyme. Such assays are described in detail by Ye et al., in Biochemistry, 1992, 31(45):11231-11235, which is incorporated herein by reference.

Thiopeptolide substrates show virtually no decomposition or hydrolysis in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of 2-morpholinoethane sulfonic acid monohydrate (MES, pH 6.0) 10 mM CaCl₂, 100 μM thiopeptolide substrate, and 1 mM 5,5′-dithio-bis-(2-nitro-benzoic acid) (DTNB). The thiopeptolide substrate concentration is varied from 10 to 800 μM to obtain Km and Kcat values. The change in absorbance at 405 nm is monitored on a Thermo Max microplate reader (moleucular Devices, Menlo Park, Calif.) at room temperature (22° C.). The calculation of the amount of hydrolysis of the thiopeptolide substrate is based on E₄₁₂=13600 m⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

In the above table, MMP-1FL refers to full-length interstitial collagenase; MMP-2FL refers to full length Gelatinase A; MMP-3CD refers to the catalytic domain of stromelysin; MMP-7FL refers to full-length matrilysin; MMP-9FL refers to full length Gelatinase B; MMP-13CD refers to the catalytic domain of collagenase 3; and MMP-14CD refers to the catalytic domain of membrane type 1 MMP. Test compounds were evaluated at various concentrations in order to determine their respective IC₅₀ values, the micromolar concentration of compound required to cause a 50% inhibition of the hydrolytic activity of the respective enzyme.

Binding of the compound of Synthesis Example 1 below is shown in FIG. 4. The molecule has first and second hydrophobic groups and first, second and third hydrogen bond acceptors. The first hydrophobic group locates in the S1′ pocket of the enzyme and its hydrophobic aryl ring interacts with the aryl rings of His222 and Tyr244. The second hydrophobic group is open to solvent and forms hydrophobic interactions with the aryl rings of e.g. Phe252 and Tyr246. The three hydrogen bond acceptors interact respectively with Thr245, Thr247 and Met 253.

Synthesis of some of the compounds referred to in Table 1a is described in the following examples. The synthesis of the other compounds in Table 1b is reported in our co-pending WO application which claims the priority application No. U.S. 60/268,780 filed on Feb. 14, 2001.

### SYNTHESIS EXAMPLE 1

### 6-Benzyl-5,7-dioxo-6,7-dihydro-5H-thiazolo[3,2-c]pyrimidine-2-carboxylic acid benzylester

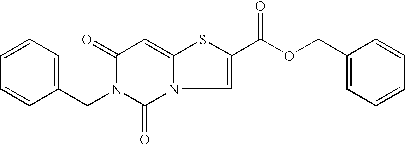

Step 1:

### 1-Benzyl-pyrimidine-2,4,6-trione

Freshly cut sodium metal (15.9 g, 690 mmol) was dissolved in 100% ethanol, diethylmalonate (53 ml, 349 mmol), and benzylurea (50.33 g, 335 mmol) were added, and the mixture was heated to reflux. The heat was reduced just below reflux and ethanol (100 ml) was added. The reaction mixture was stirred 3 days at just below ethanol reflux and was then allowed to cool. Water (300 ml) and then 2N HCl (500 ml) were added and the entire mixture was cooled to 0° C. The resulting solid was collected by filtration, washed with water, and air-dried. Two crops totalling 64.52 g (88%) were obtained. Calculated for C₁₁H₁₀N₂O₃: C, 60.55; H, 4.62; N, 12.84. Found: C, 60.65; H, 4.61; N, 12.60.

Step 2:

### 3-Benzyl-6-chloro-1H-pyrimidine-2,4-dione

Phosphorus oxychloride (240 ml) was added in small portions over ˜0.75 hour to a mixture of 1-benzyl-pyrimidine-2,4,6-trione (47.48 g, 217 mmol) and water (10 ml). Upon completing the addition the reaction mixture was heated to reflux for one hour, then allowed to cool somewhat, after which the phosphorus oxychloride was removed on a rotary evaporator. The resulting brown oil was added to ice, and the ice was allowed to slowly melt. The resulting precipitate was collected by filtration, washed with water, slurried in hexane, collected by filtration, taken up in tetrahydrofuran, dried (magnesium sulfate) filtered, concentrated, and the resulting solid collected by filtration. The product was obtained in 2 portions 38.61 g (75.2%). Calculated for C₁₁H₉ClN₂O₂: C, 55.83; H, 3.83; N, 11.84. Found: C, 55.76; H, 3.78; N, 11.62.

Step 3:

### 3-Benzyl-6-(2,2-dimethoxy-ethylsulfanyl)-1H-pyrimidine-2,4-dione

Ground sodium hydrosulfide hydrate (4.72 g, 84 mmol) was added to 3-benzyl-6-chloro-1H-pyrimidine-2,4-dione (4.72 g, 20 mmol) in dimethylformamide (20 ml), and the mixture was warmed to 45° C. for about 15 minutes, and then bromacetaldehyde dimethylacetal (11 ml, 93 mmol) was added in portions over about 30 minutes. The reaction mixture was stirred 3 days at 45° C. and was then partitioned between ethyl acetate (400 ml) and sodium bicarbonate solution (200 ml). The layers were separated, and the organic layer washed with water (200 ml) and brine (100 ml), and dried over magnesium sulfate. The solution was filtered and concentrated and triturated with hexanes/ethyl acetate and the solid collected by filtration. The solid was dissolved in methylene chloride, concentrated and triturated (1/1, hexanes/ethyl acetate), filtered, and the solid dissolved in methylene chloride, concentrated and triturated (1/1, hexanes/ethyl acetate), and filtered again to give 1.128 g of product. An additional 1.76 g was obtained by chromatography of the mother liquors on silica gel using hexanes/ethyl acetate as eluant. Total yield 44.8%. Calculated for C₁₅H₁₈N₂O₄S: C, 55.89; H, 5.63; N, 8.69. Found: C, 55.79; H, 5.32; N, 8.63.

Step 4:

### 6-Benzyl-thiazolo[3,2-c]pyrimidine-5,7-dione

To a solution of 3-benzyl-6-(2,2-dimethyloxy-ethylsulfanyl)-1H-pyrimidine-2,4-dione (1.34 g, 3.83 mmol) in xylene was added 100 mg of para-toluenesulfonic acid. The resulting solution was refluxed for 5 hours while removing water using a Dean-Stark trap. The reaction was then cooled to room temperature and purified using flash chromatography to give the desired product as a white solid (1.01 g, 100%). R_{f}=0.26 (1:1 hexane/EtOAc); ¹H NMR (CDCl₃): δ 7.20-7.55 (m, 5H), 6.47 (d, 1H, d=4.6 Hz), 6.00 (s, 1H), 5.18 (s, 2H); MS (ACPI), m/z 259.1 (M⁺+1).

Step 5:

### 6-Benzyl-5,7-dioxo-6,7-dihydro-5H-thiazolo[3,2-c]pyrimidine-2-carboxylic acid benzylester

To a solution of diisopropyiamine in THF (5 ml) at 0° C. was added n-BuLi (1.6 M, 0.15 ml, 0.24 mmol), and the resulting solution was stirred at 0° C. for 10 minutes and cooled to −78° C. for 30 minutes. A solution of 6-benzyl-thiazolo[3,2-c]pyrimidine-5,7-dione (52 mg, 0.2 mmol) in THF (5 ml) was added, and the resulting solution was stirred at −78° C. for 30 minutes. Neat benzylchloroformate (0.041 g, 0.24 mmol) was added dropwise, and the reaction was quenched by addition of NH₄Cl after 30 minutes at −78° C. After extraction with EtOAc, the organic layers were combined and washed with brine, dried, filtered, and concentrated under vacuum. The residue was purified using flash chromatograpy to give the desired product as a yellowish solid (became white after trituration with 1:1 hexane/EtOAc, 0.014 g, 18%). R_{f}=0.54 (1:1 hexane/EtOAc); ¹H NMR (CDCl₃): δ 7.84 (s, 1H), 6.92-7.18 (m, 10H), 5.64 (s, 1H), 5.00 (S, 2H), 4.82 (s, 2H); MS (ACPI), m/z 392.0 (M⁺+1).

### SYNTHESIS EXAMPLE 2

### 6-Benzyl-5,7-dioxo-6,7-dihydro-5H-thiazolo[3,2-c]pyrimidine-2-carboxylic acid benzylamide

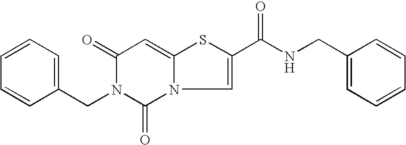

Step 1:

### 6-Benzyl-thiazolo[3,2-c]pyrimidine-5,7-dione

To a solution of 3-benzyl-6-(2,2-dimethyloxy-ethylsulfanyl)-1H-pyrimidine-2,4-dione (1.34 g, 3.83 mmol) in xylene was added 100 mg of para-toluenesulfonic acid. The resulting solution was refluxed for 5 hours while removing water using a Dean-Stark trap. The reaction was then cooled to room temperature and purified using flash chromatography to give the desired product as a white solid (1.01 g, 100%). R_{f}=0.26 (1:1 hexane/EtOAc); ¹H NMR (CDCl₃), δ 7.20-7.55 (m, 5H), 6.47 (d, 1H, d=4.6 Hz), 6.00 (s, 1H), 5.18 (s, 2H); MS (ACPI), m/z 259.1 (M⁺+1).

Step 2:

### 6-Benzyl-5,7-dioxo-6,7-dihydro-5H-thiazolo[3,2-c]pyrimidine-2-carboxylic acid benzylamide

To a solution of 6-benzyl-thiazolo[3,2-c]pyrimidine-5,7-dione (550 mg, 2.13 mmol) in THF (5 ml) was added LiN(TMS)₂ (3.0 ml, 1.0 M, 3.0 mmol), and the resulting solution was stirred at −78° C. for 30 minutes. Neat benzylisocyanate (0.34 ml, 2.77 mmol) was added dropwise, and the reaction was stirred at −78° C. for 30 minutes and quenched by addition of NH₄Cl solution. After extraction with EtOAc, the organic layers were combined and washed with brine, dried, filtered, and concentrated under vacuum. The residue was purified using flash chromatography to give the desired product as a yellowish solid (became white after trituration with 1:1 hexane/EtOAc, 0.123 g, 15%). R_{f}=0.35 (1:1 hexane/EtOAc); ¹H NMR (d₈₋THF): δ 8.16 (s, 1H), 7.99 (S, 1H), 7.06-7.32 (m, 10H), 5.88 (S, 1H), 4.96 (S, 2H), 4.38 (d, 2H, J=5.6 Hz); MS (ACPI), m/z 392.4 (M⁺+1). Calculated for C₂₁H₁₇N₃O₃S₁: C, 64.44; H, 4.38; N, 10.73. Found: C, 63.95; H, 4.46; N, 10.72.

### SYNTHESIS EXAMPLE 3

### 6-Benzyl-8-methyl-5,7-dioxo-6,7-dihydro-5H-thiazolo[3,2-c]pyrimidine-2-carboxylic acid benzyl ester

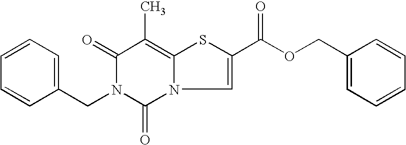

Step 1:

### 1-Benzyl-5-methyl-pyrimidine-2,4,6-trione

Sodium metal (7.68 g, 334 mmol) was dissolved in 100% ethanol (500 ml); benzylurea (25.12 g, 168 mmol) and diethylmethyl malonate (29 ml, 169 mmol) were added, and the mixture was heated at just below ethanol reflux overnight. The reaction mixture was concentrated to remove ethanol, water (200 ml) and 1N hydrochloric acid (350 ml) were added, and an oil separated. The oil would not crystallize and could not be purified by chromatography. The oil was treated with ethanol/sodium ethoxide, (400 ml/7.4 g, 322 mmol) overnight at just below ethanol reflux and was worked up as before to give an oil that would not crystallize. This material was used directly in the next step.

Step 2:

### 3-Benzyl-6-chloro-5-methyl-1H-pyrimidine-2,4-dione

The crude pyrimidinedione from above was taken up in tetrahydrofuran (˜10 ml), water (5 ml) was added, concentrated to remove tetrahydrofuran, and phosphorous oxychloride (110 ml) was added in portions over ˜45 minutes, then the mixture was heated at reflux for 2 hours, stirred at room temperature overnight, then the phosphorous oxychloride was removed on the rotary evaporatory. Crushed ice (˜300 g) was added and the mixture was allowed to slowly warm to room temperature, and the resulting dark oil solidified on standing. The solid was collected by filtration, washed with water, taken up in tetrahydrofuran, dried over magnesium sulfate, filtered, and concentrated to a brown solid. The solid was triturated with hexanes/ethyl acetate, 1/1, v/v, collected by filtration and washed with hexanes. The product was obtained in 4 portions, 14 g (33.2% for the 2 steps).

Step 3:

### 3-Benzyl-6-(2,2-dimethoxy-ethylsulfanyl)-5-methyl-H-pyrimidine-2,4-dione

The procedure for Synthesis Example 1 was used starting with 3-benzyl-6-chloro-1H-pyrimidine-2,4-dione (5.0 g, 20 mmol), sodium hydrosulfide hydrate (5.06 g, 90.4 mmol), and bromoacetaldehyde dimethylacetal (13 ml, 110 mmol) to give 3benzyl-6-(2,2-dimethoxy-ethylsulfanyl)-5-methyl-H-pyrimidine-2,4-dione in 2 portions 2.57 g. (38%). Calculated for C₁₆H₂₀N₂O₄S: C, 57.13; H, 5.49; N, 8.33. Found: C, 57.30; H, 5.50; N, 8.78.

Step 4:

### 6-Benzyl-8-methyl-thiazolo[3,2-c]pyrimidine-5,7-dione

The thioether acetal, 3-benzyl-6-(2,2-dimethoxy-ethylsulfanyl)-5-methyl-H-pyrimidine-2,4-dione (0.95 g, 2.8 mmol), was treated according to the procedure for Synthesis Example 2, to give the product 6-benzyl-8-methyl-thiazolo[3,2-c]pyrimidine-5,7-dione (0.622 g) as a light tan solid. (80.8%). Calculated for C₁₄H₁₂N₂O₂S: C, 61.75; H, 4.44; N, 10.29. Found: C, 61.63; H, 4.51; N, 10.19.

Step 5:

### 6-Benzyl-8-methyl-5,7-dioxo-6,7-dihydro-5H-thiazolo[3,2-c]pyrimidine-2-carboxylic acid benzyl ester

6-Benzyl-8-methyl-thiazolo[3,2-c]pyrimidine-5,7-dione (0.262 g, 0.96 mmol) was taken up in tetrahydrofuran (25 ml) and lithium hexamethyldisilazane (1.3 ml, 1 M in tetrahydrofuran, 1.3 mmol) was added at −78° C. The reaction was allowed to proceed for 3 minutes, then benzyl chloroformate (0.5 ml, 3.5 mmol) was added and the reaction was stirred for 10 minutes at −78° C. Ammonium chloride solution (4 ml) was added and the reaction mixture was allowed to warm until the ice in the flask melted. The reaction mixture was partitioned between ethyl acetate (200 ml) and brine (100 ml). The layers were separated, the organic layer was dried over magnesium sulfate, filtered, and concentrated. The residue was chromatographed on silica gel using hexanes/ethyl acetate, 6/4, v/v, as eluant to give the product in 2 portions, 0.158 g. (40.5%). Calculated for C₂₂H₁₈N₂O₄S: C, 64.92; H, 4.31; N, 6.63. Found: C, 65.01; H, 4.46; N, 6.89.

### SYNTHESIS EXAMPLE 4

### 6-Benzyl-5,7-dioxo-6,7-dihydro-5H-thiazolo[3,2-c]pyrimidine-2-carboxylic acid pyridin-4-ylmethyl ester hydrochloride

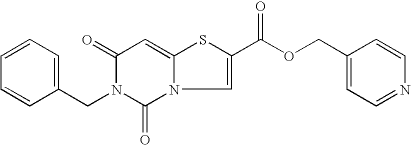

Step 1:

### 6-Benzyl-5,7-dioxo-6,7-dihydro-5H-thiazolo[3,2-c]pyrimidine-2-carboxylic acid methyl ester

The product from Synthesis Example 1, Step 4, (0.518 g, 2.0 mmol) was reacted according to the procedure of Synthesis Example 1 step 5, using methyl chloroformate (3.0 ml, 39 mmol) in the place of benzyl chloroformate to give 6-benzyl-5,7-dioxo-6,7-dihydro-5H-thiazolo[3,2-c]pyrimidine-2-carboxylic acid methyl ester (0.084 g). An additional 0.26 g of impure product was also obtained. (Total yield 54.2%). Calculated for C₁₅H₁₂N₂O₄S: C, 56.95; H, 3.82; N, 8.86. Found: C, 56.87; H, 3.75; N, 8.61.

Step 2:

### 6-Benzyl-5,7-dioxo-6,7-dihydro-5H-thiazolo[3,2-c]pyrimidine-2-carboxylic acid

6-Benzyl-5,7-dioxo-6,7-dihydro-5H-thiazolo[3,2-c]pyrimidine-2-carboxylic acid methyl ester (0.226 g, 0.71 mmol), was taken up in methanol (5 ml) and tetrahydrofuran (5 ml) and 1 M sodium hydroxide solution (0.8 ml, 0.8 mmol) was added at room temperature. The solution turned orange. Water was added until the volume reached about 25 ml and no cloudiness appeared. The reaction mixture was allowed to stand ˜10 minutes and was then poured into a separating funnel containing ethyl acetate (200 ml), brine (100 ml), and 1N HCl solution (3 ml). The layers were separated, dried over magnesium sulfate, and concentrated to a yellow solid. The solid was triturated with hexanes/ethyl acetate and the insoluble portion collected by filtration. (0.093 g). (44%). This was used directly in the next step.

Step 3:

### 6-Benzyl-5,7-dioxo-6,7-dihydro-5H-thiazolo[3,2-c]pyrimidine-2-carboxylic acid pyridin-4-ylmethyl ester hydrochloride

6-Benzyl-5,7-dioxo-6,7-dihydro-5H-thiazolo[3,2-c]pyrimidine-2-carboxylic acid (0.084 g, 0.28 mmol), 4-pyridinemethanol (0.082 g, 0.75 mmol), 4-dimethylaminopyridine (0.014 g, 0.11 mmol), and dichloromethane (5 ml) were stirred at room temperature and dicyclohexylcarbodiimide (0.059 g, 0.29 mmol) was added all at once. The reaction mixture was cooled to 0° C., allowed to slowly warm to room temperature and was stirred overnight. It was then concentrated to dryness, chromatographed on silica gel using ethyl acetate as eluant, the product-containing fractions combined and concentrated, and triturated. Dicyclohexylurea was present. The solid was taken up in tetrahydrofuran (˜3 ml) and HCl gas in ether (1 M, 1 ml, 1 mmol) was added, and a precipitate formed. The mixture was concentrated to dryness, tetrahydrofuran (˜7 ml) was added, and the insoluble portion collected by filtration and washed with tetrahydrofuran and air-dried. The product, 6-benzyl-5,7-dioxo-6,7-dihydro-5H-thiazolo[3,2-c]pyrimidine-2-carboxylic acid pyridin-4-ylmethyl ester hydrochloride, was obtained as a light yellow solid (0.0396 g) (33%). Calculated for C₂₀H₁₅N₃O₄S HCl: C, 55.88; H, 3.75; N, 9.77. Found: C, 55.49; H, 3.92; N, 9.60.

### SYNTHESIS EXAMPLE 5

### 4-[2-(4-Methoxy-benzylcarbamoyl)-8-methyl-5,7-dioxo-7H-thiazolo[3,2-c]pyrimidin-6-ylmethyl]-benzoic acid

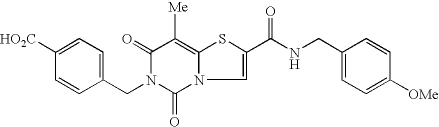

Step 1:

### 8-Methyl-5,7-dioxo-6,7-dihydro-5H-thiazolo[3,2-c]pyrimidine-2-carboxylic acid 4-methoxy-benzylamide

8-Methyl-5,7-dioxo-6,7-dihydro-5H-thiazolo[3,2-c]pyrimidine-2-carboxylic acid (10.0 g, 41 mmol) was dissolved in dimethylformamide (300 ml). To the solution was added 1-hydroxybenzotriazole hydrate (6.08 g, 45 mmol) and 1-[3-(dimethylamino)propyl]-3-ethylcarbodiimide hydrochloride (10.2 g, 53 mmol), then 4-methoxybenzylamine (5.9 ml, 45 mmol). The mixture was stirred for 22 hours at room temperature. The dimethylformamide was removed in vacuum at 60° C. The residue was stirred in water for 30 minutes then filtered. The resulting solid was stirred with 10% aqueous sodium carbonate for 30 minutes. The mixture was filtered and rinsed with water, then vacuum dried at 45° C. for 16 hours to give 8-methyl-5,7-dioxo-6,7-dihydro-5H-thiazolo[3,2-c]pyrimidine-2-carboxylic acid 4-methoxy-benzylamide (77%). MS (APCI+), m/z (%): 346(100), 303(30), 277(45).

Step 2:

### 4-Methylbenzoic acid tert-butyl ester

To a solution of pyridine (125 ml) and tert-butanol (125 ml, 1.31 mole) was added 4-methylbenzoyl chloride (171 ml, 1.29 mole). The reaction was stirred at room temperature for 88 hours, then poured into water (325 ml) and EtOAc (325 ml). The layers were separated. The EtOAc layer was washed with 0.5 M HCl (3×200 ml), water (200 ml), aqueous sodium bicarbonate, and brine. The solvent was evaporated under vacuum to give the crude ester. The material was dissolved in hexanes (250 ml) and passed through silica gel eluting with additional hexanes. The solvent was evaporated under vacuum to give 4-methylbenzoic acid tert-butyl ester (96%). ¹H-NMR (CDCl₃) δ 7.87 (d,2H), 7.20(d,2H), 2.39(s,3H), 1.58(s,9H).

Step 3:

### 4-Bromomethylbenzoic acid tert-butyl ester

Step C: The product of preceding Step 2 (50.0 g, 0.26 mole) was dissolved in carbon tetrachloride (250 ml). N-Bromosuccinimide (46.3 g, 0.26 mole) was added followed by benzoyl peroxide (0.6 g, 0.0026 mole). The mixture was heated at reflux for 4 hours. The cooled reaction was filtered, rinsing the solid with hexanes. The combined filtrate was washed with aqueous sodium bisulfite, and 0.5 M sodium hydroxide. The organic layer was dried (Na₂SO₄) and passed through silica gel eluting with hexanes. The solvent was removed under vacuum to give 4-bromomethylbenzoic acid tert-butyl ester (72%). The material could be crystallized from methanol; mp 46-48; ¹H-NMR (CDCl₃) δ 7.95(d, 2H), 7.41(d, 2H), 4.50(s, 2H), 1.59(s, 9H).

Step 4:

### 4-[2-(4-Methoxy-benzylcarbamoyl)-8-methyl-5,7-dioxo-7H-thiazolo[3,2-c]pyrimidin-6-ylmethyl]-benzoic acid tert-butyl ester

The product of the preceding Step 1 (10.0 g, 29.0 mmol) was suspended in dimethylformamide (300 ml). Cesium carbonate (9.55 g, 29.3 mmol) was added followed by the product of the preceding Step 3, namely 4-Bromomethylbenzoic acid tert-butyl ester (7.86 g, 29.0 mmol). After 17 hours, the dimethylformamide was removed in a vacuum at 70° C. The residue was mixed with tetrahydrofuran and filtered through a pad of Celite over silica gel eluting with additional tetrahydrofuran. The filtrate was evaporated under vacuum to an oil. The material was purified by chromatography on silica gel, eluting with CH₂Cl₂:tetrahydrofuran (19:1) to give 4-[2-(4-methoxy-benzylcarbamoyl)-8-methyl-5,7-dioxo-7H-thiazolo[3,2-c]pyrimidin-6-ylmethyl]-benzoic acid tert-butyl ester (80%). MS (APCI+), m/z (%): 536(35), 480(100), 317(80).

Step 5:

### 4-[2-(4-Methoxy-benzylcarbamoyl)-8-methyl-5,7-dioxo-7H-thiazolo[3,2-c]pyrimidin-6-ylmethyl]-benzoic acid

The product of the preceding Step 4 (12.2 g, 22.8 mmol) was dissolved in trifluoroacetic acid (100 ml) and stirred at room temperature for 1.5 hours. The solvent was removed under vacuum at 40° C. The resulting oil crystallized in tetrahydrofuran. The tetrahydrofuran was evaporated under vacuum. The solid was triturated with diethyl ether, then vacuum dried at 45° C. to give 4-[2-(4-methoxy-benzylcarbamoyl)-8-methyl-5,7-dioxo-7H-thiazolo[3,2-c]pyrimidin-6-ylmethyl]-benzoic acid (80%); mp>210° C.; MS (APCI+), m/z (%): 480(10), 317(100).

### SYNTHESIS EXAMPLE 6

### 4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetate

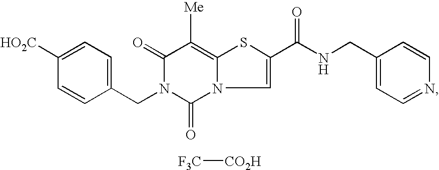

Step 1:

### 8-Methyl-5,7-dioxo-6,7-dihydro-5H-thiazolo[3,2-c]pyrimidine-2-carboxylic acid (pyridin-4-ylmethyl)-amide

8-Methyl-5,7-dioxo-6,7-dihydro-5H-thiazolo[3,2-c]pyrimidine-2-carboxylic acid was treated as in the synthesis Example 5, Step 1 using C-pyridin-4-ylmethylamine to give the desired compound. (82%); MS (APCI+), m/z (%): 317(100), 274(50), 248(95).

Step 2:

### 4-{8-Methyl-5,7-dioxo-2-[(pyridin-4-ylmethyl)-carbamoyl]-7H-thiazolo[3,2-c]pyrimidin-6-ylmethyl}-benzoic acid tert-butyl ester

The product of the preceding Step 1 was treated as in the synthesis Example 5, Step 4 to give the desired compound (47%); MS (AP+) m/z (%): 507(100), 451(35), 317(35), 147(40).

Step 3:

### 4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetate

The product of the preceding Step 2 was treated as in the synthesis Example 5, Step 5. Trituration with diethyl ether, ethyl acetate and again with diethyl ether gave the desired compound (93%); MS (APCI+), m/z (%): 451(40), 317(100), 135(30).

### SYNTHESISI EXAMPLE 7

### 6-(4-Methanesulfonyl-benzyl)-8-methyl-5,7-dioxo-6,7-dihydro-5H-thiazolo[3,2-c]pyrimidine-2-carboxylic acid (pyridin-4-ylmethyl)-amide hydrochloride

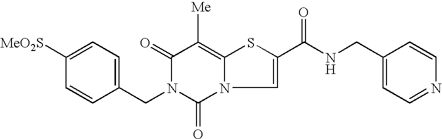

The product from synthesis Example 6, Step 1 was dissolved in dimethylformamide (5 ml), and cesium carbonate (163 mg, 0.5 mmol) was added followed by 4-methylsulfonylbenzyl chloride (102 mg, 0.5 mmol), and the mixture stirred overnight at room temperature. The dimethylformamide was removed under vacuum. The residue was partitioned between ethyl acetate and water, the layers separated, the organic layer washed with brine, dried over magnesium sulfate, filtered and concentrated. No product was in the ethyl acetate layer. The product was insoluble in both phases. The insoluble material was collected by filtration and dried under vacuum. The solid was stirred in ethereal HCl to give the desired product, 0.082 g (32%). MS (APCI+), m/z (%): 485.1(100), 351.0 (50).

### SYNTHESIS EXAMPLE 8

### 6-(3,4-Dichloro-benzyl)-5,7-dioxo-6,7-dihydro-5H-thiazolo[3,2-c]pyrimidine-2-carboxylic acid 4-methoxy-benzylamide

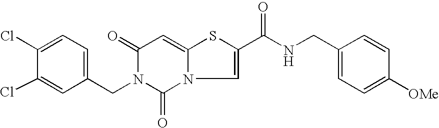

Lithium hexamethyldisilazane (0.9 ml, 1 M in THF, 0.9 mmol) was added to a solution of 6-(3,4-dichlorobenzyl)-thiazolo[3,2-c]pyrimidine-5,7-dione (0.200 g, 0.61 mmol) in tetrahydrofuran (10 ml), under nitrogen at −72° C. After 3 minutes, 1-isocyanatomethyl-4-methoxy-benzene (0.22 ml, 1.5 mmol) was added. The reaction was stirred 15 minutes, then aqueous ammonium chloride was added, and the reaction allowed to warm to room temperature. EtOAc (50 ml) was added to the reaction, water layer was removed, and the organic layer was, dried (Na₂SO₄) and evaporated. The residue was chromatographied on silica gel eluting with CH₂Cl₂:EtOAc, 9:1. The isolated product was triturated with diethyl ether and dried in vacuum to give 45.2 mg (15%) of the desired compound: mp 206-207° C.; MS (APCI+), m/z (%): 493(15), 492(80), 490(100), 329(40), 326(55), 263(30), 121(30).

Isophthalic Acid Derivatives

We have made a second group of compounds which are isophthalic acid derivatives and are inhibitors of matrix metalloproteinase enzymes, and especially MMP-13. Preferred compounds that we have made, and their ability to inhibit the activity of various matrix metalloproteinase enzymes are summarized in Table II below:

TABLE II

| | MMP01 | MMP03 | MMP13 |
|---|---|---|---|
| | IC50 | IC50 | IC50 |
| Compound | (nM) | (nM) | (nM) |
| 4-Methoxy-N,N′-bis-(4-methoxybenzyl)- | >100,000 | 82,000 | 250 |
| isophthalamide | | | |
| N,N′-Dibenzyl-4-methoxy-isophthalamide | nt | nt | 1100 |
| 4-Methoxy-isophthalic acid dibenzyl ester | >100,000 | >100,000 | 900 |
| 4-Methoxy-isophthalic acid dipyridin-4-ylmethyl ester | >100,000 | >100,000 | 255 |
| 5-Nitro-isophthalic acid dibenzyl ester | nt | nt | 1500 |
| 5-Amino-isophthalic acid dibenzyl ester | >100,000 | 73,000 | 1100 |
| Isophthalic acid bis-(4-fluoro-benzyl) ester | >100,000 | >100,000 | 2333 |
| Isophthalic acid dibenzyl ester | >100,000 | >30,000 | 2300 |
| N,N′-Bis-(4-chloro-benzyl)-isophthalamide | 79,000 | 9400 | 5500 |
| Isophthalic acid bis-(3-fluoro-benzyl) ester | >100,000 | >30,000 | 7833 |
| Isophthalic acid bis-(4-methoxy-benzyl) ester | >100,000 | 51,000 | 1075 |
| Isophthalic acid bis-(3-methoxy-benzyl) ester | >100,000 | >100,000 | 1150 |
| Isophthalic acid bis-(1,3-benzodioxol-5-ylmethyl) ester | nt | nt | 660 |
| N,N′-Bis-(4-fluoro-benzyl)-isophthalamide | >100,000 | >100,000 | 2350 |
| N,N′-Bis-(4-methoxy-benzyl)-isophthalamide | >100,000 | >30,000 | 1000 |
| N,N′-Bis-(3-fluoro-benzyl)-isophthalamide | >100,000 | >100,000 | 5650 |
| N,N′-Bis-(3-chloro-benzyl)-isophthalamide | >100,000 | 20,000 | 2300 |
| N,N′-Bis-1,3-benzodioxol-5-ylmethyl-isophthalamide | >100,000 | 69,000 | 330 |
| 4-Acetyl-isophthalic acid dibenzyl ester | >100,000 | >100,000 | 8200 |
| 4-Methoxycarbonylmethoxy-isophthalic acid dibenzyl | >100,000 | >100,000 | 9250 |
| ester | | | |
| N,N′-Bis-1,3-benzodioxol-5-ylmethyl-4-methoxy- | >100,000 | 50,000 | 185 |
| isophthalamide | | | |
| N-1,3-Benzodioxol-5-ylmethyl-4-methoxy-N′- | nt | nt | 200 |
| (4-methoxy-benzyl)-isophthalamide | | | |
| 4-Methoxy-N,N′-bis-(4-methoxy-benzyl)- | >100,000 | >100,000 | 280 |
| isophthalamide | | | |
| N-1,3-Benzodioxol-5-ylmethyl-N′-(4-chloro-benzyl)- | nt | nt | 400 |
| 4-methoxy-isophthalamide | | | |
| N-Benzyl-4-methoxy-N′-(4-methoxy-benzyl)- | nt | nt | 430 |
| isophthalamide | | | |
| N′-Benzyl-4-methoxy-N-(4-methoxy-benzyl)- | nt | nt | 810 |
| isophthalamide | | | |
| N,N′-Bis-1,3-benzodioxol-5-ylmethyl-isophthalamide | >100,000 | 81,000 | 683 |
| 4-Methoxy-N-(4-methoxy-benzyl)-N′-pyridin- | nt | nt | 1500 |
| 4-ylmethyl-isophthalamide | | | |
| N,N′-Bis-(3-methoxy-benzyl)-isophthalamide | >100,000 | >100,000 | 1350 |
| N-1,3-Benzodioxol-5-ylmethyl-N′-benzyl- | >100,000 | >100,000 | 1900 |
| isophthalamide | | | |
| N-1,3-Benzodioxol-5-ylmethyl-N′-(4-methoxy- | >100,000 | >100,000 | 1650 |
| benzyl)-isophthalamide | | | |
| N,N′-Dibenzyl-4-methoxy-isophthalamide | >100,000 | >100,000 | 1800 |
| N-Benzyl-N′-(4-methoxy-benzyl)-isophthalamide | >100,000 | >100,000 | 2425 |
| N′-1,3-Benzodioxol-5-ylmethyl-4-methoxy-N- | nt | nt | 3100 |
| (2-phenoxy-ethyl)-isophthalamide | | | |
| N-1,3-Benzodioxol-5-ylmethyl-4-methoxy-N′- | nt | nt | 4400 |
| (2-phenoxy-ethyl)-isophthalamide | | | |
| N-1,3-Benzodioxol-5-ylmethyl-N′-furan-2-ylmethyl- | >100,000 | >100,000 | 3400 |
| isophthalamide | | | |
| N′-1,3-Benzodioxol-5-ylmethyl-N-(2-ethoxy-ethyl)- | nt | nt | 5700 |
| 4-methoxy-isophthalamide | | | |
| N,N′-Bis-(4-methoxy-benzyl)-isophthalamide | >100,000 | >100,000 | 2740 |
| N,N′-Bis-(3-hydroxymethyl-phenyl)-isophthalamide | >100,000 | nt | 7800 |
| N-Benzyl-4-methoxy-N′-(2-phenoxy-ethyl)- | nt | nt | 8700 |
| isophthalamide | | | |
| 4-Methoxy-N,N′-bis-(4-methyl-benzyl)-isophthalamide | >100,000 | >100,000 | 7250 |
| 4-Methoxy-N,N′-bis-(3-methoxy-benzyl)- | >100,000 | >100,000 | 180 |
| isophthalamide | | | |
| Isophthalic acid di-(2,1,3-benzothiadiazol-5-yl)methyl | >100,000 | >30,000 | 1167 |
| ester | | | |
| N-1,3-Benzodioxol-5-ylmethyl-4-methoxy-N′- | nt | nt | 190 |
| (4-methoxy-benzyl)-isophthalamide | | | |
| 4-Amino-N1,N3-bis-1,3-benzodioxol-5-ylmethyl- | nt | nt | 4100 |
| isophthalamide | | | |
| 4-Acetylamino-N1,N3-bis-1,3-benzodioxol- | nt | nt | 5200 |
| 5-ylmethyl-isophthalamide | | | |
| N-(3-Methoxy-benzyl)-N′-pyridin-3-ylmethyl- | >100,000 | >100,000 | 7930 |
| isophthalamide | | | |
| N-(3-Methoxy-benzyl)-N′-pyridin-4-ylmethyl- | >100,000 | >100,000 | 1400 |
| isophthalamide | | | |
| N1-1,3-Benzodioxol-5-ylmethyl-N3-pyridin- | >100,000 | >100,000 | 1500 |
| 3-ylmethyl-isophthalamide | | | |
| N-(4-Chloro-benzyl)-N′-(3-methoxy-benzyl)- | >100,000 | >100,000 | 503 |
| isophthalamide | | | |
| N-(3,4-Dichloro-benzyl)-N′-(3-methoxy-benzyl)- | >100,000 | 68,000 | 555 |
| isophthalamide | | | |
| N-(4-Methoxy-benzyl)-N′-(3-methoxy-benzyl)- | >100,000 | 40,000 | 415 |
| isophthalamide | | | |
| N-(3-Methoxy-benzyl)-N′-(4-methyl-benzyl)- | >100,000 | 76,000 | 385 |
| isophthalamide | | | |
| N,N′-Bis-(4-fluoro-3-methoxy-benzyl)-isophthalamide | >100,000 | >100,000 | 930 |
| ({3-[(1,3-Benzodioxol-5-ylmethyl)-carbamoyl]- | >100,000 | >100,000 | 915 |
| benzoyl}-benzyl-amino)-acetic acid | | | |
| N-Benzo[1,3]dioxol-5-ylmethyl- | >100,000 | 30,000 | 33 |
| isophthalamic(4-hydroxymethyl-benzoic acid) ester | | | |
| N-(3,4-Dichloro-benzyl)-N′-pyridin-4-ylmethyl- | nt | nt | 2500 |
| isophthalamide | | | |
| N-(3-Methoxy-benzyl)-N′-(4-nitro-benzyl)- | >100,000 | >100,000 | 1135 |
| isophthalamide | | | |
| 4-{[3-(3-Methoxy-benzylcarbamoyl)-benzoylamino]- | >100,000 | 64,000 | 255 |
| methyl}-benzoic acid methyl ester | | | |
| N-3-Methoxybenzyl-isophthalamic(4-hydroxymethyl- | >100,000 | >100,000 | 44 |
| benzoic acid) ester | | | |
| 4-{[3-(3-Methoxy-benzylcarbamoyl)-benzoylamino]- | >100,000 | >100,000 | 77 |
| methyl}-benzoic acid | | | |
| N-(3-Amino-benzyl)-N′-(3-methoxy-benzyl)- | >100,000 | >100,000 | 935 |
| isophthalamide | | | |
| N-(3-Methoxy-benzyl)-N′-(3-nitro-benzyl)- | nt | nt | 2100 |
| isophthalamide | | | |
| 4-Ethoxy-N′1, N″3-bis-(3-methoxy-benzyl)- | >100,000 | >100,000 | 1833 |
| isophthalamide | | | |
| N1,N3-Bis-1,3-benzodioxol-5-ylmethyl-4-ethoxy- | 51,000 | 20,000 | 493 |
| isophthalamide | | | |
| N1,N3-Bis-1,3-benzodioxol-5-ylmethyl-4-propoxy- | >100,000 | 27,000 | 1450 |
| isophthalamide | | | |
| N1,N3-Bis-1,3-benzodioxol-5-ylmethyl-4-isopropoxy- | 71,000 | 30,000 | 3750 |
| isophthalamide | | | |
| N1,N3-Bis-2,1,3-benzothiadiazol-5-ylmethyl- | 30,000 | 21,000 | 155 |
| 4-methoxy-isophthalamide | | | |
| 4-Methoxy-isophthalic acid di-2,1,3-benzothiadiazol- | 30,000 | 30,000 | 370 |
| 5-ylmethyl ester | | | |

nt: not tested

In Table 2, the meanings of MMP-01, MMP-03 and MMP-13 and the methods of testing are as described above.

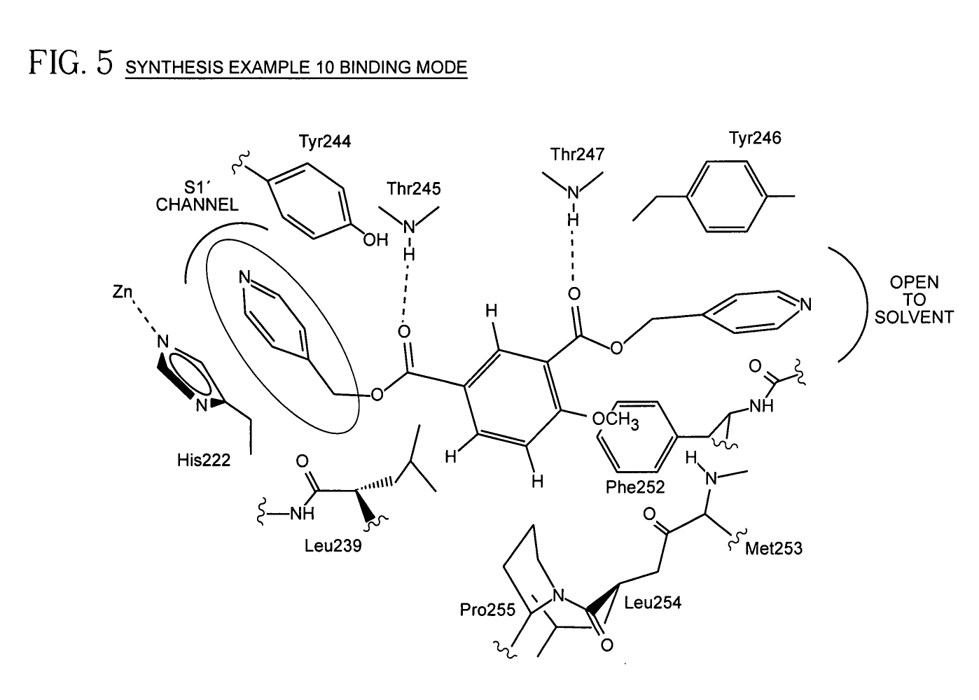

Binding of a representative example of one of the above compounds is shown in FIG. 5. It will be observed that the compounds of this series have two hydrophobic groups and two hydrogen bond acceptors. Bonding of these groups is as described for the first series of compounds. Since the third hydrogen bond acceptor is absent, the activity of the compounds in this series is on average less than that of the sulfonamide series.

Synthesis of some of the compounds referred to in Table II is described in the following further synthesis examples. The synthesis of the other compounds in the Table II is reported in our co-pending WO application which claims the priority of the application No. U.S. 60/268,736 filed on Feb. 14, 2001.

### SYNTHESIS EXAMPLE 9

### 4-Methoxy-N,N′-bis-(4-methoxybenzyl)-isophthalamide

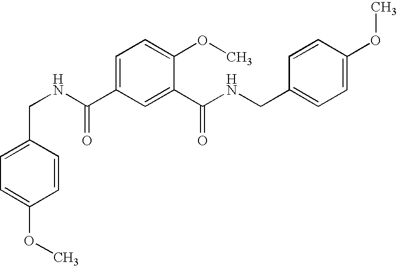

4-Methoxy-1,3-benzenedicarbonyl dichloride (1.16 g, 5.0 mmol) was added in parts to a solution of triethylamine (1.212 g, 12 mmol) and benzyl amine (1.37 g, 10 mmol) in methylene chloride (50 ml). The mixture was stirred at room temperature 18 hours and washed successively with 10% citric acid (100 ml), 1N sodium hydroxide solution (100 ml), and then brine (100 ml). The organic phase was dried over magnesium sulfate and evaporated at reduced pressure to give 1.95 g (90%) of the bisamide as a white solid. MS: M+1=435. Microanalysis (C₂₅H₂₆N₂O₅): Calculated: C, 69.11; H, 6.03; N, 6.45. Found: C, 68.82; H, 5.99; N, 6.27.

### SYNTHESIS EXAMPLE 10

### 4-Methoxy-isophthalic acid dipyridin-4-ylmethyl ester

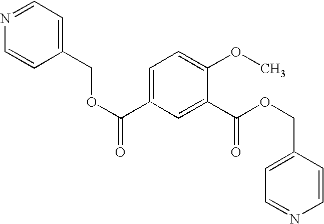

4-Methoxy-1,3-benzenedicarboxylic acid (675 mg, 3.4 mmol) and potassium carbonate (4.3 g, 31 mmol) were stirred in DMF (25 ml). To this were added in parts picolyl chloride hydrochloride (1.23 g, 7.5 mmol). The mixture was stirred at room temperature 24 hours, and then filtered free of insoluble material. The DMF solution was evaporated at reduced pressure to give a solid. This was partitioned between methylene chloride (100 ml) and saturated sodium bicarbonate solution (100 ml). The organic phase was separated and washed with water (100 ml) and then brine (100 ml). The organic phase was dried over magnesium sulfate and evaporated at reduced pressure to give 0.619 g (48%) of a tan solid. MS: M+1=379.1. Microanalysis (C₂₁H₁₈N₂O₅): Calculated: C, 66.66; H, 4.79; N, 7.40. Found: C, 66.15; H, 4.94; N, 7.53.

### SYNTHESIS EXAMPLE 11

### N,N-Bis-1,3-benzodioxol-5-ylmethyl-isophthalamide

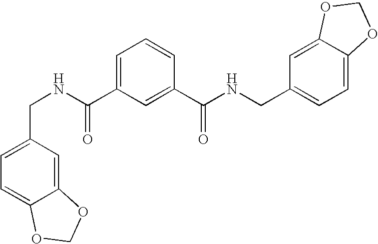

Piperonyl amine (12.8 g, 85 mmol) and triethyl amine (9.09 g, 90 mmol) were dissolved in methylene chloride (200 ml). To this was added in parts 1,3-benzenedicarbonyl dichloride (8.12 g, 40 mmol). The mixture was stirred at room temperature for 24 hours and then diluted with 1N hydrochloric acid (300 ml). The mixture was filtered to collect a solid. The solid was washed with 1N sodium hydroxide (50 ml), then water (6×100 ml) and dried at 65° C. for 3 hours at reduced pressure to give 15.08 g (87%) of a white solid. MS: M+1=433.3. Microanalysis (C₂₄H₂₀N₂O₆): Calculated: C, 66.66; H, 4.66; N, 6.48. Found: C, 66.56; H, 4.75; N, 6.46.

### SYNTHESIS EXAMPLES 12-16

General Procedures Used in the Combinatorial Array, Examples 12-16:

Loading of the Resin:

Marshall resin (15.2 g, 21.25 mmol) was swollen in DCM (300 ml) in a 500-ml resin tube (CAUTION: Slightly exothermic, the DCM will nearly boil). Once the mixture cools, cap the tube and agitate slowly for 5 minutes, venting frequently. Drain the DCM to waste. Repeat this wash two additional times. The resin was re-suspended in DCM (300 ml) and TEA (3.2 g, 32 mmol, 1.5 eq) was added slowly. The resulting mixture was swirled for 5 minutes when isophthalic acid dichloride (17.2 g, 85 mmol, 4 eq) was added in one portion. The resin tube was capped and carefully secured in a wrist shaker, and inverted for 36 hours. After 36 hours, a slight darkening of the resin was noted. The reaction solvent was drained and the resin washed three times with DCM (200 ml) and two times with diethyl ether (200 ml). The resin was dried under vacuum for 24 hours. Loading was determined both by weight gain and by total chloride determination. (Nitrogen content showed <0.05% N and therefore the absence of TEA.Cl). Typical loading was 1.1 mmol/g.

Resin Distribution:

Calibrate the Miniblock® resin loader for each resin used in the protocol. Record the milligram resin added per well, and calculate the number of millimoles per well. Using this calibration and the loading for each resin, distribute 0.15 mmol of resin per reaction tube. Close the valve on the block.

Amine Solution Prep:

Dilute the R¹ amine set to 0.5 M in DCM. Prepare a 0.2-M solution of TEA in DCM (1.5 ml per reaction). Prepare a 0.2-M solution of TEA in dioxane (1.5 ml per reaction). Dilute the R² amine set to 0.5 M in dioxane.

Addition of First Amine:

Add TEA solution in DCM from Step 2 (1.5 ml) to each reaction tube, then using the Miniblock® Map as a guide, distribute the appropriate first amine (315 μL, 1.05 eq). Shake for 24 hours. After 24 hours, place the reaction block on a filtration station without a collection block and drain the reactions to waste. Close the valve, add 2 ml DCM, shake for 2 minutes, again draining to waste. Unless Step 4 is to be carried out immediately, store the reaction blocks under vacuum.

Addition of Second Amine/Resin Cleavage:

Add TEA solution in dioxane from Step 2 (1.5 ml) to each reaction tube, then using the Miniblock® Map as a guide, distribute the appropriate second amine (300 μL, 1.05 eq). Shake for 72 hours. After 72 hours, place the reaction block on a filtration station with a labeled collection block and drain the reactions. Close the valve, add 2 ml DCM, shake for 2 minutes, drain into the collection tubes.

Analysis:

Check 25% by loop mass spec, first evaporating the DCM from the MS samples.

Concentrate:

Concentrate the crude samples in the Genevac.

### SYNTHESIS EXAMPLE 12

### N-1,3-Benzodioxol-5-ylmethyl-4-methoxy-N′-(4-methoxy-benzyl)-isophthalamide

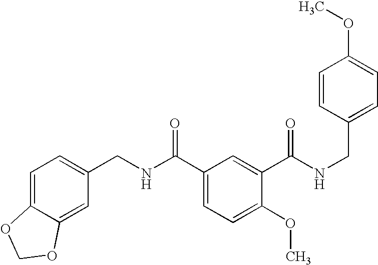

MS: Calculated: 448.22. found: 449. HPLC purity, 100%.

### SYNTHESIS EXAMPLE 13

### N,N′-Bis-1,3-benzodioxol-5-ylmethyl-4-methoxy-isophthalamide

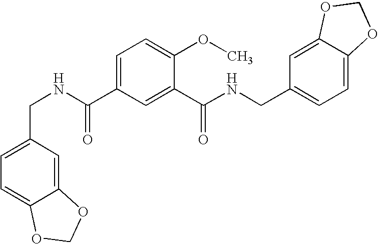

MS: Calculated: 462.1. found: 463. HPLC Purity, 100%.

### SYNTHESIS EXAMPLE 14

### N-1,3-Benzodioxol-5-ylmethyl-N′-(4-chloro-benzyl)-4-methoxy-isophthalamide

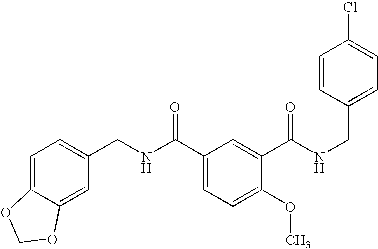

MS: Calc'd: 452.9. found: 452. HPLC purity, 100%.

### SYNTHESIS EXAMPLE 15

### N-Benzyl-4-methoxy-N′-(4-methoxy-benzyl)-isophthalamide

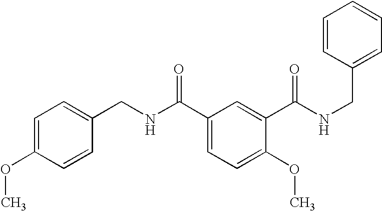

MS: Calc'd: 404.47. found: 405. HPLC purity, 100%.

### SYNTHESIS EXAMPLE 16

### 4-Methoxy-N,N′-bis-(3-methoxy-benzyl)-isophthalamide

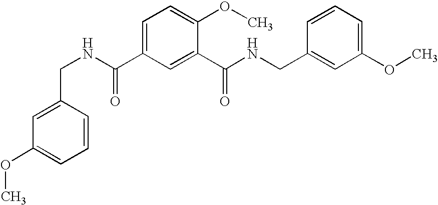

MS: Calc'd: 434.19. found: 435. HPLC purity, 100%.

Fused Bicyclic Pyrimidones

We have made a third group of compounds which are fused cyclic pyrimidones and are inhibitors of matrix metalloproteinase enzymes, and especially MMP-13. Preferred compounds that we have made, and their ability to inhibit the activity MMP-13 are summarized in Table III below:

TABLE III

| | MMP 13 IC₅₀ |
|---|---|
| Compound | μM |
| 3-Benzyl-2,4-dioxo-1,2,3,4-tetrahydro-thieno[2,3-d]pyrimidine- | 0.74 |
| 6-carboxylic acid benzyl ester | |
| 3-Benzyl-2,4-dioxo-1,2,3,4-tetrahydro-thieno[2,3-d]pyrimidine- | 0.31 |
| 6-carboxylic acid pyridin-4-ylmethyl ester | |
| 5-Methyl-2,4-dioxo-3-p-tolyl-1,2,3,4-tetrahydro-thieno[2,3-d]- | 10.0 |
| 6-carboxylic acid benzyl ester | |
| 3-Benzyl-1-methyl-2,4-dioxo-1,2,3,4-tetrahydro-thieno[2,3-d]pyrimidine- | 0.007 |
| 6-carboxylic acid benzyl ester | |
| 3-Benzyl-2,4-dioxo-1,2,3,4-tetrahydro-thieno[2,3-d]pyrimidine- | 0.068 |
| 6-carboxylic acid 1,3-benzodioxol-5-ylmethyl ester | |
| 3-Benzyl-1-metbyl-2,4-dioxo-1,2,3,4-tetrahydro-thieno[2,3-d]pyrimidine- | 0.47 |
| 6-carboxylic acid benzyl amide | |
| 3-Benzyl-2,4-dioxo-1,2,3,4-tetrahydro-thieno[2,3-d]pyrimidine-6- | 7.5 |
| carboxylic acid furfuryl-(5-carboxaldelhyde) ester | |
| 3-Benzyl-1-methyl-2,4-dioxo-1,2,3,4-tetrahydro-thieno[2,3-d]pyrimidine- | 1.45 |
| 6-carboxylic acid benzofuran-2-ylmethyl ester | |
| 3-(4-Bromo-benzyl)-2,4-dioxo-1,2,3,4-tetrahydro-thieno[2,3- | 0.26 |
| d]pyrimidine-6-carboxylic acid pyridin-4-ylmethyl ester | |
| 3-Benzyl-2,4-dioxo-1,2,3,4-tetrahydro-thieno[2,3-d]pyrimidine-6- | 0.0875 |
| carboxylic acid 4-methoxy-benzyl ester | |
| 4-{1-Methyl-2,4-dioxo-6-[(pyridin-4-ylmethyl)-carbamoyl]-1,4-dihydro- | 0.0205 |
| 2H-thieno[2,3-d]pyrimidin-3-ylmethyl}-benzoic acid; compound with | |
| trifluoro-acetic acid | |
| 4-[6-(4-Methoxy-benzylcarbamoyl)-1-methyl-2,4-dioxo-1,4-dihydro-2H- | 0.00395 |
| thieno[2,3-d]pyrimidin-3-ylmethyl]-benzoic acid | |
| 4-[6-(3,4-Dimethoxy-benzylcarbamoyl)-1-methyl-2,4-dioxo-1,4-dihydro- | 4.5 |
| 2H-thieno[2,3-d]pyrimidin-3-ylmethyl]-benzoic acid | |
| 4-[6-(4-Bromo-benzylcarbamoyl)-1-methyl-2,4-dioxo-1,4-dihydro-2H- | 0.011 |
| thieno[2,3-d]pyrimidin-3-ylmethyl]-benzoic acid | |
| 4-[6-(3,5-Bis-trifluoromethyl-benzylcarbamoyl)-1-methyl-2,4-dioxo-1,4- | 5.6 |
| dihydro-2H-thieno[2,3-d]pyrimidin-3-ylmethyl]-benzoic acid | |
| 4-[6-(4-Chloro-benzylcarbamoyl)-1-methyl-2,4-dioxo-1,4-dihydro-2H- | 0.0115 |
| thieno[2,3-d]pyrimidin-3-ylmethyl]-benzoic acid | |
| 4-[1-Methyl-2,4-dioxo-6-(4-sulfamoyl-benzylcarbamoyl)-1,4-dihydro-2H- | 2 |
| thieno[2,3-d]pyrimidin-3-ylmethyl]-benzoic acid | |
| 3-(4-Fluoro-benzyl)-1-methyl-2,4-dioxo-1,2,3,4-tetrahydro-thieno[2,3- | 0.16 |
| d]pyrimidine-6-carboxylic acid 4-methoxy-benzylamide | |
| 3-(4-Iodo-benzyl)-1-methyl-2,4-dioxo-1,2,3,4-tetrahydro-thieno[2,3- | 0.045 |
| d]pyrimidine-6-carboxylic acid 3-methoxy-benzylamide | |
| 3-(4-Dimethylsulfamoyl-benzyl)-1-methyl-2,4-dioxo-1,2,3,4-tetrahydro- | 0.0535 |
| thieno[2,3-d]pyrimidine-6-carboxylic acid 4-methoxy-benzylamide | |
| 3-(3-Methoxy-benzyl)-1-methyl-2,4-dioxo-1,2,3,4-tetrahydro-thieno[2,3- | 0.11 |
| d]pyrimidine-6-carboxylic acid 4-methoxy-benzylamide | |
| 3-(4-Cyano-benzyl)-1-methyl-2,4-dioxo-1,2,3,4-tetrahydro-thieno[2,3- | 0.062 |
| d]pyrimidine-6-carboxylic acid 4-methoxy-benzylamide | |
| 3-(4-Acetylamino-benzyl)-1-methyl-2,4-dioxo-1,2,3,4-tetrahydro- | 0.0535 |
| thieno[2,3-d]pyrimidine-6-carboxylic acid 3-methoxy-benzylamide | |
| 5-[6-(3-Methoxy-benzylcarbamoyl)-1-methyl-2,4-dioxo-1,4-dihydro-2H- | 1.05 |
| thieno[2,3-d]pyrimidin-3-ylmethyl]-furan-2-carboxylic acid ethyl ester | |
| 3-(4-Cyano-benzyl)-2,4-dioxo-1,2,3,4-tetrahydro-thieno[2,3-d]pyrimidine- | 0.0275 |
| 6-carboxylic acid 3-methoxy-benzyl ester | |
| 2,4-Dioxo-3-[4-(2H-tetrazol-5-yI)-benzyl]-1,2,3,4-tetrahydro-thieno[2,3- | 0.00168 |
| d]pyrimidine-6-carboxylic acid 3-methoxy-benzyl ester | |
| 4-[6-(3-Methoxy-benzylcarbamoyl)-1-methyl-2,4-dioxo-1,4-dihydro-2H- | 0.0635 |
| thieno[2,3-d]pyrimidin-3-ylmethyl]-benzoic acid 2-dimethylamino-ethyl | |
| ester | |
| 3-Cylcohexylmethyl-1-methyl-2,4-dioxo-1,2,3,4-tetrahydro-thieno[2,3- | 0.057 |
| d]pyrimidine-6-carboxylic acid-3methoxy-benzylamide | |
| 3-cylcohexylmethyl-1-methyl-2,4-dioxo-1,2,3,4-tetrahydro-thieno[2,3- | 0.1185 |
| d]pyrimidine-6-carboxylic acid-4methoxy-benzylamide | |
| 3-Benzyl-1-methyl-2,4-dioxo-1,2,3,4-tetrahydro-thieno[2,3-d]pyrimidine- | 0.345 |
| 6-carboxylic acid (pyridin-4-ylmethyl)-amide | |
| 4-[6-(3-Difluoromethoxy-benzylcarbamoyl)-1-methyl-2,4-dioxo-1,4- | 0.00655 |
| dihydro-2H-thieno[2,3-d]pyrimidin-3-ylmethyl]-benzoic acid | |
| 4-[6-(3-Difluoromethoxy-benzylcarbamoyl)-1-methyl-2,4-dioxo-1,4- | 0.900 |
| dihydro-2H-thieno[2,3-d]pyrimidin-3-ylmethyl]-benzoic acid tert-butyl | |
| ester | |
| 4-[6-(3-Methoxy-benzylcarbarnoyl)-1-methyl-2,4-dioxo-1,4-dihydro-2H- | 0.00205 |
| thieno[2,3-d]pyrimidin-3-ylmethyl]-benzoic acid | |
| 4-[6-(4-Methanesulfonyl-benzylcarbamoyl)-1-methyl-2,4-dioxo-1,4- | 3.899 |
| dihydro-2H-thieno[2,3-d]pyrimidin-3-ylmethyl]-benzoic acid | |
| 4-[1-Methyl-2,4-dioxo-6-(2-pyridin-4-yl-ethylcarbamoyl)-1,4-dihydro-2H- | 3.700 |
| thieno[2,3-d]pyrimidin-3-ylmethyl]-benzoic acid | |
| 1-Metbyl-2,4-dioxo-3-(4-trifluoromethoxy-benzyl)-1,2,3,4-tetrahydro- | 0.140 |
| thieno[2,3-d]pyrimidine-6-carboxylic acid 3-methoxy-benzylamide | |
| 4-[6-(3-Methoxy-benzylcarbamoyl)-1-methyl-2,4-dioxo-1,4-dihydro-2H- | 0.02050 |
| thieno[2,3-d]pyrimidin-3-ylmethyl]-benzoic acid methyl ester | |
| 3-(2,3-Dihydro-benzofuran-6-ylmethyl)-1-methyl-2,4-dioxo-1,2,3,4- | 0.04750 |
| tetrahydro-thieno[2,3-d]pyrimidine-6-carboxylic acid 3-methoxy- | |
| benzylamide | |
| 1-Methyl-3-(2-methyl-thiazol-5-ylmethyl)-2,4-dioxo-1,2,3,4-tetrahydro- | 1.3999 |
| thieno[2,3-d]pyrimidine-6-carboxylic acid 3-methoxy-benzylamide | |
| 1-Methyl-2,4-dioxo-3-[4-(1H-tetrazol-5-yl)-benzyl]-1,2,3,4-tetrahydro- | 0.0185 |
| thieno[2,3-d]pyrimidine-6-carboxylic acid 4-fluoro-benzylamide | |
| 3-Benzyl-2-methoxy-4-oxo-3,4-dihydro-thieno[2,3-d]pyrimidine-6- | 3.149 |
| carboxylic acid benzyl ester | |
| 4-[6-(3-Methoxy-benzylcarbamoyl)-1-methyl-2,4-dioxo-1,4-dihydro-2H- | 0.1135 |
| thieno[2,3-d]pyrimidin-3-ylmethyl]-benzoic acid 2,2-dimethyl- | |
| propionyloxymethyl ester | |
| 4-[6-(3-Methoxy-benzylcarbamoyl)-1-methyl-2,4-dioxo-1,4-dihydro-2H- | 0.00543 |
| thieno[2,3-d]pyrimidin-3-ylmethyl]-cyclohexanecarboxylic acid | |
| 4-[6-(3-Methoxy-benzylcarbamoyl)-1-methyl-2,4-dioxo-1,4-dihydro-2H- | 0.0496 |
| thieno[2,3-d]pyrimidin-3-ylmethyl]-cyclohexanecarboxylic acid methyl | |
| ester | |
| 1-{4-[6-(3-Methoxy-benzylcarbamoyl)-1-methyl-2,4-dioxo-1,4-dihydro- | 0.0109 |
| 2H-thieno[2,3-d]pyrimidin-3-ylmethyl]-phenyl}-cyclopropanecarboxylic | |
| acid methyl ester | |
| 1-{4-[6-(3-Methoxy-benzylcarbamoyl)-1-methyl-2,4-dioxo-1,4-dihydro- | 0.111 |
| 2H-thieno[2,3-d]pyrimidin-3-ylmethyl]-phenyl}-cyclopropanecarboxylic | |
| acid tert-butyl ester | |
| 1-{4-[6-(3-Methoxy-benzylcarbamoyl)-1-methyl-2,4-dioxo-1,4-dihydro- | 0.005349 |
| 2H-thieno[2,3-d]pyrimidin-3-ylmethyl]-phenyl}-cyclopropanecarboxylic | |
| acid | |
| 2-{4-[6-(3-Methoxy-benzylcarbamoyl)-1-methyl-2,4-dioxo-1,4-dihydro- | 0.10349 |
| 2H-thieno[2,3-d]pyrimidin-3-ylmethyl]-phenoxy}-2-methyl-propionic | |
| acid tert-butyl ester | |
| 2-{4-[6-(3-Methoxy-benzylcarbamoyl)-1-methyl-2,4-dioxo-1,4-dihydro- | 0.01849 |
| 2H-thieno[2,3-d]pyrimidin-3-ylmethyl]-phenoxy}-2-methyl-propionic | |
| acid | |
| 3-(3-Methoxy-benzyl)-1-methyl-2,4-dioxo-1,2,3,4-tetrahydro-thieno[2,3- | 0.063 |
| d]pyrimidine-6-carboxylic acid benzyl ester | |
| 3-Benzyl-1-methyl-2,4-dioxo-1,2,3,4-tetrahydro-thieno[2,3-d]pyrimidine- | 0.16 |
| 6-carboxylic acid benzyl ester | |
| 3-Biphenyl-4-ylmethyl-1-methyl-2,4-dioxo-1,2,3,4-tetrahydro-thieno[2,3- | 0.61 |
| d]pyrimidine-6-carboxylic acid benzyl ester | |
| 3-(4-Methanesulfonyl-benzyl)-1-methyl-2,4-dioxo-1,2,3,4-tetrahydro- | 0.034 |
| thieno[2,3-d]pyrimidine-6-carboxylic acid benzyl ester | |
| 1-Methyl-3-(4-methyl-benzyl)-2,4-dioxo-1,2,3,4-tetrahydro-thieno[2,3- | 0.03 |
| d]pyrimidine-6-carboxylic acid benzyl ester | |
| 1-Methyl-2,4-dioxo-3-phenethyl-1,2,3,4-tetrahydro-thieno[2,3- | 1.1 |
| d]pyrimidine-6-carboxylic acid benzyl ester | |
| 3-(4-Amino-6-phenylamino-1,3,5-triazin-2-ylmethyl)-1-methyl-2,4-dioxo- | 0.52 |
| 1,2,3,4-tetrahydro-thieno[2,3-d]pyrimidine-6-carboxylic acid benzyl ester | |
| 1-Methyl-2,4-dioxo-3-(4-trifluoromethyl-benzyl)-1,2,3,4-tetrahydro- | 0.59 |
| thieno[2,3-d]pyrimidine-6-carboxylic acid benzyl ester | |
| 3-(6-Cyano-hexyl)-1-methyl-2,4-dioxo-1,2,3,4-tetrahydro-thieno[2,3- | 2.4 |
| d]pyrimidine-6-carboxylic acid benzyl ester | |
| 3-[2-(2,5-Dimethoxy-phenyl)-2-oxo-ethyl]-1-methyl-2,4-dioxo-1,2,3,4- | 1.7 |
| tetrahydro-thieno[2,3-d]pyrimidine-6-carboxylic acid benzyl ester | |
| 3-(3-Iodo-benzyl)-1-methyl-2,4-dioxo-1,2,3,4-tetrahydro-thieno[2,3- | 0.94 |
| d]pyrimidine-6-carboxylic acid benzyl ester | |
| 1-Methyl-2,4-dioxo-3-(3-trifluoromethyl-benzyl)-1,2,3,4-tetrahydro- | 0.42 |
| thieno[2,3-d]pyrimidine-6-carboxylic acid benzyl ester | |
| 3-(2,4-Bis-trifluoromethyl-benzyl)-1-methyl-2,4-dioxo-1,2,3,4-tetrahydro- | 3.2 |
| thieno[2,3-d]pyrimidine-6-carboxylic acid benzyl ester | |
| 3-[2-(1,3-Dioxo-1,3-dihydro-isoindol-2-yl)-ethyl]-1-methyl-2,4-dioxo- | 2.9 |
| 1,2,3,4-tetrahydro-thieno[2,3-d]pyrimidine-6-carboxylic acid benzyl ester | |
| 3-[2-(1,3-Dioxo-1,3-dihydro-isoindol-2-yl)-ethyl]-1-methyl-2,4-dioxo- | 2.9 |
| 1,2,3,4-tetrahydro-thieno[2,3-d]pyrimidine-6-carboxylic acid benzyl ester | |
| 3-(2-Carboxy-allyl)-1-methyl-2,4-dioxo-1,2,3,4-tetrahydro-thieno[2,3- | 0.33 |
| d]pyrimidine-6-carboxylic acid benzyl ester | |
| 3-(1,3-Dioxo-1,3-dihydro-isoindol-2-ylmethyl)-1-methyl-2,4-dioxo- | 0.036 |
| 1,2,3,4-tetrahydro-thieno[2,3-d]pyrimidine-6-carboxylic acid benzyl ester | |
| 3-(4-Fluoro-benzyl)-1-methyl-2,4-dioxo-1,2,3,4-tetrahydro-thieno[2,3- | 0.015 |
| d]pyrimidine-6-carboxylic acid benzyl ester | |
| 1-Methyl-3-oxiranylmethyl-2,4-dioxo-1,2,3,4-tetrahydro-thieno[2,3- | 0.51 |
| d]pyrimidine-6-carboxylic acid benzyl ester | |
| 1-Methyl-3-(2-methyl-butyl)-2,4-dioxo-1,2,3,4-tetrahydro-thieno[2,3- | 0.13 |
| d]pyrimidine-6-carboxylic acid benzyl ester | |
| 1-Methyl-2,4-dioxo-3-(4-phenoxy-butyl)-1,2,3,4-tetrahydro-thieno[2,3- | 0.25 |
| d]pyrimidine-6-carboxylic acid benzyl ester | |
| 3-(2-Cyano-benzyl)-1-methyl-2,4-dioxo-1,2,3,4-tetrahydro-thieno[2,3- | 4.5 |
| d]pyrimidine-6-carboxylic acid benzyl ester | |
| 1-Methyl-2,4-dioxo-3-(3-phenoxy-propyl)-1,2,3,4-tetrahydro-thieno[2,3- | 7.8 |
| d]pyrimidine-6-carboxylic acid benzyl ester | |
| 3-Hex-5-enyl-1-metbyl-2,4-dioxo-1,2,3,4-tetrahydro-thieno[2,3- | 0.11 |
| d]pyrimidine-6-carboxylic acid benzyl ester | |
| 1-Methyl-2,4-dioxo-3-pyridin-3-ylmethyl-1,2,3,4-tetrahydro-thieno[2,3- | 0.09 |
| d]pyrimidine-6-carboxylic acid benzyl ester | |
| 1,3-Dimethyl-2,4-dioxo-1,2,3,4-tetrahydro-thieno[2,3-d]pyrimidine-6- | 3.9 |
| carboxylic acid benzyl ester | |
| 3-Cyclobutylmetbyl-1-metbyl-2,4-dioxo-1,2,3,4-tetrahydro-thieno[2,3- | 0.19 |
| d]pyrimidine-6-carboxylic acid benzyl ester | |
| 3-Allyl-1-methyl-2,4-dioxo-1,2,3,4-tetrahydro-thieno[2,3-d]pyrimidine-6- | 0.16 |
| carboxylic acid benzyl ester | |
| 1-Methyl-2,4-dioxo-3-prop-2-ynyl-1,2,3,4-tetrahydro-thieno[2,3- | 0.097 |
| d]pyrimidine-6-carboxylic acid benzyl ester | |
| 3-But-2-ynyl-1-methyl-2,4-dioxo-1,2,3,4-tetrahydro-thieno[2,3- | 0.019 |
| d]pyrimidine-6-carboxylic acid benzyl ester | |
| 1-Methyl-2,4-dioxo-3-(2-phenoxy-ethyl)-1,2,3,4-tetrahydro-thieno[2,3- | 0.074 |
| d]pyrimidine-6-carboxylic acid benzyl ester | |
| 3-(3-Hydroxy-2-methyl-propyl)-1-methyl-2,4-dioxo-1,2,3,4-tetrahydro- | 1.5 |
| thieno[2,3-d]pyrimidine-6-carboxylic acid benzyl ester | |
| 3-Isobutyl-1-metbyl-2,4-dioxo-1,2,3,4-tetrahydro-thieno[2,3- | 0.086 |
| d]pyrimidine-6-carboxylic acid benzyl ester | |
| 3-(6-Chloro-pyridin-3-ylmethyl)-1-methyl-2,4-dioxo-1,2,3,4-tetrahydro- | 0.051 |
| thieno[2,3-d]pyrimidine-6-carboxylic acid benzyl ester | |
| 3-(2-Benzenesulfonylmetbyl-benzyl)-1-methyl-2,4-dioxo-1,2,3,4- | 8.3 |
| tetrahydro-thieno[2,3-d]pyrimidine-6-carboxylic acid benzyl ester | |
| 1-Methyl-3-naphthalen-1-ylmethyl-2,4-dioxo-1,2,3,4-tetrahydro- | 0.66 |
| thieno[2.3-d]pyrimidine-6-carboxylic acid benzyl ester | |
| thieno[2,3-d]pyrimidine-6-carboxylic acid benzyl ester | |
| 1-Methyl-2,4-dioxo-3-(2-trifluoromethyl-benzyl)-1,2,3,4-tetrahydro- | 0.25 |
| thieno[2,3-d]pyrimidine-6-carboxylic acid benzyl ester | |
| 3-(3-Chloro-benzyl)-1-methyl-2,4-dioxo-1,2,3,4-tetrahydro-thieno[2,3- | 0.017 |
| d]pyrimidine-6-carboxylic acid benzyl ester | |
| 3-(4-Methoxycarbonyl-butyl)-1-methyl-2,4-dioxo-1,2,3,4-tetrahydro- | 0.15 |
| thieno[2,3-d]pyrimidine-6-carboxylic acid benzyl ester | |
| 3-Ethyl-1-methyl-2,4-dioxo-1,2,3,4-tetrahydro-thieno[2,3-d]pyrimidine-6- | 0.39 |
| carboxylic acid benzyl ester | |
| 1-Methyl-2,4-dioxo-3-(3-phenyl-propyl)-1,2,3,4-tetrahydro-thieno[2,3- | 0.28 |
| d]pyrimidine-6-carboxylic acid benzyl ester | |
| 3-[2-(4-Chloro-benzenesulfonyl)-ethyl]-1-methyl-2,4-dioxo-1,2,3,4- | 0.003 |
| tetrahydro-thieno[2,3-d]pyrimidine-6-carboxylic acid benzyl ester | |
| 3-(2-Acetoxy-ethyl)-1-methyl-2,4-dioxo-1,2,3,4-tetrahydro-thieno[2,3- | 1.3 |
| d]pyrimidine-6-carboxylic acid benzyl ester | |
| 3-Benzyl-1-methyl-2,4-dioxo-1,2,3,4-tetrahydro-thieno[2,3-d]pyrimidine- | 0.16 |
| 6-carboxylic acid benzyl ester | |
| 3-Benzyl-1-methyl-2,4-dioxo-1,2,3,4-tetrahydro-thieno[2,3-d]pyrimidine- | 0.54 |
| 6-carboxylic acid benzyl amide | |
| 3-Benzyl-1-methyl-2,4-dioxo-1,2,3,4-tetrabydro-thieno[2,3-d]pyrimidine- | 9.9 |
| 6-carboxylic acid 2-diethylamino-1-methyl-ethyl ester | |
| 3-Benzyl-1-methyl-2,4-dioxo-1,2,3,4-tetrahydro-thieno[2,3-d]pyrimidine- | 0.004 |
| 6-carboxylic acid 4-fluoro-benzyl ester | |
| 3-Benzyl-1-methyl-2,4-dioxo-1,2,3,4-tetrahydro-thieno[2,3-d]pyrimidine- | 0.18 |
| 6-carboxylic acid 4-trifluoromethyl-benzyl ester | |
| 3-Benzyl-1-methyl-2,4-dioxo-1,2,3,4-tetrahydro-thieno[2,3-d]pyrimidine- | 0.016 |
| 6-carboxylic acid pyridin-3-ylmethyl ester | |
| 3-Benzyl-1-methyl-2,4-dioxo-1,2,3,4-tetrahydro-thieno[2,3-d]pyrimidine- | 0.92 |
| 6-carboxylic acid 4-methoxy-benzyl ester | |
| 3-Benzyl-1-methyl-2,4-dioxo-1,2,3,4-tetrahydro-thieno[2,3-d]pyrimidine- | 0.72 |
| 6-carboxylic acid 2-benzyloxy-ethyl ester | |
| 3-Benzyl-1-methyl-2,4-dioxo-1,2,3,4-tetrahydro-thieno[2,3-d]pyrimidine- | 0.25 |
| 6-carboxylic acid 4-nitro-benzyl ester | |
| 3-Benzyl-1-methyl-2,4-dioxo-1,2,3,4-tetrahydro-thieno[2,3-d]pyrimidine- | 8.6 |
| 6-carboxylic acid 3-phenoxy-benzyl ester | |
| 3-Benzyl-1-methyl-2,4-dioxo-1,2,3,4-tetrahydro-thieno[2,3-d]pyrimidine- | 0.014 |
| 6-carboxylic acid 4-chloro-benzyl ester | |
| 3-Benzyl-1-methyl-2,4-dioxo-1,2,3,4-tetrahydro-thieno[2,3-d]pyrimidine- | 4.5 |
| 6-carboxylic acid 1-ethyl-piperidin-3-yl ester | |
| 3-Benzyl-1-methyl-2,4-dioxo-1,2,3,4-tetrahydro-thieno[2,3-d]pyrimidine- | 4.9 |
| 6-carboxylic acid 3-(4-methoxy-phenyl)-propyl ester | |
| 3-Benzyl-1-methyl-2,4-dioxo-1,2,3,4-tetrahydro-thieno[2,3-d]pyrimidine- | 2.3 |
| 6-carboxylic acid tetrahydro-furan-3-yl ester | |
| 3-Benzyl-1-methyl-2,4-dioxo-1,2,3,4-tetrahydro-thieno[2,3-d]pyrimidine- | 0.0034 |
| 6-carboxylic acid 3-methoxy-benzyl ester | |
| 3-Benzyl-1-methyl-2,4-dioxo-1,2,3,4-tetrahydro-thieno[2,3-d]pyrimidine- | 0.067 |
| 6-carboxylic acid 3-chloro-benzyl ester | |
| 3-Benzyl-1-methyl-2,4-dioxo-1,2,3,4-tetrahydro-thieno[2,3-d]pyrimidine- | 0.3 |
| 6-carboxylic acid 1,3-benzodioxol-5-ylmethyl ester | |
| 3-Benzyl-1-methyl-2,4-dioxo-1 2,3,4-tetrahydro-thieno[2,3-d]pyrimidine- | 0.36 |
| 6-carboxylic acid 4-methylsulfanyl-benzyl ester | |
| 3-Benzyl-1-methyl-2,4-dioxo-1,2,3,4-tetrahydro-thieno[2,3-d]pyrimidine- | 0.072 |
| 6-carboxylic acid 3,4-dichloro-benzyl ester | |
| 3-Benzyl-1-methyl-2,4-dioxo-1,2,3,4-tetrahydro-thieno[2,3-d]pyrimidine- | 0.2 |
| 6-carboxylic acid furan-3-ylmethyl ester | |
| 3-Benzyl-1-methyl-2,4-dioxo-1,2,3,4-tetrahydro-thieno[2,3-d]pyrimidine- | 0.1 |
| 6-carboxylic acid but-3-enyl ester | |
| 3-Benzyl-1-methyl-2,4-dioxo-1,2,3,4-tetrahydro-thieno[2,3-d]pyrimidine- | 1.2 |
| 6-carboxylic acid 2-ethoxy-ethyl ester | |
| 3-Benzyl-1-methyl-2,4-dioxo-1,2,3,4-tetrahydro-thieno[2,3-d]pyrimidine- | 2.1 |
| 6-carboxylic acid cyano-phenyl-methyl ester | |
| 3-Benzyl-1-methyl-2,4-dioxo-1,2,3,4-tetrahydro-thieno[2,3-d]pyrimidine- | 0.67 |
| 6-carboxylic acid 4-trifluoromethyl-benzylamide | |
| 3-Benzyl-1-methyl-2,4-dioxo-1,2,3,4-tetrahydro-thieno[2,3-d]pyrimidine- | 1.7 |
| 6-carboxylic acid 4-methyl-benzylamide | |
| 1-Methyl-2,4-dioxo-3-(4-sulfamoyl-benzyl)-1,2,3,4-tetrahydro-thieno[2,3- | 0.0785 |
| d]pyrimidine-6-carboxylic acid 4-methoxy-benzylamide | |
| 3-[4-(N-Hydroxycarbamimidoyl)-benzyl]-1-methyl-2,4-dioxo-1,2,3,4- | 0.061 |
| tetrahydro-thieno[2,3-d]pyrimidine-6-carboxylic acid 4-methoxy- | |
| benzylamide | |
| 1-Methyl-2,4-dioxo-3-[4-(5-oxo-4,5-dihydro-1,2,4-oxadiazol-3-yl)- | 0.0046 |
| benzyl]-1,2,3,4-tetrahydro-thieno[2,3-d]pyrimidine-6-carboxylic | |
| acid 4-methoxy-benzylamide | |
| 1-Methyl-2,4-dioxo-3-[4-(5-thioxo-4,5-dihydro-1,2,4-oxadiazol-3-yl)- | 0.0042 |
| benzyl]-1,2,3,4-tetrahydro-thieno[2,3-d]pyrimidine-6-carboxylic acid 4- | |
| methoxy-benzylamide | |
| 3-Cyanomethyl-1-methyl-2,4-dioxo-1,2,3,4-tetrahydro-thieno[2,3- | 0.783 |
| d]pyrimidine-6-carboxylic acid 3-methoxy-benzylamide | |
| (E)-4-[6-(4-Methoxy-benzylcarbamoyl)-1-methyl-2,4-dioxo-1,4-dihydro- | 0.225 |
| 2H-thieno[2,3-d]pyrimidin-3-yl]-but-2-enoic acid methyl ester | |
| 2-Methoxy-4-[6-(4-methoxy-benzylcarbamoyl)-1-methyl-2,4-dioxo-1,4- | 0.435 |
| dihydro-2H-thieno[2,3-d]pyrimidin-3-ylmethyl]-benzoic acid methyl ester | |
| 3-(2-Methoxymethyl-1,1,3-trioxo-2,3-dihydro-1H-1λ⁶-1,2-benzisothiazol- | 0.68 |
| 6-ylmethyl)-1-methyl-2,4-dioxo-1,2,3,4-tetrahydro-thieno[2,3- | |
| d]pyrimidine-6-carboxylic acid 4-methoxy-benzylamide | |
| 1-Methyl-3-oct-2-ynyl-2,4-dioxo-1,2,3,4-tetrahydro-thieno[2,3- | 0.077 |
| d]pyrimidine-6-carboxylic acid 4-methoxy-benzylamide | |
| 3-[2-(4-Bromo-phenoxy)-ethyl]-1-methyl-2,4-dioxo-1,2,3,4-tetrahydro- | 0.175 |
| thieno[2,3-d]pyrimidine-6-carboxylic acid 4-methoxy-benzylamide | |
| 3-[2-(4-Bromo-phenoxy)-ethyl]-1-methyl-2,4-dioxo-1,2,3,4-tetrahydro- | 0.069 |
| thieno[2,3-d]pyrimidine-6-carboxylic acid 3-methoxy-benzylamide | |
| 3-[2-(4-Fluoro-phenoxy)-ethyl]-1-methyl-2,4-dioxo-1,2,3,4-tetrahydro- | 0.15 |
| thieno[2,3-d]pyrimidine-6-carboxylic acid 4-methoxy-benzylamide | |
| 3-[2-(4-fluoro-phenoxy)-ethyl]-1-methyl-2,4-dioxo-1,2,3,4-tetrahydro- | 0.0495 |
| thieno[2,3-d]pyrimidine-6-carboxylic acid 3-methoxy-benzylamide | |
| 3-[2-(4-chloro-phenoxy)-ethyl]-1-methyl-2,4-dioxo-1,2,3,4-tetrahydro- | 0.0925 |
| thieno[2,3-d]pyrimidine-6-carboxylic acid 3-methoxy-benzylamide | |
| 4-[6-(4-Methoxy-benzylcarbamoyl)-1-methyl-2,4-dioxo-1,4-dihydro-2H- | 0.0555 |
| thieno[2,3-d]pyrimidin-3-ylmethyl]-2-methyl-benzoic acid methyl ester | |
| 4-[6-(4-Methoxy-benzylcarbamoyl)-1-methyl-2,4-dioxo-1,4-dihydro-2H- | 0.0585 |
| thieno[2,3-d]pyrimidin-3-ylmethyl]-benzoic acid methyl ester | |
| 2-Methoxy-4-[6-(3-methoxy-benzylcarbamoyl)-1-methyl-2,4-dioxo-1,4- | 0.18 |
| dihydro-2H-thieno[2,3-d]pyrimidin-3-ylmethyl]-benzoic acid methyl ester | |
| 4-[6-(3-Methoxy-benzylcarbamoyl)-1-methyl-2,4-dioxo-1,4-dihydro-2H- | 0.0195 |
| thieno[2,3-d]pyrimidin-3-ylmethyl]-2-methyl-benzoic acid methyl ester | |
| 1-Methyl-2,4-dioxo-3-(3-oxo-3-phenyl-propyl)-1,2,3,4-tetrahydro- | 3 |
| thieno[2,3-d]pyrimidine-6-carboxylic acid 4-methoxy-benzylamide | |
| 1-Methyl-2,4-dioxo-3-(3-oxo-3-phenyl-propyl)-1,2,3,4-tetrahydro- | 1.4 |
| thieno[2,3-d]pyrimidine-6-carboxylic acid 3-methoxy-benzylamide | |
| 1-Methyl-2,4-dioxo-3-[2-(3-trifluoromethyl-benzenesulfonyl)-ethyl]- | 1.25 |
| 1,2,3,4-tetrahydro-thieno[2,3-d]pyrimidine-6-carboxylic acid 3-methoxy- | |
| benzylamide | |
| 3-[2-(4-Chloro-benzenesulfonyl)-ethyl]-1-methyl-2,4-dioxo-1,2,3,4- | 5.65 |
| tetrahydro-thieno[2,3-d]pyrimidine-6-carboxylic acid 3-methoxy- | |
| benzylamide | |
| 3-[2-(4-Chloro-benzenesulfonyl)-ethyl]-1-methyl-2,4-dioxo-1,2,3,4- | 7.2 |
| tetrahydro-thieno[2,3-d]pyrimidine-6-carboxylic acid 4-methoxy- | |
| benzylamide | |
| 4-[6-(3-hydroxy-benzylcarbamoyl)-1-methyl-2,4-dioxo-1,4-dihydro-2H- | 0.00765 |
| thieno[2,3-d]pyrimidin-3-ylmethyl]-2-methyl-benzoic acid | |
| 4-(6-Carbamoyl-1-methyl-2,4-dioxo-1,4-dihydro-2H-thieno[2,3- | 0.655 |
| d]pyrimidin-3-ylmethyl)-2-hydroxy-benzoic acid | |
| 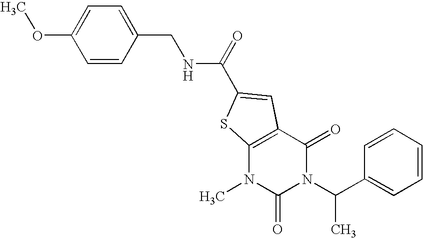 | 0.81 |
| 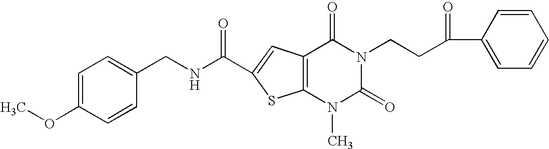 | 1.5 |
| 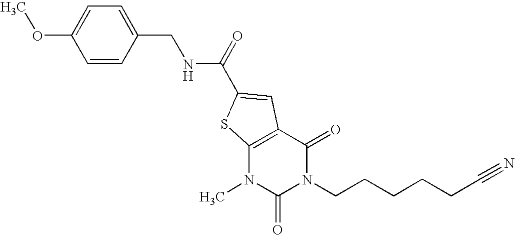 | 1.5 |
| 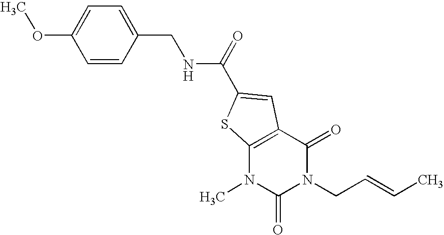 | 0.27 |
| 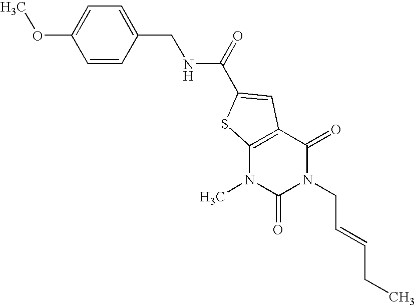 | 0.063 |
| 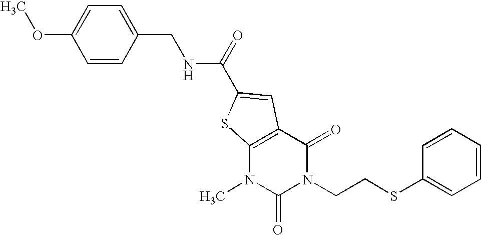 | 0.58 |
| 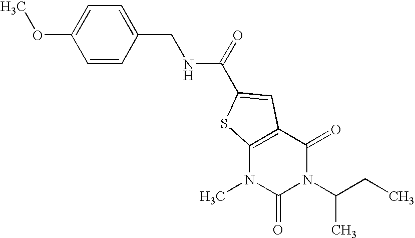 | 3.4 |
| 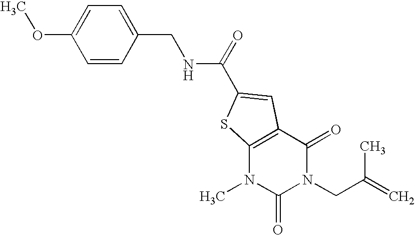 | 2.15 |
| 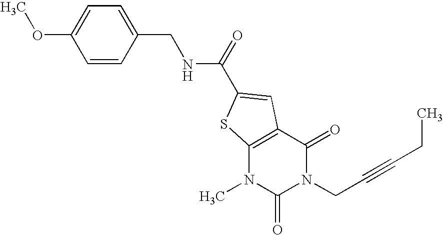 | 0.038 |
| 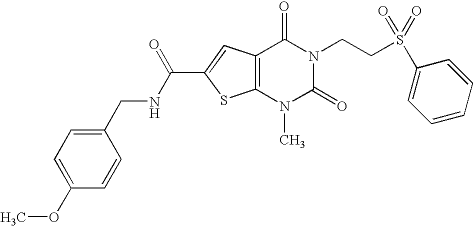 | 4 |
| 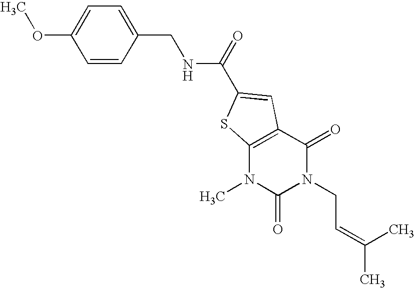 | 1.1 |
| 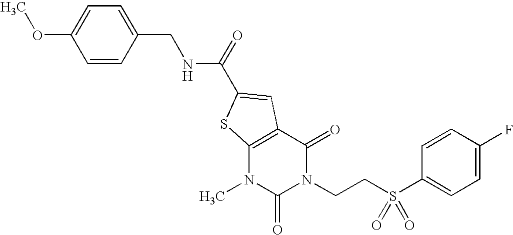 | 3.6 |
| 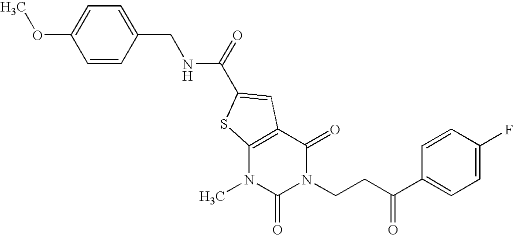 | 1.8 |
| 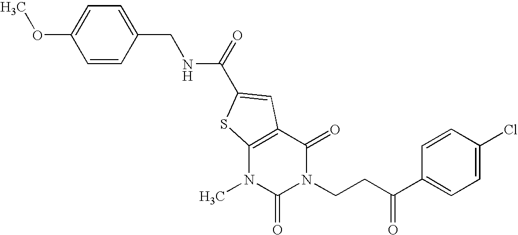 | 5.9 |
| 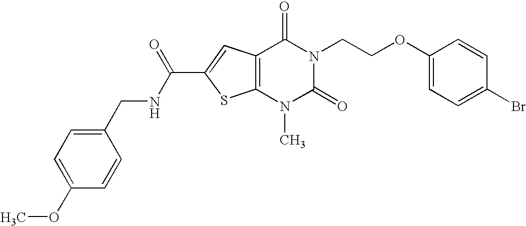 | 0.059 |
| 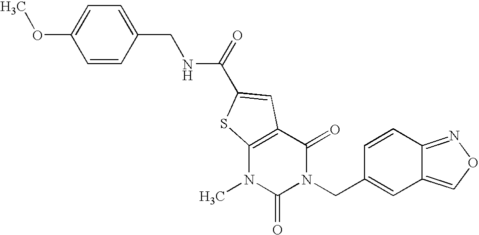 | 0.018 |
| 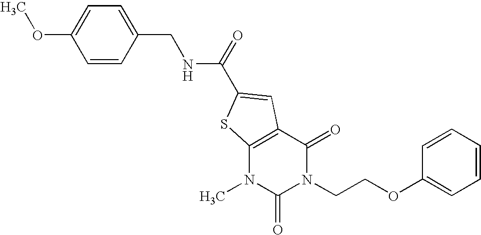 | 0.036 |
| 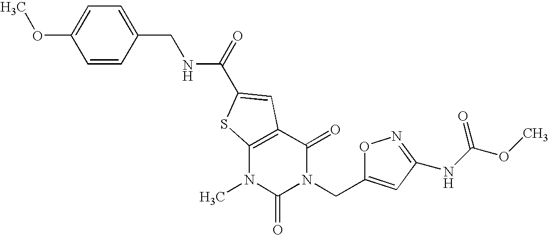 | 0.23 |
| 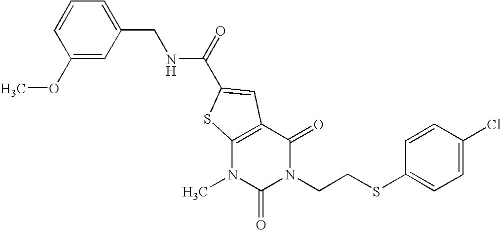 | 7.6 |
| 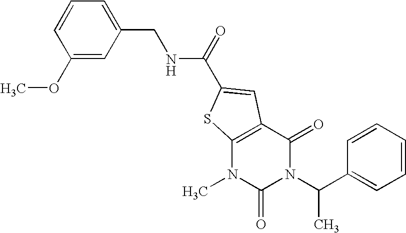 | 3.5 |
| 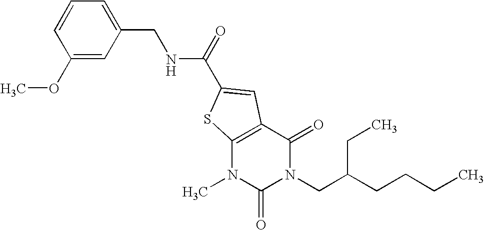 | 8.9 |
| 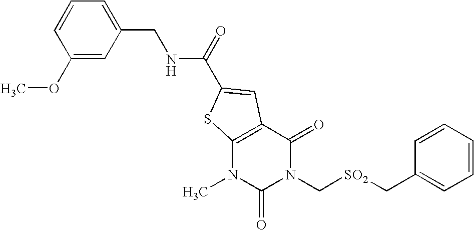 | 1.7 |
| 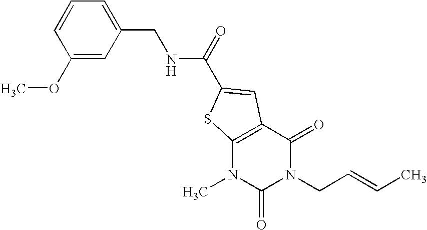 | 1.5 |
| 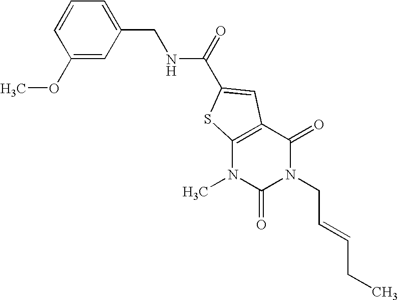 | 0.27 |
| 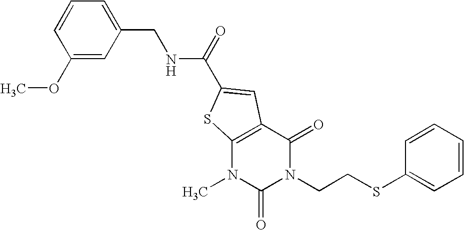 | 1.9 |
| 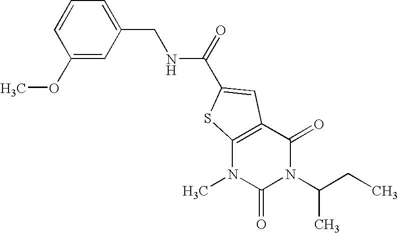 | 4.2 |
| 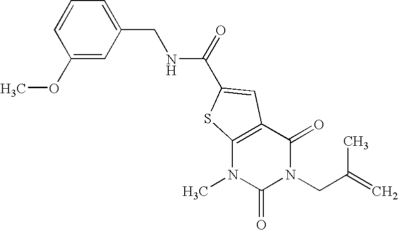 | 2.7 |
| 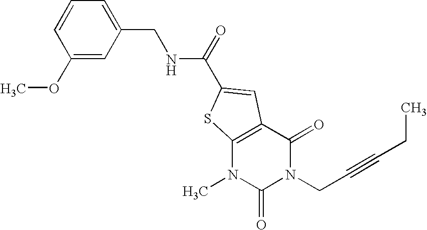 | 0.12 |
| 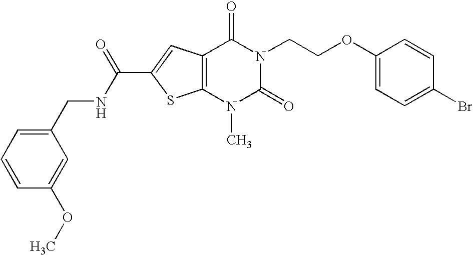 | 0.23 |
| 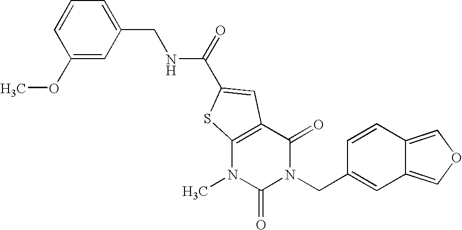 | 0.0505 |
| 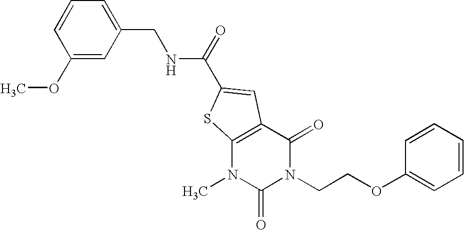 | 0.057 |
| 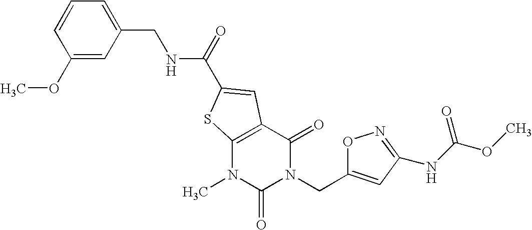 | 0.49 |
| 3-Benzyl-1-methyl-2,4-dioxo-1,2,3,4-tetrahydro-thieno[2,3-d]pyrimidine- | 0.0036 |
| 6-carboxylic acid pyridin-4-ylmethyl ester | |
| 3-Benzyl-1-methyl-2,4-dioxo-1,2,3,4-tetrahydro-thieno[2,3-d]pyrimidine- | 3.1 |
| 6-carboxylic acid benzo[b]thiophen-2-ylmethyl ester | |
| 3-(1,3-Benzodioxol-5-ylmethyl)-1-methyl-2,4-dioxo-1,2,3,4-tetrahydro- | 0.0052 |
| thieno[2,3-d]pyrimidine-6-carboxylic acid benzyl ester | |
| 1-Methyl-2,4-dioxo-3-pyridin-4-ylmethyl-1,2,3,4-tetrahydro-thieno[2,3- | 0.00715 |
| d]pyrimidine-6-carboxylic acid benzyl ester | |
| 3-(4-tert-Butyl-benzyl)-1-methyl-2,4-dioxo-1,2,3,4-tetrahydro-thieno[2,3- | 0.056 |
| d]pyrimidine-6-carboxylic acid benzyl ester | |
| 1-Methyl-2,4-dioxo-3-(4-trifluoromethoxy-benzyl)-1,2,3,4-tetrahydro- | 0.0845 |
| thieno[2,3-d]pyrimidine-6-carboxylic acid benzyl ester | |
| 1-Methyl-3-naphthalen-2-ylmethyl-2,4-dioxo-1,2,3,4-tetrahydro- | 0.0275 |
| thieno[2,3-d]pyrimidine-6-carboxylic acid benzyl ester | |
| 3-(4-Cyano-benzyl)-1-methyl-2,4-dioxo-1,2,3,4-tetrahydro-thieno[2,3- | 0.00645 |
| d]pyrimidine-6-carboxylic acid benzyl ester | |
| 3-Benzyl-1-methyl-2,4-dioxo-1,2,3,4-tetrahydro-thieno[2,3-d]pyrimidine- | 0.0185 |
| 6-carboxylic acid benzofuran-5-ylmethyl ester | |
| 3-(3,5-Dimethoxy-benzyl)-1-methyl-2,4-dioxo-1,2,3,4-tetrahydro- | 0.0205 |
| thieno[2,3-d]pyrimidine-6-carboxylic acid benzyl ester | |
| 3-(4-Carboxy-benzyl)-1-methyl-2,4-dioxo-1,2,3,4-tetrahydro-thieno[2,3- | 8 |
| d]pyrimidine-6-carboxylic acid 2-ethoxy-benzyl ester | |
| 3-Benzyl-1-methyl-2,4-dioxo-1,2,3,4-tetrahydro-thieno[2,3-d]pyrimidine- | 2.8 |
| 6-carboxylic acid [2-(3-ethoxy-phenyl)-ethyl]-amide | |
| 3-Benzyl-1-methyl-2,4-dioxo-1,2,3,4-tetrahydro-thieno[2,3-d]pyrimidine- | 2.7 |
| 6-carboxylic acid 3-chloro-4-fluoro-benzylamide | |
| 3-Benzyl-1-methyl-2,4-dioxo-1,2,3,4-tetrahydro-thieno[2,3-d]pyrimidine- | 1 |
| 6-carboxylic acid 3-chloro-benzylamide | |
| 3-Benzyl-1-methyl-2,4-dioxo-1,2,3,4-tetrahydro-thieno[2,3-d]pyrimidine- | 0.25 |
| 6-carboxylic acid 3-trifluoromethyl-benzylamide | |
| 3-Benzyl-1-methyl-2,4-dioxo-1,2,3,4-tetrahydro-thieno[2,3-d]pyrimidine- | 0.38 |
| 6-carboxylic acid (pyridin-3-ylmethyl)-amide | |
| 3-Benzyl-1-methyl-2,4-dioxo-1,2,3,4-tetrahydro-thieno[2,3-d]pyrimidine- | 0.12 |
| 6-carboxylic acid 4-methoxy-benzylamide | |
| 3-Benzyl-1-methyl-2,4-dioxo-1,2,3,4-tetrahydro-thieno[2,3-d]pyrimidine- | 0.044 |
| 6-carboxylic acid 3-methoxy-benzylamide | |
| 3-Benzyl-1-methyl-2,4-dioxo-1,2,3,4-tetrahydro-thieno[2,3-d]pyrimidine- | 3.6 |
| 6-carboxylic acid (thiophen-2-ylmethyl)-amide | |
| 3-Benzyl-1-methyl-2,4-dioxo-1,2,3,4-tetrahydro-thieno[2,3-d]pyrimidine- | 9.9 |
| 6-carboxylic acid (5-methyl-furan-2-ylmethyl)-amide | |
| 3-Benzyl-1-methyl-2,4-dioxo-1,2,3,4-tetrahydro-thieno[2,3-d]pyrimidine- | 0.93 |
| 6-carboxylic acid 4-bromo-benzylamide | |
| 3-Benzyl-1-methyl-2,4-dioxo-1,2,3,4-tetrahydro-thieno[2,3-d]pyrimidine- | 2 |
| 6-carboxylic acid [2-(1H-indol-3-yl)-ethyl]-amide | |
| 3-Benzyl-1-methyl-2,4-dioxo-1,2,3,4-tetrahydro-thieno[2,3-d]pyrimidine- | 10 |
| 6-carboxylic acid 2,4-dimethoxy-benzylamide | |
| 3-Benzyl-1-methyl-2,4-dioxo-1,2,3,4-tetrahydro-thieno[2,3-d]pyrimidine- | 0.32 |
| 6-carboxylic acid 4-chloro-benzylamide | |
| 3-Benzyl-1-methyl-2,4-dioxo-1,2,3,4-tetrahydro-thieno[2,3-d]pyrimidine- | 1 |
| 6-carboxylic acid 3,4-dichloro-benzylamide | |
| 3-Benzyl-1-methyl-2,4-dioxo-1,2,3,4-tetrahydro-thieno[2,3-d]pyrimidine- | 0.27 |
| 6-carboxylic acid 4-fluoro-3-trifluoromethyl-benzylamide | |
| 3-Benzyl-1-methyl-2,4-dioxo-1,2,3,4-tetrahydro-thieno[2,3-d]pyrimidine- | 7.7 |
| 6-carboxylic acid (2-pyridin-2-yl-ethyl)-amide | |
| 3-Cyanomethyl-1-methyl-2,4-dioxo-1,2,3,4-tetrahydro-thieno[2,3- | 1.55 |
| d]pyrimidine-6-carboxylic acid 4-methoxy-beazylamide | |
| 3-(4-Cyclopropylsulfamoyl-benzyl)-1-methyl-2,4-dioxo-1,2,3,4- | 0.00825 |
| tetrahydro-thieno[2,3-d]pyrimidine-6-carboxylic acid 3-methoxy- | |
| benzylamide | |
| 1-Methyl-3-(6-nitro-pyridin-3-ylmethyl)-2,4-dioxo-1,2,3,4-tetrahydro- | 0.735 |
| thieno[2,3-d]pyrimidine-6-carboxylic acid 3-methoxy-benzylamide | |
| 1-Methyl-3-(6-nitro-pyridin-3-ylmethyl)-2,4-dioxo-1,2,3,4-tetrahydro- | 1.04 |
| thieno[2,3-d]pyrimidine-6-carboxylic acid 4-methoxy-benzylamide | |
| 1-Methyl-3-(6-nitro-pyridin-3-ylmethyl)-2,4-dioxo-1,2,3,4-tetrahydro- | 1.17 |
| thieno[2,3-d]pyrimidine-6-carboxylic acid (2-methoxy-pyridin-4- | |
| ylmethyl)-amide | |
| 3-Cyclohexylmethyl-1-methyl-2,4-dioxo-1,2,3,4-tetrahydro-thieno[2,3- | 0.22 |
| d]pyrimidine-6-carboxylic acid (2-methoxy-pyridin-4-ylmethyl)-amide | |
| 3-(6-Amino-pyridin-3-ylmethyl)-1-methyl-2,4-dioxo-1,2,3,4-tetrahydro- | 0.66 |
| thieno[2,3-d]pyrimidine-6-carbaxylic acid 3-methoxy-benzylamide | |
| 1-Methyl-2,4-dioxo-3-(3-phenyl-prop-2-ynyl)-1,2,3,4-tetrahydro- | 0.007 |
| thieno[2,3-d]pyrimidine-6-carboxylic acid 4-methoxy-benzylamide | |

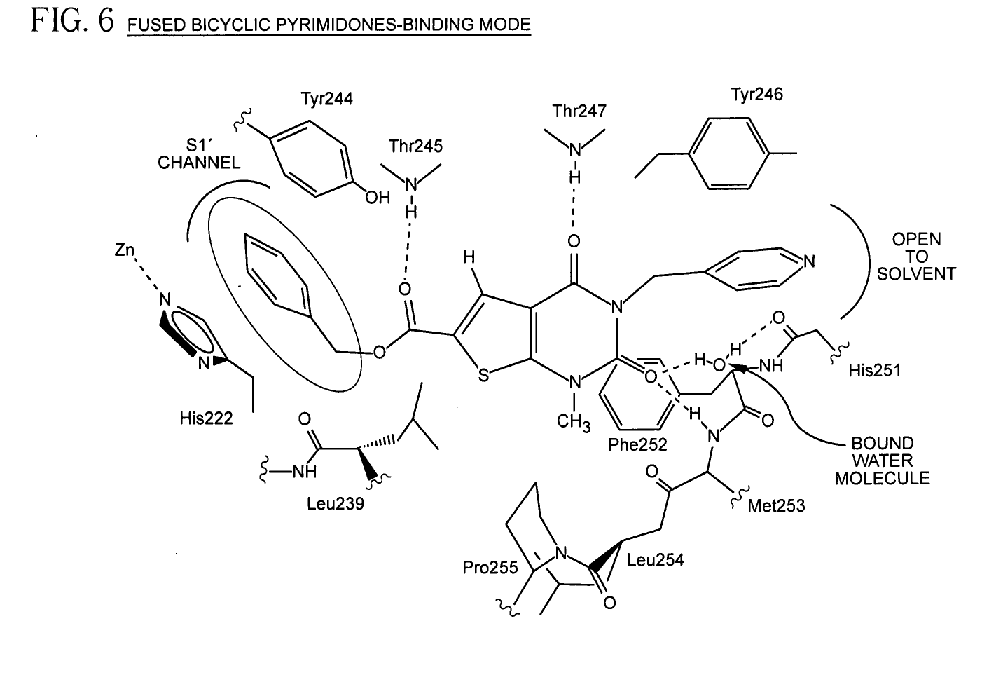

Binding of a representative compound of the above series is shown in FIG. 6. Again, binding for this compound is through two hydrophobic groups and three hydrogen bond acceptors, the third hydrogen bond acceptor binding to Met 253 and also via a bridging water molecule to the backbone carbonyl of His251.

Synthesis of some of the compounds referred to in Table III is described in the following further synthesis examples. The synthesis of the other compounds in the Table III is reported in our co-pending WO application which claims the priority of the application No. U.S. 60/268,756 filed on Feb. 14, 2001.

### Preparation 1

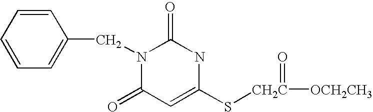

### (1-Benzyl-2,6-dioxo-1,2,3,6-tetrahydro-pyrimidin-4-ylsulfanyl)-acetic acid ethyl ester

To 250 ml of ethanol in a round bottom flask was added 3-benzyl-6-chloro-1H-pyrimidine-2,4-dione (11.55 g, 48.94 mmol), sodium carbonate (5.19 g, 48.94 mmol), and mercapto-acetic acid ethyl ester (6.47 g, 53.83 mmol). The mixture is stirred at reflux for 5 hours. The reaction solution is filtered, and the filtrate is chromatographied on a silica gel column, eluting with 4:1 Hexane:Ethyl Acetate (400 ml) followed by 1000 ml of 4:1 Dichloromethane:Ethyl Acetate. Removing the solvents by vacuum yielded 10.5 g of white powder identified as the titled product (67%). ¹H NMR (DMSO), δ 1.16 (t, J=7.1 Hz, 3H), 4.06 (s, 2H), 4.12 (q, J=7.1 Hz, 2H), 4.88 (s, 2H), 5.54 (s, 1H), 7.22-7.30 (m, 5H), 11.71 (broad s, 1H). MS (APCI−), m/z 321 (M⁺).

### Preparation 2

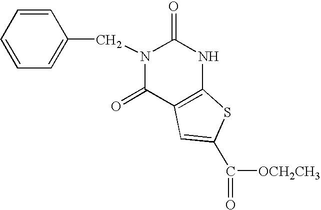

### 3-Benzyl-2,4-dioxo-1,2,3,4-tetrahydro-thieno[2,3-d]pyrimidine-6-carboxylic acid ethyl ester

To a solution of (1-benzyl-2,6-dioxo-1,2,3,6-tetrahydro-pyrimidin-4-ylsulfanyl)-acetic acid ethyl ester from Preparation 1 (6.37 g, 19.8 mmol) in anhydrous DMF (60 ml) was added POCl₃ (9.11 g, 59.5 mmol) dropwise. The reaction is then stirred at room temperature overnight, and then heated to 70° C. for 30 minutes. The reaction is cooled to room temperature and poured into 600 ml of stirring ice water. The product is filtered and washed with water to yield 6.2 g (95%) very light yellow powder as the titled compound. ¹H NMR (DMSO), δ 1.27 (t, J=7.1 Hz, 3H), 4.26 (q, J=7.1 Hz, 2H), 5.00 (s, 2H), 7.19-7.29 (m, 5H), 7.76 (s, 1H), 12.6 (broad s, 1H). MS (APCI−), m/z 331 (M⁺).

### Preparation 3

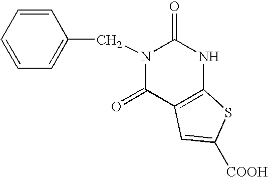

### 3-Benzyl-2,4-dioxo-1,2,3,4-tetrahydro-thieno[2,3-d]pyrimidine-6-carboxylic acid

To a solution of 3-benzyl-2,4-dioxo-1,2,3,4-tetrahydro-thieno[2,3-d]pyrimidine-6-carboxylic acid ethyl ester from Preparation 2 (2.9 g, 8.79 mmol) in a solution of 90% THF:10% water (v/v) was added lithium hydroxide (3.69 g, 87.9 mmol). The solution is refluxed for 2 hours. The solvent was removed by vacuum, and the residual was diluted with water (100 ml). HCl was added until the solution has a pH of 1. The solution was extracted with ethyl acetate (3×100 ml). The combined organic layer was concentrated to yield 2.62 g of white powder as product (96%). ¹H NMR (DMSO), δ 4.99 (s, 2H), 7.19-7.29 (m, 5H), 7.68 (s, 1H). MS (APCI−), m/z 331 (M⁺).

### SYNTHESIS EXAMPLE 17

### 3-Benzyl-2,4-dioxo-1,2,3,4-tetrahydro-thieno[2,3-d]pyrimidine-6-carboxylic acid benzyl ester

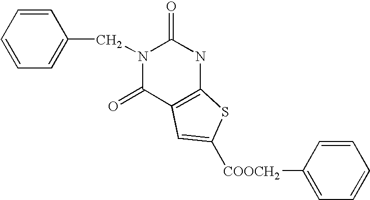

A dichloromethane (30 ml) solution of 3-benzyl-2,4-dioxo-1,2,3,4-tetrahydro-thieno[2,3-d]pyrimidine-6-carboxylic acid (0.8 g, 2.65 mmol), from Preparation 3, 1-cyclohexyl-3-(2-morpholinoethyl)carbodiimide metho-p-toluenesulfonate (CMC, 1.35 g, 3.18 mmol), and benzyl alcohol (0.32 g, 2.91 mmol) is refluxed for 3 hours. The solution is then diluted with dichloromethane (100 ml) and washed with water (3×100 ml). The organic layer is concentrated and purified by chromatography over a silica gel column using 2:1 Hexane:Ethyl Acetate to yield 120 mg of white solid as product (12%). MP: 195-197° C.; ¹H NMR (CDCl₃), δ 5.18 (s, 2H), 5.33 (s, 2H), 7.26-7.49 (m, 10H), 8.03 (s, 1H), 10.84 (s, 1H). MS (APCI−), m/z 303 (M⁺). Calculated for C₂₁H₁₆N₂O₄S₁: C, 64.27; H, 4.11; N, 7.14. Found: C, 64.24; H, 3.80; N, 7.04.

### SYNTHESIS EXAMPLE 18

### 3-Benzyl-2,4-dioxo-1,2,3,4-tetrahydro-thieno[2,3-d]pyrimidine-6-carboxylic acid pyridin-4-ylmethyl ester

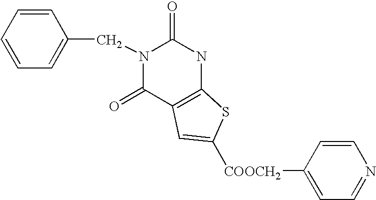

The procedure of Synthesis Example 17 was repeated, except that benzyl alcohol is replaced with 4-pyridyl methyl alcohol to provide 3-benzyl-2,4-dioxo-1,2,3,4-tetrahydro-thieno[2,3-d]pyrimidine-6-carboxylic acid pyridin-4-ylmethyl ester as a white powder. (32%). MP: 248-250° C.; ¹H NMR (DMSO), δ 5.00 (s, 2H), 5.36 (s, 2H), 7.22-7.34 (m, 5H), 7.41 (d, J=5.7 Hz, 2H), 7.91 (s, 1H), 8.57 (d, J=5.7 Hz, 2H), 12.62 (broad s,1H). MS (APCI−), m/z 394 (M⁺).

### SYNTHESIS EXAMPLE 19

### 3-Benzyl-1-methyl-2,4-dioxo-1,2,3,4-tetrahydro-thieno[2,3-d]pyrimidine-6-carboxylic acid benzyl ester

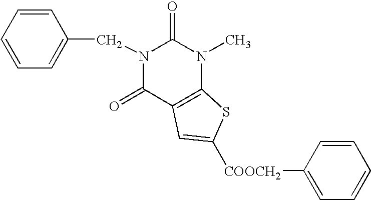

To a solution of 3-benzyl-2,4-dioxo-1,2,3,4-tetrahydro-thieno[2,3-d]-pyrimidine-6-carboxylic acid benzyl ester (300 mg, 0.765 mmol) in DMF was added NaH (46 mg, 1.5 mmol). After 5 minutes, MeI (0.15 ml, 2.3 mmol) was added, and the reaction mixture was stirred at room temperature for 30 minutes. After removal of all volatiles, the residue was purified using flash chromatography to give the desired product as a white solid (204 mg, 66%). R_{f}=0.51 (2:1 hexane/EtOAc). MP: 143-145° C. Calculated for C₂₂H₁₈N₂O₄S₁: C, 65.01; H, 4.46; N, 6.89. Found: C, 64.61; H, 4.31; N, 6.74.

### SYNTHESIS EXAMPLE 20

### 3-Benzyl-2,4-dioxo-1,2,3,4-tetrahydro-thieno[2,3-d]pyrimidine-6-carboxylic acid 1,3-benzodioxol-5-ylmethyl ester

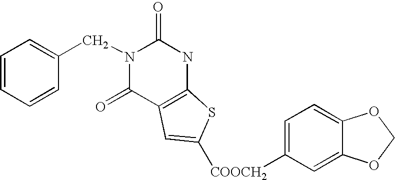

The procedure of Synthesis Example 17 was repeated, except that benzyl alcohol is replaced with benzo[1,3]dioxol-5-yl-methanol to give 3-benzyl-2,4-dioxo-1,2,3,4-tetrahydro-thieno[2,3-d]pyrimidine-6-carboxylic acid 1,3-benzodioxol-5-ylmethyl ester as a white solid. ¹H NMR (d₈-THF), δ 10.86 (s, 1H), 7.89 (s, 1H), 6.80-7.49 (m, 8H), 5.96 (s, 2H), 5.21 (s, 2H), 5.09 (s, 2H). MS (APCI−), m/z 393.2 (M⁺+1).

### SYNTHESIS EXAMPLE 21

### 3-Benzyl-1-methyl-2,4-dioxo-1,2,3,4-tetrahydro-thieno[2,3-d]pyrimidine-6-carboxylic acid benzyl amide

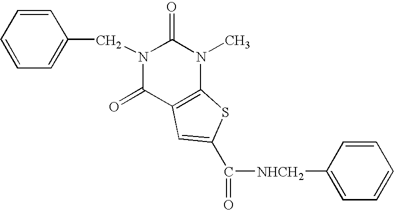

A dichloromethane (30 ml) solution of 3-benzyl-1-methyl-2,4-dioxo-1,2,3,4-tetrahydro-thieno[2,3-d]pyrimidine-6-carboxylic acid (367 mg, 1.16 mmol), CMC (392 g, 0.92 mmol), and benzylamnine (149 mg, 1.39 mmol) is refluxed for 3 hours. The solution is then diluted with dichloromethane (100 ml) and washed with water (3×100 ml). The organic layer is concentrated and purified by chromatography over a silica gel column using 1:1 Hexane:Ethyl Acetate to yield 200 mg of white solid as product. ¹H NMR (d₈-THF), δ 9.23 (t, 1H), 8.11 (s, 1H), 7.20-7.38 (m, 10H), 5.04 (s, 2H), 4.43 (s, 2H), 3.46 (s, 3H). MS (APCI−), m/z 406.1 (M⁺+1).

Substituted Quinazolines

We have made a fourth group of compounds which are substituted quinazolines and are inhibitors of matrix metalloproteinase enzymes, and especially MMP-13. Preferred compounds that we have made, and their ability to inhibit the activity of MMP-13 are summarized in Table IVa and Table IVb below:

TABLE IVa

| | | MMP13 |
|---|---|---|
| Name | Structure | IC₅₀ μM |
| 3-Benzyl-2,4-dioxo-1,2,3,4- tetrahydroquinazoline-6-carboxylic acid benzylamide | 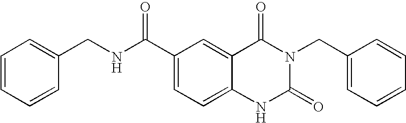 | 0.193 |
| 3-Benzyl-2,4-dioxo-1,2,3,4- tetrahydroquinazoline-6-carboxylic acid (4-pyridylmethyl)amide | 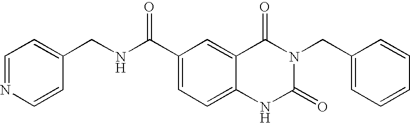 | 0.183 |
| 3-Benzyl-2,4-dioxo-1,2,3,4- tetrahydroquinazoline-6-carboxylic acid (benzo[1,3]dioxol-5- ylmethyl)amide | 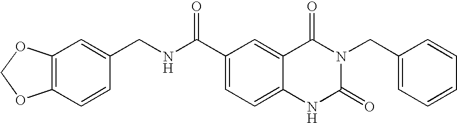 | 0.021 |
| 3-Benzyl-2,4-dioxo-1,2,3,4- tetrahydroquinazoline-6-carboxylic acid (2-thienylmethyl)amide | 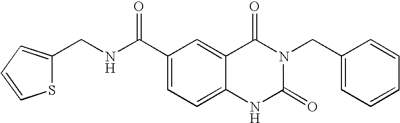 | 1.87 |
| 3-Benzyl-2,4-dioxo-1,2,3,4- tetrahydroquinazoline-6-carboxylic acid (3-pyridylmethyl)amide | 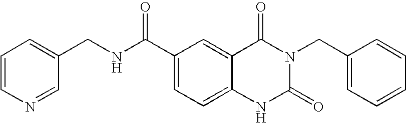 | 0.366 |
| 3-Benzyl-2,4-dioxo-1,2,3,4- tetrahydroquinazoline-6-carboxylic acid 4-methoxybenzylamide | 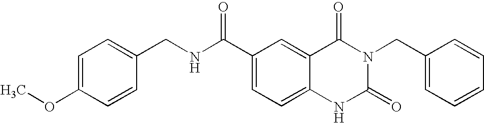 | 0.049 |
| 3-Benzyl-2,4-dioxo-1,2,3,4- tetrahydroquinazoline-6-carboxylic acid 4-chlorobenzylamide | 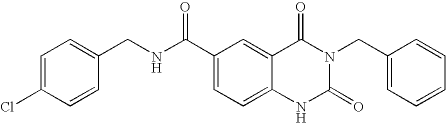 | 0.167 |
| 3-Benzyl-2,4-dioxo-1,2,3,4- tetrahydroquinazoline-6-carboxylic acid 4-methylbenzylamide | 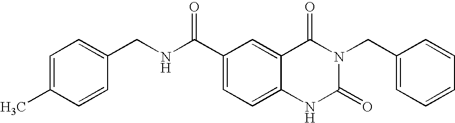 | 1.32 |
| 3-Benzyl-1-methyl-2,4-dioxo-1,2,3,4- tetrahydroquinazoline-6-carboxylic acid (benzo[1,3]dioxol-5- ylmethyl)amide | 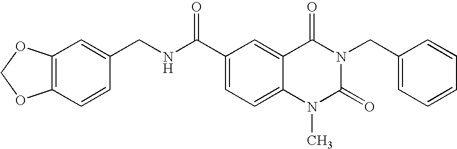 | 0.005 |
| 3-Benzyl-1-methyl-2,4-dioxo-1,2,3,4- tetrahydroquinazoline-6-carboxylic acid benzylamide | 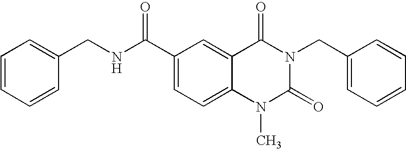 | 0.057 |
| Methyl 4-({[1-(3-benzyl-1-methyl- 2,4-dioxo-1,2,3,4-tetrahydroquin- azolin-6-yl)methanoyl]amino}methyl)benzoate | 2.25 | |
| 3-Benzyl-1-methyl-2,4-dioxo-1,2,3,4- tetrahydroquinazoline-6-carboxylic acid 4-hydroxy-3- methoxybenzylamide | 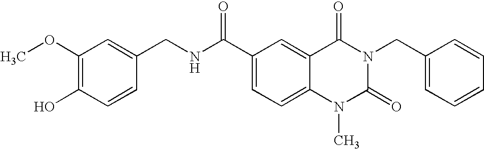 | 0.051 |
| 3-Benzyl-1-methyl-2,4-dioxo-1,2,3,4- tetrahydroquinazoline-6-carboxylic acid 4-methoxybenzylamide | 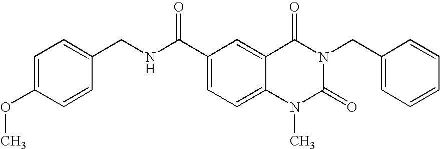 | 0.012 |
| 3-Benzyl-1-methyl-2,4-dioxo-1,2,3,4- tetrahydroquinazoline-6-carboxylic acid (4-pyridylmethyl)amide | 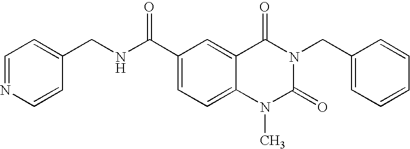 | 0.051 |
| 1-Methyl-2,4-dioxo-3-phenethyl- 1,2,3,4-tetrahydroquinazoline-6- carboxylic acid (benzo[1,3]dioxol- 5-ylmethyl)amide | 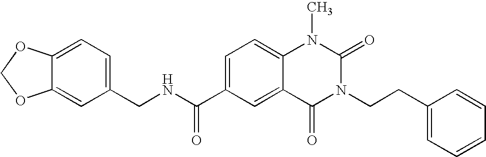 | 0.7 |
| 3-(4-Methoxybenzyl)-2,4-dioxo- 1,2,3,4-tetrahydroquinazoline-6- carboxylic acid (benzo[1,3]dioxol- 5-ylmethyl)amide | 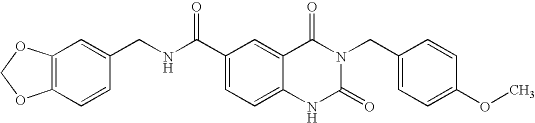 | 0.015 |
| 3-(4-Methoxybenzyl)-1-methyl-2,4- dioxo-1,2,3,4-tetrahydroquin- azoline-6-carboxylic acid (benzo[1,3]dioxol-5- ylmethyl)amide | 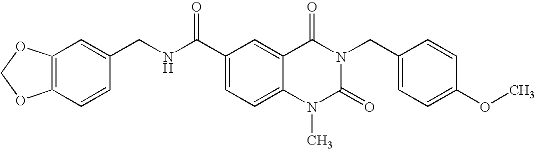 | 0.009 |
| 3-(4-Methoxybenzyl)-1-methyl-2,4- dioxo-1,2,3,4-tetrahydro-quinazoline-6-carboxylic acid 4- methoxybenzylamide | 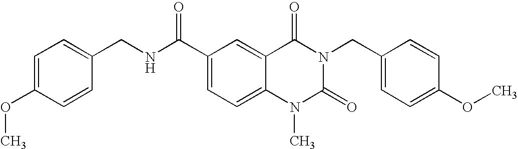 | 0.01 |
| 2,4-Dioxo-3-pyrid-4-ylmethyl-1,2,3,4- tetrahydroquinazoline-6-carboxylic acid (benzo[1,3]dioxol-5- ylmethyl)amide | 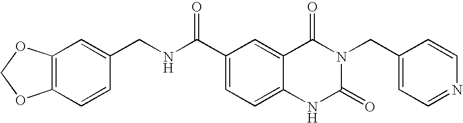 | 0.051 |
| 2,4-Dioxo-3-thien-2-ylmethyl-1,2,3,4- tetrahydroquinazoline-6-carboxylic acid benzylamide | 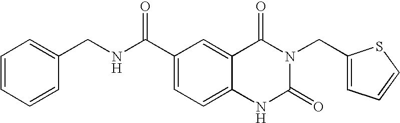 | 0.3 |
| 1-Methyl-2,4-dioxo-3-thien-2- ylmethyl-1,2,3,4- tetrahydroquinazoline-6-carboxylic acid benzylamide | 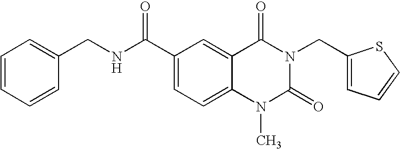 | 0.096 |
| 2,4-Dioxo-3-thien-2-ylmethyl- 1,2,3,4-tetrahydroquinazoline- 6-carboxylic acid (benzo[1,3]dioxol-5- ylmethyl)amide | 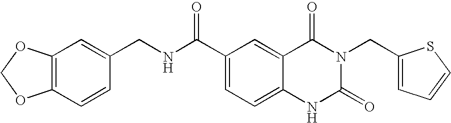 | 0.029 |
| 1-Methyl-2,4-dioxo-3-thien-2- ylmethyl-1,2,3,4- tetrahydroquinazoline-6-carboxylic acid (benzo[1,3]dioxol-5- ylmethyl)amide | 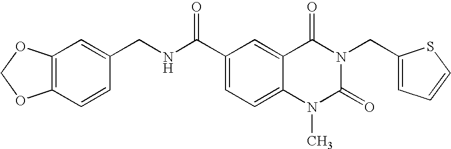 | 0.009 |
| 3-(4-Chlorobenzyl)-2,4-dioxo- 1,2,3,4-tetrahydroquinazoline-6- carboxylic acid (benzo[1,3]dioxol-5-ylmethyl)amide | 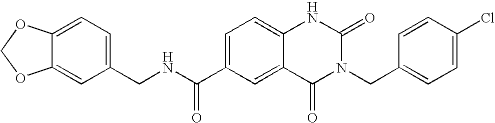 | 0.028 |
| 3-(4-Chlorobenzyl)-1-methyl-2,4- dioxo-1,2,3,4-tetrahydroquin-azoline- 6-carboxylic acid (benzo[1,3]dioxol-5-ylmethyl)amide | 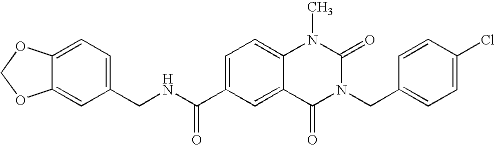 | 0.009 |
| 1,3-Dimethyl-2,4-dioxo-1,2,3,4- tetrahydroquinazoline-6-carboxylic acid (benzo[1,3]dioxol-5- ylmethyl)amide | 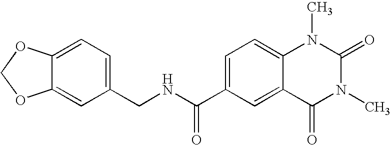 | 1.7 |
| 3-Benzo[1,3]dioxol-5-ylmethyl-2,4- dioxo-1,2,3,4-tetrahydroquin- azoline-6-carboxylic acid (benzo[1,3]dioxol-5-ylmethyl)amide | 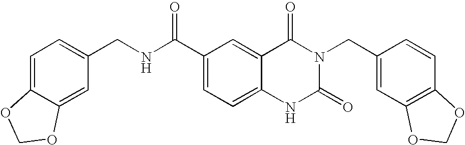 | 0.017 |
| 3-Benzo[1,3]dioxol-5-ylmethyl-1- methyl-2,4-dioxo-1,2,3,4- tetrahydroquinazoline-6-carboxylic acid (benzo[1,3]dioxol-5- ylmethyl)amide | 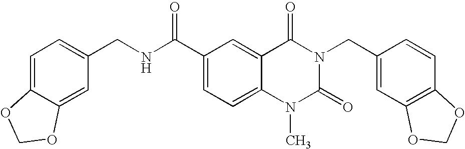 | 0.003 |
| 3-Benzyl-1-ethyl-2,4-dioxo-1,2,3,4- tetrahydroquinazoline-6-carboxylic acid (benzo[1,3]dioxol-5- ylmethyl)amide | 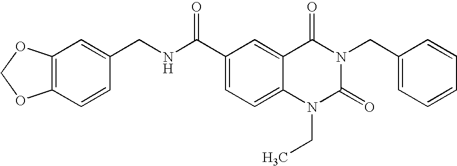 | 0.026 |
| 3-Benzyl-1-cyclopropylmethyl-2,4- dioxo-1,2,3,4-tetrahydroquin- azoline-6-carboxylic acid (benzo[1,3]dioxol-5- ylmethyl)amide | 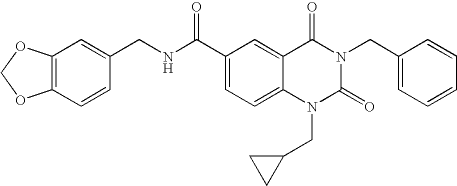 | 0.157 |
| 3-Benzyl-1-isobutyl-2,4-dioxo- 1,2,3,4-tetrahydroquinazoline- 6-carboxylic acid (benzo[1,3]dioxol-5-ylmethyl)amide | 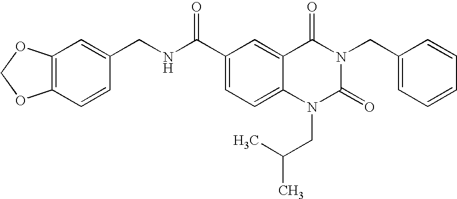 | 0.6 |
| 1-Methyl-2,4-dioxo-1,2,3,4- tetrahydroquinazoline-6-carboxylic acid (benzo[1,3]dioxol-5- ylmethyl)amide | 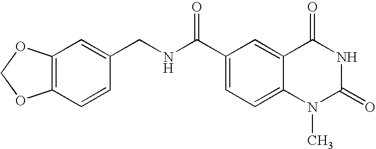 | 0.92 |
| 4-[6-(4-Methoxy-benzylcarbamoyl)- 1-methyl-2,4-dioxo-1,4- dihydro-2H-quinazolin-3- ylmethyl]-benzoic acid methyl ester | 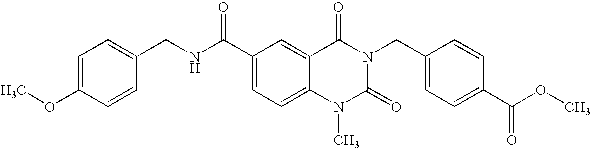 | 0.004 |
| 4-[6-(4-Methoxy-benzylcarbamoyl)- methyl-2,4-dioxo-1,4-dihydro-2H]- quinazolin-3-ylmethyl]-benzoic acid | 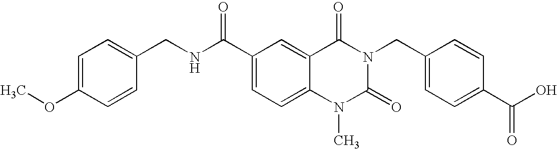 | 0.001 |
| 1-Methyl-2,4-dioxo-3-((E)-3- phenylallyl)-1,2,3,4-tetrahydroquin- azoline-6-carboxylic acid benzo[1,3]dioxol-5-ylmethyl)amide | 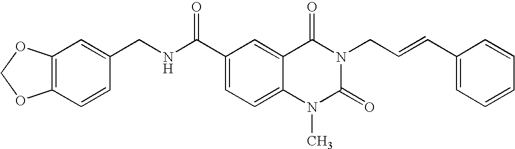 | 0.022 |
| Benzyl 3-benzyl-2,4-dioxo-1,2,3,4- tetrahydroquinazoline-6-carboxylate | 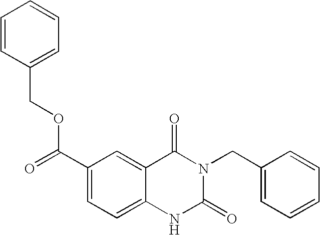 | 0.029 |
| Benzyl 3-benzyl-1-methyl-2,4-dioxo- 1,2,3,4-tetrahydroquinazoline-6- carboxylate | 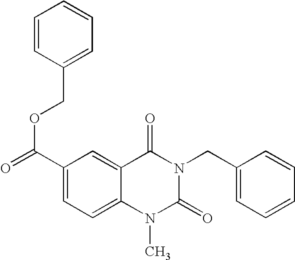 | 0.031 |
| 4-Pyridylmethyl 3-benzyl-2,4-dioxo- 1,2,3,4-tetrahydroquinazoline-6- carboxylate | 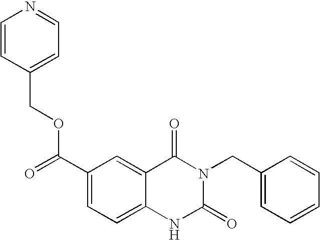 | 0.011 |
| 4-Pyridylmethyl 3-benzyl-1-methyl- 2,4-dioxo-1,2,3,4-tetrahydro- quinazoline-6-carboxylate | 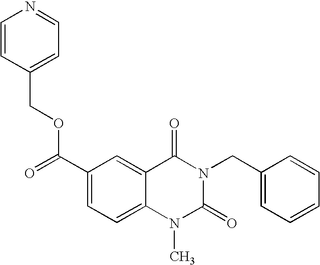 | 0.004 |
| Benzo[1,3]dioxol-5-ylmethyl 3- benzyl-2,4-dioxo-1,2,3,4- tetrahydro-quinazoline- 6-carboxylate | 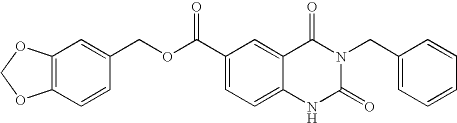 | 0.007 |
| Benzo[1,3]dioxol-5-ylmethyl 3- benzyl-1-methyl-2,4-dioxo- 1,2,3,4-tetrahydroquinazoline- 6-carboxylate | 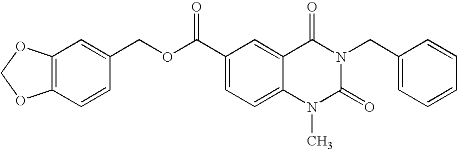 | 0.0025 |
| Benzyl 1-benzyl-2,4-dioxo-3-pyrid-4- ylmethyl-1,2,3,4-tetrahydroquin- azoline-6-carboxylate | 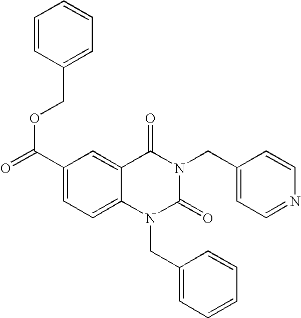 | 1.21 |
| 4-Pyridylmethyl 2,4-dioxo-3-thien-2- ylmethyl-1,2,3,4-tetrahydroquin- azoline-6-carboxylate | 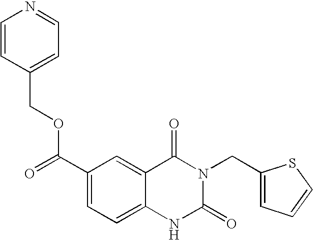 | 0.016 |
| 4-Pyridylmethyl 3-benzo[1,3]dioxol- 5-ylmethyl-2,4-dioxo-1,2,3,4- tetrahydroquinazoline-6-carboxylate | 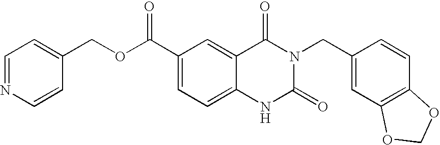 | 0.007 |
| Benzyl 3-benzyl-2,4-dioxo-1,2,3,4- tetrahydropyrido[2,3-d]pyrimidine-6-carboxylate | 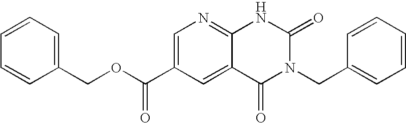 | 0.096 |
| 4-Pyridylmethyl 3-benzyl-2,4-dioxo- 1,2,3,4-tetrahydropyrido[2,3-d]- pyrimidine-6-carboxylate | 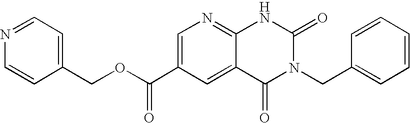 | 0.062 |
| 3-Benzyl-4-oxo-2-thioxo-1,2,3,4- tetrahydroquinazoline-6-carboxylic acid (benzo[1,3]dioxol-5- ylmethyl)amide | 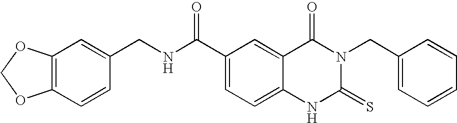 | 0.014 |
| 3-Benzyl-1-methyl-2,4-dioxo-1,2,3,4- tetrahydro-pyrido[2,3-d]pyrimidine-6-carboxylic acid (1,3-benzodioxol-5- ylmethyl)-amide | 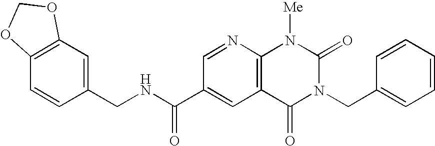 | 0.007 |
| 4-[6-(4-Methoxy-benzylcarbamoyl)- 1-methyl-2,4-dioxo-1,4-dihydro-2H- pyrido[2,3-d]pyrimidin-3-ylmethyl]- benzoic acid | 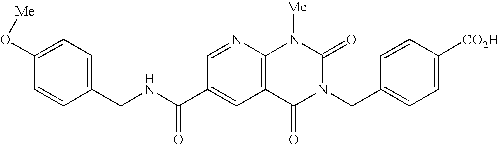 | 0.0016 |
| 3-(4-Cyano-benzyl)-1-methyl-2,4- dioxo-1,2,3,4-tetrahydro- pyrido[2,3-d]pyrimidine-6- carboxylic acid 4- methoxy-benzylamide | 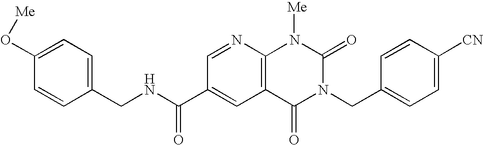 | 0.016 |
| 3-(4-Fluoro-benzyl)-1-methyl-2,4- dioxo-1,2,3,4-tetrahydro- pyrido[2,3-d]pyrimidine-6- carboxylic acid 4- methoxy-benzylamide | 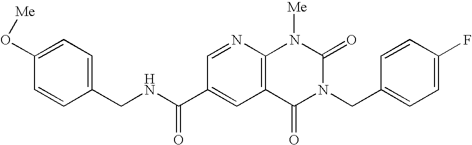 | 0.032 |
| 3-Benzyl-1-methyl-2,4-dioxo-1,2,3,4- tetrahydro-pyrido[3,4-d9 pyrimidine-6-carboxylic acid (1,3-benzodioxol-5- ylmethyl)-amide | 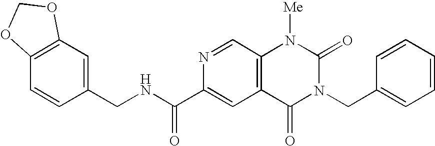 | 0.001 |
| Methyl 4-[6-(4-Methoxy-benzyl carbamoyl)-1-methyl-2,4-dioxo-1,4- dihydro-2H-pyrido[3,4-d]pyrimidin-3-ylmethyl]- benzoate | 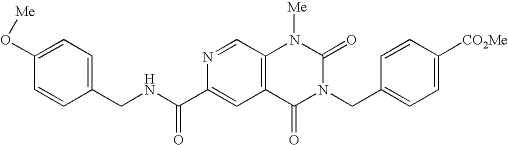 | 0.0017 |

TABLE IVb

| | MMP13 |
|---|---|
| Compound | IC₅₀ μM |
| 3-Benzyl-4-oxo-2-thioxo-1,2,3,4-tetrahydroquinazoline-6-carboxylic acid | 0.0140 |
| (benzo[1,3]dioxol-5-ylmethyl)amide | |
| 4-[6-(4-Hydroxy-benzylcarbamoyl)-1-methyl-2,4-dioxo-1,4-dihydro-2H- | 0.0020 |
| quinazolin-3-ylmethyl]-benzoic acid | |
| 3-(4-Dimethylcarbamoyl-benzyl)-1-methyl-2,4-dioxo-1,2,3,4- | 0.0120 |
| tetrahydroquinazoline-6-carboxylic acid 4-methoxy-benzylamide | |
| 1-Methyl-3-(4-methylcarbamoyl-benzyl)-2,4-dioxo-1,2,3,4-tetrahydro- | 0.0064 |
| quinazoline-6-carboxylic acid 4-methoxy-benzylamide | |
| 3-Allyl-1-methyl-2,4-dioxo-1,2,3,4-tetrahydro-quinazoline-6-carboxylic acid | 1.0100 |
| 4-methoxy-benzylamide | |
| 1-Methyl-2,4-dioxo-3-(2-pyrrol-1-yl-ethyl)-1,2,3,4-tetrahydro-quinazoline-6- | 1.4500 |
| carboxylic acid 4-methoxy-benzylamide | |
| 1-Methyl-2,4-dioxo-3-prop-2-ynyl-1,2,3,4-tetrahydro-quinazoline-6- | 0.6800 |
| carboxylic acid 4-methoxy-benzylamide | |
| 1-Methyl-3-(3-methyl-but-2-enyl)-2,4-dioxo-1,2,3,4-tetrahydro-quinazoline-6- | 0.2000 |
| carboxylic acid 4-methoxy-benzylamide | |
| 1-Methyl-2,4-dioxo-3-pyridin-2-ylmethyl-1,2,3,4-tetrahydro-quinazoline-6- | 0.4300 |
| carboxylic acid 4-methoxy-benzylamide | |
| 3-Carbamoylmethyl-1-methyl-2,4-dioxo-1,2,3,4-tetrahydro-quinazoline-6- | 1.9500 |
| carboxylic acid 4-methoxy-benzylamide, | |
| 1-Methyl-2,4-dioxo-3-pyridin-3-ylmethyl-1,2,3,4-tetrahydro-quinazoline-6- | 0.0460 |
| carboxylic acid 4-methoxy-benzylamide | |
| 1-Methyl-3-(1-methyl-piperidin-3-ylmethyl)-2,4-dioxo-1,2,3,4- | 5.4000 |
| tetrahydroquinazoline-6-carboxylic acid 4-methoxy-benzylamide | |
| 3-(4-Cyano-benzyl)-1-methyl-2,4-dioxo-1,2,3,4-tetrahydro-quinazoline-6- | 0.0080 |
| carboxylic acid 4-methoxy-benzylamide | |
| 3-(3-Cyano-benzyl)-1-methyl-2,4-dioxo-1,2,3,4-tetrahydro-quinazoline-6- | 0.0270 |
| carboxylic acid 4-methoxy-benzylamide | |
| 3-(2-Methoxy-ethyl)-1-methyl-2,4-dioxo-1,2,3,4-tetrahydro-quinazoline-6- | 1.3500 |
| carboxylic acid 4-methoxy-benzylamide | |
| 3-(3-Methoxy-benzyl)-1-methyl-2,4-dioxo-1,2,3,4-tetrahydro-quinazoline-6- | 0.0230 |
| carboxylic acid 4-methoxy-benzylamide | |
| 3-Cyclopropylmethyl-1-methyl-2,4-dioxo-1,2,3,4-tetrahydro-quinazoline-6- | 0.9500 |
| carboxylic acid 4-methoxy-benzylamide | |
| 1-Methyl-3-(2-morpholin-4-yl-ethyl)-2,4-dioxo-1,2,3,4-tetrahydro- | 9.3000 |
| quinazoline-6-carboxylic acid 4-methoxy-benzylamide | |
| 3-Cyclohexylmethyl-1-methyl-2,4-dioxo-1,2,3,4-tetrahydro-quinazoline-6- | 0.0420 |
| carboxylic acid 4-methoxy-benzylamide | |
| 1-Methyl-2,4-dioxo-3-(3-phenyl-propyl)-1,2,3,4-tetrahydro-quinazoline-6- | 0.5900 |
| carboxylic acid 4-methoxy-benzylamide | |
| 3-(4-Fluoro-benzyl)-1-methyl-2,4-dioxo-1,2,3,4-tetrahydro-quinazoline-6- | 0.0145 |
| carboxylic acid 4-methoxy-benzylamide | |
| 3-[2-(4-Diethylamino-phenyl)-2-oxo-ethyl]-1-methyl-2,4-dioxo-1,2,3,4- | 3.6400 |
| tetrahydro-quinazoline-6-carboxylic acid 4-methoxy-benzylamide | |
| Ethyl [6-(4-methoxy-benzylcarbamoyl)-1-methyl-2,4-dioxo-1,4-dihydro-2H- | 0.6500 |
| quinazolin-3-yl]-acetate | |
| 3-(2-Hydroxy-ethyl)-1-methyl-2,4-dioxo-1,2,3,4-tetrahydroquinazoline-6- | 6.3500 |
| carboxylic acid 4-methoxy-benzylamide | |
| Methyl 3-[6-(4-methoxy-benzylcarbamoyl)-1-methyl-2,4-dioxo-1,4-dihydro- | 2.1000 |
| 2H-quinazolin-3-yl]-propionate | |
| 4-[6-(4-Methoxy-benzylcarbamoyl)-1-methyl-2,4-dioxo-1,4-dihydro-2H- | 9.7000 |
| quinazolin-3-yl]-propionic acid | |
| Ethyl 4-[6-(4-methoxy-benzylcarbamoyl)-1-methyl-2,4-dioxo-1,4-dihydro-2H- | 2.5500 |
| quinazolin-3-yl]-butyrate | |
| 4-[6-(4-Methoxy-benzylcarbamoyl)-1-methyl-2,4-dioxo-1,4-dihydro-2H- | 1.1500 |
| quinazolin-3-yl]-butyric acid | |
| Methyl {4-[6-(4-methoxy-benzylcarbamoyl)-1-methyl-2,4-dioxo-1,4-dihydro- | 0.0034 |
| 2H-quinazolin-3-ylmethyl]-phenyl}-acetate | |
| {4-[6-(4-Methoxy-benzylcarbamoyl)-1-methyl-2,4-dioxo-1,4-dihydro-2H- | 0.0022 |
| quinazolin-3-ylmethyl]-phenyl}-acetic acid | |
| 3-(4-Dimethylcarbamoylmethyl-benzyl)-1-methyl-2,4-dioxo-1,2,3,4- | 0.0080 |
| tetrahydro-quinazoline-6-carboxylic acid 4-methoxy-benzylamide | |
| 1-Methyl-2,4-dioxo-3-[(E)-3-(pyridin-3-yl)-allyl]-1,2,3,4-tetrahydro- | 0.0950 |
| quinazoline-6-carboxylic acid 4-methoxy-benzylamide | |
| 1-Methyl-2,4-dioxo-3-[(E)-3-(pyridin-4-yl)-allyl]-1,2,3,4-tetrahydro- | 0.0350 |
| quinazoline-6-carboxylic acid 4-methoxy-benzylamide | |
| 1-Methyl-2,4-dioxo-3-(4-sulfamoyl-benzyl)-1,2,3,4-tetrahydroquinazoline-6- | 0.0080 |
| carboxylic acid 4-methoxy-benzylamide | |
| 3-(4-Methanesulfonyl-benzyl)-1-methyl-2,4-dioxo-1,2,3,4-tetrahydro- | 0.0125 |
| quinazoline-6-carboxylic acid 4-methoxy-benzylamide | |
| 3-(4-Dimethylsulfamoyl-benzyl)-1-methyl-2,4-dioxo-1,2,3,4-tetrahydro- | 0.0070 |
| quinazoline-6-carboxylic acid 4-methoxy-benzylamide | |
| 3-[4-(2-Dimethylamino-ethylsulfamoyl)-benzyl]-1-methyl-2,4-dioxo-1,2,3,4- | 0.0550 |
| tetrahydro-quinazoline-6-carboxylic acid 4-methoxy-benzylamide | |
| 1-Methyl-3-(4-methylsulfamoyl-benzyl)-2,4-dioxo-1,2,3,4-tetrahydro- | 0.0044 |
| quinazoline-6-carboxylic acid 4-methoxy-benzylamide | |
| Methyl 3-[6-(4-Methoxy-benzylcarbamoyl)-1-methyl-2,4-dioxo-1,4-dihydro- | 0.1200 |
| 2H-quinazolin-3-ylmethyl]-benzoate | |
| 3-[6-(4-Methoxy-benzylcarbamoyl)-1-methyl-2,4-dioxo-1,4-dihydro-2H- | 0.0180 |
| quinazolin-3-ylmethyl]-benzoic acid | |
| (E) Methyl-4-[6-(4-methoxy-benzylcarbamoyl)-1-methyl-2,4-dioxo-1,4- | 0.3150 |
| dihydro-2H-quinazolin-3-yl]-but-2-enoate | |
| 4-[6-(4-Methoxy-benzylcarbamoyl)-1-methyl-2,4-dioxo-1,4-dihydro-2H- | 1.4000 |
| quinazolin-3-yl]-but-2-enoic acid | |
| Methyl 5-[6-(4-methoxy-benzylcarbamoyl)-1-methyl-2,4-dioxo-1,4-dihydro- | 0.2900 |
| 2H-quinazolin-3-ylmethyl]-furan-2-carboxylate | |
| 5-[6-(4-Methoxy-benzylcarbamoyl)-1-methyl-2,4-dioxo-1,4-dihydro-2H- | 0.0570 |
| quinazolin-3-ylmethyl]-furan-2-carboxylic acid | |
| Methyl 5-[6-(4-methoxy-benzylcarbamoyl)-1-methyl-2,4-dioxo-1,4-dihydro- | 0.0210 |
| 2H-quinazolin-3-ylmethyl]-thiophene-2-carboxylate | |
| 5-[6-(4-Methoxy-benzylcarbamoyl)-1-methyl-2,4-dioxo-1,4-dihydro-2H- | 0.0084 |
| quinazolin-3-ylmethyl]-thiophene-2-carboxylic acid | |
| 1-Methyl-3-(4-nitro-benzyl)-2,4-dioxo-1,2,3,4-tetrahydro-quinazoline-6- | 0.0140 |
| carboxylic acid 4-methoxy-benzylamide | |
| 3-(4-Amino-benzyl)-1-methyl-2,4-dioxo-1,2,3,4-tetrahydro-quinazoline-6- | 0.0093 |
| carboxylic acid 4-methoxy-benzylamide | |
| 3-(4-Dimethylamino-benzyl)-1-methyl-2,4-dioxo-1,2,3,4-tetrahydro- | 0.0280 |
| quinazoline-6-carboxylic acid 4-methoxy-benzylamide | |
| 3-(4-Acetylamino-benzyl)-1-methyl-2,4-dioxo-1,2,3,4-tetrahydro-quinazoline- | 0.0090 |
| 6-carboxylic acid 4-methoxy-benzylamide | |
| 3-[4-(N,N-methylsulfonylamino)-benzyl]-1-methyl-2,4-dioxo-1,2,3,4- | 0.0750 |
| tetrahydro-quinazoline-6-carboxylic acid 4-methoxy-benzylamide | |
| 3-Benzofurazan-5-ylmethyl-1-methyl-2,4-dioxo-1,2,3,4-tetrahydro- | 0.0180 |
| quinazoline-6-carboxylic acid 4-methoxy-benzylamide | |
| 3-[2-(4-Fluorophenoxy)-ethyl]-1-methyl-2,4-dioxo-1,2,3,4-tetrahydro- | 0.1500 |
| quinazoline-6-carboxylic acid 4-methoxy-benzylamide | |
| 3-(2-Benzenesulfonyl-ethyl)-1-methyl-2,4-dioxo-1,2,3,4-tetrahydro- | 0.8400 |
| quinazoline-6-carboxylic acid 4-methoxy-benzylamide | |
| 3-(3-fluoro-4-methoxy-benzyl)-1-methyl-2,4-dioxo-1,2,3,4-tetrahydro- | 0.0058 |
| quinazoline-6-carboxylic acid 4-methoxy benzylamine | |
| 1-Methyl-2,4-dioxo-3-[4-(2H-tetrazol-5-yl)-benzyl]-1,2,3,4-tetrahydro- | 0.0009 |
| quinazoline-6-carboxylic acid 4-methoxy-benzylamide | |
| 1-Methyl-3-[4-(5-methyl-1,2,4-oxadiazol-3-yl)-benzyl]-2,4-dioxo-1,2,3,4- | 0.0049 |
| tetrahydro-quinazoline-6-carboxylic acid 4-methoxy-benzylamide | |
| 1-Methyl-3-[4-(3-methyl-1,2,4-oxadiazol-5-yl)-benzyl]-2,4-dioxo-1,2,3,4- | 0.0029 |
| tetrahydro-quinazoline-6-carboxylic acid 4-methoxy-benzylamide | |
| Methyl 2-chloro-4-[6-(4-methoxy-benzylcarbamoyl)-1-methyl-2,4-dioxo-1,4- | 0.1400 |
| dihydro-2H-quinazolin-3-ylmethyl]-benzoate | |
| 2-Chloro-4-[6-(4-methoxy-benzylcarbamoyl)-1-methyl-2,4-dioxo-1,4-dihydro- | 0.0040 |
| 2H-quinazolin-3-ylmethyl]-benzoic acid | |
| 1-Methyl-3-[4-(1-methyl-1H-tetrazol-5-yl)-benzyl]-2,4-dioxo-1,2,3,4- | 0.0023 |
| tetrahydro-quinazoline-6-carboxylic acid 4-methoxy-benzylamide | |
| 1-Methyl-3-[4-(2-methyl-2H-tetrazol-5-yl)-benzyl]-2,4-dioxo-1,2,3,4- | 0.0040 |
| tetrahydro-quinazoline-6-carboxylic acid 4-methoxy-benzylamide, | |
| Methyl 2-methoxy-4-[6-(4-methoxy-benzylcarbamoyl)-1-methyl-2,4-dioxo- | 0.0500 |
| 1,4-dihydro-2H-quinazolin-3-ylmethyl]-benzoate | |
| 2-Methoxy-4-[6-(4-methoxy-benzylcarbamoyl)-1-methyl-2,4-dioxo-1,4- | 0.0045 |
| dihydro-2H-quinazolin-3-ylmethyl]-benzoic acid | |
| Methyl 2-hydroxy-4-[6-(4-methoxy-benzylcarbamoyl)-1-methyl-2,4-dioxo- | 0.0043 |
| 1,4-dihydro-2H-quinazolin-3-ylmethyl]-benzoate | |
| 2-Hydroxy-4-[6-(4-methoxy-benzylcarbamoyl)-1-methyl-2,4-dioxo-1,4- | 0.0016 |
| dihydro-2H-quinazolin-3-ylmethyl]-benzoic acid | |
| Methyl 2-methyl-4-[6-(4-methoxy-benzylcarbamoyl)-1-methyl-2,4-dioxo-1,4- | 0.0077 |
| dihydro-2H-quinazolin-3-ylmethyl]-benzoate | |
| 2-Methyl-4-[6-(4-methoxy-benzylcarbamoyl)-1-methyl-2,4-dioxo-1,4- | 0.0018 |
| dihydro-2H-quinazolin-3-ylmethyl]-benzoic acid | |
| 1-Methyl-2,4-dioxo-3-(pyridin-4-methyl)-1,2,3,4-tetrahydro-quinazoline- | 0.0110 |
| carboxylic acid (benzo[1,3]dioxol-5-ylmethyl)-amide | |
| 1-Methyl-2,4-dioxo-3-(pyridin-4-ylmethyl)-1,2,3,4-tetrahydro-quinazoline- | 0.0210 |
| carboxylic acid 4-methoxy-benzylamide | |
| 1-Methyl-2,4-dioxo-3-(pyridin-4-ylmethyl)-1,2,3,4-tetrahydro-quinazoline-6- | 0.0510 |
| carboxylic acid 4-hydroxy-benzylamide | |
| Methyl 4-[6-(3-methoxy-benzylcarbamoyl)-1-methyl-2,4-dioxo-1,4-dihydro- | 0.0030 |
| 2H-quinazolin-3-ylmethyl]-benzoate | |
| 4-[6-(3-Methoxy-benzylcarbamoyl)-1-methyl-2,4-dioxo-1,4-dihydro-2H- | 0.0009 |
| quinazolin-3-ylmethyl]-benzoic acid | |
| Methyl 4-[1-methyl-6-(4-methylsulfanyl-benzylcarbamoyl)-2,4-dioxo-1,4- | 0.0230 |
| dihydro-2H-quinazolin-3-ylmethyl]-benzoate, | |
| 4-[1-Methyl-6-(4-methylsulfanyl-benzylcarbamoyl)-2,4-dioxo-1,4-dihydro- | 0.0029 |
| 2H-quinazolin-3-ylmethyl]-benzoic acid | |
| Methyl 4-[1-ethyl-2,4-dioxo-6-(4-trifluoromethoxy-benzylcarbamoyl)-1,4- | 0.3400 |
| dihydro-2H-quinazolin-3-ylmethyl]-benzoate | |
| Methyl 4-[6-(4-fluoro-benzylcarbamoyl)-1-methyl-2,4-dioxo-1,4-dihydro-2H- | 0.0100 |
| quinazolin-3-ylmethyl]-benzoate | |
| 4-[6-(4-Fluoro-benzylcarbamoyl)-1-methyl-2,4-dioxo-1,4-dihydro-2H- | 0.0018 |
| quinazolin-3-ylmethyl]-benzoic acid | |
| Methyl 4-{6-[(benzofurazan-5-ylmethyl)-carbamoyl]-1-methyl-2,4-dioxo-1,4- | 0.0350 |
| dihydro-2H-quinazolin-3-ylmethyl}-benzoate | |
| 4-{6-[(Benzofurazan-5-ylmethyl)-carbamoyl]-1-methyl-2,4-dioxo-1,4- | 0.0030 |
| dihydro-2H-quinazolin-3-ylmethyl}-benzoic acid | |
| Methyl 4-[6-(4-methoxy-benzylcarbamoyl)-2,4-dioxo-1,4-dihydro-2H- | 0.0090 |
| quinazolin-3-ylmethyl]-benzoate | |
| Methyl 4-[1-ethyl-6-(4-methoxy-benzylcarbamoyl)-2,4-dioxo-1,4-dihydro-2H- | 0.0310 |
| quinazolin-3-ylmethyl]-benzoate | |
| 4-[1-Ethyl-6-(4-methoxy-benzylcarbamoyl)-2,4-dioxo-1,4-dihydro-2H- | 0.0030 |
| quinazolin-3-ylmethyl]-benzoic acid | |
| 3-(4-Methoxy-benzyl)-1-methyl-2,4-dioxo-1,2,3,4-tetrahydroquinazoline-6- | 0.0600 |
| carboxylic acid (pyridin-4-ylmethyl)-amide | |
| 3-(4-Hydroxy-benzyl)-1-methyl-2,4-dioxo-1,2,3,4-tetrahydro-quinazoline-6- | 0.0570 |
| carboxylic acid (pyridin-4-ylmethyl)-amide | |
| 3-(4-Cyano-benzyl)-1-methyl-2,4-dioxo-1,2,3,4-tetrahydro-quinazoline-6- | 0.0530 |
| carboxylic acid (pyridin-4-ylmethyl)-amide | |
| 1-Methyl-2,4-dioxo-3-(3-pyridin-4-yl-allyl)-1,2,3,4-tetrahydro-quinazoline-6- | 0.2400 |
| carboxylic acid (pyridin-4-ylmethyl)-amide | |
| Methyl 4-{1-methyl-2,4-dioxo-6-[(pyridin-4-ylmethyl)-carbamoyl]-1,4- | 0.0230 |
| dihydro-2H-quinazolin-3-ylmethyl}-benzoate | |
| 4-{1-Methyl-2,4-dioxo-6-[(pyridin-4-ylmethyl)-carbamoyl]-1,4-dihydro-2H- | 0.0057 |
| quinazolin-3-ylmethyl}-benzoic acid | |
| Methyl (4-{1-methyl-2,4-dioxo-6-[(pyridin-4-ylmethyl)-carbamoyl]-1,4- | 0.0200 |
| dihydro-2H-quinazolin-3-ylmethyl}-phenyl)-acetate | |
| (4-{1-Methyl-2,4-dioxo-6-[(pyridin-4-ylmethyl)-carbamoyl]-1,4-dihydro-2H- | 0.0110 |
| quinazolin-3-ylmethyl}-phenyl)-acetic acid | |
| Methyl 4-{1-methyl-2,4-dioxo-6-[(1-oxy-pyridin-4-ylmethyl)carbamoyl]-1,4- | 0.1000 |
| dihydro-2H-quinazolin-3-ylmethyl}-benzoate | |
| Methyl{6-[(1,3-Benzodioxol-5-ylmethyl)-carbamoyl]-3-benzyl-2,4-dioxo-1,4- | 0.1600 |
| dihydro-2H-quinazolin-1-yl}-acetate | |
| {6-[(1,3-Benzodioxol-5-ylmethyl)-carbamoyl]-3-benzyl-2,4-dioxo-3,4- | 0.1050 |
| dihydro-2H-quinazolin-1-yl}-acetic acid, | |
| Methyl 4-{6-[(1,3-benzodioxol-5-ylmethyl)-carbamoyl]-1-methyl-2,4-dioxo- | 0.0028 |
| 1,4-dihydro-2H-quinazolin-3-ylmethyl}-benzoate | |
| 4-{6-[(1,3-Benzodioxol-5-ylmethyl)-carbamoyl]-1-methyl-2,4-dioxo-1,4- | 0.0009 |
| dihydro-2H-quinazolin-3-ylmethyl}-benzoic acid | |
| 3-Benzyl-1-methyl-2,4-dioxo-1,2,3,4-tetrahydro-quinazoline-6-carboxylic acid | 0.0260 |
| [3-(pyridin-4-ylsulfanyl)-propyl]-amide | |
| 3-Benzyl-1-methyl-2,4-dioxo-1,2,3,4-tetrahydro-quinazoline-6-carboxylic acid | 0.0200 |
| 4-hydroxy-benzylamine | |
| Ethyl (4-{[(3-benzyl-1-methyl-2,4-dioxo-1,2,3,4-tetrahydro-quinazoline-6- | 0.7200 |
| carbonyl)-amino]-methyl}-phenoxy)-acetate | |
| (4-{[(3-Benzyl-1-methyl-2,4-dioxo-1,2,3,4-tetrahydro-quinazoline-6- | 2.9000 |
| carbonyl)amino]-methyl}-phenoxy)-acetic acid | |
| 3-Benzyl-1-methyl-2,4-dioxo-1,2,3,4-tetrahydro-quinazoline-6-carboxylic acid | 0.3400 |
| 4-cyano-benzylamide | |
| 3-(4-Dimethylamino-benzyl)-2,4-dioxo-1,2,3,4-tetrahydro-quinazoline-6- | 0.0750 |
| carboxylic acid 4-methoxy-benzylamide | |
| 3-[4-(N-methylsulfonylamino)-benzyl]-1-methyl-2,4-dioxo-1,2,3,4-tetrahydro- | 0.0040 |
| quinazoline-6-carboxylic acid 4-methoxy-benzylamide | |
| tert-Butyl {5-[6-(4-Methoxy-benzylcarbamoyl)-1-methyl-2,4-dioxo-1,4- | 0.0150 |
| dihydro-2H-quinazolin-3-ylmethyl]-pyridin-2-yl}-carbamate | |
| 3-(6-Amino-pyridin-3-ylmethyl)-1-methyl-2,4-dioxo-1,2,3,4-tetrahydro- | 0.0530 |
| quinazoline-6-carboxylic acid 4-methoxy-benzylamide | |
| 1,3-Dimethyl-2,4-dioxo-1,2,3,4-tetrahydro-pyrido[2,3-d]pyrimidine-6- | 3.8500 |
| carboxylic acid (1,3-benzodioxol-5-ylmethyl)-amide | |
| 1,3-Dimethyl-2,4-dioxo-1,2,3,4-tetrahydro-pyrido[3,4-d]pyrimidine-6- | 0.1800 |
| carboxylic acid (1,3-benzodioxol-5-ylmethyl)-amide | |
| 3-Benzyl-1-methyl-2,4-dioxo-1,2,3,4-tetrahydro-pyrido[2,3-d] pyrimidine-6- | 0.0070 |
| carboxylic acid (1,3-benzodioxol-5-ylmethyl)-amide | |
| 4-[6-(4-Methoxy-benzylcarbamoyl)-1-methyl-2,4-dioxo-1,4-dihydro-2H- | 0.0016 |
| pyrido[2,3-d] pyrimidin-3-ylmethyl]-benzoic acid | |
| 3-(4-Cyano-benzyl)-1-methyl-2,4-dioxo-1,2,3,4-tetrahydro-pyrido[2,3-d] | 0.0160 |
| pyrimidine-6-carboxylic acid 4-methoxy-benzylamide | |
| 3-(4-Fluoro-benzyl)-1-methyl-2,4-dioxo-1,2,3,4-tetrahydro-pyrido[2,3- | 0.0320 |
| d]pyrimidine-6-carboxylic acid 4-methoxy-benzylamide | |
| 3-Benzyl-1-methyl-2,4-dioxo-1,2,3,4-tetrahydro-pyrido[3,4-d]pyrimidine-6- | 0.00078 |
| carboxylic acid (1,3-benzodioxol-5-ylmethyl)-amide | |
| Methyl 4-[6-(4-Methoxy-benzylcarbamoyl)-1-methyl-2,4-dioxo-1,4-dihydro- | 0.0017 |
| 2H-pyrido[3,4-d]pyrimidin-3-ylmethyl]-benzoate | |
| 4-[6-(4-Methoxy-benzylcarbamoyl)-1-methyl-2,4-dioxo-1,4-dihydro-2H- | 0.00074 |
| pyrido[3,4-d]pyrimidin-3-ylmethyl]-benzoic acid | |
| 4-[6-(3-Methoxy-benzylcarbamoyl)-1-methyl-2,4-dioxo-1,4-dihydro-2H- | 0.0011 |
| pyrido[3,4-d]pyrimidin-3-ylmethyl]-benzoic acid | |
| 3-(4-Cyano-benzyl)-1-methyl-2,4-dioxo-1,2,3,4-tetrahydro-pyrido[3,4- | 0.0011 |
| d]pyrimidine-6-carboxylic acid 4-methoxy-benzylamide | |
| 3-Benzyl-1-methyl-6-(3-phenyl-propionyl)-1H-quinazoline-2,4-dione | 2.9000 |
| 3-Benzyl-1-methyl-2,4-dioxo-1,2,3,4-tetrahydro-quinazoline-6-carboxylic acid | 0.3850 |
| (E)-3-pyridin-4-yl-allyl ester | |
| 3-Benzyl-1-methyl-2,4-dioxo-1,2,3,4-tetrahydro-quinazoline-6-carboxylic acid | 0.3400 |
| (E)-3-pyridin-3-yl-allyl ester | |
| 3-Benzyl-1-methyl-6-[2-(pyridin-4-ylsulfanyl)-acetyl]-1H-quinazoline-2,4- | 3.4000 |
| dione | |
| 3-(4-Aminomethyl-benzyl)-1-methyl-2,4-dioxo-1,2,3,4-tetrahydro- | 0.0700 |
| quinazoline-6-carboxylic acid 4-methoxy-benzylamide | |
| 3-(2′-Cyano-biphenyl-4-ylmethyl)-1-methyl-2,4-dioxo-1,2,3,4- | 0.0250 |
| tetrahydroquinazoline-6-carboxylic acid 4-methoxy-benzylamide | |
| 1-Methyl-2,4-dioxo-3-[2′-(1H-tetrazol-5-yl)-biphenyl-4-ylmethyl]-1,2,3,4- | 0.0250 |
| tetrahydro-quinazoline-6-carboxylic acid 4-methoxy-benzylamide | |
| Methyl 4′-[6-(4-methoxy-benzylcarbamoyl)-1-methyl-2,4-dioxo-1,4-dihydro- | 0.0840 |
| 2H-quinazolin-3-ylmethyl]-biphenyl-2-carboxylate | |
| 4′-[6-(4-Methoxy-benzylcarbamoyl)-1-methyl-2,4-dioxo-1,4-dihydro-2H- | 0.0130 |
| quinazolin-3-ylmethyl]-biphenyl-2-carboxylic acid | |
| Ethyl 2-Fluoro-4-[6-(4-methoxy-benzylcarbamoyl)-1-methyl-2,4-dioxo-1,4- | 0.0090 |
| dihydro-2H-quinazolin-3-ylmethyl]-benzoate | |
| 2-Fluoro-4-[6-(4-methoxy-benzylcarbamoyl)-1-methyl-2,4-dioxo-1,4-dihydro- | 0.0010 |
| 2H-quinazolin-3-ylmethyl]-benzoic acid | |
| 2-Methoxy-4-[6-(4-methoxy-benzylcarbamoyl)-1-methyl-2,4-dioxo-1,4- | 0.5000 |
| dihydro-2H-quinazolin-3-ylmethyl]-benzoic acid 2-dimethylamino-ethyl ester | |
| 4-[6-(4-Methoxy-benzylcarbamoyl)-1-methyl-2,4-dioxo-1,4-dihydro-2H- | 0.1100 |
| quinazolin-3-ylmethyl]-2-methyl-benzoic acid 2-dimethylamino-ethyl ester | |
| 1-Methyl-2,4-dioxo-3-[4-(5-oxo-4,5-dihydro-1,2,4-oxadiazol-3-yl)-benzyl]- | 0.0015 |
| 1,2,3,4-tetrahydro-quinazoline-6-carboxylic acid 4-methoxy-benzylamide | |
| {4-[6-(4-Methoxy-benzylcarbamoyl)-1-methyl-2,4-dioxo-1,4-dihydro-2H- | 8.0000 |
| quinazolin-3-yl]-phenyl}-acetic acid | |
| 1-Methyl-3-(1-naphthalen-1-yl-ethyl)-2,4-dioxo-1,2,3,4-tetrahydro- | 9.4000 |
| quinazoline-6-carboxylic acid (1,3-benzodioxol-5-ylmethyl)-amide | |
| 3-(3-Fluoro-benzyl-1-methyl-2,4-dioxo-1,2,3,4-tetrahydro-quinazoline-6- | 0.0170 |
| carboxylic acid (pyridin-4-ylmethyl)-amide | |
| 3-(3-Fluoro-benzyl)-1-methyl-2,4-dioxo-1,2,3,4-tetrahydro-quinazoline-6- | 0.0058 |
| carboxylic acid (2-methoxy-pyridin-4-ylmethyl)-amide | |
| 3-(3-Fluoro-benzyl)-1-methyl-2,4-dioxo-1,2,3,4-tetrahydro-quinazoline-6- | 0.0670 |
| carboxylic acid (pyridin-3-ylmethyl)-amide | |
| 3-(3-Fluoro-benzyl)-1-methyl-2,4-dioxo-1,2,3,4-tetrahydro-quinazoline-6- | 0.0079 |
| carboxylic acid 4-methoxy-benzylamide | |
| 3-(3-Fluoro-benzyl)-1-methyl-2,4-dioxo-1,2,3,4-tetrahydro-quinazoline-6- | 0.0210 |
| carboxylic acid 3-methoxy-benzylamide | |
| 1-Ethyl-3-(3-fluoro-benzyl)-2,4-dioxo-1,2,3,4-tetrahydro-quinazoline-6- | 0.1000 |
| carboxylic acid (pyridin-4-ylmethyl)-amide | |
| 1-Ethyl-3-(3-fluoro-benzyl)-2,4-dioxo-1,2,3,4-tetrahydro-quinazoline-6- | 0.3600 |
| carboxylic acid (pyridin-3-ylmethyl)-amide | |
| 3-(4-Bromo-benzyl)-1-methyl-2,4-dioxo-1,2,3,4-tetrahydro-quinazoline-6- | 0.0130 |
| carboxylic acid 4-methoxy-benzylamide | |
| 3-(4-Bromo-benzyl)-1-methyl-2,4-dioxo-1,2,3,4-tetrahydro-quinazoline-6- | 0.0076 |
| carboxylic acid (2-methoxy-pyridin-4-ylmethyl)-amide | |
| 3-(3,4-Difluoro-benzyl)-1-methyl-2,4-dioxo-1,2,3,4-tetrahydro-quinazoline-6- | 0.0670 |
| carboxylic acid (pyridin-3-ylmethyl)-amide | |
| 3-(3,4-Difluoro-benzyl)-1-methyl-2,4-dioxo-1,2,3,4-tetrahydro-quinazoline-6- | 0.0320 |
| carboxylic acid (pyridin-4-ylmethyl)-amide | |
| 3-(3,4-Difluoro-benzyl)-1-methyl-2,4-dioxo-1,2,3,4-tetrahydro-quinazoline-6- | 0.0098 |
| carboxylic acid 4-methoxy-benzylamide | |
| 3-(3-chloro-4-fluoro-benzyl)-1-methyl-2,4-dioxo-1,2,3,4-tetrahydro- | 0.0260 |
| quinazoline-6-carboxylic acid (pyridin-4-ylmethyl)-amide, | |
| 3-(3-Chloro-4-fluoro-benzyl)-1-methyl-2,4-dioxo-1,2,3,4-tetrahydro- | 0.0120 |
| quinazoline-6-carboxylic acid 4-methoxy-benzylamide | |
| 4-[6-(4-Methoxy-benzylcarbamoyl)-1-methyl-2,4-dioxo-1,4-dihydro-2H- | 0.0010 |
| quinazolin-3-ylmethyl]-benzoate(2-hydroxy-ethyl)-trimethyl-ammonium | |
| 4-[6-(4-Methoxy-benzylcarbamoyl)-1-methyl-2,4-dioxo-1,4-dihydro-2H- | 0.0010 |
| quinazolin-3-ylmethyl]-benzoic acid hemicalcium | |
| 4-[6-(4-Methoxy-benzylcarbamoyl)-1-methyl-2,4-dioxo-1,4-dihydro-2H- | 0.0011 |
| quinazolin-3-ylmethyl]-benzoic acid hemimagnesium | |
| 3-(4-Chloro-benzyl)-1-methyl-2,4-dioxo-1,2,3,4-tetrahydro-quinazoline-6- | 0.0530 |
| carboxylic acid (pyridin-4-ylmethyl)-amide | |
| 3-(4-Fluoro-benzyl)-1-methyl-2,4-dioxo-1,2,3,4-tetrahydro-quinazoline-6- | 0.0710 |
| carboxylic acid (pyridin-4-ylmethyl)-amide | |
| 3-(4-Fluoro-benzyl)-1-methyl-2,4-dioxo-1,2,3,4-tetrahydro-quinazoline-6- | 0.1200 |
| carboxylic acid (pyridin-3-ylmethyl)-amide | |
| 3-(4-Chloro-benzyl)-1-methyl-2,4-dioxo-1,2,3,4-tetrahydroquinazoline-6- | 0.0930 |
| carboxylic acid (pyridin-3-ylmethyl)-amide | |
| 3-(4-Fluoro-benzyl)-1-methyl-2,4-dioxo-1,2,3,4-tetrahydroquinazoline-6- | 0.0046 |
| carboxylic acid 3-methoxy-benzylamide | |
| 3-(4-Chloro-benzyl)-1-methyl-2,4-dioxo-1,2,3,4-tetrahydroquinazoline-6- | 0.0043 |
| carboxylic acid 3-methoxy-benzylamide | |
| 3-(4-Fluoro-benzyl)-1-methyl-2,4-dioxo-1,2,3,4-tetrahydroquinazoline-6- | 0.0110 |
| carboxylic acid (2-methoxy-pyridin-4-ylmethyl)-amide | |
| 3-(4-Chloro-benzyl)-1-methyl-2,4-dioxo-1,2,3,4-tetrahydroquinazoline-6- | 0.0110 |
| carboxylic acid (2-methoxy-pyridin-4-ylmethyl)-amide | |
| tert-Butyl 1-{4-[6-(4-methoxy-benzylcarbamoyl)-1-methyl-2,4-dioxo-1,4- | 0.0665 |
| dihydro-2H-quinazolin-3-ylmethyl]-phenyl)-cyclopropanecarboxylate | |
| 1-{4-[6-(4-Methoxy-benzylcarbamoyl)-1-methyl-2,4-dioxo-1,4-dihydro-2H- | 0.0033 |
| quinazolin-3-ylmethyl]-phenyl}-cyclopropanecarboxylic acid | |
| 3-Benzyl-6-benzylsulfanyl-1-methyl-1H-quinazoline-2,4-dione | 4.1000 |
| 4-[6-(4-methoxy-benzylcarbamoyl)-1-methyl-2,4-dioxo-1,4-dihydro-2H- | 0.0880 |
| quinazoline-3-ylmethyl]-benzoic acid tert-butoxycarbonylmethl ester | |
| 4-[6-(4-methoxy-benzylcarbamoyl)-1-methyl-2,4-dioxo-1,4-dihydro-2H- | 0.0600 |
| quinazoline-3-ylmethyl]-benzoic acid dimethlamino-dimethyl-propyl ester | |
| 4-[6-(4-methoxy-benzylcarbamoyl)-1-methyl-2,4-dioxo-1,4-dihydro-2H- | 0.0600 |
| quinazoline-3-ylmethyl]-benzoic acid dimethlamino-methyl-propyl ester | |
| 4-[6-(4-methoxy-benzylcarbamoyl)-1-methyl-2,4-dioxo-1,4-dihydro-2H- | 0.0370 |
| quinazoline-3-ylmethyl]-benzoic acid 2-dimethylamio-ethyl ester | |
| 4-[6-(4-methoxy-benzylcarbamoyl)-1-methyl-2,4-dioxo-1,4-dihydro-2H- | 0.0390 |
| quinazoline-3-ylmethyl]-benzoic acid 2-(2-amino-3-methyl-butanoylamino)- | |
| 3-methyl-butanoyloxymethyl ester | |

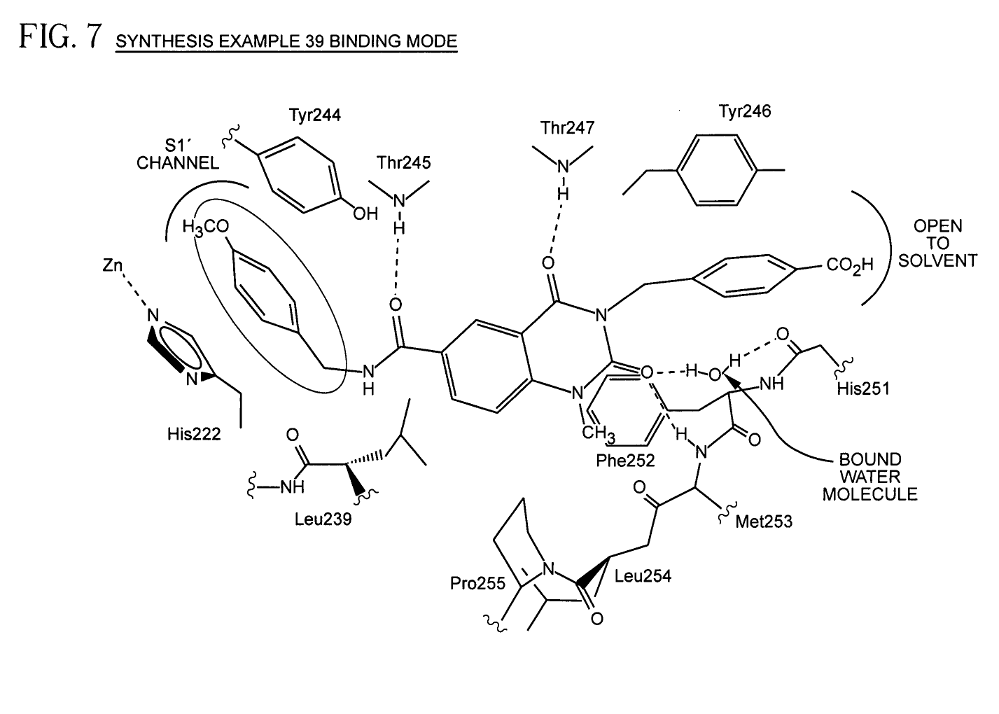

Binding of the compound of Synthesis Example 35 is shown in FIG. 7 and is based on two hydrophobic groups and three hydrogen bond acceptors. As in the previous series of compounds the third hydrogen bond acceptor binds both to Met 253 and via a bridging water molecule to the backbone carbonyl oxygen of His 251. It will also be noted from the above table that some compounds in this series do not have a second hydrophobic group but nevertheless bind to MMP-13 and exhibit a useful inhibitory activity.

Synthesis of some of the compounds referred to in Table IVa and Table IVb is described in the following further synthesis examples. The synthesis of the other compounds in the Table IVa and Table IVb is reported in our co-pending WO application which claims the priority of the application No. U.S. 60/268,661 filed on Feb. 14, 2001.

### SYNTHESIS EXAMPLE 22

### 3-Benzyl-2,4-dioxo-1,2,3,4-tetrahydroquinazoline-6-carboxylic acid benzylamide

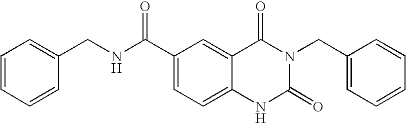

1st Stage:

### 4-Nitroisophthalic acid

25 g (138 mmol) of 5-methyl-2-nitrobenzoic acid are suspended in 300 ml of water. 5 g (89.1 mmol) of KOH are added for dissolution. The medium is heated to 90° C. and 158 g of KMnO₄ (414 mmol) are added portionwise, rinsing with H₂O. After 3 hours, the reaction medium is filtered through Celite and the filtrate is acidified to pH 1 with concentrated HCl. The precipitate obtained is filtered off and dried under vacuum. Weight=15.3 g, yield=53% NMR: DMSO ¹H δ (ppm) 5.7-5.62 (d, 1H); 7.88 (d, 1H); 8.16 (s, 1H).

2nd Stage:

### Dimethyl 4-nitroisophthalate

12.75 g (60.4 mmol) of 4-nitroisophthalic acid from the above stage and 13 ml of H₂SO₄ and 100 ml of methanol are maintained at reflux overnight. After cooling, the methanol is removed under vacuum. The residue is dissolved in 400 ml of EtOAc. The organic phase is washed with 50 ml of H₂O and then with 50 ml of 5% NaHCO₃ solution. Drying over MgSO₄ and concentration under vacuum gives a crystalline residue. Weight=12.17 g, yield=84%, NMR: DMSO ¹H δ (ppm) 3.86 (s,3H); 3.91 (s,3H); 8.16 (d,1H); 8.29-8.34 (m,2H).

3rd Stage:

### Dimethyl 4-aminoisophthalate (Dimethyl Intermediate 1)

The compound from the above stage is reduced with H₂ in the presence of Pd as catalyst. Filtration through Celite and concentration gives the above compound: Weight=5.12 g, yield=70%, m.p.=127-128° C., NMR: CDCl₃ ¹H δ (ppm) 3.87 (s,3H); 3.88 (s,3H); 6.30 (brs,2H); 6.65 (d,1H); 7.89 (dd,1H); 8.57 (d,1H).

4th Stage:

### Methyl 3-benzyl-2,4-dioxo-1,2,3,4-tetrahydroquinazoline-6-carboxylate

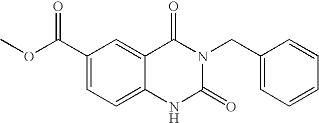

4 g (19.1 mmol) of dimethyl 4-aminoisophthalate and 40 ml of pyridine are successively introduced into a 50 ml three-necked flask fitted with a reflux condenser and protected from moisture, followed by addition of 3.2 g (24 mmol) of benzyl isocyanate. The colourless solution is stirred and heated at 95-100° C. After 6 hours at this temperature, 1 ml of benzyl isocyanate is added and stirring is then continued at 100° C. overnight. The next day, the reaction medium is cooled and poured into 400 ml of a water+ice mixture, it is left stirring for about 30 minutes and the precipitate obtained is then filtered off. The product is re-slurried at reflux in 150 ml of ethanol. After cooling, the product is filtered off. The product is obtained as follows: Weight=3.7 g, yield=62% NMR: DMSO ¹H δ (ppm): 3.75 (s,3H); 4.95 (s,2H); 7.1-7.2 (m,6H); 8.05 (d,1H); 8.35 (s,1H); 11.8 (bs,1H).

5th Stage:

### 3-Benzyl-2,4-dioxo-1,2,3,4-tetrahydroquinazoline-6-carboxylic acid (Intermediate 2)

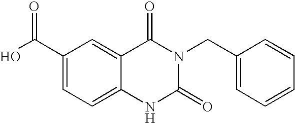

1.5 g (4.84 mmol) of methyl 3-benzyl-2,4-dioxo-1,2,3,4-tetrahydroquinazoline-6-carboxylate, 14 ml of dioxane and 48 ml of H₂O are introduced into a 100 ml round-bottomed flask fitted with a reflux condenser. 0.41 g (9.68 mmol) of hydrated lithium hydroxide is added to the suspension with stirring. The mixture is brought to reflux and maintained for about 1 hour (solution). After cooling in an ice bath, the medium is acidified to pH 1 with concentrated hydrochloric acid. The very fine precipitate obtained is filtered off, to give the above compound: Weight: 1.3 g, yield=96% NMR: DMSO ¹H δ (ppm): 5.1 (s,2H); 7.2-7.35 (m,6H); 8.15 (d,1H); 8.48 (s,1H); 11.85 (s,1H); 13.1 (bs,1H)

6th Stage:

### 3-Benzyl-2,4-dioxo-1,2,3,4-tetrahydroquinazoline-6-carboxylic acid benzylamide

0.150 g (0.51 mmol) of 3-benzyl-2,4-dioxo-1,2,3,4-tetrahydroquinazoline-6-carboxylic acid (Intermediate 2) and 8.0 ml of anhydrous dimethylformamide are introduced into a stirred 25 ml one-necked flask protected from moisture. 0.0547 g (56 μl, 0.51 mmol) of benzylamine and 0.17 g (0.51 mmol) of O-[(ethoxycarbonyl)cyanomethylenamino]-N,N,N′,N′-tetramethyluronium tetrafluoroborate (TOTU) are added to this solution. The solution is cooled in a bath to 0° C. 0.132 g (0.18 ml, 1.02 mmol) of N,N-diisopropylethylamine is then added. The mixture is warmed to room temperature and stirred overnight. After monitoring by TLC (90/10 CH₂Cl₂/MeOH), the DMF is removed under vacuum. The crystalline residue obtained is taken up in dichloromethane with the amount of methanol required for total dissolution. The organic phase is washed successively with 40 ml of 1N HCl, 40 ml of H₂O, 40 ml of saturated NaHCO₃ solution and finally 40 ml of H₂O. The organic phase is dried over Na₂SO₄ and the solvents are removed under vacuum. 0.140 g of product is obtained, which is recrystallized from 30 ml of acetonitrile: Weight: 0.110 g, yield=56% TLC: CH₂Cl₂/MeOH 90/10 Rf=0.65, NMR: DMSO ¹H δ (ppm): 4.45 (d,2H); 5.1 (s,2H); 7.1-7.4 (m,11H); 8.1 (d,1H); 8.5 (s,1H); 9.15 (m,1H); 11.75 (bs,1H), IR: 3425, 2364, 1722, 1640, 1509, 1442, 1304, 1261, 1078, 927, 845 cm⁻¹, m.p.=241.2° C., HPLC: 98.3%

### SYNTHESIS EXAMPLE 23

### 3-Benzyl-2,4-dioxo-1,2,3,4-tetrahydroquinazoline-6-carboxylic acid (benzo[1,3]dioxol-5-ylmethyl)amide

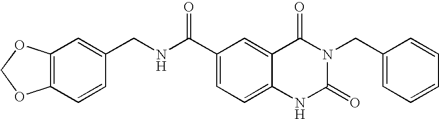

With the same procedure as in the sixth stage of Synthesis Example 22, but using piperonylamine, and after crystallization from acetonitrile, the above compound is obtained: Weight: 0.140 g, yield=64%, TLC: CH₂Cl₂/MeOH 90/10 Rf=0.65, NMR: DMSO ¹H δ (ppm): 4.35 (d,2H); 5.1 (s,2H); 5.95 (s,2H); 6.7-6.95 (m,3H); 7.15-7.4 (m,6H); 8.15 (d,1H); 8.5 (s,1H); 9.1 (t,1H); 11.7 (bs,1H), IR: 3200, 1727, 1636, 1493, 1444, 1299, 1261, 1041, 938, 841, 763, 726 cm⁻¹, m.p.=256° C. HPLC: 99%.

### SYNTHESIS EXAMPLE 24

### 3-Benzyl-1-methyl-2,4-dioxo-1,2,3,4-tetrahydroquinazoline-6-carboxylic acid (benzo[1,3]dioxol-5-ylmethyl)amide

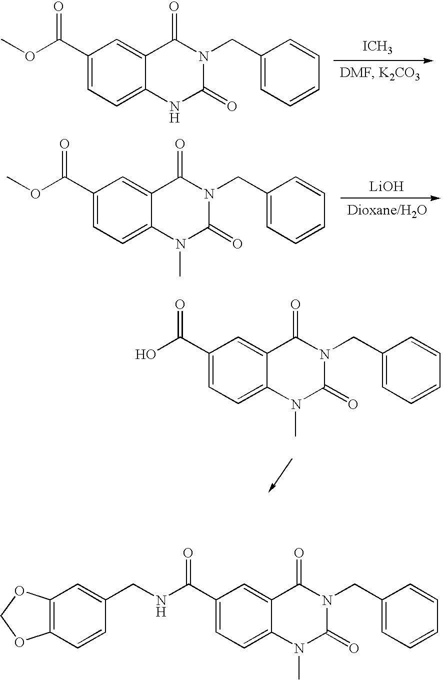

Stage 1:

### Methyl 3-benzyl-1-methyl-2,4-dioxo-1,2,3,4-tetrahydroquinazoline-6-carboxylate

11.8 g (38.0 mmol) of methyl 3-benzyl-2,4-dioxo-1,2,3,4-tetrahydroquinazoline-6-carboxylate (preparation: see the 4th stage of Synthesis Example 22), 120 ml of dimethylformamide and 7.9 g (57 mmol) of K₂CO₃ are introduced into a 250 ml three-necked flask. The suspension is stirred for 15 minutes at about room temperature. 27 g (12 ml, 190 mmol) of iodo-methane are added over 2 minutes. The suspension is stirred at room temperature for 30 to 45 minutes. The solvent is removed under vacuum and the residue is taken up in 500 ml of dichloromethane and washed with 3 times 300 ml of water. The organic phase is dried and the solvent is removed. The product obtained is as follows: Weight: 12 g, yield=97.4%, TLC: CH₂Cl₂/acetone 98/2 Rf=0.60, m.p.=179.3° C., NMR: DMSO ¹H δ (ppm) 3.6 (s,3H); 3.90 (s,3H); 5.1 (s,2H); 7.2-7.4 (m,5H); 7.55 (d,1H); 8.25 (d,1H); 8.6 (s,1H).

Stage 2:

### 3-Benzyl-1-methyl-2,4-dioxo-1,2,3,4-tetrahydroquinazoline-6-carboxylic acid (Intermediate 3)

9.5 g (29.3 mmol) of the product from the preceding stage are hydrolysed using the same procedure as for the fifth stage of Synthesis Example 22 to give the above compound as follows: Weight: 10 g, yield=100%, TLC: CH₂Cl₂/MeOH 90/10 Rf=0.50, m.p.=227.2° C., NMR: DMSO ¹H δ (ppm) 3.55 (s,3H); 5.15 (s,2H); 7.2-7.4 (m,5H); 7.55 (d,1H); 8.25 (d,1H); 8.6 (s,1H); 13.2 (bs,1H)

Stage 3:

### 3-Benzyl-1-methyl-2,4-dioxo-1,2,3,4-tetrahydroquinazoline-6-carboxylic acid (benzo[1,3]dioxol-5-ylmethyl)amide

0.500 g (1.61 mmol) of 3-benzyl-1-methyl-2,4-dioxo-1,2,3,4-tetrahydroquinazoline-6-carboxylic acid (Intermediate 3) and 25 ml of anhydrous dimethylformamide are introduced into a stirred 50 ml one-necked flask protected from moisture. 0.244 g (0.201 ml, 1.61 mmol) of piperonylamine and 0.531 g (1.61 mmol) of TOTU are added to this solution. The solution is cooled in a cold bath to 0° C. 0.415 g (0.564 ml, 3.22 mmol) of N,N-diisopropylethylamine is then added. The mixture is warmed to room temperature and stirred overnight. After, monitoring by TLC (90/10 CH₂Cl₂/MeOH), the DMF is removed under vacuum. The crystalline residue obtained is taken up in dichloromethane. The organic phase is washed successively with 1N HCl, H₂O, saturated NaHCO₃ and finally H₂O. The organic phase is dried over Na₂SO₄ and the solvent is removed under vacuum. 0.540 g of product, recrystallized from 30 ml of acetonitrile, is obtained as follows: Weight: 0.390 g, yield=54.6%, TLC: CH₂Cl₂/acetone 90/10 Rf=0.40, NMR: DMSO ¹H δ (ppm): 3.55 (s,3H); 4.35 (d,2H); 5.15 (s,2H); 6.0 (s,2H); 6.75-6.95 (m,3H); 7.2-7.4 (m,5H); 7.55 (d,1H); 8.25 (d,1H); 8.65 (s,1H); 9.2 (t,1H), IR: 3303, 1703, 1656, 1637, 1498, 1444, 1322, 1254, 1040, 932, 845 cm⁻¹, m.p.=215.1° C., HPLC: 99.5%

### SYNTHESIS EXAMPLE 25

### 3-Benzyl-1-methyl-2,4-dioxo-1,2,3,4-tetrahydroquinazoline-6-carboxylic acid 4-hydroxy-3-methoxybenzylamide

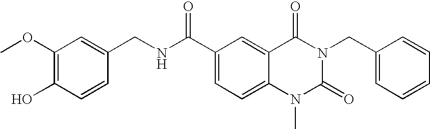

The final step of Synthesis Example 24 is repeated, but using 4-hydroxy-3-methoxybenzylamine hydrochloride and 3.5 equivalents of N,N-diisopropylethylamine. The crude product is purified by chromatography on silica, using a 95/5 CH₂Cl₂/MeOH gradient. After solidification in ether, the product is obtained as follows: Weight: 0.090 g, yield=42%, TLC: CH₂Cl₂/MeOH 95/5 Rf=0.59, NMR: DMSO ¹H δ (ppm) 3.55 (s,3H); 3.75 (s,3H); 4.4 (d,2H); 5.15 (s,2H); 6.75 (s,2H); 6.95 (s,1H); 7.2-7.40 (m,6H); 7.55 (d,1H); 8.3 (d,1H); 8.65 (s,1H); 8.8 (s,1H); 9.15 (t,1H), IR: 1707, 1655, 1618, 1502, 1477, 1277, 704 cm⁻¹, m.p.=183° C., HPLC: 87.1%.

### SYNTHESIS EXAMPLE 26

### 3-Benzyl-1-methyl-2,4-dioxo-1,2,3,4-tetrahydroquinazoline-6-carboxylic acid 4-methoxybenzylamide

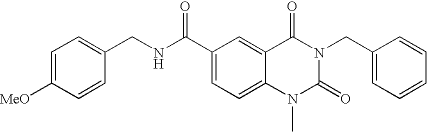

The final stage of Synthesis Example 24 is repeated but using 4-methoxybenzylamine. The crude product is purified by chromatography on silica, using 97/3 CH₂Cl₂/MeOH as eluent. The desired fractions are combined and concentrated. The product is solidified in ether and then filtered off. The product is obtained as follows: Weight: 0.320 g, yield=77.7%, TLC: CH₂Cl₂/MeOH 90/10 Rf=0.8, NMR: DMSO ¹H δ (ppm) 3.55 (s,3H); 3.75 (s,3H); 4.45 (d,2H); 5.2 (s,2H); 6.9 (d,2H); 7.2-7.4 (m,7H); 7.6 (d,1H); 8.3 (d,1H); 8.65 (s,1H); 9.25 (t,1H); IR: 1705, 1660, 1636, 1505, 1251, 750 cm⁻1, m.p.=191° C., HPLC: 97.3%.

### SYNTHESIS EXAMPLE 27

### 3-(4-Methoxybenzyl)-2,4-dioxo-1,2,3,4-tetrahydroquinazoline-6-carboxylic acid (benzo[1,3]dioxol-5-ylmethyl)amide

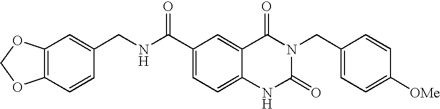

1st Stage:

### Dimethyl 4-amino-1-hydroxycyclohexa-3,5-diene-1,3-dicarboxylate

526 ml of benzene and 250 ml of methyl acrylate are introduced into a 1-litre three-necked flask fitted with a reflux condenser, placed under inert atmosphere and protected from moisture, followed by 10 g (70.8 mmol) of methyl 5-amino-2-furoate. The mixture is brought to reflux and maintained for 24 hours. The reaction medium is concentrated to dryness at 50° C. under a vacuum of 20 mm Hg. The residue obtained is purified by flash chromatography using dichloromethane progressively enriched with ethyl acetate as solvent. The product is obtained as follows: Weight=15 g of a yellow precipitate, yield=93%, TLC: CH₂Cl₂/EtOAc 70/30 v/v Rf=0.35, m.p.=101.3° C., NMR: CDCl₃ ¹H δ (ppm) 2.87 (d,1h); 2.93 (d,1H); 3.20 (s,1H); 3.71 (s,3H); 3.82 (s,3H); 6.02 (d,1H); 5.60-6.40 (brs,2H); 6.17 (d,1H)

2nd Stage:

### Dimethyl isophthalate (Intermediate 1)

15 g (66 mmol) of dimethyl 4-amino-1-hydroxycyclohexa-3,5-diene-1,3-dicarboxylate obtained in the preceding stage and 600 ml of benzene are introduced into a 1-litre three-necked flask fitted with a reflux condenser, placed under an inert atmosphere and protected from moisture. 13.8 g (12 ml, 98 mmol) of BF₃ etherate are added with stirring. The mixture is refluxed for 2 minutes and then cooled to room temperature and, after addition of saturated NaHCO₃ solution (pH 9), the phases are separated by settling. The aqueous phase is re-extracted twice with dichloromethane. The organic phases are combined and dried over Na₂SO₄. After removal of the solvents under vacuum, the 13.8 g of residue are purified by chromatography using dichloromethane as elution solvent. The product is obtained as follows: Weight=8.5 g of a crystallyne residue, yield=62%, TLC: CH₂Cl₂. Rf=0.30, m.p.=130.1° C., NMR: CDCl₃ ¹H δ (ppm) 3.87 (s,3H); 3.88 (s,3H); 6.30 (brs,2H); 6.65 (d,1H); 7.89 (dd,1H); 8.57 (d,1H).

3rd Stage:

### Methyl 3-(4-methoxybenzyl)-2,4-dioxo-1,2,3,4-tetrahydroquinazoline-6-carboxylate

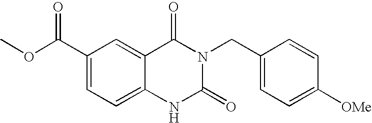

0.750 g (3.6 mmol) of Intermediate 1 and 7.5 ml of pyridine are introduced into a round-bottomed flask. 3.6 mmol of 4-methoxybenzyl isocyanate is added. The mixture is maintained at 100° C. overnight. Since the reaction is incomplete, 2 additions of phenethyl isocyanate, i.e. 2 equivalents, are carried out. After precipitation with H₂O, filtration and purification by reslurrying in hot ethanol, the product is obtained as follows: Weight: 0.750 g, yield=61.3%, NMR: DMSO ¹H δ (ppm): 3.7 (s,3H); 3.8 (s,3H); 5.0 (s,2H); 6.8-6.85 (m,2H); 7.2-7.3 (m,3H); 8.1-8.2 (m,1H); 8.5 (s,1H); 11.9 (bs,1H).

4th Stage:

### 3-(4-Methoxybenzyl)-2,4-dioxo-1,2,3,4-tetrahydroquinazoline-6-carboxylic acid

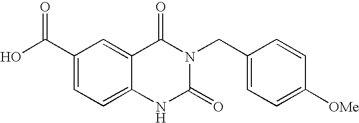

The product from the preceding stage is hydrolysed using hydrated LiOH in a dioxane/H₂O mixture) to give the above product as follows: Weight: 0.680 g, Yield=94.8%, NMR: DMSO ¹H δ (ppm): 3.7 (s,3H); 5.0 (s,2H); 6.8-7.9 (m,2H); 7.2-7.3 (m,3H); 8.1-8.2 (m,1H); 8.5 (s,1H); 11.8 (s,1H); 13.1 (bs,1H).

5th Stage:

### 3-(4-Methoxybenzyl)-2,4-dioxo-1,2,3,4-tetrahydroquinazoline-6-carboxylic acid (benzo[1,3]dioxol-5-ylmethyl)amide

Starting with 200 mg (0.6 mmol) of the preceding product, using the procedure described in the final stage of Synthesis Example 24 with piperonylamine, and after solidification of the crude product in dichloromethane, the above product is obtained as follows: Weight: 0.220 g, Yield=79.9%, NMR: DMSO ¹H δ (ppm) 3.7 (s,3H); 4.35 (d,2H); 5.0 (s,2H); 5.95 (s,2H); 6.75-6.9 (m,5H); 7.2-7.3 (m,3H); 8.1 (d,1H); 8.5 (s,1H); 9.1 (t,1H); 11.75 (s,1H), IR: 1720, 1648, 1634, 1504, 1442, 1300, 1250, 1036, 766 cm⁻¹, m.p.=252° C., HPLC: 96.2%

### SYNTHESIS EXAMPLE 28

### 3-(4-Methoxybenzyl)-1-methyl-2,4-dioxo-1,2,3,4-tetrahydroquinazoline-6-carboxylic acid (benzo[1,3]dioxol-5-ylmethyl)amide

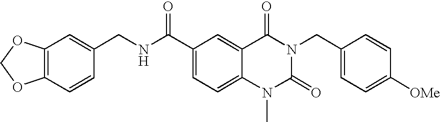

The alkylation with methyl iodide of the product obtained in Synthesis Example 22 is carried out using dimethylformamide, K₂CO₃ and iodomethane. After crystallization from ether, the product is obtained as follows: Weight: 0.080 g, Yield=70.4%, NMR: DMSO ¹H δ (ppm) 3.55 (s,3H); 3.7 (s,3H); 4.4 (d,2H); 5.05 (s,2H); 5.95 (s,2H); 6.8-6.95 (m,5H); 7.3 (d,2H); 7.55 (d,1H); 8.25 (d,1H); 8.6 (s,1H); 9.2 (t,1H), IR: 3265, 1704, 1662, 1634, 1504, 1443, 1320, 1248, 1040, 771 cm⁻¹, m.p.=178° C., HPLC: 99.2%.

### SYNTHESIS EXAMPLE 29

### 3-(4-Methoxybenzyl)-1-methyl-2,4-dioxo-1,2,3,4-tetrahydroquinazoline-6-carboxylic acid 4-methoxybenzylamide

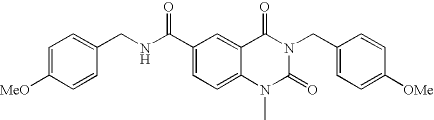

Step 1:

### 3-(4-Methoxybenzyl)-2,4-dioxo-1,2,3,4-tetrahydroquinazoline-6-carboxylic acid (4-methoxybenzyl)amide

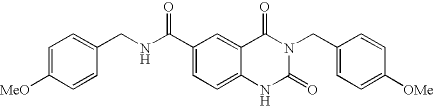

0.240 g (0.74 mmol) of 3-(4-methoxybenzyl)-2,4-dioxo-1,2,3,4-tetrahydroquinazoline-6-carboxylic acid is treated as in the final stage of Synthesis Example 24 with 4-methoxybenzylamine. The product is obtained as follows: Weight: 0.270 g, Yield=82%, NMR: DMSO ¹H δ (ppm): 3.7 (2s,6H); 4.4 (d,2H); 5.0 (s,2H); 6.8-6.95 (m,4H); 7.2-7.35 (m,5H); 8.15 (d,2H); 8.5 (s,1H); 9.15 (t,1H); 11.75 (bs,1H).

Step 2:

### 3-(4-Methoxybenzyl)-1-methyl-2,4-dioxo-1,2,3,4-tetrahydroquinazoline-6-carboxylic acid 4-methoxybenzylamide (above)

The alkylation with methyl iodide of the product obtained in Step 1 is carried out with dimethylformamide, K₂CO₃ and iodomethane. After crystallization from ether, the product is obtained as follows: Weight: 0.260 g, Yield=94.4%, NMR: DMSO ¹H δ (ppm) 3.6 (s,3H); 3.7 (dd,6H); 4.45 (d,2H); 5.1 (s,2H); 6.8-6.95 (m,4H); 7.25-7.40 (m,4H); 7.55 (d,1H); 8.25 (d,1H); 8.65 (s,1H); 9.2 (t,1H), IR: 1705, 1655, 1641, 1614, 1510, 1247, 1175, 1033 cm⁻¹, m.p.=195° C., HPLC: 99.5%.

### SYNTHESIS EXAMPLE 30

### 2,4-Dioxo-3-(thien-2-ylmethyl)-1,2,3,4-tetrahydroquinazoline-6-carboxylic acid (benzo[1,3]dioxol-5-ylmethyl)amide

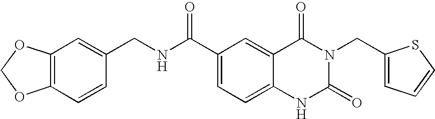

Step 1:

### Methyl N-benzyl-6-(3-thien-2-ylmethylureido)isophthalate

Intermediate 1 (above) according to method B (1st Stage) in anhydrous toluene containing animal charcoal is treated with triphosgene and refluxed for 2 hours. The reaction medium is then filtered through infusorial earth and evaporated to dryness under vacuum. The residue in anhydrous toluene is treated with 2-thiophene methylamine, and toluene is added as necessary to facilitate stirring. The resulting product is filtered off, washed successively with toluene and with ether and dried under vacuum. NMR: DMSO ¹H δ (ppm): 3.8 (s,3H); 3.9 (s,3H); 4.5 (d,2H); 6.9-7.0 (m,2H); 7.4 (m,1H); 8.0-8.05 (m,1H); 8.4 (t,1H); 8.5 (s,1H); 8.6-8.65 (m,1H); 10.15 (s,1H)

Step 2:

### Methyl 2,4-dioxo-3-thien-2-ylmethyl-1,2,3,4-tetrahydroquinazoline-6-carboxylate

The urea from step 1 is cyclized in methanolic MeONa to obtained a product as follows: NMR: DMSO ¹H δ (ppm): 3.8 (s,3H); 5.25 (s,2H); 6.9 (d,1H); 7.1 (s,1H); 7.25 (d,1H); 7.4 (d,1H); 8.1-8.15 (m,1H); 8.5 (s,1H); 11.9 (bs,1H)

Step 3:

### 2,4-Dioxo-3-thien-2-ylmethyl-1,2,3,4-tetrahydroquinazoline-6-carboxylic acid

The product from step 2 is hydrolyzed with hydrated LiOH in a dioxane/H₂O mixture according to the procedure described in the 2nd Stage of method A. The product is obtained as follows: NMR: DMSO ¹H δ (ppm): 5.25 (s,2H); 6.95 (d,1H); 7.15 (d,1H); 7.2-7.3 (m,1H); 7.4 (d,1H); 8.1-8.2 (m,1H); 8.5 (s,1H); 11.9 (s,1H); 13.1 (bs,1H)

Step 4:

### 2,4-Dioxo-3-(thien-2-ylmethyl)-1,2,3,4-tetrahydroquinazoline-6-carboxylic acid (benzo[1,3]dioxol-5-ylmethyl)amide

The product from step 3 is reacted with piperonylamine using the method described in Synthesis Example 22. The crude product is solidified in dichloromethane and is as follows: Weight: 0.170 g, yield=59%, TLC: CH₂Cl₂/MeOH 95/5 Rf=0.4, NMR: DMSO ¹H δ (ppm) 4.40 (d,2H); 5.25 (s,2H); 6.0 (s,2H); 6.75-7.0 (m,4H); 7.1 (s,1H); 7.25 (d,1H); 7.40 (d,1H); 8.2 (d,1H); 8.55 (s,1H); 9.20 (t,1H); 11.8 (s,1H), IR: 3185, 1727, 1632, 1502, 1445, 1300, 1259, 1040, 936, 846, 765 cm⁻¹, m.p.=270.1° C., HPLC: 95.2%.

### SYNTHESIS EXAMPLE 31

1-Methyl-2,4-dioxo-3-(thien-2-ylmethyl)-1,2,3,4-tetrahydroquinazoline-6-carboxylic acid (benzo[1,3]dioxol-5-ylmethyl)amide

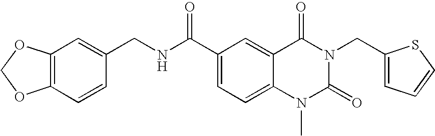

The product of Synthesis Example 30 is dissolved in dimethyl formamide, and potassium carbonate is added. After stirring for 15 minutes at room temperature iodomethane is added, and stirring is continued for a further 30-45 minutes. The solvent is then removed under vacuum, and the residue is taken up in dichloromethane and washed with water. The solution is then concentrated under vacuum and purified by chromatography on silica using a 98/2 dichloromethane/methanol gradient. The product obtained was as follows: Weight: 0.085 g, yield=79.7%, TLC: CH₂Cl₂/MeOH 95/5 Rf=0.8, NMR: DMSO ¹H δ (ppm) 3.6 (s,3H); 4.40 (d,2H); 5.30 (s,2H); 6.0 (s,2H); 6.8-7.0 (m,4H); 7.2 (d,1H); 7.40 (d,1H); 7.5-7.6 (m,1H); 8.2-8.30 (m,1H); 8.6 (s,1H); 9.20 (t,1H), IR: 3251, 1705, 1659, 1635, 1501, 1446, 1328, 1253, 1041, 926, 784 cm⁻¹, m.p.=224.2° C., HPLC: 99.8%.

### SYNTHESIS EXAMPLE 32

### 3-(4-Chlorobenzyl)-2,4-dioxo-1,2,3,4-tetrahydroquinazoline-6-carboxylic acid (benzo[1,3]dioxol-5-ylmethyl)amide

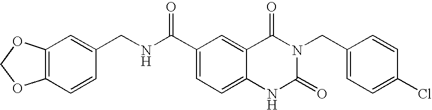

The product of this example was synthesized as described in Synthesis Example 22 from Intermediate 1 using 4-chlorobenzyl isocyanate, followed by amidation with piperonylamine. After solidification in dichloromethane, the product is obtained as follows: Weight: 0.170 g, yield=67.8%, NMR: DMSO ¹H δ (ppm) 4.35 (t,2H); 5.1 (s,2H); 5.95 (s,2H); 6.75-6.9 (m,3H); 7.25 (d,1H); 7.35 (s,4H); 8.15 (d,1H); 8.5 (s,1H); 9.15 (t,1H); 11.8 (bs,1H), IR: 3265, 1734, 1653, 1633, 1504, 1440, 1254, 1041, 811, 761 cm⁻¹, m.p.=290° C., HPLC: 99.2%.

### SYNTHESIS EXAMPLE 33

### 3-(4-Chlorobenzyl)-1-methyl-2,4-dioxo-1,2,3,4-tetrahydroquinazoline-6-carboxylic acid (benzo[1,3]dioxol-5-ylmethyl)amide

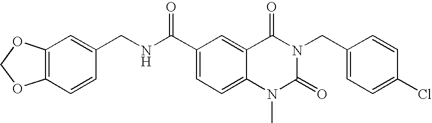

The product of Synthesis Example 32 is alkylated with methyl iodide by the method used in Synthesis Example 31. After crystallization from ether, the product is obtained as follows: Weight: 0.085 g, yield=88.9%, NMR: DMSO ¹H δ (ppm) 3.55 (s,3H); 4.40 (t,2H); 5.15 (s,2H); 5.95 (s,2H); 6.75-6.9 (m,3H); 7.35 (s,4H); 7.55 (d,1H); 8.25 (d,1H); 8.65 (s,1H); 9.20 (t,1H) IR: 3249, 1704, 1658, 1636, 1488, 1251, 810, 753 cm⁻¹, m.p.=231° C., HPLC: 99.6%.

### SYNTHESIS EXAMPLE 34

### 3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide

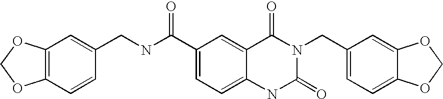

The above compound was prepared as described in Synthesis Example 30.

Step 1:

### Dimethyl 4-(3-benzo[1,3]dioxol-5-ylmethylureido)isophthalate

NMR: CDCl3 ¹H δ (ppm): 3.9 (s,6H); 4.4 (s,2H); 5.1 (t,1H); 6.95 (s,2H); 6.7-6.85 (m,3H); 8.1-8.2 (m,1H); 8.6-8.7 (m,2H); 10.6 (bs,1H)

Step 2:

### Methyl 3-(benzo[1,3]dioxol-5-ylmethyl)-2,4-dioxo-1,2,3,4-tetrahydroquinazoline-6-carboxylate (intermediate)

The resulting urea is cyclized in methanolic MeONa to obtained a product as follows: NMR: DMSO ¹H δ (ppm): 3.8 (s,3H); 5.0 (s,2H); 5.9 (s,2H); 6.8 (s,2H); 6.9 (s,1H); 7.25 (d,1H); 8.15 (d,1H); 8.5 (s,1H); 11.8 (bs,1H)

Step 3:

### 3-(Benzo[1,3]dioxol-5-ylmethyl)-2,4-dioxo-1,2,3,4-tetrahydroquinazoline-6-carboxylic acid

The product obtained in step 2 is hydrolyzed with hydrated LiOH in a dioxane/H₂O mixture according to the procedure described above. The product is obtained as follows: NMR: DMSO ¹H δ (ppm): 5.0 (s,2H); 6.0 (s,2H); 6.8 (s,2H); 6.9 (s,1H); 7.3 (d,1H); 8.2 (d,1H); 8.5 (s,1H); 11.85 (s,1H); 13.05 (bs,1H)

Step 4:

### 3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide

The compound required is prepared from the product of Step 3 with piperonylamine. Weight: 0.040 g, yield=36%, TLC: CH₂Cl₂/MeOH 95/5 Rf=0.70, NMR: DMSO ¹H δ (ppm) 4.40 (s,2H); 5.0 (s,2H); 5.9 (s,4H); 6.75-6.95 (m,6H); 7.20-7.30 (m,1H); 8.05-8.15 (m,1H); 8.45-8.55 (m,1H); 9.1 (m,1H); 10.3 (m,1H), IR: 3271, 1739, 1649, 1630, 1503, 1440, 1250, 1041, 926, 759 cm⁻¹, m.p.=245.2° C., HPLC: 81.5%

### SYNTHESIS EXAMPLE 35

### 3-(Benzo[1,3]dioxol-5-ylmethyl)-1-methyl-2,4-dioxo-1,2,3,4-tetrahydroquinazoline-6-carboxylic acid (benzo[1,3]dioxol-5-ylmethyl)amide

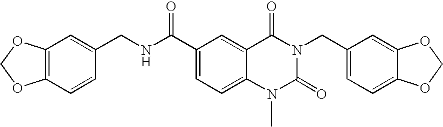

The above product is made from the product of Synthesis Example 34 by alkylation according to the method described above. Weight: 0.050 g, yield=40.5%, TLC: CH₂Cl₂/MeOH 90/10 Rf=0.80 NMR: DMSO ¹H δ (ppm) 3.55 (s,3H); 4.35 (s,2H); 5.0 (s,2H); 6.0 (s,4H); 6.80-7.0 (m,6H); 7.5 (d,1H); 8.25 (d,1H); 8.6 (s,1H); 9.15-9.2 (m,1H), IR: 3302, 1703, 1663, 1630, 1490, 1247, 1041, 929, 807, 785 cm⁻¹, m.p.=197.5° C., HPLC: 100%.

### SYNTHESIS EXAMPLE 36

### 3-Benzyl-1-ethyl-2,4-dioxo-1,2,3,4-tetrahydroquinazoline-6-carboxylic acid (benzo[1,3]dioxol-5-ylmethyl)amide

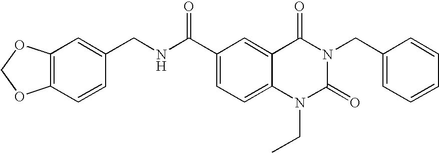

0.150 g (0.35 mmol) of 3-benzyl-2,4-dioxo-1,2,3,4-tetrahydroquinazoline-6-carboxylic acid (benzo[1,3]dioxol-5-ylmethyl)amide was prepared as described in Synthesis Example 23, and then 3 ml of anhydrous DMF are introduced into a stirred round-bottomed flask protected from moisture. 0.075 g (0.525 mmol) of K₂CO₃ is added to the stirred solution. The mixture is stirred for 15 minutes and 0.273 g (0.14 ml, 1.75 mmol) of iodoethane is then added. Stirring is continued for about 1 hour. After the solvent has been removed under vacuum, the residue is dissolved in 50 ml of dichloromethane and washed with 2×50 ml of H₂O. After drying over Na₂SO₄ and concentration under vacuum, the product is crystallized from 8 ml of acetonitrile. The product is obtained as follows: Weight: 0.070 g, Yield=43.7%, TLC: CH₂Cl₂/MeOH 95/5 Rf=0.70, NMR: DMSO ¹H δ (ppm) 1.25 (t,3H); 4.2 (q,2H); 4.4 (d,2H); 5.15 (s,2H); 5.95 (s,2H); 6.75-6.95 (m,3H); 7.2-7.4 (m,5H); 7.65 (d,1H); 8.25 (d,1H); 8.65 (s,1H); 9.15 (t,1H), IR: 1701, 1658, 1633, 1506, 1488, 1458, 1246, 1217, 1038, 926, 803 cm⁻¹, m.p.=176.5° C., HPLC: 99%.

### SYNTHESIS EXAMPLE 37

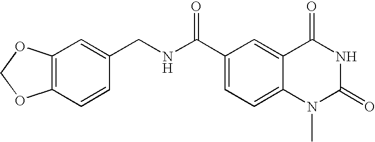

### 1-Methyl-2,4-dioxo-1,2,3,4-tetrahydroquinazoline-6-carboxylic acid (benzo[1,3]dioxol-5-ylmethyl)amide

0.870 g (2.7 mmol) of methyl 3-benzyl-1-methyl-2,4-dioxo-1,2,3,4-tetrahydroquinazoline-6-carboxylate prepared in the 1st Stage of Intermediate 3, 20 ml of benzene and 2.1 g (16.1 mmol) of AlCl₃ are maintained at 50° C. for 7 hours. After cooling, the medium is precipitated on a water and ice mixture. The insoluble material is dissolved in dichloromethane and purified by flash chromatography, eluting with a gradient of CH₂Cl₂/acetone. 0.510 g of methyl 1-methyl-2,4-dioxo-1,2,3,4-tetrahydroquinazoline-6-carboxylate is obtained. The saponification of the ester is carried out with LiOH in a dioxane/H₂O mixture as for the preceding examples. Amidation with piperonylamine gives the desired product. Weight: 0.160 g, TLC: CH₂Cl₂/MeOH 90/10 Rf=0.45, NMR: DMSO ¹H δ (ppm) 3.45 (s,3H); 4.4 (d,2H); 6.0 (s,2H); 6.75-6.95 (m,3H); 7.5 (d,1H); 8.25 (d,1H); 8.55 (s,1H); 9.2 (t,1H); 11.7 (s,1H), IR: 3290, 1697, 1635, 1503, 1484, 1324, 1258, 1040, 844 cm⁻¹, m.p.=279° C., HPLC: 98.7%.

### SYNTHESIS EXAMPLE 38

38a:

### 1-Methyl-2,4-dioxo-1,2,3,4-tetrahydro-quinazoline-6-carboxylic acid 4-methoxy-benzylamide

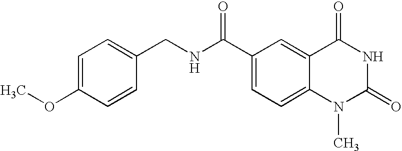

Preparation identical to that of Synthesis Example 37, using 1-Methyl-2,4-dioxo-1,2,3,4-tetrahydro-quinazoline-6-carboxylic acid (NMR: DMSO ¹H δ (ppm) 3.50 (s,3H); 7.5 (d,1H); 8.20 (d,1H); 8.50 (s,1H); 11.75 (bs,1H); 13.1 (bs,1H)) and 4 methoxy-benzylamine in DMF with TOTU and DIPEA. The product is obtained as follows: NMR: DMSO ¹H δ (ppm) 3.50 (s,3H); 3.70 (s,3H); 4.40 (d,2H); 6.90 (d,2H); 7.25 (d,2H); 7.50 (d,1H); 8.20 (d,1H); 8.55 (s,1H); 9.20 (t,1H); 11.65 (bs,1H).

38b:

### 4-[6-(4-Methoxy-benzylcarbamoyl)-1-methyl-2,4-dioxo-1,4-dihydro-2H-quinazolin-3-ylmethyl]-benzoic acid methyl ester

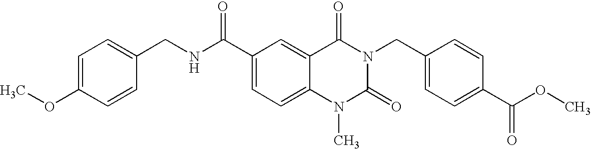

0.8 g (2.36 mmoles) of the product of previous stage and 8 ml of DMF anhydrous DMF are stirred with 1.15 g (3.54 mmol) of cesium carbonate. Stirring is continued for 15 minutes and then 0.81 g (3.54 mmol) of Methyl4-(bromomethyl)benzoate are added. The mixture is maintained at 90° C. for 1 h 15 min and then stirred overnight. 15 ml of water are added and then extracted with dichloromethane. The organic phase is washed with water and concentrated to dryness under vacuum. The product obtained is purified with flash chromatography eluting with a gradient of CH₂Cl₂/MeOH. The product is obtained as follows: Weight: 0.220 g, TLC: CH₂Cl₂/MeOH 90/10 Rf=.0.85, NMR: DMSO ¹H δ (ppm) 3.55 (s,3H); 3.7 (s,3H); 3.85 (s,3H); 4.4 (d,2H); 5.25 (s,2H); 6.9 (d,2H); 7.25 (d,2H); 7.45 (d,2H); 7.55 (d,1H); 7.9 (d,2H); 8.25 (dd,1H); 8.6 (s,1H); 9.2 (t,1H), IR: 3387, 1709, 1658, 1642, 1508, 1286, 1248, 1110, 1032, 835, 750 cm⁻¹, m.p=189.2° C., HPLC: 96.5%.

### SYNTHESIS EXAMPLE 39

### 4-[6-(4-Methoxy-benzylcarbamoyl)-1-methyl-2,4-dioxo-1,4-dihydro-2H]-quinazolin-3-ylmethyl]-benzoic acid

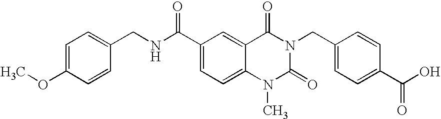

0.16 g (3.3 mmoles) of the product obtained in Example 34 are hydrolyzed in a mixture of 1.2 ml of dioxane and 4.2 ml of water with 28mg of LiOH monohydrate. The mixture is maintained at reflux for 10 minutes to complete the reaction. The mixture is acidified to pH 1 with concentrated HCl, the precipitate is filtered off and the product is obtained as follows: Weight: 0.120 g, TLC: CH₂Cl₂/MeOH 90/10 Rf=.0.50, NMR: DMSO ¹H δ (ppm) 3.55 (s,3H); 3.75 (s,3H); 4.4 (d,2H); 5.20 (s,2H); 6.9 (d,2H); 7.25 (d,2H); 7.40 (d,2H); 7.60 (d,1H); 7.85 (d,2H); 8.25 (dd,1H); 8.65 (s,1H); 9.2 (t,1H) 12.9 (bs,1H), IR: 3378, 1702, 1658, 1645, 1616, 1506, 1297, 1248, 1125, 839, 788, 751 cm⁻¹, m.p=262.5° C., HPLC: 100%.

### SYNTHESIS EXAMPLE 40

### 1-Methyl-2,4-dioxo-3-((E)-3-phenylallyl)-1,2,3,4-tetrahydroquinazoline-6-carboxylic acid (benzo[1,3]dioxol-5-ylmethyl)amide

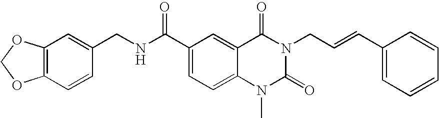

0.100 g (0.28 mmol) of 1-methyl-2,4-dioxo-1,2,3,4-tetrahydroquinazoline-6-carboxylic acid (benzo[1,3]dioxol-5-ylmethyl)amide (Synthesis Example 37) and 1 ml of anhydrous DMF are stirred with 0.060 g (0.42 mmol) of K₂CO₃. The mixture is maintained for 15 min, followed by addition of 0.085 g (0.42 mmol) of cinnamyl bromide. The mixture is maintained at 70° C. for 2 hours, concentrated under vacuum, after which the residue is taken up in dichloromethane, washed with H₂O and then dried over Na₂SO₄. The solvent is removed and the product is purified by flash chromatography, eluting with a 95/5 gradient of CH₂Cl₂/MeOH. The pure product obtained is solidified in ether: Weight: 0.070 g, Yield=51%, TLC: CH₂Cl₂/MeOH 95/5 Rf=0.46, NMR: DMSO ¹H δ (ppm) 3.55 (s,3H); 4.4 (d,2H); 4.75 (d,2H); 6.0 (s,2H); 6.3-6.4 (m,1H); 6.6 (d,1H); 6.80-6.95 (m,3H); 7.2-7.35 (m,3H); 7.4 (d,2H); 7.55 (d,1H); 8.25 (d,1H); 8.65 (s,1H); 9.25 (t,1H); IR: 1659, 1643, 1503, 1477, 1246, 754 cm⁻¹ m.p.=174° C., HPLC: 98.4%.

### SYNTHESIS EXAMPLE 41

### Benzyl 3-benzyl-2,4-dioxo-1,2,3,4-tetrahydroquinazoline-6-carboxylate

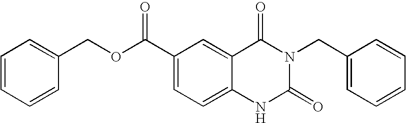

A mixture of 0.5 g (1.7 mmol) of 3-benzyl-2,4-dioxo-1,2,3,4-tetrahydroquinazoline-6-carboxylic acid (Intermediate 2), 0.44 g (1.7 mmol) of triphenylphosphine and 0.44 ml (4.3 mmol) of benzyl alcohol is stirred in 20 ml of THF. A solution of 0.27 ml (1.7 mmol) of DEAD in 10 ml of THF is added dropwise with stirring. Stirring is continued overnight at room temperature. The precipitate formed is filtered through Celite and the filtrate is concentrated under vacuum. The residue is dissolved in 50 ml of ethyl acetate and washed successively with H₂O and then with saturated NaCl solution. After drying over MgSO₄ and concentration under vacuum, the crude product obtained is purified by flash chromatography on silica, eluting with a 50/50 mixture of hexane/EtOAc. The desired fractions are combined and the solvent is removed under vacuum. A crystalline residue is obtained. Weight: 0.190 g, Yield=29%, MS: m/z 387.2 (M+H)+, NMR: DMSO ¹H δ (ppm) 5.06 (s,2H); 5.34 (s,2H); 7.22-7.46 (m,10H); 8.20 (d,1H); 8.48 (s,1H); 11.89 (s,1H), CHN (C₂₃H₁₈N₂O₄) calc: C=71.49; H=4.70; N=7.25. found: C=71.28; H=4.94; N=7.11.

### SYNTHESIS EXAMPLE 42

### Benzyl 3-benzyl-1-methyl-2,4-dioxo-1,2,3,4-tetrahydroquinazoline-6-carboxylate

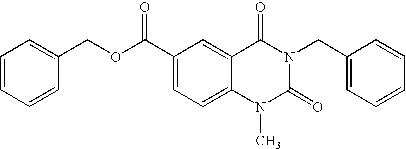

0.084 g (0.217 mmol) of the product of Synthesis Example 41 above is stirred with anhydrous THF in apparatus protected from moisture and under an inert atmosphere. 0.14 ml of 1.6M BuLi in hexane (0.224 mmol) is introduced. The mixture is stirred for 10 minutes, followed by addition of 0.04 ml (0.642 mmol) of methyl iodide. The THF is removed under vacuum. The residue is dissolved in EtOAc and washed successively with H₂O and then with saturated NaCl solution. After drying over MgSO₄ and concentration under vacuum, the crude product obtained is purified by flash chromatography on silica, eluting with a 50/50 mixture of hexane/EtOAc. The desired fractions are combined and the solvent is removed under vacuum. The pale yellow product is solidified in ether: Weight: 0.049 g, yield=56%, MS: m/z 401.2 (M+H)+, NMR: DMSO ¹H δ (ppm) 3.31 (s,3H); 5.12 (s,2H); 5.37 (s,2H); 7.21-7.60 (m,11H); 8.28 (d,1H); 8.58 (s,1H), CHN (C₂₄H₂₀N₂O₄) calc: C=71.99; H=5.03; N=7.00. found: C=71.71; H=5.25; N=6.87.

### SYNTHESIS EXAMPLE 43

### 4-Pyridylmethyl 3-benzyl-2,4-dioxo-1,2,3,4-tetrahydroquinazoline-6-carboxylate

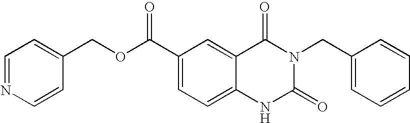

Using the same method as in Synthesis Example 41, but using dichloromethane as solvent, the product is obtained as follows: MS: m/z 388.2 (M+H)+, NMR: DMSO ¹H δ (ppm) 5.07 (s,2H); 5.41 (s,2H); 7.20-7.32 (m,6H); 7.43 (d,2H); 8.26 (d,1H); 8.53-8.58 (m,3H); 11.93 (s,1H), CHN (C₂₂H₁₇N₃O₄.0.3H₂O) calc: C=67.27; H=4.52; N=10.70. found: C=67.32; H=4.40; N=10.47.

### SYNTHESIS EXAMPLE 44

### 4-Pyridylmethyl 3-benzyl-1-methyl-2,4-dioxo-1,2,3,4-tetrahydroquinazoline-6-carboxylate

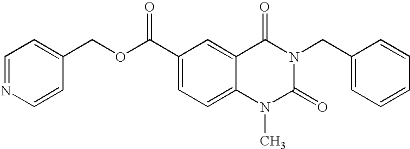

Starting with 3-benzyl-1-methyl-2,4-dioxo-1,2,3,4-tetrahydroquinazoline-6-carboxylic acid (Intermediate 3) using triphenylphosphine, diethyl azodicarboxylate (DEAD) and 4-pyridylcarbinol, the product is obtained as follows: MS: m/z 402.3 (M+H)+, NMR: DMSO ¹H δ (ppm) 3.55 (s,3H); 5.14 (s,2H); 5.42 (s,2H); 7.23-7.33 (m,5H); 7.43-7.45 (m,2H); 7.60 (d,1H); 8.32-8.36 (m,1H); 8.57-8.64 (m,3H), CHN (C₂₃H₁₉N₃O₄.0.14H₂O): calc: C=68.39; H=4.81; N=10.40. found: C=68.40; H=4.71; N=10.38.

### SYNTHESIS EXAMPLE 45

### Benzo[1,3]dioxol-5-ylmethyl 3-benzyl-2,4-dioxo-1,2,3,4-tetrahydroquinazoline-6-carboxylate

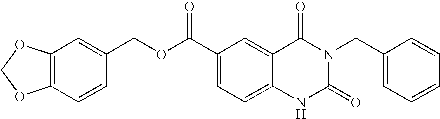

0.100 g (0.337 mmol) of 3-benzyl-2,4-dioxo-1,2,3,4-tetrahydroquinazoline-6-carboxylic acid (Intermediate 2) and 1 ml of anhydrous THF are placed in a round-bottomed flask protected from moisture. The suspension is stirred and 0.24 g (0.150 ml, 2.025 mmol) of thionyl chloride is added. The mixture is refluxed for 1 h 30 min. The solution is cooled, concentrated to dryness under vacuum, and the 0.110 g of acid chloride obtained is used in the next stage without further purification. 0.080 g (0.51 mmol) of piperonyl alcohol, 1 ml of dichloromethane and 0.051 g (0.070 ml, 0.51 mmol) of triethylaamine are introduced into a round-bottomed flask protected from moisture. The solution is cooled to 0° C. The above acid chloride suspended in 2.5 ml of dichloromethane is added to the solution and the mixture is stirred at room temperature for 48 hours. The precipitate obtained is filtered off. The resulting product is purified by recrystallization from acetonitrile. Weight: 0.025 g, yield=17%, TLC: CH₂Cl₂/MeOH 95/5 Rf=0.85, NMR: DMSO ¹H δ (ppm) 5.1 (s,2H); 5.25 (s,2H); 6.05 (s,2H); 6.9-7.4 (m,9H); 8.2 (d,1H); 8.5 (s,1H); 11.9 (bs,1H), IR: 1715, 1650, 1624, 1446, 1285, 1262, 1080, 928, 865, 764 cm⁻¹, m.p.=238.5° C., HPLC: 99.7%.

### SYNTHESIS EXAMPLE 46

### Benzo[1,3]dioxol-5-ylmethyl 3-benzyl-1-methyl-2,4-dioxo-1,2,3,4-tetrahydroquinazoline-6-carboxylate

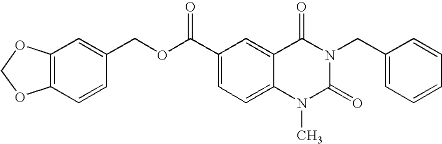

3-Benzyl-1-methyl-2,4-dioxo-1,2,3,4-tetrahydroquinazoline-6-carboxylic acid (Intermediate 3) is treated firstly with thionyl chloride/THF and then in dichloromethane with piperonyl alcohol and triethylamine to give the above product as follows: Weight: 0.140 g, TLC: CH₂Cl₂/MeOH 95/5 Rf=0.85, NMR: DMSO ¹H δ (ppm) 3.55 (s,3H); 5.15 (s,2H); 5.30 (s,2H); 6.05 (s,2H); 6.9-7.4 (m,8H); 7.6 (d,1H); 8.25 (d,1H); 8.6 (s,1H); IR: 1716, 1703, 1659, 1618, 1447, 1294, 1227, 1103, 935, 813, 763 cm⁻¹, m.p.=199.5° C., HPLC: 98.8%.

### SYNTHESIS EXAMPLE 47

### 4-Pyridylmethyl 2,4-dioxo-3-thien-2-ylmethyl-1,2,3,4-tetrahydroquinazoline-6-carboxylate

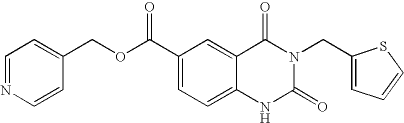

Step 1:

### Methyl N-benzyl-6-(3-thien-2-ylmethylureido)isophthalate

The above compound was prepared from Intermediate 1 according to Synthesis Example 30 described above, using 2-thiophene methylamine. NMR: DMSO ¹H δ (ppm): 3.8 (s,3H); 3.9 (s,3H); 4.5 (d,2H); 6.9-7.0 (m,2H); 7.4 (m,1H); 8.0-8.05 (m,1H); 8.4 (t,1H); 8.5 (s,1H); 8.6-8.65 (m,1H); 10.15 (s,1H).

Step 2:

### Methyl 2,4-dioxo-3-thien-2-ylmethyl-1,2,3,4-tetrahydroquinazoline-6-carboxylate

The resulting urea is cyclized in methanolic MeONa to obtained the a product as follows: NMR: DMSO ¹H δ (ppm): 3.8 (s,3H); 5.25 (s,2H); 6.9 (d,1H); 7.1 (s,1H); 7.25 (d,1H); 7.4 (d,1H); 8.1-8.15 (m,1H); 8.5 (s,1H); 11.9 (bs,1H).

Step 3:

### 2,4-Dioxo-3-thien-2-ylmethyl-1,2,3,4-tetrahydroquinazoline-6-carboxylic acid

The product obtained is hydrolyzed with hydrated LiOH in a dioxane/H₂O mixture according to the procedure described in the 2nd Stage of method A. The product is obtained as follows: NMR: DMSO ¹H δ (ppm): 5.25 (s,2H); 6.95 (d,1H); 7.15 (d,1H); 7.2-7.3 (m,1H); 7.4 (d,1H); 8.1-8.2 (m,1H); 8.5 (s,1H); 11.9 (s,1H); 13.1 (bs,1H).

Step 4:

### 4-Pyridylmethyl 2,4-dioxo-3-thien-2-ylmethyl-1,2,3,4-tetrahydroquinazoline-6-carboxylate

0.69 g (2.3 mmol) of 2,4-dioxo-3-thien-2-ylmethyl-1,2,3,4-tetrahydroquinazoline-6-carboxylic acid is treated according to method F, using 4-pyridylcarbinol. The product is obtained as follows: MS: m/z 394.2 (M+H)+, NMR: DMSO ¹H δ (ppm) 5.21 (s,2H); 5.40 (s,2H); 6.93 (d,1H); 7.11 (m,1H); 7.28 (d,1H); 7.40 (d,1H); 7.40 (m,2H); 8.24 (d,1H); 8.49-8.59 (m,3H), CHN (C₂₀H₁₅N₃O₄S.0.13CH₂Cl₂.0.03 (ether)) calc: C=59.81; H=3.86; N=10.33. found: C=59.79; H=3.82; N=10.32.

### SYNTHESIS EXAMPLE 48

### 4-Pyridylmethyl 3-(benzo[1,3]dioxol-5-ylmethyl)-2,4-dioxo-1,2,3,4-tetrahydroquinazoline-6-carboxylate

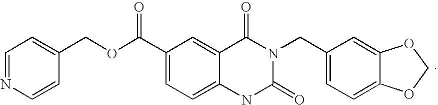

3-Benzo[1,3]dioxol-5-ylmethyl-2,4-dioxo-1,2,3,4-tetrahydroquinazoline 6-carboxylic acid (Example 34, step 3) in tetrahydrofuran is treated with thionyl chloride and the resulting acid chloride is treated with 4-pyridylcarbinol in dichloromethane in the presence of triethylamine. The product is crystallized from methanol: Weight: 0.040 g, TLC: CH₂Cl₂/MeOH 90/10 Rf=0.70, NMR: DMSO ¹H δ (ppm) 5.0 (s,2H); 5.70 (s,2H); 6.0 (s,2H); 6.85 (s,2H); 7.0 (s,1H); 7.4 (d,1H); 7.95-8.05 (m,2H); 8.3-8.35 (m,1H); 8.60 (s,1H); 8.8-8.95 (m,2H); 12.0 (m,1H), IR: 1710, 1670, 1622, 1501, 1440, 1279, 1236, 1041, 923; 764 cm⁻¹, m.p.=204.4° C., HPLC: 92.4%.

### SYNTHESIS EXAMPLE 49

### 3-Benzyl-1-methyl-2,4-dioxo-1,2,3,4-tetrahydro-pyrido[2,3-d]pyrimidine-6-carboxylic acid (1,3-benzodioxol-5-ylmethyl)-amide

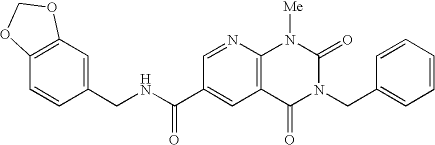

Step 1:

### N′-(1-Benzyl-3-methyl-2,6-dioxo-1,2,3,6-tetrahydro-pyrimidin-4-yl)-N,N-dimethyl-formamidine

0.56 g (2.5 mmol) of 6-amino-3-benzyl-1H-pyrimidine-2,4-dione (Tetrahedron Letters, 1991, 32(45), 6534-6540) in 20 ml of DMF are strirred under inert atmosphere. 1 ml (7.5 mmol) of N,N′-dimethylformamide dimethyl acetal is added to this solution and the mixture is heated to reflux for 20 minutes. After cooling and concentration under vacuum, the residue is taken up in dichloromethane, and the organic phase is washed with water, dried over Na₂SO₄, and concentrated under vacuum until a low volume. Then the crude product is precipitate by addition of ether. After filtration 0.680 g (yield: 72.6%) of the desired compound is obtained.

TLC: CH₂Cl₂/MeOH 90/10 Rf=0.80 NMR: DMSO ¹H δ (ppm): 3.0 (s,3H); 3.15 (s,3H); 3.30 (s,3H); 4.90 (s,2H); 5.20 (s,1H); 2-7.35 (m,5H); 8.10 (s,1H)

Step 2:

### N′-(1-Benzyl-5-iodo-3-methyl-2,6-dioxo-1,2,3,6-tetrahydro-pyrimidin-4-yl)-N,N-dimethyl-formamidine

To a stirred solution of 0.68 g (2.38 mmol) of the compound obtained in the preceding Step 1 in 24 ml of anhydrous dichloromethane is added 0.64 g (2.85 mmol) of N-iodosuccinimide. After 30 minutes of reflux, the reaction mixture is cooled and the organic phase is washed with water, dried over Na₂SO₄, and concentrated under vacuum. The crude product is precipitated in ether to obtain 0.680 g (yield: 69.3%) of the desired compound.

NMR: CDCl₃ ¹H δ (ppm): 3.05 (s,3H); 3.15 (s,3H); 3.40 (s,3H); 5.20 (s,2H); 7.2-7.30 (m,3H); 7.5-7.55 (m,2H); 7.7 (s,1H). M.P.=186.3° C.

Step 3:

### 3-Benzyl-1-methyl-2,4-dioxo-1,2,3,4-tetrahydro-pyrido[2,3-d]pyrimidine-6-carboxylic acid ethyl ester

To a stirred solution of 0.68 g (1.65 mmol) of the compound obtained in the preceding Step 2 in 45 ml of anhydrous DMF are added successively 18 mg Pd(OAc)₂, 8 mg of CuI, 330 mg of K₂CO₃, and 0.22 ml of ethyl acrylate. After 30 minutes under reflux, the reaction mixture is concentrated under vacuum. The residue is taken up in dichloromethane. The organic phase is filtered, washed two times with water, dried over Na₂SO₄ and then concentrated under vacuum. The crude product is purified by chromatography over silica gel (dichloromethane/methanol: 97/3) and then crystallized from ether to give 0.320 g (yield: 57%) of the desired compound.

TLC: CH₂Cl₂/MeOH 97.5/2.5 Rf=0.50 NMR: CDCl₃ ¹H δ (ppm): 1.40 (t,3H); 3.70 (s,3H); 4.40 (q,2H); 5.30 (s,2H); 7.2-7.30 (m,3H); 7.5-7.55 (m,2H); 9.0 (s,1H); 9.2 (s,1H)

Step 4:

### 3-Benzyl-1-methyl-2,4-dioxo-1,2,3,4-tetrahydro-pyrido[2,3-d]pyrimidine-6-carboxylic acid

The compound is obtained by hydrolysis, in a mixture of dioxan/water in presence of LiOH, of the compound obtained in the preceding Step 3.

TLC: CH₂Cl₂/MeOH 90/10 Rf=0.10 NMR: DMSO ¹H δ (ppm): 3.60 (s,3H); 5.20 (s,2H); 7.2-7.40 (m,5H); 8.75 (s,1H); 9.2 (s,1H); 13.5 (bs,1H) HPLC=100%

Step 5:

### 3-Benzyl-1-methyl-2,4-dioxo-1,2,3,4-tetrahydro-pyrido[2,3-d]pyrimidine-6-carboxylic acid (1,3-benzodioxol-5-ylmethyl)-amide

The compound is obtained according to the procedure of the synthesis Example 22 using the compound obtained in the preceding Step 4 and piperonylamine.

TLC: CH₂Cl₂/MeOH 95/5 Rf=0.60 NMR: DMSO ¹H δ (ppm): 3.60 (s,3H); 4.40 (d,2H); 5.2 (s,2H); 5.95 (s,2H); 6.75-6.95 (m,3H); 7.2-7.40 (m,5H); 8.85 (s,1H); 9.2 (s,1H); 9.25 (t,1H). IR: 3271, 1709, 1665, 1630, 1614, 1488, 1248, 1042, 937, 795 cm⁻¹ M.P.=174.9° C. HPLC: 97.5%

### SYNTHESIS EXAMPLE 50

### 4-[6-(4-Methoxy-benzylcarbamoyl)-1-methyl-2,4-dioxo-1,4-dihydro-2H-pyrido[2,3-d]pyrimidin-3-ylmethyl]-benzoic acid

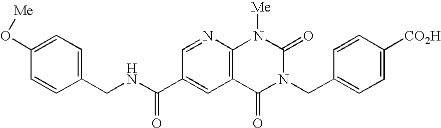

Step 1:

### 1-Methyl-2,4-dioxo-1,2,3,4-tetrahydro-pyrido[2,3-d]pyrimidine-6-carboxylic acid

A solution of 1.3 g (4.17 mmol) of the compound obtained in the Step 4 of the synthesis Example 49 and 3.1 g (23 mmol) of AlCl₃ in 44 ml of benzene is stirred 2 hours at room temperature. After addition of a mixture water/ice, the reaction mixture is extracted successively with ethyl acetate and dichloromethane. The aqueous layer is acidified at pH 1 by addition of concentrated HCl. The precipitate obtained is filtered off and washed with 10 ml of methanol and 10 ml of dichloromethane to provide the desired compound (yield: 62.9%)

NMR: DMSO ¹H δ (ppm): 3.50 (s,3H); 8.60 (s,1H); 9.10 (s,1H); 11.9 (bs,1H); 13.5 (bs,1H) HPLC=100%

Step 2:

### 1-Methyl-2,4-dioxo-1,2,3,4-tetrahydro-pyrido[2,3-d]pyrimidine-6-carboxylic acid 4-methoxy-benzylamide

The compound is obtained according to the procedure of the synthesis Example 22 using the compound obtained in the preceding Step 2 and 4-methoxybenzylamine.

TLC: CH₂Cl₂/MeOH 95/5 Rf=0.45 NMR: DMSO ¹H δ (ppm): 3.50 (s,3H); 3.7 (s,3H); 4.40 (d,2H); 6.85-6.95 (m,2H); 7.25-7.30 (m,2H); 8.80 (s,1H); 9.15 (s,1H); 9.30 (t,1H); 11.85 (bs,1H) HPLC=92%

Step 3:

### Methyl 4-[6-(4-Methoxy-benzylcarbamoyl)-1-methyl-2,4-dioxo-1,4-dihydro-2H-pyrido[2,3-d]pyrimidin-3-ylmethyl]-benzoate

The compound is obtained according to the procedure of the Step 2 of synthesis Example 38 using the compound obtained in the preceding Step 2 and methyl-4-(bromomethyl)benzoate. After concretization in ether 0.41 g (yield: 71.1%) of the desired compound is isolated.

TLC: CH₂Cl₂/MeOH 95/5 Rf=0.80 NMR: DMSO ¹H δ (ppm): 3.60 (s,3H); 3.80 (s,3H); 3.90 (s,3H); 4.45 (d,2H); 5.2 (s,2H); 6.90 (dd,2H); 7.30 (dd,2H); 7.50 (dd,2H); 7.90 (dd,2H); 8.90 (s,1H); 9.20 (s,1H); 9.30 (t,1H); HPLC=96.8%

Step 4:

### 4-[6-(4-Methoxy-benzylcarbamoyl)-1-methyl-2,4-dioxo-1,4-dihydro-2H-pyrido[2,3-d]pyrimidin-3-ylmethyl]-benzoic acid

The compound is obtained according to the procedure of synthesis Example 39 using the compound obtained in the preceding Step 3.

NMR: DMSO ¹H δ (ppm): 3.60 (s,3H); 3.70 (s,3H); 4.45 (d,2H); 5.20 (s,2H); 6.90 (d,2H); 7.25 (d,2H); 7.45 (d,2H); 7.90 (d,2H); 8.85 s,1H); 9.20 (s,1H); 9.30 (t,1H); 12.90 (bs,1H) IR: 3292, 1718, 1695, 1667, 1633, 1609, 1497, 1301, 1242, 797 cm⁻¹ M.P.=229.5° C. HPLC: 93.6%

### SYNTHESIS EXAMPLE 51

### 3-(4-Cyano-benzyl)-1-methyl-2,4-dioxo-1,2,3,4-tetrahydro-pyrido[2,3-d]pyrimidine-6-carboxylic acid 4-methoxy-benzylamide

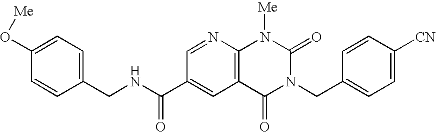

The compound is obtained (0.11 g; yield=68.4%) according to the procedure of the Step 2 of the synthesis Example 38 using the compound obtained in Step 2 of synthesis Example 50 and 4-(bromomethyl)benzonirile.

TLC: CH₂Cl₂/MeOH 95/5 Rf=0.70 NMR: DMSO ¹H δ (ppm): 3.60 (s,3H); 3.70 (s,3H); 4.40 (d,2H); 5.20 (s,2H); 6.90 (d,2H); 7.30 (d,2H); 7.55 (d,2H); 7.80 (d,2H); 8.85 (s,1H); 9.20 (s,1H); 9.30 (t,1H) IR: 3230, 2230, 1710, 1673, 1635, 1609, 1494, 1303, 1252, 794 cm⁻¹ M.P.=197° C. HPLC: 97.2%

### SYNTHESIS EXAMPLE 52

### 3-(4-Fluoro-benzyl)-1-methyl-2,4-dioxo-1,2,3,4-tetrahydro-pyrido[2,3-d]pyrimidine-6-carboxylic acid 4-methoxy-benzylamide

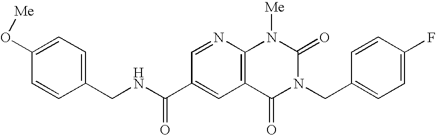

The compound is obtained according to the procedure of the Step 2 of the synthesis Example 38 using the compound obtained in Step 2 of synthesis Example 50 and 4-fluorobenzyl bromide.

TLC: CH₂Cl₂/MeOH 95/5 Rf=0.70 NMR: DMSO ¹H δ (ppm): 3.60 (s,3H); 3.70 (s,3H); 4.40 (d,2H); 5.10 (s,2H); 6.8-6.90 (m,2H); 7.1-7.2 (m,2H); 7.25-7.35 (m,2H); 7.4-7.50 (m,2H); 8.85 (s,1H); 9.15 (s,1H); 9.30 (t,1H). IR: 3260, 1709, 1664, 1616, 1497, 1245, 1221, 1035, 796 cm⁻¹ M.P.=211.5° C. HPLC: 98.3%

### SYNTHESIS EXAMPLE 53

### 3-Benzyl-1-methyl-2,4-dioxo-1,2,3,4-tetrahydro-pyrido[3,4-d]pyrimidine-6-carboxylic acid (1,3-benzodioxol-5-ylmethyl)-amide

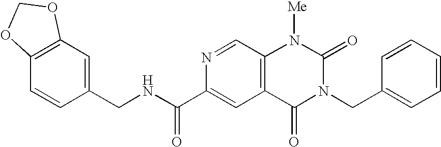

Step 1:

### 1-Benzyl-2,6-dioxo-1,2,3,6-tetrahydro-pyrimidine-4-carbaldehyde

A solution of 9.5 g (43.9 mmol) of 3-benzyl-6-methyl-1H-pyrimidine-2,4-dione (Synthetic Communications 1991, 2181-2188) and 129 ml of cold acetic acid are stirred 5 minutes, and 5.75 g of SeO₂ are added. The reaction mixture is heated to reflux for 2 h 30, filtered and concentrated under vacuum. The residue is taken up in dichloromethane. The unsoluble part is eliminated and the filtrate is concentrated under vacuum. A chromatography over silica gel (dichloromethane/methanol: 95/5) provides 4.0 g of the desired compound (yield: 39.5%).

NMR: CDCl₃ ¹H δ (ppm): 5.20 (s,2H); 6.30 (s,1H); 7.2-7.30 (m,3H); 7.40-7.50 (m,2H); 9.0 (bs,1H); 9.60 (s,1H)

Step 2:

### 1-Benzyl-2,6-dioxo-1,2,3,6-tetrahydro-pyrimidine-4-carbaldehyde dimethylhydrazone

To a stirred solution of 3.6 g (15.6 mmol) of the compound obtained in the preceding Step 1 in 80 ml of anhydrous DMF are added 1.2 ml (0.94 g, 15.6 mmol) of dimethylhydrazine. After 1 hour of stirring at room temperature, the solvent is removed under vacuum and the residue is taken up in dichloromethane. The organic layer is washed, dried over Na₂SO₄ and concentrated. A chromatography over silica gel (dichloromethane/methanol: 97/3) provides 2.5 g (yield: 59%) of the desired compound.

NMR: CDCl₃ ¹H δ (ppm) 3.10 (s,6H); 5.10 (s,2H); 5.55 (s,1H); 6.50 (s,1H); 7.2-7.30 (m,3H); 7.40-7.50 (m,2H); 8.50 (bs,1H)

Step 3:

### 1-Benzyl-2,6-dioxo-3-methyl-1,2,3,6-tetrahydro-pyrimidine-4-carbaldehyde dimethylhydrazone

To a stirred solution of 2.3 g (8.45 mmol) of the compound obtained in the preceding Step 2 in 58 ml of anhydrous DMF are added 2.3 ml (2.0 g, 1.69 mmol) of N,N′-dimethylformamide acetal. The reaction mixture is maintained at 100° C. for 10 minutes and concentrated under vacuum. The residue is taken up in dichloromethane and the product is precipitated by addition of ether to provide 1.75 g (yield: 72.3%) of the desired compound.

NMR: CDCl₃ ¹H δ (ppm) 3.20 (s,6H); 3.50 (s,3H); 5.15 (s,2H); 6.10 (s,1H); 6.60 (s,1H); 7.2-7.30 (m,3H); 7.40-7.50 (m,2H)

Step 4:

### Methyl 1-benzyl-2,6-dioxo-3-methyl-1,2,3,6-tetrahydro-pyrimidine-4-(carbaldehyde dimethylhydrazone)-5-carboxylate

To a stirred solution of 1.7 g (5.94 mmol) of the compound obtained in the preceding Step 3 in 61 ml of anhydrous acetonitrile are added successively 1.68 g (7.1 mmol) of Pd(OAc)₂ and 0.613 g (7.1 mmol) of methyl acrylate. After 20 minutes od stirring under reflux the reaction mixture is filtered off and concentrated under vacuum. The residue is chromatographied over silica gel (dichloromethane/methanol: 97/3) to provide 1.40 g (yield: 63.6%) of the desired compound.

NMR: CDCl₃ ¹H δ (ppm): 3.20 (s,6H); 3.55 (s,3H); 3.75 (s,3H); 5.20 (s,2H); 6.70 (s,1H); 7.1-7.70 (m,7H).

Step 5:

### 3-Benzyl-1-methyl-2,4-dioxo-1,2,3,4-tetrahydro-pyrido[3,4-d]pyrimidine-6-carboxylic acid methyl ester

A solution of 1.4 g (3.78 mmol) of the compound obtained in the preceding Step 4, 18 ml of chlorobenzene and 3.6 ml of acetic acid is stirred under reflux for 3 hours, and concentrated under vacuum to provide 1.4 g of a precipitate. The desired compound (0.76 g; yield: 62%) is obtained by recrystallization of the crude product in 120 ml of ethyl acetate.

NMR: CDCl₃ ¹H δ (ppm): 3.70 (s,3H); 4.0 (s,3H); 5.30 (s,2H); 7.2-7.35 (m,3H); 7.45-7.55 (m,2H); 8.80 (s,1H); 8.85 (s,1H).

Step 6:

### 3-Benzyl-1-methyl-2,4-dioxo-1,2,3,4-tetrahydro-pyrido[3,4-d]pyrimidine-6-carboxylic acid

0.76 g (2.34 mmol) of the compound obtained in the preceding Step 5, 7.6 ml of methanol, 7.6 ml of water and 0.646 g (4.67 mmol) of K₂CO₃ are stirred overnight at room temperature and then heated to reflux for 5 minutes. After cooling and addition of water the acification to pH 1 of the mixture provides a precipitate which is dissolved in a mixture of methanol/dichloromethane. The organic layer is washed with water, dried and concentrated under vacuum. The residue obtained is concretized in a mixture of dichloromethane/ether to give 0.54 g (yield: 74%) of the desired compound.

NMR: DMSO ¹H δ (ppm) 3.60 (s,3H); 5.20 (s,2H); 7.2-7.40 (m,5H); 8.50 (s,1H); 9.0 (s,1H); 13.3 (bs,1H) M.P.=240° C. HPLC=100%

Step 7:

### 3-Benzyl-1-methyl-2,4-dioxo-1,2,3,4-tetrahydro-pyrido[3,4-d]pyrimidine-6-carboxylic acid (1,3-benzodioxol-5-ylmethyl)-amide

The compound is obtained according to the procedure of the synthesis Example 22 using the compound obtained in the preceding Step 6 and piperonylamine.

TLC: CH₂Cl₂/MeOH 95/5 Rf=0.60 NMR: DMSO ¹H δ (ppm): 3.65 (s,3H); 4.40 (d,2H); 5.15 (s,2H); 5.95 (s,2H); 6.75-6.85 (m,2H); 6.90 (s,1H); 7.2-7.40 (m,5H); 8.45 (s,1H); 8.90 (s,1H); 9.25 (t,1H). IR: 3387, 1716, 1662, 14875, 1442, 1250, 1239, 1040, 789 cm⁻¹ M.P.=197.5° C. HPLC: 100%

### SYNTHESIS EXAMPLE 54

### Methyl 4-[6-(4-Methoxy-benzylcarbamoyl)-1-methyl-2,4-dioxo-1,4-dihydro-2H-pyrido[3,4-d]pyrimidin-3-ylmethyl]-benzoate

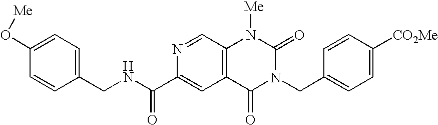

Step 1:

### 1-Methyl-2,4-dioxo-1,2,3,4-tetrahydro-pyrido[3,4-d]pyrimidine-6-carboxylic acid

3.3 g (10.6 mmol) of the compound obtained in the Step 6 of the synthesis Example 53 are treated according to the procedure described in the Step 1 of the synthesis Example 46 to give 2.0 g (yield: 85.3%) of the desired compound.

NMR: DMSO ¹H δ (ppm): 3.60 (s,3H); 8.40 (s,1H); 8.95 (s,1H); 12.0 (s,1H); 12.90 (bs,1H) HPLC=100%

Step 2:

### 1-Methyl-2,4-dioxo-1,2,3,4-tetrahydro-pyrido[3,4-d]pyrimidine-6-carboxylic acid 4-methoxy-benzylamide

The compound is obtained (yield: 78%) according to the procedure of the synthesis Example 22 using the compound obtained in the preceding Step 1 and 4-methoxybenzylamine.

TLC: CH₂Cl₂/MeOH 95/5 Rf=0.50 NMR: DMSO ¹H δ (ppm): 3.60 (s,3H); 3.75 (s,3H); 4.40 (d,2H); 6.85 (dd,2H); 7.25 (dd,2H); 8.40 (s,1H); 8.85 (s,1H); 9.20 (t,1H); 12.0 (s,1H) HPLC=99%

Step 3:

### Methyl 4-[6-(4-methoxy-benzylcarbamoyl)-1-methyl-2,4-dioxo-1,4-dihydro-2H-pyrido[3,4-d]pyrimidin-3-ylmethyl]-benzoate

The compound is obtained (0.2 g; yield: 77%) according to the procedure of the Step 2 of synthesis Example 38 using the compound obtained in the preceding Step 2 and methyl-4-(bromomethyl)benzoate.

TLC: CH₂Cl₂/MeOH 95/5 Rf=0.80 NMR: DMSO ¹H δ (ppm): 3.60 (s,3H); 3.70 (s,3H); 3.85 (s,3H); 4.50 (d,2H); 5.20 (s,2H); 6.85 (d,2H); 7.20 (d,2H); 7.50 (d,2H); 7.90 (d,2H); 8.5 (s,1H); 8.90 (s,1H); 9.20 (t,1H) IR: 3396, 1719, 1661, 1439, 1279, 1250, 1110, 753 cm⁻¹ M.P.=211.1° C. HPLC: 99.5%

Cyclised Quinazolines

We have made a fifth group of compounds which are cyclized quinazolines and are inhibitors of matrix metalloproteinase enzymes, and especially MMP-13. Preferred compounds that we have made, and their ability to inhibit the activity of MMP-13 are summarized in Table V below:

TABLE V

| | | IC50 |
|---|---|---|
| Compound name | Structure | μM |
| 4-Benzyl-5-oxo-4,5-dihydro- [1,2,4]triazolo[4,3- a]quinazoline-7- carboxylic acid benzyl ester | 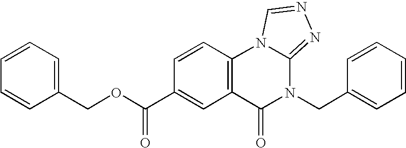 | 0.0034 |
| 4-Benzyl-5-oxo-4,5-dihydro- [1,2,4]triazolo[4,3- a]quinazoline-7- carboxylic acid pyridin-4- ylmethyl ester | 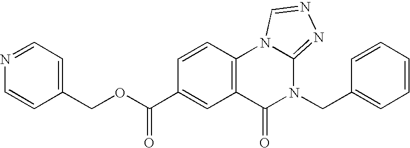 | 0.0023 |
| 3-Benzyl-5-oxo-4,5-dihydro- [1,2,4]triazolo[4,3- a]quinazoline-7- carboxylic acid (benzo[1,3]dioxol-5-ylmethyl)- amide | 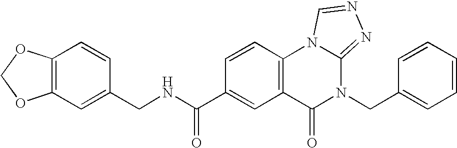 | 0.0040 |
| 4-Benzyl-5-oxo-4,5-dihydro- [1,2,4]triazolo[4,3- a]quinazoline-7- carboxylic acid (pyridin-4- ylmethyl)-amide | 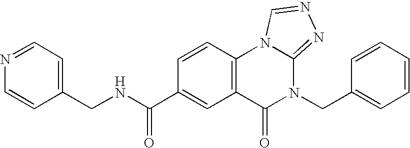 | 0.040 |
| 4-Benzyl-5-oxo-4,5-dihydro- imidazo[1,2-a]quinazoline-7- carboxylic acid (benzo[1,3]dioxol-5- ylmethyl)-amide | 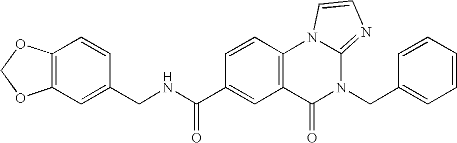 | 0.165 |
| 4-Benzyl-5-oxo-4,5-dihydro- imidazo[1,2-a]quinazoline-7- carboxylic acid (pyridin-4- ylmethyl)-amide | 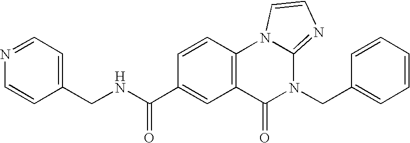 | 2.1 |
| N-(4-Methoxybenzyl)-4-benzyl- 5-oxo-4,5-dihydro[1,2,4]triazolo [4,3-a]quinazoline-7- carboxamide | 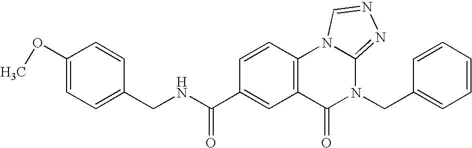 | 0.0055 |
| N-[3-(4-Pyridylsulphanyl) propyl]-4-benzyl-5-oxo-4,5- dihydro[1,2,4]triazolo[4,3-a]quinazoline-7-carboxamide | 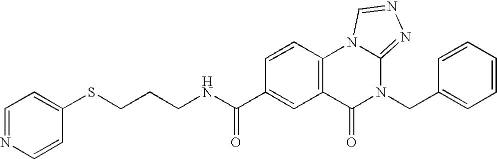 | 0.185 |
| N-(3,4-Methylenedioxybenzyl)- 4-(4-cyanobenzyl)-5-oxo-4H- [1,2,4]triazolo[4,3-a] quinazol- 7-ylcarboxamide | 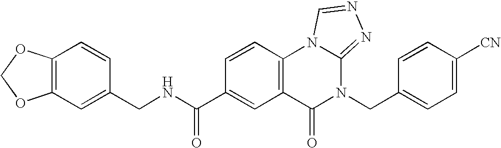 | 0.0023 |
| Methyl 4-{7-[(1,3-benzodioxol- 5-ylmethyl)-carbamoyl]-5-oxo- 5H-[1,2,4]triazolo[4,3-a]quinazol-4-ylmethyl}benzoate | 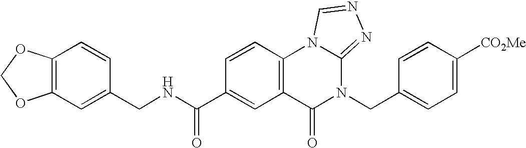 | 0.0011 |
| Methyl 4-{7-[(4-methoxy benzyl)-carbamoyl]-5-oxo-5H- [1,2,4]triazolo[4,3-a] quinazol- 4-ylmethyl} benzoate | 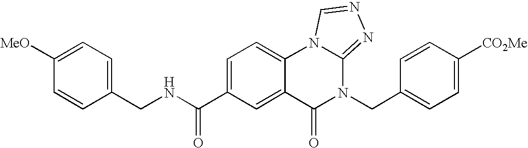 | 0.0026 |
| Methyl 4-{7-[(pyridin-4- ylmethyl)-carbamoyl]-5-oxo- 5H-[1,2,4]triazolo[4,3-a]quinazol-4-ylmethyl}benzoate | 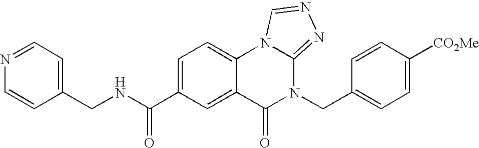 | 0.012 |
| (2-Dimethylamino-ethyl) 4-[7- (4-fluoro-benzylcarbamoyl)-5- oxo-5H-[1,2,4]triazolo [4,3-a]quinazol-4-ylmethyl] benzoate | 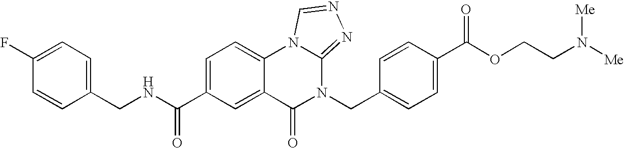 | nt |
| 4-(4-Dimethylcarbamoyl- benzyl)-5-oxo-4,5-dihydro- [1,2,4]triazolo[4,3-a]quinazoline-7-carboxylic acid 4-methoxy-benzylamide | 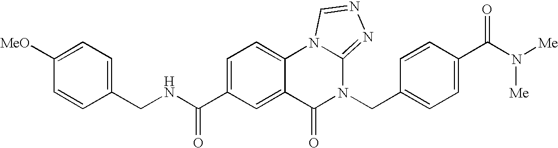 | 0.0087 |
| N-(pyridin-4ylmethyl)-4-(4- cyanobenzyl)-5-oxo-4H- [1,2,4]triazolo[4,3-a]quinazol-7- ylcarboxamide | 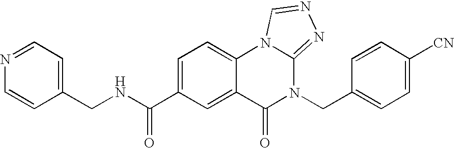 | 0.021 |
| Methyl (4-{7-[(1,3- benzodioxol-5-ylmethyl)- carbamoyll-5-oxo- 5H-[1,2,4]triazolo[4,3-a]quinazolin-4-ylmethyl}- phenyl)-acetate | 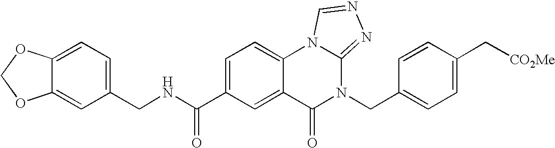 | 0.0022 |
| Methyl (4-{7-[(4-methoxy)- benzylcarbamoyl]-5-oxo-5H- [1,2,4]triazolo[4,3-a]quinazolin-4-ylmethyl}- phenyl)-acetate | 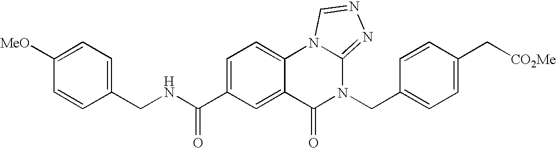 | 0.0029 |
| Methyl (4-{7-[(pyridin-4-yl)- methylcarbamoyl]-5-oxo-5H- [1,2,4]triazolo[4,3-a]quinazolin-4-ylmethyl}- phenyl)-acetate | 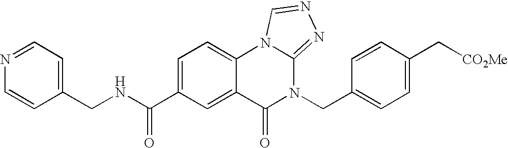 | 0.013 |
| N-(pyridin-4-ylmethyl) 4-[3- (pyridin-4-yl)-2-propen-1-yl]-5- oxo-4H-[1,2,4]triazolo[4,3-a]quinazol-7-ylcarboxamide | 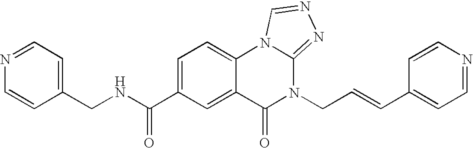 | 0.350 |
| 4-[2-(4-Chloro-phenoxy)-ethyl]- 5-oxo-4,5-dihydro-[1,2,4]triazolo[4,3-a]quinazoline-7- carboxylic acid 4-methoxy- benzylamide | 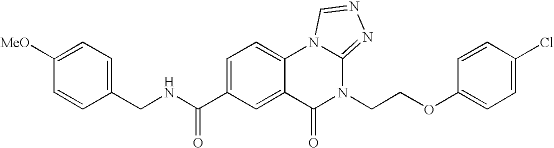 | 0.0865 |
| 4-{7-[(4-Methoxybenzyl)- carbamoyl]-5-oxo-5H- [1,2,4]triazolo[4,3-a]quinazol-4- ylmethyl} benzoic acid | 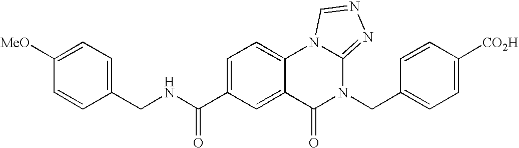 | 0.0011 |
| 4-{7-[(1,3-Benzodioxol-5- ylmethyl)-carbamoyl]-5-oxo- 5H-[1,2,4]triazolo[4,3-a]quinazol-4-ylmethyl}benzoic acid | 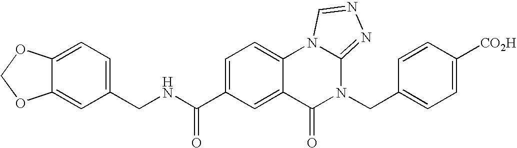 | 0.0009 |
| 4-{7-[(Pyridin-4-ylmethyl)- carbamoyl]-5-oxo-5H-[1,2,4]triazolo[4,3-a]quinazol-4- ylmethyl} benzoic acid | 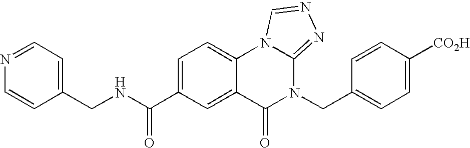 | 0.0042 |
| 4-{7-[(4-Fluoro)-benzyl carbamoyl]-5-oxo-5H- [1,2,4]triazolo[4,3-a]quinazol-4-ylmethyl}benzoic acid | 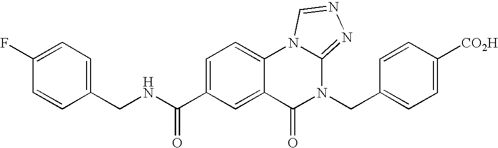 | 0.0011 |
| (4-{7-[(4-Methoxy)-benzyl carbamoyl]-5-oxo-5H- [1,2,4]triazolo[4,3-a]quinazolin-4-ylmethyl}- phenyl)-acetic acid | 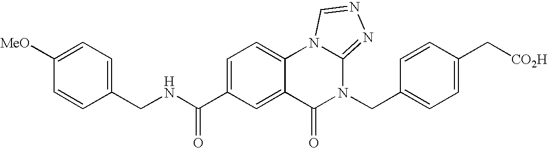 | 0.0013 |
| (4-{7-[(1,3-Benzodioxol-5- ylmethyl)-carbamoyl]-5-oxo- 5H-[1,2,4]triazolo[4,3-a]quinazolin-4-ylmethyl}- phenyl)-acetic acid | 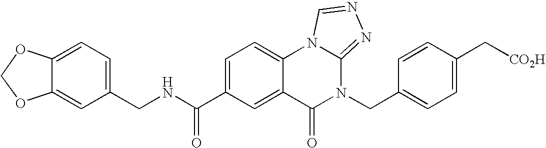 | 0.0011 |
| (4-{7-[(Pyridin-4-yl)- methylcarbamoyl]-5-oxo-5H- [1,2,4]triazolo[4,3-a]quinazolin- 4-ylmethyl}-phenyl)-acetic acid | 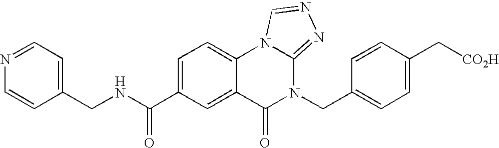 | 0.0062 |

nt: not tested

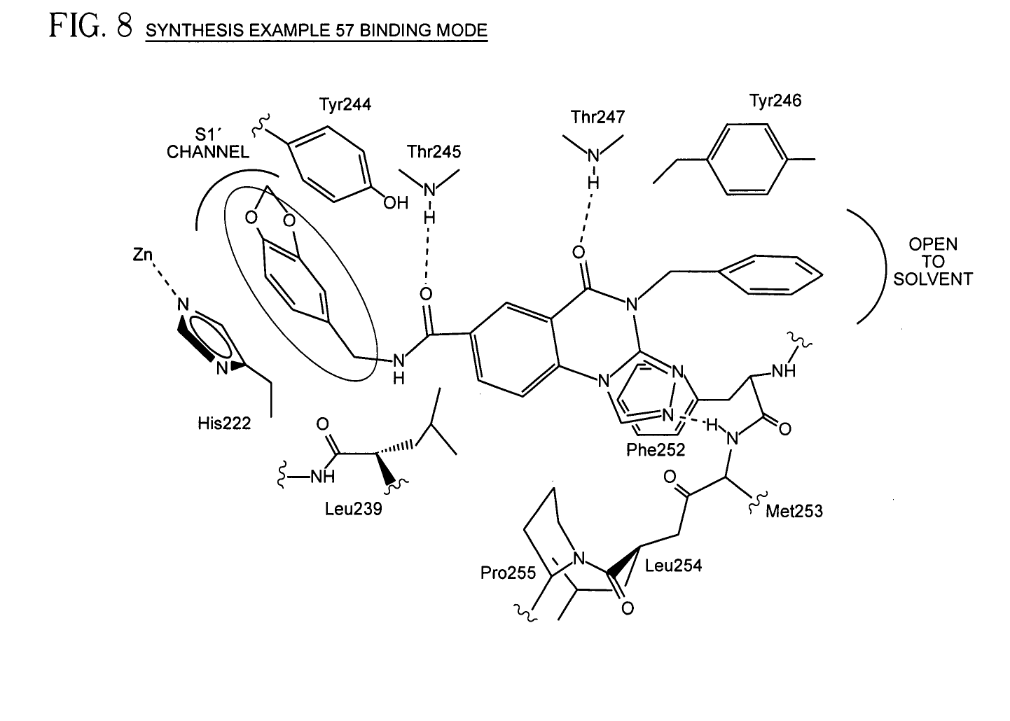

Binding of a representative compound in this series, Synthesis Example 57 is shown in FIG. 8 and involves first and second hydrophobic groups and first, second and third hydrogen bond acceptors as for the compounds of the previous series.

Synthesis of some of the compounds referred to in Table V is described in the following synthesis examples. The synthesis of the other compounds in the Table V is reported in our co-pending WO application which claims the priority of the application No. U.S. 60/268,757 filed on Feb. 14, 2001.

Starting Materials

For preparation of the starting material for Step 1 of Synthesis Example 57 below, 5-bromo-2-hydrazino benzoic acid may be treated with a cyanoimidate to give a 4-benzyl-6-bromo-4,5-dihydrotriazolo[2,3-a]quinazolin-5-one in a single step. The compound may then be converted to a 4-N-substituted analogue by reaction with a halide in the presence of a base, e.g. cesium carbonate, in a solvent such as dimethylformamide. The bromine in position 7 is replaced by cyanide by exchange with copper cyanide in a solvent such as N-methylpyrrolidone. For preparation of the carboxylic acid used as starting material in Synthesis Example 59, the cyano-compound is hydrolysed by acid, e.g. sulphuric acid.

### SYNTHESIS EXAMPLE 55

### Benzyl 4-benzyl-5-oxo-4H-[1,2,4]triazolo[4,3-a]quinazol-7-ylcarboxylate

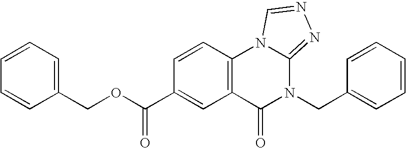

Step 1:

### 1,2,3,4-Tetrahydro-4-benzyl-7-cyano-4H-[1,2,4]triazolo[4,3-a]quinazolin-5-one

26.5 g (0.08 mol) of 1,2,3,4-tetrahydro-4-benzyl-7-bromo-4H-[1,2,4]triazolo[4,3-a]quinazolin-5-one and 12.15 g (0.14 mol) of copper cyanide are placed in 250 ml of N-methylpyrrolidinone in a reactor fitted with a stirring system and a condenser equipped with a potassium hydroxide guard tube. The mixture obtained is stirred and gradually heated to 220° C. and this temperature is then maintained for 3 hours. After partial cooling, the solvent is evaporated off under vacuum; the residue obtained is partitioned between dilute aqueous ammonia and methylene chloride, and the insoluble material in the two phases is removed by filtration after washing several times with aqueous ammonia and methylene chloride. The organic phase is separated out after settling has taken place, washed with saturated sodium chloride solution, dried over sodium sulphate and then concentrated under vacuum. The residual solid is taken up in 50 ml of ethanol and the insoluble material is spin-filtered and dried under vacuum to give 15.75 g, which is pure by TLC. The ¹H NMR spectrum is compatible with the expected structure. Yield=65% TLC (CH₂Cl₂ 95/CH₃OH 5): R_{f}=0.75.

Step 2:

### 1,2,3,4-Tetrahydro-4-benzyl-4H-[1,2,4]triazolo[4,3-a]5-oxoquinazolin-7-ylcarboxylic acid.

A solution of 150 ml of concentrated sulphuric acid in 150 ml of water is prepared, in a round-bottomed flask fitted with a stirrer and a condenser, while cooling externally with an ice bath. 7.0 g (0.023 mol) of 1,2,3,4-tetrahydro-4-benzyl-7-cyano-4H-[1,2,4]triazolo[4,3-a]quinazolin-5-one (intermediate of general formula (5b)) are added and the mixture is then refluxed with stirring for 2 h 30 min. After cooling, the mixture is filtered and 500 ml of ice-cold water are added to the acidic solution obtained. The precipitate is filtered off, washed several times with water to neutral pH and dried under vacuum to give 5.1 g of solid. The ¹H NMR spectrum is compatible with the expected structure. Yield=69%.

Step 3:

### Benzyl 4-benzyl-5-oxo-4H-[1,2,4]triazolo[4,3-a]quinazol-7-ylcarboxylate

0.64 g (0.002 mol) of 1,2,3,4-tetrahydro-4-benzyl-4H-[1,2,4]triazolo[4,3-a]-5-oxoquinazolin-7-ylcarboxylic acid are placed in 100 ml of DMF in a reactor equipped with a condenser and a magnetic stirrer. 0.276 g (0.002 mol) of K₂CO₃ is added and the mixture is stirred at room temperature for 30 minutes. 0.342 g (0.002 mol) of benzyl bromide is then added and the mixture is heated to 100° C. and then stirred at this temperature for 15 hours. After evaporating off the solvent under vacuum, the residue is taken up in a mixture of water and ethyl acetate; the insoluble solid in the 2 phases is filtered off, washed with water and an additional small amount of ethyl acetate and then dried under vacuum to give 0.45 g of crude compound (55% of the theoretical amount). This product is purified by chromatography on a column of silica, eluting with a CH₂Cl₂ 99/CH₃OH 1 mixture: 0.2 g of compound, which is pure by TLC, is obtained. Recrystallization from acetonitrile gives colourless crystals, m.p. (Tottoli)=221° C., TLC (CH₂Cl₂ 98/CH₃OH 2 ): R_{f}=0.4, ¹H NMR δ (ppm) [DMSO]: 5.4 (s, 2H); 5.45 (s, 2H); 7.3-7.55 (m, 10H); 8.35 (d, 1H); 8.5 (d, 1H); 8.75 (s, 1H); 9.6 (s, 1H). Elemental analysis: Calculated: C, 70.23; H, 4.42; N, 13.65; O, 11.69. Found: C, 69.81; H, 4.32; N, 13.58; O 11.92.

### SYNTHESIS EXAMPLE 56

### 4-Pyridylmethyl 4-benzyl-5-oxo-4H-[1,2,4]triazolo[4,3-a]quinazol-7-yl-carboxylate

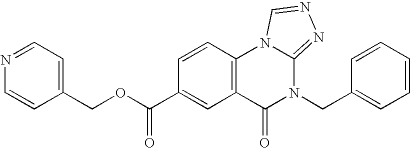

The above compound is prepared according to the method described in Synthesis Example 55, using 4-bromomethylpyridine in step 1. Yield=46%, m.p. (Tottoli)=232° C., ¹H NMR δ (ppm) [DMSO]: 5.4 (s, 2H); 5.5 (s, 2H); 7.25-7.4 (m, 3H); 7.45-7.55 (m, 4H); 8.4 (d, 1H); 8.55 (d, 1H); 8.65 (d, 2H); 8.8 (s, 1H); 9.65 (s, 1H).

### SYNTHESIS EXAMPLE 57

### N-(3,4-Methylenedioxybenzyl)-4-benzyl-5-oxo-4H-[1,2,4]triazolo[4,3-a]quinazol-7-ylcarboxamide

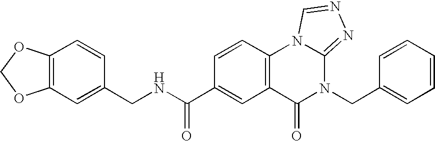

0.32 g (0.001 mol) of 4-benzyl-5-oxo-4H-[1,2,4]triazolo[4,3-a]quinazol-7-yl-carboxylic acid is dissolved in 15 ml of dry DMF in a reactor protected from moisture, equipped with a stirring system and a thermometer. 0.124 ml (0.001 mol) of 3,4-methylenedioxybenzylamine and 0.328 g (0.001 mol) of TOTU are then added, the mixture is stirred, the solution obtained is cooled to 0-5° C. and 0.258 mg (0.002 mol) of DIPEA is then added. The solution is stirred under cold conditions for a few minutes and then at room temperature for 15 hours. After evaporating off the solvent under vacuum, the residue is taken up in methylene chloride and the insoluble material is separated out by filtration, washed with a small additional amount of CH₂Cl₂ and then dried under vacuum to give 0.35 g of crude compound (77% of theoretical amount). 0.3 g of this product is recrystallized from dioxane to give 0.15 g of product which is pure by TLC. (R_{f}=0.35; eluent: CH₂Cl₂ (80)/CH₃OH (20)). m.p. (Tottoli)=273° C. (dec) ¹H NMR δ (ppm) [DMSO]: 4.45 (d, 2H); 5.45 (s, 2H); 6.0 (s, 2H); 6.8-7.0 (m, 3H); 7.25-7.4 (m, 3H); 7.5 (m, 2H); 8.3 (d, 1H); 8.4 (d, 1H); 8.8 (s, 1H); 9.35 (t, 1H); 9.6 (s, 1H).

### SYNTHESIS EXAMPLE 58

### N-(3,4-Methylenedioxybenzyl)-4-(4-cyanobenzyl)-5-oxo-4H-[1,2,4]triazolo[4,3-a]quinazol-7-ylcarboxamide

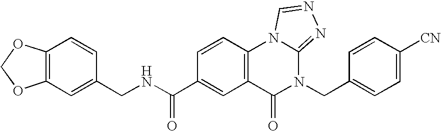

0.7 g (1.9 mmol) of N-(3,4-methylenedioxybenzyl)-4H-[1,2,4]triazolo[4,3-a]-5-oxo-quinazol-7-yl carboxamide in suspension in 20 ml of dimethylformamide and 0.62 g (1.9 mmol) of cesium carbonate are placed in a reactor fitted with a stirring system. The mixture is stirred 15 minutes at room temperature and 0.372 g (1.9 mmol) of 4-cyanobenzyl bromide is added. The reaction mixture is stirred at 90° C. for 12 hours and concentrated under vacuum. The residu obtained is taken up in a mixture of water and dichloromethane. The organic phase is separated, washed with brine and evaporated under vacuum. A chromatography of the residu over silica gel (dichloromethane/methanol: 95/5) yield 0.55 g (60%) of the desired compound pure on TLC. A recrystallisation from acetonitrile give 0.32 of uncolourless crystals.

m.p. (Tottoli)=215° C. ¹H NMR δ (ppm) [DMSO]: 4.4 (d, 2H); 5.45 (s, 2H); 6.0 (s, 2H); 6.8-6.9 (m, 2H); 6.95 (s, 1H); 7.6 (m, 2H); 7.8 (m, 2H); 8.3 (m, 2H); 8.4 (m, 1H); 8.8 (s, 1H); 9.3 (t, 1H); 9.6 (s, 1H).

### SYNTHESIS EXAMPLE 59

### Methyl 4-{7-[(4-methoxybenzyl)-carbamoyl]-5-oxo-5H-[1,2,4]triazolo[4,3-a]quinazol-4-ylmethyl}benzoate

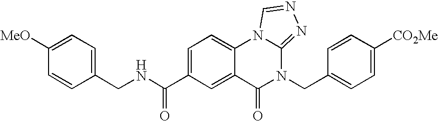

m.p. (Tottoli)=210° C. ¹H NMR δ (ppm) [DMSO]: 3.7 (s, 3H); 3.8 (s, 3H); 4.4 (d, 2H); 5.4 (s, 2H); 6.9 (d, 2H); 7.3 (d, 2H); 7.6 (d, 2H); 7.9 (d, 2H); 8.3 (d, 1H); 8.4 (d, 1H); 8.75 (s, 1H); 9.35 (t, 1H); 9.55 (s, 1H).

### SYNTHESIS EXAMPLE 60

### 4-{7-[(4-Methoxybenzyl)-carbamoyl]-5-oxo-5H-[1,2,4]triazolo[4,3-a]quinazol-4-ylmethyl}benzoic acid

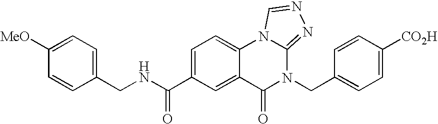

8.8 g (17.7 mmol) of compound obtained in the Synthesis Example 59 in suspension in 900 ml of a mixture (water/methanol: 50/50) and 2.45 g (17.7 mmol) of potassium carbonate are placed in a reactor fitted with a stirring system. The mixture is heated under reflux for 45 minutes and 2.45 g (17.7 mmol) of potassium carbonate are added. After 30 minutes of stirring under reflux, the reaction mixture is partially concentrated under vacuum and a mixture of ice acetic acid and ice is added to provide a precipitate which is filtered, washed with water until neutral pH, and then with methanol. After dried under vacuum, 6.1 g (yield=61%) of the uncolourless desired product are obtained.

¹H NMR δ (ppm) [DMSO]: 3.8 (s, 3H); 4.45 (d, 2H); 5.45 (s, 2H); 6.9 (d, 2H); 7.3 (d, 2H); 7.55 (d, 2H); 8.3 (d, 2H); 8.4 (d, 1H); 8.75 (s, 1H); 9.4 (t, 1H); 9.55 (s, 1H); 12.9 (s, 1H).

### SYNTHESIS EXAMPLE 61

### 4-{7-[(1,3-Benzodioxol-5-ylmethyl)-carbamoyl]-5-oxo-5H-[1,2,4]triazolo[4,3-a]quinazol-4-ylmethyl}benzoic acid

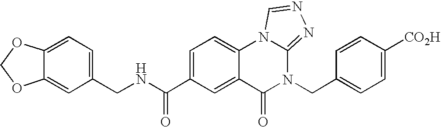

m.p. (Tottoli)=235° C. ¹H NMR δ (ppm) [DMSO]: 4.4 (d, 2H); 5.4 (s, 2H); 6.0 (s, 2H); 6.8 (m, 2H); 6.9 (d, 2H); 7.5 (d, 2H); 7.9 (d, 2H); 8.3 (d, 2H); 8.4 (d, 2H); 8.75 (s, 1H); 9.4 (t, 1H); 9.6 (s, 1H).

### 1,1-dioxy-benzo-(1,2,4)-thiadiazine

We have made a sixth group of compounds which are 1,1-dioxy-benzo-(1,2,4)-thiadiazines and are inhibitors of matrix metalloproteinase enzymes, and especially MMP-13. Synthesis of some of the compounds referred to in Table V is described in the following synthesis examples. The synthesis of the other compounds in the Table V is reported in our co-pending WO application which claims the priority of the application No. U.S. 60/268,782 filed on Feb. 14, 2001.

TABLE VI

| Synthesis | MMP01 | MMP02 | MMP03 | MMP07 | MMP09 | MMP013 | MMP014 |
|---|---|---|---|---|---|---|---|
| example | (μM) | (μM) | (μM) | (μM) | (μM) | (μM) | (μM) |
| 62 | >100 | >100 | 85 | 44 | >100 | 0.2 | >100 |
| 63 | >100 | >30 | >30 | >30 | >30 | 0.88 | >30 |
| 64 | nt | nt | nt | nt | nt | 0.51 | nt |
| 65 | >30 | nt | 16 | >30 | >30 | 0.615 | >30 |

| | MMP01 | MMP03 | MMP13 |
|---|---|---|---|
| | IC50 | IC50 | IC50 |
| Other Compounds | (nM) | (nM) | (nM) |
| 2-Benzyl-4-methyl-1,1,3-trioxo-1,2,3,4-tetrahydro-1λ⁶- | >30 | >30 | 0.17 |
| benzo[1,2,4]thiadiazine-7-carboxylic acid 4-methoxy- | | | |
| benzylamide | | | |
| 4-(7-Benzylcarbamoyl-4-methyl-1,1,3-trioxo-3,4- | >100 | >100 | 0.066 |
| dihydro-H-1λ⁶-benzo[1,2,4]thiadiazin-2-ylmethyl)- | | | |
| benzoic acid | | | |
| 4-[7-(4-Methoxy-benzylcarbamoyl)-4-methyl-1,1,3- | >100 | 64 | 0.011 |
| trioxo-3,4-dihydro-1H-1λ⁶-benzo[1,2,4]thiadiazin-2- | | | |
| ylmethyl]-benzoic acid | | | |
| 2-(4-Carbamoyl-benzyl)-4-methyl-1,1,3-trioxo-1,2,3,4- | >30 | >100 | 0.155 |
| tetrahydro-1λ⁶-benzo[1,2,4]thiadiazine-7-carboxylic | | | |
| acid 4-methoxy-benzylamide | | | |
| 2-Benzyl-4-methyl-1,1,3-trioxo-1,2,3,4-tetrahydro-1λ⁶- | >100 | >100 | 0.345 |
| benzo[1,2,4]thiadiazine-7-carboxylic acid 4-fluoro- | | | |
| benzylamide | | | |
| 4-Methyl-2-(4-methylsulfamoyl-benzyl)-1,1,3-trioxo- | >30 | 10 | 0.31 |
| 1,2,3,4-tetrahydro-1λ⁶-benzo[1,2,4]thiadiazine-7- | | | |
| carboxylic acid 4-methoxy-benzylamide | | | |
| 4-Methyl-2-[4-(morpholine-4-sulfonyl)-benzyl]-1,1,3- | >30 | 11 | 0.23 |
| trioxo-1,2,3,4-tetrahydro-1λ⁶-benzo[1,2,4]thiadiazine- | | | |
| 7-carboxylic acid 4-methoxy-benzylamide | | | |
| 4-[7-(4-Fluoro-benzylcarbamoyl)-4-methyl-1,1,3- | >30 | <30 | 0.385 |
| trioxo-3,4-dihydro-1H-1λ⁶-benzo[1,2,4]thiadiazin-2- | | | |
| ylmethyl]-benzoic acid methyl ester | | | |
| 2-Benzyl-4-methyl-1,1,3-trioxo-1,2,3,4-tetrahydro-1λ⁶- | >30 | >30 | 0.155 |
| benzo[1,2,4]thiadiazine-7-carboxylic acid (2-methoxy- | | | |
| pyridin-4-ylmethyl)-amide | | | |
| 4-Methyl-2-naphthalen-2-ylmethyl-1,1,3-trioxo- | >30 | >30 | 0.62 |
| 1,2,3,4-tetrahydro-1λ⁶-benzo[1,2,4]thiadiazine-7- | | | |
| carboxylic acid 4-methoxy-benzylamide | | | |
| 2-Benzyl-4-methyl-1,1,3-trioxo-1,2,3,4-tetrahydro-1λ⁶- | >30 | 13 | 0.125 |
| benzo[1,2,4]thiadiazine-7-carboxylic acid (2,1,3- | | | |
| benzothiadiazol-5-ylmethyl)-amide | | | |
| 4-[7-(4-Fluoro-benzylcarbamoyl)-4-methyl-1,1,3- | >100 | >30 | 0.019 |
| trioxo-3,4-dihydro-1H-1λ⁶-benzo[1,2,4]thiadiazin-2- | | | |
| ylmethyl]-benzoic acid | | | |
| 4-[7-(4-Methoxy-benzylcarbamoyl)-4-methyl-1,1,3- | nt | nt | 2.2 |
| trioxo-3,4-dihydro-1H-1λ⁶-benzo[1,2,4]thiadiazin-2- | | | |
| ylmethyl]-benzoic acid 2-dimethylamino-ethyl ester | | | |
| hydrochloride | | | |
| 4-Methyl-1,1,3-trioxo-2-[4-(piperidine-1-carbonyl)- | >30 | 10 | 0.29 |
| benzyl]-1,2,3,4-tetrahydro-1λ⁶-benzo[1,2,4]thiadiazine- | | | |
| 7-carboxylic acid 4-methoxy-benzylamide | | | |
| 2-{4-[7-(4-Methoxy-benzylcarbamoyl)-4-methyl-1,1,3- | >100 | >30 | 0.25 |
| trioxo-3,4-dihydro-1λ⁶-benzo[1,2,4]thiadiazin-2- | | | |
| ylmethyl]-benzoylamino}-3-methyl-butyric acid | | | |
| {4-[7-(4-Methoxy-benzylcarbamoyl)-4-methyl-1,1,3- | >100 | >30 | 0.0355 |
| trioxo-3,4-dihydro-1H-1λ⁶-benzo[1,2,4]thiadiazin-2- | | | |
| ylmethyl]-phenyl}-acetic acid | | | |
| 2-(4-cyano-benzyl)-4-methyl-1,1,3-trioxo-1,2,3,4- | >30 | 10 | 0.13 |
| tetrahydro-1λ⁶-benzo[1,2,4]thiadiazine-7-carboxylic | | | |
| acid 4-methoxy-benzylamide | | | |
| 4-[7-(3-Methoxy-benzylcarbamoyl)-4-methyl-1,1,3- | >100 | >30 | 0.0048 |
| trioxo-3,4-dihydro-1H-1λ⁶-benzo[1,2,4]thiadiazin-2- | | | |
| ylmethyl]-benzoic acid | | | |
| 4-Methyl-1,1,3-trioxo-2-[4-(2H-tetrazol-5-yl)-benzyl]- | >100 | 15 | 0.0062 |
| 1,2,3,4-tetrahydro-1λ⁶-benzo[1,2,4]thiadiazine-7- | | | |
| carboxylic acid 4-methoxy-benzylamide | | | |
| 2-Benzyl-4-methyl-1,1,3-trioxo-1,2,3,4-tetrahydro-1λ⁶- | >30 | >100 | 0.0625 |
| benzo[1,2,4]thiadiazine-7-carboxylic acid 3-methoxy- | | | |
| benzylamide | | | |
| 4-methyl-1,1,3-trioxo-2-pent-2-ynyl-1,2,3,4-tetrahydro- | nt | nt | 1.4 |
| 1λ⁶-benzo[1,2,4]thiadiazine-7-carboxylic acid 4- | | | |
| methoxy-benzylamide | | | |
| 4-Methyl-1,1,3-trioxo-2-(1-phenyl-ethyl)-1,2,3,4- | nt | nt | 6.3 |
| tetrahydro-1λ⁶-benzo[1,2,4]thiadiazine-7-carboxylic | | | |
| acid 4-methoxy-benzylamide | | | |
| 2-(5-Cyano-pentyl)-4-methyl-1,1,3-trioxo-1,2,3,4- | nt | nt | 3.2 |
| tetrahydro-1λ⁶-benzo[1,2,4]thiadiazine-7-carboxylic | | | |
| acid 4-methoxy-benzylamide | | | |
| 2-(E)-But-2-enyl-4-methyl-1,1,3-trioxo-1,2,3,4- | nt | nt | 2.2 |
| tetrahydro-1λ⁶-benzo[1,2,4]thiadiazine-7-carboxylic | | | |
| acid 4-methoxy-benzylamide | | | |
| 4-Methyl-1,1,3-trioxo-2-(E)-pent-2-enyl-1,2,3,4- | nt | nt | 1.5 |
| tetrahydro-1λ⁶-benzo[1,2,4]thiadiazine-7-carboxylic | | | |
| acid 4-methoxy-benzylamide | | | |
| 4-Methyl-2-(2-methyl-allyl)-1,1,3-trioxo-1,2,3,4- | nt | nt | 1.7 |
| tetrahydro-1λ⁶-benzo[1,2,4]thiadiazine-7-carboxylic | | | |
| acid 4-methoxy-benzylamide | | | |
| 4-Methyl-2-(3-methyl-but-2-enyl)-1,1,3-trioxo-1,2,3,4- | nt | nt | 1.9 |
| tetrahydro-1λ⁶-benzo[1,2,4]thiadiazine-7-carboxylic | | | |
| acid 4-methoxy-benzylamide | | | |
| 2-Benzo[1,2,5]oxadiazol-5-ylmethyl-4-methyl-1,1,3- | nt | nt | 0.7 |
| trioxo-1,2,3,4-tetrahydro-1λ⁶-benzo[1,2,4]thiadiazine- | | | |
| 7-carboxylic acid 4-methoxy-benzylamide | | | |
| {5-[7-(4-Methoxy-benzylcarbamoyl)-4-methyl-1,1,3- | nt | nt | 1.7 |
| trioxo-3,4-dihydro-1H-1λ⁶-benzo[1,2,4]thiadiazin-2- | | | |
| ylmethyl]-isoxazol-3-yl}-carbamic acid methyl ester | | | |

nt: not tested

### SYNTHESIS EXAMPLE 62

### 2-Benzyl-4-methyl-1,1,3-trioxo-1,2,3,4-tetrahydro-1λ⁶-benzo[1,2,4]thiadiazine-7-carboxylic acid benzyl ester

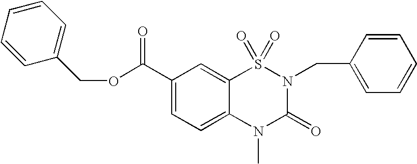

Step 1:

### Synthesis of 4-Methyl-1,1,3-trioxo-1,2,3,4-tetrahydro-1λ⁶-benzo[1,2,4]thiadiazine-7-carboxylic acid methyl ester

Methyl-4-methylaminobenzoate (4.96 g, 30 mmoles) was dissolved in 20 ml of nitromethane and this solution was added dropwise to a solution of 3.13 ml N-chlorosulfonyl isocyanate in 5 ml of nitromethane at 0° C. The resulting solution was stirred for 15 min and then 5.2 g (39 mmol) of solid aluminum trichloride was added. The resulting mixture was heated to reflux for 1 hour. The reaction was concentrated in vacuum and the residue was carefully quenched with ice water. The resulting yellowish solid was collected by filtration and recrystallized from ethyl acetate to give 3.95 g (49%) of the title compound as an off-white powder. ¹HNMR (CDCl₃): δ 8.47 (s, 1H), 8.22 (d, 1H), 7.24 (d, 2H), 3.89 (s, 3H), and 3.46 (s, 3H) ppm. MS: M⁺+1=271.1 Da.

Step 2:

### Synthesis of 2-Benzyl-4-methyl-1,1,3-trioxo-1,2,3,4-tetrahydro-1λ⁶-benzo[1,2,4]thiadiazine-7-carboxylic acid methyl ester

4-Methyl-1,1,3-trioxo-1,2,3,4-tetrahydro-1λ⁶-benzo[1,2,4]thiadiazine-7-carboxylic acid methyl ester (1.00 g, 3.7 mmoles) was mixed with benzyl bromide (0.66 ml, 5.6 mmoles) in 25 ml of acetonitrile. 0.83 ml (5.6 mmoles) of 1,8-diazabicyclo[5.4.0]undec-7-ene was added and the resulting mixture was stirred for 16 hours at room temperature. The mixture was concentrated under vacuum and partitioned between 1M HCl and ethyl acetate. The organic layer was dried (magnesium sulfate) and concentrated to give the product as an off-white solid. Triturated with hexanes to give 0.98 g (73%) of the title compound. ¹H-NMR (CDCl₃); δ 8.58 (s, 1H), 8.30 (d, 1H), 7.44 (d, 2H), 7.27 (m, 4H), 5.07 (s, 2H), 3.96 (s, 3H), and 3.53 (s, 3H) ppm. Anal. (C₁₇H₁₆N₂O₅S₁) C,H,N. MS: M⁺+1=361.0 Da

Step 3:

### Synthesis of 2-Benzyl-4-methyl-1,1,3-trioxo-1,2,3,4-tetrahydro-1λ⁶-benzo[1,2,4]thiadiazine-7-carboxylic acid

2-Benzyl-4-methyl-1,1,3-trioxo-1,2,3,4-tetrahydro-1λ⁶-benzo[1,2,4]thiadiazine-7-carboxylic acid methyl ester (0.87 g, 2.4 mmoles) was mixed with 3 ml of 1 M NaOH in 25 ml of methanol. This was stirred for 60 hours and then concentrated under vacuum. The residue was partitioned between water and dichloromethane. The aqueous layer was acidified with conc. HCl and the resulting suspension was collected and dried on the vacuum filter to give 0.60 g (73%) of the title compound as an off-white solid. ¹H-NMR (CDCl₃); δ 8.67 (s, 1H), 8.37 (d, 1H), 7.46 (d, 2H), 7.30 (m, 4H), 5.08 (s, 2H), and 3.56 (s, 3H) ppm. MS: M⁺+1=347.1 Da

Step 4:

### Synthesis of 2-Benzyl-4-methyl-1,1,3-trioxo-1,2,3,4-tetrahydro-1λ⁶-benzo[1,2,4]thiadiazine-7-carboxylic acid benzyl ester

2-Benzyl-4-methyl-1,1,3-trioxo-1,2,3,4-tetrahydro-1λ⁶-benzo[1,2,4]thiadiazine-7-carboxylic acid (0.25 g, 0.7 mmoles) was suspended in 20 ml of dichloromethane. Oxalyl chloride (0.076 ml, 0.87 mmoles) was added followed by 2 drops of DMF. The resulting effervescent mixture was stirred for 3 hours. The resulting clear solution was then concentrated to dryness. Benzyl alcohol (0.082 ml, 0.79 mmoles) was added and the mixture was dissolved in 5 ml of pyridine. 40 ml of water was added and the resulting milky mixture was stirred for 2 hours. The suspension was collected and chromatographed on silica to give 0.10 g (33%) of the title compound as a white solid. ¹H-NMR (CDCl₃); δ 8.59 (s, 1H), 8.33 (d, 1H), 7.36 (m, 8H), 5.39 (s, 2H), 5.07 (s, 2H), and 3.53 (s, 3H) ppm. Anal. (C₂₃H₂₀N₂O₅S₁) C,H,N. MS: M⁺+1=437.1 Da

### SYNTHESIS EXAMPLE 63

### 2-Benzyl-4-methyl-1,1,3-trioxo-1,2,3,4-tetrahydro-1λ⁶-benzo[1,2,4]thiadiazine-7-carboxylic acid benzylamide

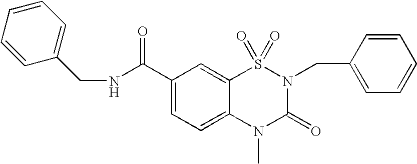

2-Benzyl-4-methyl-1,1,3-trioxo-1,2,3,4-tetrahydro-1λ⁶-benzo[1,2,4]thiadiazine-7-carboxylic acid (0.20 g, 0.6 mmoles, synthesis Example 62, step 3) was suspended in 20 ml of dichloromethane. Oxalyl chloride (0.06 ml, 0.7 mmoles) was added followed by 2 drops of DMF. The resulting effervescent mixture was stirred for 3 hours. The resulting clear solution was then concentrated to dryness. The residue was redissolved in 15 ml dichloromethane and 0.063 ml of benzylamine (0.6 mmoles) was added followed by 0.16 ml (1.2 mmoles) of triethylamine. This mixture was stirred for 16 hrs. at room temperature an then partitioned between 1 M HCl and dichloromethane. The organic layer was dried (magnesium sulfate) and concentrated to give an off white solid. Chromatography on silica gel gave 0.14 g of the title compound as a white solid. ¹H-NMR (CDCl₃); δ 8.23 (s, 1H), 8.17 (d, 1H), 7.35 (m, 11H), 6.47 (bs, 1H), 5.05 (s, 2H), 4.65 (d, 2H), and 3.52 (s, 3H) ppm. Anal. (C₂₃H₂₁N₃O₄S₁.0.25H₂O) C,H,N. MS: M⁺+1=436.1 Da

### SYNTHESIS EXAMPLE 64

### 2-Benzyl-4-methyl-1,1,3-trioxo-1,2,3,4-tetrahydro-1λ⁶-benzo[1,2,4]thiadiazine-7-carboxylic acid (pyridin-4-ylmethyl)-amide

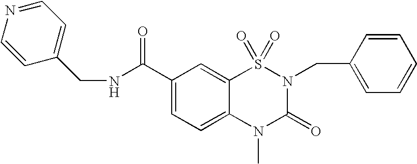

When in the procedure of synthesis Example 63, 4-(aminomethyl)pyridine is substituted for benzylamine, the title compound is obtained. ¹H-NMR (CDCl₃); δ 8.59 (d, 2H), 8.29 (s, 1H), 8.21 (d, 1H), 7.42 (d, 2H), 7.30 (m, 6H), 5.06 (s, 2H), 4.67 (d, 2H), and 3.54 (s, 3H) ppm. Anal. (C₂₂H₂₀N₄O₄S₁.0.5C₄H₈O₂) C,H,N. MS: M⁺+1=437.1 Da

### SYNTHESIS EXAMPLE 65

### 4-Methyl-2-(4-nitro-benzyl)-1,1,3-trioxo-1,2,3,4-tetrahydro-1λ⁶-benzo[1,2,4]thiadiazine-7-carboxylic acid 4-methoxy-benzylamide

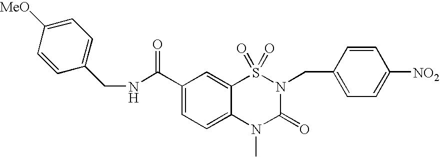

Step 1:

### 4-Methyl-1,1,3-trioxo-1,2,3,4-tetrahydro-1λ⁶-benzo[1,2,4]thiadiazine-7-carboxylic acid

4-Methyl-1,1,3-trioxo-1,2,3,4-tetrahydro-1λ⁶-benzo[1,2,4]thiadiazine-7-carboxylic acid methyl ester (10.0 g, synthesis Example 62, Step 1) was dissolved in 200 ml of methanol with 75 ml of 1M NaOH. Stirred for 4 hours and concentrated under vacuum to remove the methanol. The residue was acidified with concentrated HCl, filtered, and washed with water. Air dried on the vacuum filter to give 9.5 g of the title compound as a tan solid. ¹H-NMR (DMSO-d₆); δ 8.04 (s, 1H), 7.94 (dd, 1H), and 7.17 (d, 1H) ppm. MS: M⁺−1=255.1 Da

Step 2:

### 4-Methyl-1,1,3-trioxo-1,2,3,4-tetrahydro-1λ⁶-benzo[1,2,4]thiadiazine-7-carboxylic acid 4-methoxy-benzylamide

4-Methyl-1,1,3-trioxo-1,2,3,4-tetrahydro-1λ⁶-benzo[1,2,4]thiadiazine-7-carboxylic acid (2.5 g, Step 1) was mixed with 4-methoxybenzylamine (1.32 g) and 1-hydroxybenzotriazole in 50 ml of N,N-dimethylformamide. 1-(3-Dimethylaminopropyl)-3-ethylcarbodiimide hydrochloride (1.87 g) was added, and the mixture was allowed to stir at room temperature for 16 hours. The reaction was partitioned between 1M HCl and ethyl acetate. The organic layer was extracted with saturated sodium bicarbonate. The bicarbonate layer was then acidified and filtered. The white solid was washed with diethyl ether to give the title compound (2.26 g). ¹H-NMR (CDCl₃); δ 9.25 (t, 1H), 8.35 (d, 1H), 8.21 (dd, 1H), 7.57 (d, 1H), 7.22 (d, 2H), 6.86 (dd, 2H), 4.39 (d, 2H), 3.69 (s, 3H), 3.42 (s, 3H) and 2.47 (bs, 1H) ppm. MS: M⁺+1=376.1 Da

Step 3:

### 4-Methyl-2-(4-nitro-benzyl)-1,1,3-trioxo-1,2,3,4-tetrahydro-1λ⁶-benzo[1,2,4]thiadiazine-7-carboxylic acid 4-methoxy-benzylamide

4-Methyl-1,1,3-trioxo-1,2,3,4-tetrahydro-1λ⁶-benzo[1,2,4]thiadiazine-7-carboxylic acid 4-methoxy-benzylamide (1.0 g), and cesium carbonate (0.87 g) were mixed in 50 ml of N,N-dimethylformamide. 4-Nitrobenzylbromide (0.58 g) was added, and the resulting mixture was stirred for 16 hours at room temperature. The reaction was diluted with 1M HCl and filtered to give a gummy solid. Recrystallization from ethyl alcohol gave the title compound as a white solid (0.77 g). ¹H-NMR (CDCl₃); □ 8.48 (s, 1H), 8.26 (d, 1H), 8.10 (m, 3H), 7.54 (d, 2H), 7.25 (m, 4H), 6.82 (t, 2H), 5.05 (s, 2H), 4.50 (d, 2H), 3.73 (d, 3H), and 3.48 (s, 3H) ppm. Anal. (C₂₄H₂₂N₄O₇S₁.1.0H₂O) C,H,N. MS: M⁺+1=511.2 Da

Alkynylated Quinazolines

We have made a seventh group of compounds which are alkynylated analogs of substituted quinazolines (fourth group) and cyclized quinazolines (fifth group) and are inhibitors of matrix metalloproteinase enzymes, and especially MMP-13. Preferred compounds that we have made and their ability to inhibit the activity of MMP-13 are summarized in Table VII below.

TABLE VII

| | | IC50 |
|---|---|---|
| Compound name | Structure | μM |
| Methyl 4-{6-[3-(4-methoxy phenyl)-prop-1-ynyl]-1- methyl-2,4-dioxo-1,4- dihydro-2H-quinazolin-3- ylmethyl}-benzoate | 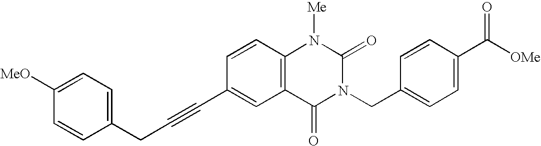 | 0.010 |
| 4-[1-Methyl-2,4-dioxo-6-(3- phenyl-prop-1-ynyl)-1,4- dihydro-2H-quinazolin-3- ylmethyl]-benzoic acid | 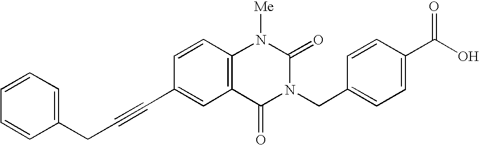 | 0.0009 |
| 4-{6-[3-(4-Methoxy- phenyl)-prop-1-ynyl]-1- methyl-2,4-dioxo-1,4- dihydro-2H-quinazolin-3- ylmethyl}-benzoic acid | 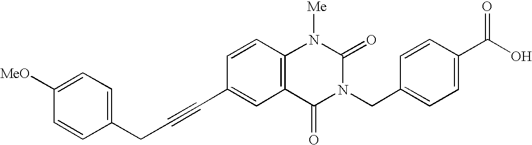 | 0.0006 |
| 4-[1-Methyl-2,4-dioxo-6-(3- phenyl-prop-1-ynyl)-1,4- dihydro-2H-pyrido[3,4-d]pyrimidin-3-ylmethyl]- benzoic acid | 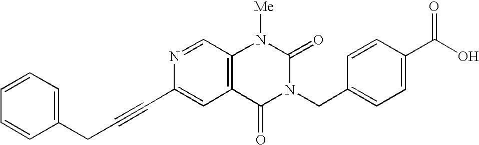 | 0.0065 |
| 4-{6-[3-(4-Methoxy- phenyl)-prop-1-ynyl]-1- methyl-2,4-dioxo-1,4- dihydro-2H-pyrido [3,4- d]pyrimidin-3-ylmethyl}- benzoic acid | 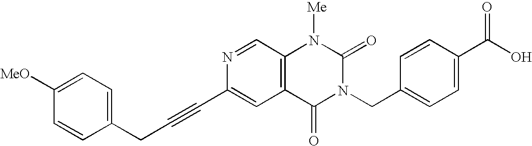 | 0.0012 |
| 4-Benzyl-7-(3-phenyl-prop- 1-ynyl)-4H- [1,2,4]triazolo[4,3-a]quinazolin-5-one | 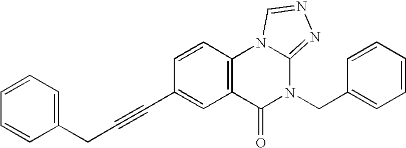 | 0.0055 |
| 4-Benzyl-7-[(4- methoxyphenyl)-prop-1- ynyl]-4H-[1,2,4]- triazolo[4,3-a]quinazolin-5- one | 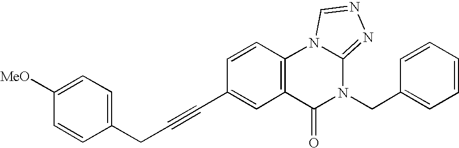 | 0.0015 |
| Methyl 4-{7-[3-(4-methoxy- phenyl)-prop-1-ynyl]-5-oxo- 5H-[1,2,4]triazolo[4,3-a]quinazolin-4-ylmethyl}- benzoate | 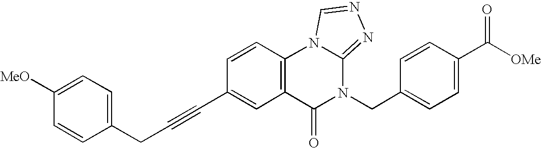 | 0.0017 |
| 4-[5-Oxo-7-(3-phenyl-prop- 1-ynyl)-5H- [1,2,4]triazolo[4,3-a]quinazolin-4-ylmethyl]- benzoic acid | 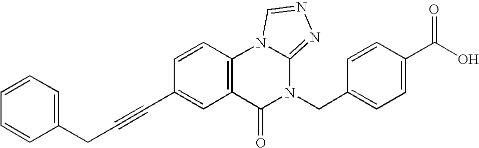 | 0.0010 |
| 4-[1-Methyl-2,4-dioxo-6-(2- phenylethynyl)-1,4-dihydro- 2H-quinazolin-3-ylmethyl]- benzoic acid | 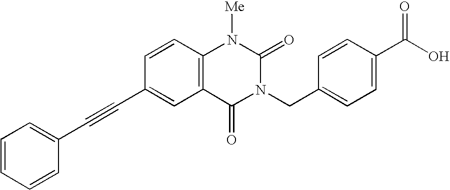 | 1.340 |

The alkyne group between the first scaffold ring and the first hydrophobic group forms part of the first hydrogen bond acceptor.

Synthesis of the compounds referred to in Table VII is described in the following further synthesis examples. The preparations are useful for the synthesis of compounds. The synthesis of the compound in the Table VII is also described in our co-pending WO application PCT/EP01/11824 filed on Oct. 12, 2001. This WO application, more specifically claims a compound selected from those of general formula (I): 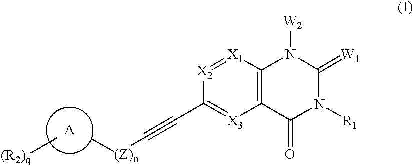
wherein:
W₁ represents an oxygen atom, a sulfur atom, or a —NR₃ group in which R₃ represents hydrogen atom, (C₁-C₆)alkyl, hydroxyl or cyano,
W₂ represents a group selected from:
   hydrogen atom, trifluoromethyl, amino, mono(C₁-C₁₀)alkylamino, di(C₁-C₁₀)alkylamino, each alkyl moiety being identical or different,
   (C₁-C₆)alkyl, (C₃-C₆)alkenyl, (C₃-C₆)alkynyl, (C₅-C₁₀)aryl, (C₅-C₁₀)aryl(C₁-C₁₀)alkyl, (C₃-C₁₀)cycloalkyl(C₁-C₁₀)alkyl, and the residue of an aromatic or non aromatic heterocycle comprising 5 or 6 ring members including from 1 to 4 hetero atoms selected from nitrogen, oxygen and sulfur, these groups being optionally substituted by one or more groups, which may be identical or different, selected from halogen, amino, mono(C₁-C₁₀)alkylamino, di(C₁-C₁₀)alkylamino, each alkyl moiety being identical or different, cyano, trihalogeno(C₁-C₆)alkyl, (C₁-C₆)acyl, —C(═O)OR₄, —OR₄ and —SR₄, R₄ representing a hydrogen atom or a (C₁-C₆)alkyl group,
or W₁ and W₂ form together a group of formula N—X₄═W₃ (in which the nitrogen atom is bonded on the place of the group W₁ and the group W₃ is bonded on the place of the group W₂) wherein:
   W₃ represents a nitrogen atom or a group —CR₅ in which R₅ is selected from:
      a hydrogen atom,
      —OR₆, —SR₆ in which R₆ is selected from hydrogen, (C₁-C₆)alkyl and (C₅-C₁₀)aryl(C₁-C₁₀)alkyl;
      (C₁-C₆)alkyl, (C₃-C₁₀)cycloalkyl, the residue of a saturated heterocycle comprising from 3 to 8 ring members including one hetero atom selected from oxygen, sulfur and nitrogen, (C₅-C₁₀)aryl, (C₅-C₁₀)heteroaryl comprising from 1 to 4 hetero atoms selected from oxygen, sulfur and nitrogen, and (C₅-C₁₀)aryl(C₁-C₁₀)alkyl, these groups being optionally substituted by —(CH₂)ₚ—OH or —(CH₂)ₚ—NH₂, wherein p is an integer from 0 to 4 inclusive,
   X₄ represents a nitrogen atom or a group —CR₇ in which R₇ is selected from hydrogen, —NR₈R₉, —OR₈, —SR₈, (C₁-C₆)alkyl, (C₃-C₁₀)cycloalkyl, the residue of a saturated heterocycle comprising from 3 to 8 ring members including one hetero atom selected from oxygen, sulfur and nitrogen, (C₅-C₁₀)aryl, (C₅-C₁₀)heteroaryl comprising from 1 to 4 hetero atoms selected from oxygen, sulfur and nitrogen, and (C₅-C₁₀)aryl(C₁-C₁₀)alkyl, these groups being optionally substituted by —(CH₂)ₚ—OH or —(CH₂)ₚ—NH₂ wherein p is an integer from 0 to 4 inclusive,
   and in which R₈ and R₉, identical or different, are selected from hydrogen, (C₁-C₆)alkyl and (C₅-C₁₀)aryl(C₁-C₁₀)alkyl,
X₁, X₂ and X₃ represent, independently of each other, a nitrogen atom or a carbon atom, the said carbon atom being unsubstituted or substituted with a group selected from:
   (C₁-C₆)alkyl, hydroxyl, (C₁-C₆)alkoxy, halogen, trifluoromethyl, cyano, nitro,
   —S(O)ₙ₁R₄ wherein n₁ represents an integer from 0 to 2 inclusive and R₄ represents an hydrogen atom or a (C₁-C₆)alkyl group,
   and —NR₁₀R₁₁ wherein R₁₀ and R₁₁, which may be identical or different, represent a group selected from hydrogen atom, (C₁-C₆)alkyl, and (C₅-C₁₀)aryl(C₁-C₁₀)alkyl, or R₁₀ and R₁₁ form together with the nitrogen atom to which there are bonded, a 5- or 6-ring members which can optionally contain a second hetero atom selected from nitrogen and oxygen,
with the proviso that not more than two of the groups X₁, X₂ and X₃ simultaneously represent a nitrogen atom,
n is an integer from 0 to 8 inclusive,
Z represents —CR₁₂R_{13,} wherein R₁₂ and R₁₃ independently of each other, represent a group selected from hydrogen, (C₁-C₆)alkyl, trihalogeno(C₁-C₆)alkyl, halogen, amino, mono(C₁-C₆)alkylamino, di(C₁-C₆)alkylamino in which each alkyl moiety is identical or different, —OR₄, —SR₄, and —C(═O)OR₄, R₄ being as defined hereinbefore, or —CR₁₂R₁₃ form together a carbonyl group, and
   when n is greater than or equal to 2, the hydrocarbon chain Z optionally contains one or more multiple bonds,
   and/or one of the carbon atoms in the hydrocarbon chain Z may be replaced with an oxygen atom, a sulfur atom which is unsubstituted or substituted with one or two oxygen, or a nitrogen atom which is unsubstituted or substituted with a (C₁-C₆)alkyl,
A represents the residue of an aromatic or non-aromatic 5- or 6-membered monocycle comprising from 0 to 4 hetero atoms selected from nitrogen, oxygen and sulfur, or a bicycle composed of two aromatic or non-aromatic 5- or 6-membered rings, which may be identical or different, comprising from 0 to 4 hetero atoms selected from nitrogen, oxygen and sulfur,
the group(s) R₂, which may be identical or different, are selected from hydrogen, (C₁-C₆)alkyl, halogen, cyano, nitro, trihalogeno(C₁-C₆)alkyl, —NR₁₀R₁₁, —OR₁₄, —SR₁₄, —SOR₁₄, —SO₂R₁₄, (C₁-C₆)acyl, —(CH₂)ₖNR₁₀R₁₁, —X₅(CH₂)ₖNR₁₀R₁₁, —(CH₂)ₖSO₂NR₁₄R₁₅, —X₅(CH₂)ₖC(═O)OR₁₄, —(CH₂)ₖC(═O)OR₁₄, —X₅(CH₂)ₖC(═O)NR₁₄R₁₅, —(CH₂)ₖC(═O)NR₁₄R₁₅ and —X₆—R₁₆ in which:
   X₅ represents an oxygen atom, a sulfur atom, a —NH group, or a —N(C₁-C₆)alkyl group,
   k is an integer from 0 and 3 inclusive,
   R₁₀ and R₁₁ are as defined hereinbefore,
   R₁₄ and R₁₅, identical or different, represent hydrogen or (C₁-C₆)alkyl,
   X₆ represents a single bond, —CH₂—, an oxygen atom or a sulfur atom which is unsubstituted or substituted with one or two oxygen atoms,
   R₁₆ represents the residue of an aromatic or non-aromatic, heterocyclic or non-heterocyclic, 5- or 6-membered ring which is unsubstituted or substituted with one or more groups, which may be identical or different, selected from (C₁-C₆)alkyl, halogen, trihalogeno(C₁-C₆)alkyl, hydroxyl, (C₁-C₆)alkoxy, mercapto, (C₁-C₆)alkylthio, amino, mono(C₁-C₆)alkylamino, di(C₁-C₆)alkylamino each alkyl moiety being identical or different, and when the ring is heterocyclic, it comprises from 1 to 4 hetero atoms selected from nitrogen, oxygen and sulfur,
q is an integer from 0 to 7 inclusive,
R₁ represents a group selected from hydrogen, (C₁-C₆)alkyl, (C₃-C₆)alkenyl, and (C₃-C₆)alkynyl, the groups alkyl, alkenyl and alkynyl being optionally substituted with one or more groups, which may be identical or different, selected from amino, mono(C₁-C₆)alkylamino, di(C₁-C₆)alkylamino in which each alkyl moiety is identical or different, (C₁-C₆)alkyl, cyano, trihalogeno(C₁-C₆)alkyl, —C(═O)OR₄, —OR₄, —SR₄, in which R₄ is as defined above, and the group of formula: 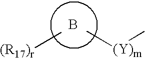
in which:
   m is an integer from 0 to 8 inclusive,
   Y represents —CR₁₈R₁₉, wherein R₁₈ and R₁₉ independently of each other, represent a group selected from hydrogen, (C₁-C₆)alkyl, phenyl, trihalogeno(C₁-C₆)alkyl, halogen, amino, mono(C₁-C₆)alkylamino, di(C₁-C₆)alkylamino in which each alkyl moiety is identical or different, —OR₄, —SR₄ or —C(═O)OR₄ wherein R₄ is as defined above, and
      when m is greater than or equal to 2, the hydrocarbon chain Y optionally contains one or more multiple bonds,
      and/or one of the carbon atoms in the hydrocarbon chain Y may be replaced with an oxygen atom, a sulfur atom which is unsubstituted or substituted with one or two oxygen, or a nitrogen atom which is unsubstituted or substituted with (C₁-C₆)alkyl,
   B represents a group selected from the residue of an aromatic or non-aromatic, 5- or 6-membered monocycle comprising from 0 to 4 hetero atoms selected from nitrogen, oxygen and sulfur, and a bicycle, composed of two aromatic or non-aromatic, 5- or 6-membered rings, which may be identical or different, comprising from 0 to 4 hetero atoms selected from nitrogen, oxygen and sulfur,
   r is an integer from 0 to 7 inclusive,
   the group(s) R₁₇ which may be identical or different are selected from hydrogen, (C₁-C₆)alkyl, halogen, cyano, nitro, trihalogeno(C₁-C₆)alkyl, —NR₁₀R₁₁, —OR₁₄, —SR₁₄, —SOR₁₄, —SO₂R₁₄, (C₁-C₆)acyl, —(CH₂)ₖNR₁₀R₁₁, —X₅(CH₂)ₖNR₁₀R₁₁, —(CH₂)ₖSO₂NR₁₄R₁₅, —X₅(CH₂)ₖC(═O)OR₁₄, —(CH₂)ₖC(═O)OR₁₄, —X₅(CH₂)ₖC(═O)NR₁₄R₁₅, —(CH₂)ₖC(═O)NR₁₄R₁₅ and the group of formula —X₆—R₁₆ in which X₅, k, R₁₀, R₁₁, R₁₄, R₁₅, X₆ and R₁₆ are as defined hereinbefore, and
optionally, its optical isomers, N-oxides, and addition salts thereof with a pharmaceutically-acceptable acid or base,

In formula (I), it is understood that:
a (C₁-C₆)alkyl group and a (C₁-C₁₀)alkyl group denote a linear or branched group containing respectively from 1 to 6 or from 1 to 10 carbon atoms; example of such groups, without implying any limitation are methyl, ethyl, propyl, isopropyl, tert-butyl, neopentyl, hexyl, heptyl, 3-methyl-hexyl, . . .
a (C₃-C₆)alkenyl group denotes a linear or branched group containing from 3 to 6 carbon atoms, and one or more double bonds; examples of such groups without implying any limitation are allyl, 3-buten-1-yl, 2-methyl-buten-1-yl, hexenyl, . . .
a (C₃-C₆)alkynyl group denotes a linear or branched group containing from 3 to 6 carbon atoms, and one or more triple bonds; examples of such groups without implying any limitation are 3-butyn-1-yl, 2-methyl-butyn-1-yl, hexynyl, . . .
a (C₁-C₆)alkoxy group means the alkyl group as mentioned above bound through an oxygen atom; examples of such compounds without implying any limitation are metoxy, ethoxy, n-propyloxy, tert-butyloxy, . . .
a (C₁-C₆)alkylamino or (C₁-C₁₀)alkylamino means the alkyl groups as defined above bound through a nitrogen atom; example of such groups, without implying any limitation are methyl amino, isobutyl amino, dimethylamino, ethylamino, diethylamino, . . .
a (C₅-C₁₀)aryl group denotes an aromatic system containing from 5 to 8 carbon atoms; examples of such groups without implying any limitation are cyclopentadienyl, phenyl, naphthyl, indenyl, . . .
a (C₅-C₁₀)heteroaryl group denotes an aromatic system as described above in which 1 to 4 carbon atoms are replaced by 1 to 4 hetero atoms selected from oxygen, sulfur and nitrogen; examples of such groups without implying any limitation are furyl, thienyl, pyrrolyl, pyrazolyl, pyridyl, pyrimidyl, pyrazinyl, benzofuryl, benzothienyl, indolyl, quinolyl, isoquinolyl, benzodioxolyl, benzodioxinyl, benzo[1,2,5]thiadiazolyl, benzo[1,2,5]oxadiazolyl, . . .
a (C₃-C₁₀)cycloalkyl group denotes a cyclic system containing from 3 to 10 carbon atoms; examples of such groups without implying any limitation are cyclopropyl, cyclobutyl, cyclopentyl, cyclohexyl, cyclooctyl, cycloheptyl, adamantyl, decalinyl, norbornyl, . . .
a trihalogeno(C₁-C₆)alkyl group denotes an alkyl group as defined above which contains a trihalogeno group; examples of such groups without implying any limitation are trifluoromethyl, 2,2,2-trifluoroethyl, . . .
a (C₁-C₆)acyl group denotes an alkyl group or a aryl group as defined above bound through a carbonyl group; examples of such groups without implying any limitation are acetyl, ethylcarbonyl, benzoyl, . . .
a multiple bond denotes double bond or triple bond,
optical isomers refer to racemates, enantiomers and diastereoisomers.

Our co-pending WO application PCT/EP01/11824 claimed more particularly a compound according to formula (I), which is selected from:
methyl 4-{6-[3-(4-methoxyphenyl)-prop-1-ynyl]-1-methyl-2,4-dioxo-1,4-dihydro-2H-quinazolin-3-ylmethyl}-benzoate,
4-[1-methyl-2,4-dioxo-6-(3-phenyl-prop-1-ynyl)-1,4-dihydro-2H-quinazolin-3-ylmethyl]-benzoic acid,
4-{6-[3-(4-methoxy-phenyl)-prop-1-ynyl]-1-methyl-2,4-dioxo-1,4-dihydro-2H-quinazolin-3-ylmethyl}-benzoic acid,
4-[1-methyl-2,4-dioxo-6-(3-phenyl-prop-1-ynyl)-1,4-dihydro-2H-pyrido[3,4-d]pyrimidin-3-ylmethyl]-benzoic acid,
4-{6-[3-(4-methoxy-phenyl)-prop-1-ynyl]-1-methyl-2,4-dioxo-1,4-dihydro-2H-pyrido[3,4-d]pyrimidin-3-ylmethyl}-benzoic acid,
4-benzyl-7-(3-phenyl-prop-1-ynyl)-4H-[1,2,4]triazolo[4,3-a]quinazolin-5-one,
4-benzyl-7-[(4-methoxyphenyl)-prop-1-ynyl]-4H-[1,2,4]-triazolo[4,3-a]quinazolin-5-one,
methyl 4-{7-[3-(4-methoxy-phenyl)-prop-1-ynyl]-5-oxo-5H-[1,2,4]triazolo[4,3-a]quinazolin-4-ylmethyl}-benzoate,
4-[5-oxo-7-(3-phenyl-prop-1-ynyl)-5H-[1,2,4]triazolo[4,3-a]quinazolin-4-ylmethyl]-benzoic acid,
and 4-(1-methyl-2,4-dioxo-6-(2-phenylethynyl)-1,4-dihydro-2H-quinazolin-3-ylmethyl)-benzoic acid.

Our co-pending WO application PCT/EP01/11824 claims also a method for treating a living body afflicted with a disease where the inhibition of type-13 matrix metalloprotease is involved, comprising the step of administering to the living body an amount of a compound of formula (I) which is effective for alleviation of said conditions.

More particularly, our co-pending WO application PCT/EP01/11824 claims a method for treating a living body afflicted with a disease selected from arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease, age-related macular degeneration, and cancers, comprising the step of administering to the living body an amount of a compound of formula (I) which is effective for alleviation of said conditions.

Our co-pending WO application PCT/EP01/11824 claims also a pharmaceutical composition comprising as active ingredient an effective amount of a compound as claimed in formula (I), alone or in combination with one or more pharmaceutically-acceptable excipients or carriers.

Synthesis and Preparations of the Compounds Described in Table VII:

### Preparation A

### 4-(6-Iodo-1-methyl-2,4-dioxo-1,4-dihydro-2H-quinazolin-3-ylmethyl)-benzoic acid

Step 1:

### Methyl 4-[(2-amino-5-iodo-benzoylamino)-methyl]-benzoate

To a stirred solution of 15 g (74.4 mmol) of methyl 4-(aminomethyl)benzoate hydrochloride, 300 ml of dimethylformamide and 10.3 ml (7.53 g, 74.4 mmol) of triethylamine were added, at room temperature, followed by 10.06 g (74.4 mmol) of 1-hydroxybenzotriazole hydrate, 19.6 g (74.4 mmol) of 2-amino-5-iodobenzoic acid and 14.3 g (74.4 mmol) of 1-(3-dimethylaminopropyl)-3-ethyl carbodiimide hydrochloride. After stirring at room temperature overnight, the mixture was concentrated and the residue was dissolved in 300 ml of dichloromethane. The organic phase was washed with 150 ml H₂O, 150 ml HCl 1N, and 150 ml H₂O, dried over sodium sulfate and concentrated. The residue was recrystallized from 170 ml acetonitrile to afford after filtration 19.6 g of the desired product (yield: 70%).

N.M.R: DMSO ¹H δ (ppm): 3.8 (s,3H); 4.45 (d,2H); 6.5-6.6 (m,3H); 7.3-7.45 (m,3H); 7.8-7.95 (m,3H); 8.9 (t,1H) Purity (HPLC): 99.1%

Step 2:

### Methyl 4-(6-iodo-2,4-dioxo-1,4-dihydro-2H-quinazolin-3-ylmethyl)-benzoate

To a solution of 21.35 g (52 mmol) of the compound obtained in Step 1 in 400 ml of dry tetrahydrofurane were added 9.3 g (57.2 mmol) of 1,1′-carbonyldiimidazole. The solution was heated overnight to 60° C. After cooling the precipitate was filtered and dried to afford 19.6 g of the desired product (yield: 68.3%).

N.M.R: DMSO ¹H δ (ppm): 3.8 (s,3H); 5.1 (s,2H); 6.95-7.05 (m,1H); 7.35-7.45 (m,2H); 7.8-7.90 (m,2H); 7.9-8.0 (m,1H); 8.2 (s,1H); 11.6 (bs,1H) Purity (HPLC): 99.5%

Step 3:

### Methyl 4-(6-iodo-1-methyl-2,4-dioxo-1,4-dihydro-2H-quinazolin-3-ylmethyl)-benzoate

To a stirred suspension of 11 g (25.2 mmol) of the compound obtained in Step 2 and 110 ml of dry DMF were added 5.22 g (37.8 mmol) of K₂CO₃, at room temperature. After 15 minutes, 7.85 ml (17.9 g, 126 mmol) of iodomethane were added. The reaction mixture was stirred for 2 hours and the precipitate filtered off and dissolved in a mixture of dichloromethane/methanol. The organic phase was washed with H₂O, dried over Na₂SO₄ and concentrated to afford a precipitate corresponding to the desired product (10.1 g; yield: 89%).

N.M.R: DMSO ¹H δ (ppm): 3.5 (s,3H); 3.8 (s,3H); 5.2 (s,2H); 7.30 (d,1H); 7.45 (d,2H); 7.90 (d,2H); 8.1 (d,1H); 8.3 (s,1H) Purity (HPLC): 96.7%

Step 4:

### 4-(6-Iodo-1-methyl-2,4-dioxo-1,4-dihydro-2H-quinazolin-3-ylmethyl)-benzoic acid

A mixture of 3.0 g (6.66 mmol) of the compound obtained in Step 3, 30 ml of dioxane, 120 ml H₂O, and 0.56 g (13.3 mmol) of LiOH, H₂O was heated to reflux over 1 hour. After cooling and acidification with concentrated hydrochloric acid, the precipitate obtained was filtered off and recrystallized in dioxane/ether to afford 1.85 g of the desired product (yield: 64.2%).

N.M.R: DMSO ¹H δ (ppm): 3.5 (s,3H); 5.2 (s,2H); 7.30 (d,1H); 7.40 (d,2H); 7.85 (d,2H); 8.1 (d,1H); 8.30 (s,1H); 12.9 (bs,1H) Purity (HPLC): 98.0%

### Preparation B

### 4-(1-Methyl-2,4-dioxo-6-trifluoromethanesulfonyloxy-1,4-dihydro-2H-pyrido[3,4-d]pyrimidin-3-ylmethyl)-benzoic acid

Step 1:

### 5-(tert-Butoxycarbonylamino)-2-methoxypyridine-4-carboxylic acid

The compound 5-(tert-butoxycarbonylamino)-2-methoxypyridine-4-carboxylic acid was prepared using the procedure described in J. Chem. Soc., Perkin Trans I, 1996, 18, 2221-2226.

Step 2:

### Methyl 4-{[(5-tert-butoxycarbonylamino-2-methoxy-pyridine-4-carbonyl)-amino]-methyl}-benzoate

9 g (33.5 mmol ) of the compound obtained in Step 1, 320 ml of dichloromethane, 11 g (33.5 moles) of TOTU and 6.1 g (36.9 mmol) of methyl-(4-aminomethyl)benzoate were stirred and cooled to 0° C., and then 11.6 ml (8.6 g, 67 mmol) of diisopropylamine added. The mixture was stirred for 15 minutes at 0° C. and then overnight at room temperature. The reaction mixture was washed successively with 200 ml NH₄OH, 200 ml H₂O, 200 ml HCl 10%, 200 ml H₂O, 200 ml NaHCO₃, and 200 ml H₂O. The organic phase was dried over Na₂SO₄, filtered, and concentrated under vacuum. The residue was crystallized in a mixture of dichloromethane/ether to afford 10.5 g of the desired product (yield: 73.3%).

TLC: CH₂Cl₂/MeOH: 95/5 v/v Rf=0.60 N.M.R: CDCl₃ ¹H δ (ppm): 1.50 (s,9H); 3.90 (2s,6H); 4.60 (d,2H); 6.70 (s,1H); 7.0 (bs,1H); 7.4 (d,2H); 8.0 (d,2H); 8.75 (bs,1H); 8.9 (s,1H)

Step 3:

### Methyl 4-{[(5-amino-2-methoxy-pyridine-4-carbonyl)-aminomethyl}-benzoate

To a solution of 4.8 g (11.5 mmol) of the compound obtained in Step 2 in 100 ml of dichloromethane were added 20 ml of trifluoroacetic acid. The reaction was heated to 40° C. for 1 hour, and then concentrated under vacuum. The residue was taken up in a mixture of dichloromethane and H₂O then basified with NaOH. After separation by decantation, the organic phase was washed, dried over Na₂SO₄, and concentrated under vacuum to afford 3.5 g of a yellow precipitate corresponding to the desired product (yield: 97%).

TLC: CH₂Cl₂/MeOH 95/5 v/v Rf=0.40 N.M.R: CDCl₃ ¹H δ (ppm): 3.8 (s,3H); 3.9 (s,3H); 4.6 (d,2H); 4.7 (s,2H); 6.7 (s,1H); 6.75-6.85 (m,1H); 7.40 (d,2H); 7.75 (s,2H); 8.0 (d,2H)

Step 4:

### Methyl 4-(6-methoxy-2,4-dioxo-1,4-dihydro-2H-pyrido[3,4-d]-pyrimidin-3-ylmethyl)-benzoate

To a solution of 2.5 g (7.9 mmol) of the compound obtained in Step 3 in 110 ml of dry THF were added 2 g (12.4 mmol) of 1,1′-carbonyldiimidazole. The reaction mixture was heated to 60° C. for 24 hours. After cooling, 50 ml H₂O were added and the mixture was stirred for 30 minutes to 0° C. The precipitate was filtered and washed successively with H₂O, MeOH and dichloromethane to afford 2.38 g of the desired product (yield: 88.3%).

TLC: CH₂Cl₂/MeOH 95/5 v/v Rf=0.45 N.M.R: DMSO ¹H δ (ppm): 3.80 (s,3H); 3.90 (s,3H); 5.10 (s,2H); 7.2 (s,1H); 7.45 (d,2H); 7.90 (d,2H); 8.25 (s,1H); 11.6 (s,1H)

Step 5:

### Methyl 4-(6-methoxy-1-methyl-2,4-dioxo-1,4-dihydro-2H-pyrido[3,4-d]pyrimidin-3-ylmethyl)-benzoate

2.38 g (7 mmol) of the compound obtained in Step 4 and 52 ml of dry DMF were stirred and heated until dissolution. After cooling to 25° C., 1.45 g (10 mmol) of K₂CO₃ and 2.2 ml (5.7 g, 35 mmol) of iodomethane were added. The mixture was stirred for 30 minutes at room temperature, then concentrated under vacuum. The residue was treated with H₂O and the precipitate filtered off, washed with methanol, then dissolved in dichloromethane. The organic phase was washed with H₂O, dried over Na₂SO₄ and concentrated under vacuum. The product was crystallised in ether and filtered to afford 2.0 g of the desired product (yield: 80%).

TLC: CH₂Cl₂/MeOH 95/5 v/v Rf=0.95 Purity (HPLC): 98.5% N.M.R: DMSO ¹H δ (ppm): 3.50 (s,3H); 3.80 (s,3H); 3.90 (s,3H); 5.20 (s,2H); 7.3 (s,1H); 7.45 (d,2H); 7.90 (d,2H); 8.50 (s,1H)

Step 6:

### 4-(6-Hydroxy-1-methyl-2,4-dioxo-1,4-dihydro-2H-pyrido[3,4-d]pyrimidin-3-ylmethyl)-benzoic acid

1.4 g (3.93 mmol) of compound obtained in Step 5, and 14 ml of hydrobromic acid were heated to reflux for 1 hour. After cooling, 30 ml of H₂O were added and the precipitate was filtered off and washed with H₂O and MeOH to afford 1.1 g of the desired product (yield: 85.5%)

TLC: CH₂Cl₂/MeOH 90/10 v/v Rf=0.10 N.M.R: DMSO ¹H δ (ppm) 3.50 (s,3H); 5.20 (s,2H); 7.05 (s,1H); 7.40 (d,2H); 7.90 (d,2H); 8.20 (s,1H); 10.4-13.0 (bs,2H)

Step 7:

### 4-(1-Methyl-2,4-dioxo-6-trifluoromethanesulfonyloxy-1,4-dihydro-2H-pyrido[3,4-d]pyrimidin-3-ylmethyl)-benzoic acid

A solution of 1.2 g of compound obtained in Step 6 in 14 ml of dry pyridin was stirred and cooled to 0° C., and then 1.5 ml (2.52 g, 9 mmol) of trifluoromethanesulfonic anhydride were added. The reaction was allowed to stir at 0° C. for 30 minutes then quenched with 30 ml of H₂O and dichloromethane. The organic phase was washed with H₂O, HCl 10%, and H₂O. After concentration the residue was crystallised in a mixture dichloromethane/ether to afford 0.5 g of the desired product (yield: 30%).

TLC: CH₂Cl₂/MeOH 90/10 v/v Rf=0.55 N.M.R: DMSO ¹H δ (ppm): 3.55 (s,3H); 5.20 (s,2H); 7.45 (d,2H); 7.90 (d,2H); 8.10 (s,1H); 8.80 (s,1H); 12.9 (bs,1H)

### Preparation C

### Methyl 4-(5-oxo-7-(Trifluoromethylsulfonyloxy)-5H-[1,2,4]triazol[4,3-a]quinazolin-4-ylmethyl)-benzoate

Step 1:

### 4-Benzyl-7-(trifluoromethylsulfonyloxy)-4H-[1,2,4]triazolo[4,3a]quinazolin-5-one

To a suspension of 41.3 g (141.3 mmol) of 4-benzyl-7-hydroxy-4H-[1,2,4]triazolo[4,3-a]quinazolin-5-one (obtained as described in WO 00/66584) in 500 ml of CH₂Cl₂, 25 g (148.3 mmol) of trifluoromethylsulfonylchloride were added under stirring. Then, 22.5 g (222.5 mmol) of triethylamine were added dropwise while maintaining the internal temperature between 15 and 20° C. After the completion of addition, stirring was continued at room temperature for 4 hours. After removal of the insoluble solid by filtration, the organic solution was washed with water and brine, then dried over Na₂SO₄ and concentrated, providing 33.1 g of crude solid, which was purified by chromatography (cyclohexane/AcOEt: 25/75 v/v) to afford 22.5 g of the desired compound (yield: 37.5%).

TLC: CH₂Cl₂/MeOH 95/5 v/v Rf=0.45

Step 2:

### 7-(Trifluoromethylsulfonyloxy)-4H-[1,2,4]triazolo[4,3-a]quinazolin-5-one

A suspension of 10.0 g (23.5 mmol) of the compound obtained in Step 1 and 18.8 g (141 mmol) of aluminium chloride in 200 ml anhydrous benzene was heated at 50° C., under stirring, for 1 h 30. After cooling, the mixture obtained was poured on water/ice. After stirring and homogenization, the insoluble solid was isolated by filtration, washed with several portions of water until neutral pH and dried, then finally washed with a portion of CH₂Cl₂, leaving 7.95 g (99%) of the desired compound.

TLC: CH₂Cl₂/MeOH 95/5 v/v Rf=0.10

Step 3:

### Methyl 4-(5-oxo-7-(Trifluoromethylsulfonyloxy)-5H-[1,2,4]triazolo[4,3-a]quinazolin-4-ylmethyl)-benzoate

To a stirred solution of 7.9 g (24.3 mmol) of the compound obtained in Step 2 in 100 ml of DMF were added 7.93 g (24.3 mmol) of cesium carbonate, and then 5.56 g (24.3 mmol) of methyl 4-(bromomethyl)benzoate. The mixture was stirred overnight and the solvent was removed under vacuum. The resulting residue was partitioned between H₂O and a mixture of dichloromethane and ethyl acetate. A first portion (5.9 g) of product insoluble in the two phases was obtained by filtration then recrystallized in methanol to give 4.85 g of the pure title compound. The organic phase was separated, washed with water and brine, and dried over anhydrous sodium sulfate. Concentration under reduced pressure afforded 4.5 g of crude product that was recrystallized in methanol to provide 2.2 g of pure compound. An additional portion of 2.5 g was finally obtained after column chromatography on silica gel of the residues gathered from the organic phases (dichloromethane/methanol 98/2 v/v). All in all, 9.55 g (yield: 81.5%) of the desired product were obtained.

TLC: CH₂Cl₂/CH3OH 95/5 v/v Rf=0.35

### Preparation D

### 4-(5-oxo-7-(Trifluoromethylsulfonyloxy)-5H-[1,2,4]triazolo[4,3-a]quinazolin-4-ylmethyl)-benzoic acid

Step 1:

### tert-Butyl 4-(5-oxo-7-(Trifluoromethylsulfonyloxy)-5H-[1,2,4]triazolo[4,3-a]quinazolin-4-ylmethyl)-benzoate

The product is obtained with a yield of 60.5% (0.95 g) according to the procedure of Step 3 of Preparation C using 1.0 g (2.99 mmol) of compound obtained in Step 1 of Preparation C and 0.81 g (2.99 mmol) of tert-butyl-4-(bromomethyl)benzoate.

Step 2:

### 4-(5-oxo-7-(Trifluoromethylsulfonyloxy)-5H-[1,2,4]triazolo[4,3-a]quinazolin-4-ylmethyl)-benzoic acid

To a suspension of 0.27 g (0.515 mmol) of compound obtained in Step 1 in 30 ml of dichloromethane, 2.7 ml of trifluoroacetic acid were added and stirring was continued at room temperature for 16 hours. The reaction mixture was poured into water and the resulting mixture stirred for 15 minutes. The ensuing precipitate was filtered off, washed with water until neutral pH and dried at 50° C. under vacuum to provide 0.21 g of the desired product.

TLC: dichloromethane/methanol 90/10 v/v Rf=0.30

### SYNTHESIS EXAMPLE 66

### Methyl 4-{6-[3-(4-methoxyphenyl)-prop-1-ynyl]-1-methyl-2,4-dioxo-1,4-dihydro-2H-quinazolin-3-ylmethyl}-benzoate

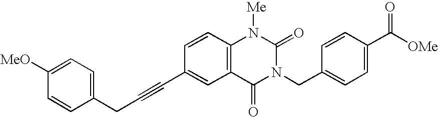

To a stirred suspension of 1.5 g (3.33 mmol) of compound obtained in Step 3 of Preparation A in 110 ml of triethylamine were added, under nitrogen atmosphere, 0.6 g (4 mmol) of 3-(4-methoxyphenyl)-prop-1-yne (described in the literature: J. Prakt. Chem., 1966, 33, 84-95) in 10 ml of triethylamine, 47 mg (0.06 mmol) of dichlorobis(triphenylphosphine)palladium (II) and 26 mg (0.13 mmol) of CuI. The mixture was heated to 60° C. over 3 hours (uncomplete reaction). The mixture was then concentrated under vacuum and the residue purified by flash chromatography to afford 0.130 mg of the desired product (yield: 6%) which was crystallized in a mixture of dichloromethane/methanol.

TLC: CH₂Cl₂/Acetone 99/1 v/v Rf=0.9 N.M.R: DMSO ¹H δ (ppm); 3.5 (s,3H); 3.75 (s,3H); 3.8 (s,5H); 5.2 (s,2H); 6.9 (d,2H); 7.35 (s,2H); 7.45 (m,3H); 7.85 (d,1H); 7.9 (d,2H); 8.0 (s,1H) IR: 2361, 1702, 1656, 1612, 1508, 1475, 1279, 1249, 117, 1102, 958, 805 cm⁻¹ Mp=168.5° C. Purity (HPLC): 97.9%

### SYNTHESIS EXAMPLE 67

### 4-[1-Methyl-2,4-dioxo-6-(3-phenyl-prop-1-ynyl)-1,4-dihydro-2H-quinazolin-3-ylmethyl]-benzoic acid

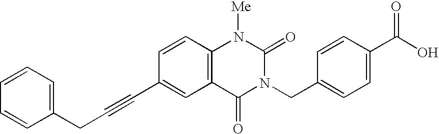

To a stirred solution of 0.68 g (1.56 mmol) of compound obtained in Step 4 of Preparation A in 6.8 ml of dry DMF, were added successively, under nitrogen atmosphere, 1.2 ml (0.8 g, 6.24 mmol) of diisopropylethylamine, 56.8 mg (0.078 mmol) of dichlorobis (triphenylphosphine)palladium (II), a catalytic amount of CuI and 0.273 ml (0.253 g, 2.18 mmol) of 3-phenyl-1-propyne. The reaction mixture was heated to 50° C. over approximately 4 hours. Then, the mixture is concentrated under vacuum and the residue purified by flash chromatography (dichloromethane/MeOH 90/10 v/v) to afford, after crystallization in a mixture of dichloromethane/ether, 0.270 g of the desired product (yield: 40.8%).

TLC: CH₂Cl₂/MeOH 9/1 v/v Rf=0.50 N.M.R: DMSO ¹H δ (ppm); 3.5 (s,3H); 3.9 (s,2H); 5.2 (s,2H); 7.20-7.50 (m,8H); 7.80 (m,3H); 8.05 (s,1H); 12.8 (bs,1H); IR: 2894, 1700, 1660, 1616, 1508, 1314, 1295, 1097, 825, 795, 747 cm⁻¹ Mp=258° C. Purity (HPLC): 98.6%

### SYNTHESIS EXAMPLE 68

### 4-{6-[3-(4-Methoxy-phenyl)-prop-1-ynyl]-1-methyl-2,4-dioxo-1,4-dihydro-2H-quinazolin-3-ylmethyl}-benzoic acid

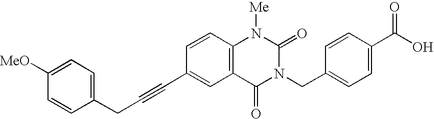

This compound was obtained according to the procedure described in Example 67 using as reagent 3-(4-methoxyphenyl)-prop-1-ynyl. The crude product was crystallized in dioxane to afford the desired compound.

TLC: CH₂Cl₂/MeOH 9/1 v/v Rf=0.50 N.M.R: DMSO ¹H δ (ppm); 3.55 (s,3H); 3.75 (s,3H); 3.8 (s,2H); 5.15 (s,2H); 6.9 (d,2H); 7.30 (d,2H); 7.40 (m,3H); 7.85 (m,3H); 8.00 (s,1H); 12.85 (bs,1H); IR: 2646, 1687, 1659, 1508, 1477, 1422, 1325, 1242, 1177, 1040, 950, 812 cm⁻¹ Mp=262° C. Purity (HPLC): 95.4%

### SYNTHESIS EXAMPLE 69

### 4-[1-Methyl-2,4-dioxo-6-(3-phenyl-prop-1-ynyl)-1,4-dihydro-2H-pyrido[3,4-d]pyrimidin-3-ylmethyl]-benzoic acid

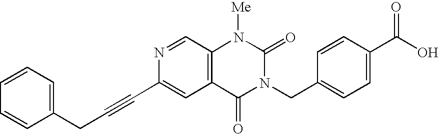

To a stirred solution of 0.1 g (0.22 mmol) of the compound of Preparation B in 1 ml of dry DMF were added successively 0.2 ml (0.14 g, 1.1 mmol) of diisopropylethylamine, 9 mg (0.012 mmol) of dichlorobis (triphenylphosphine)palladium (II), a catalytic amount of CuI and 0.046 ml (0.043 g, 1.1 mmol) of 3-phenyl-1-propyne. The reaction was stirred overnight at room temperature and then H₂O and CH₂Cl₂ were added. The organic layer was separated and washed with HCl 10% and H₂O, then dried over sodium sulfate and concentrated under vacuum. The residue was crystallized in a mixture of dichloromethane/ether to afford 0.040 g of the desired product (yield: 43%).

TLC: CH₂Cl₂/MeOH 9/1 v/v Rf=0.50 N.M.R: DMSO ¹H δ (ppm); 3.6 (s,3H); 3.95 (s,2H); 5.2 (s,2H); 7.20-7.50 (m,7H); 7.80-7.95 (m,2H); 7.95 (s,1H); 8.90 (s,1H); 12.8 (bs,1H) IR: 1720, 1695, 1678, 1612, 1490, 1279, 1100, 759, 732 cm ⁻¹ Mp=236.2° C. Purity (HPLC): 96.7%

### SYNTHESIS EXAMPLE 70

### 4-{6-[3-(4-Methoxy-phenyl)-prop-1-ynyl]-1-methyl-2,4-dioxo-1,4-dihydro-2H-pyrido[3,4-d]pyrimidin-3-ylmethyl}-benzoic acid

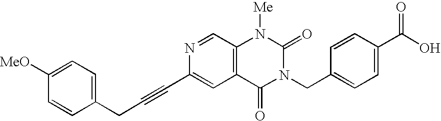

The compound is obtained according to the procedure described in Example 69 using the compound of Preparation B and the 3-(4-methoxyphenyl)-prop-1-yne.

TLC: CH₂Cl₂/MeOH 9/1 v/v Rf=0.60 N.M.R: DMSO ¹H δ (ppm); 3.60 (s,3H); 3.75 (s,3H); 3.85 (s,2H); 5.20 (s,2H); 6.9-7.0 (m,2H); 7.30-7.40 (m,2H); 7.45-7.50 (m,2H); 7.80-7.90 (m,3H); 8.90 (s,1H); 12.9 (bs,1H) IR: 1721, 1670, 1511, 1477, 1421, 1325, 1245, 1178, 1037, 792 cm⁻¹ Mp=262° C. Purity (HPLC): 95.9%

### SYNTHESIS EXAMPLE 71

### 4-Benzyl-7-(3-phenyl-prop-1-ynyl)-4H-[1,2,4]triazolo[4,3-a]quinazolin-5-one

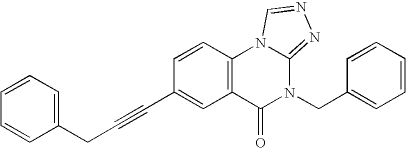

To a suspension of 1.5 g (3.53 mmol) of compound obtained in Step 1 of Preparation C in 12 ml of DMF were added, under inert atmosphere of nitrogen, 0.574 g (4.94 mmol) of 3-phenylprop-1-yne, 1.45 g (14.4 mmol) of triethylamine and 0.1 g of dichlorobis (triphenylphosphin)palladium (II). The reaction mixture was then stirred and heated at 50° C. for 5 hours. After cooling at room temperature, H₂O was added and the mixture extracted several times with AcOEt. The organic phase was washed with water and brine and then dried (Na₂SO₄) and concentrated, leaving 1.5 g of crude solid that was chromatographied on a silica column (CH₂Cl₂/CH₃OH 98.5/1.5 v/v) to afford 0.25 g (yield: 18%) of an off-white solid pure in TLC. A sample was purified by recrystallization in methanol.

Mp=238° C. N.M.R. DMSO ¹H δ (ppm): 3.85 (s, 2H); 5.55 (s, 2H); 7.25-7.45 (m, 8H); 7.6 (d, 1H); 7.65-7.75 (m, 2H); 7.85 (d, 1H); 8.5 (s, 1H); 8.7 (s, 1H).

### SYNTHESIS EXAMPLE 72

### 4-Benzyl-7-[(4-methoxyphenyl)-prop-1-ynyl]-4H-[1,2,4]-triazolo[4,3-a]quinazolin-5 -one

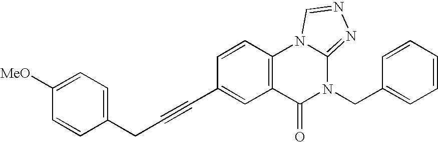

The compound was obtained according to the procedure described in Example 71 using the same substrate (Preparation C, Step 1) and 0.48 g of 3-(4-methoxyphenyl)-prop-1-yne. The crude product was purified by chromatography on a silica column (CH₂Cl₂/CH₃OH 98/2 v/v). A treatment of the resultant solid with boiling AcOEt gave 0.15 g (yield: 15%) of an off-white solid pure in TLC.

Mp=267° C. N.M.R: CDCl₃ ¹H δ (ppm): 3.8 (s, 2H); 3.8 (s, 3H); 5.5 (s, 2H); 6.9 (d, 2H); 7.2-7.35 (m, 5H); 7.6 (d, 1H); 7.68 (d, 2H); 7.8 (d, 1H); 8.4 (s, 1H); 8.7 (s, 1H).

### SYNTHESIS EXAMPLE 73

### Methyl 4-{7-[3-(4-methoxy-phenyl)-prop-1-ynyl]-5-oxo-5H-[1,2,4]triazolo[4,3-a]quinazolin-4-ylmethyl}-benzoate

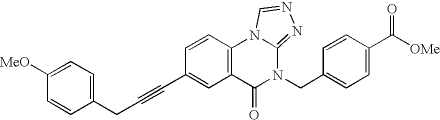

The compound was obtained according to the procedure described in Example 71 using the compound of the Preparation C Step 3, 1.1 g of 3-(4-methoxyphenyl)prop-1-yne, and 2.72 g of N-ethyl-N,N-diisopropylamine. The crude product was purified by chromatography on a silica column (CH₂Cl₂/CH₃OH 98/2 v/v). A treatment of the resultant solid with boiling AcOEt gave 1.5 g (yield: 59%) of an off-white solid pure in TLC.

Mp=249° C. N.M.R: CDCl₃ ¹H δ (ppm): 3.79 (s, 2H); 3.81 (s, 3H); 3.88(s, 3H); 5.56 (s, 2H); 6.89 (d, 2H); 7.30 (d, 2H); 7.60 (d, 1H); 7.70 (d, 2H); 7.82 (d, 1H); 7.97 (d, 2H); 8.44 (s, 1H); 8.7 (s, 1H).

### SYNTHESIS EXAMPLE 74

### 4-[5-Oxo-7-(3-phenyl-prop-1-ynyl)-5H-[1,2,4]triazolo[4,3-a]quinazolin-4-ylmethyl]-benzoic acid

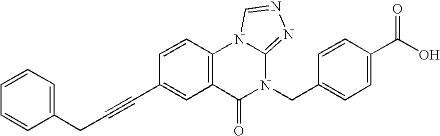

The compound was obtained according to the procedure described in Example 71 using the compound of the Preparation D (0.195 g), 0.067 g of 3-phenylprop-1-yne, and 0.215 g of N-ethyl-N,N-diisopropylamine. The crude product was purified by chromatography on a silica column (CH₂Cl₂/CH₃OH 90/10 then 85/15 v/v) to afford 0.14 g (yield: 77%) of an off-white solid pure in TLC corresponding to the desired product.

Mp=262° C. N.M.R: DMSO ¹H δ (ppm): 3.96 (s, 2H); 5.42 (s, 2H); 7.27 (t, 1H); 7.37 (t, 2H); 7.44 (d, 2H); 7.52 (d, 2H); 7.87 (d, 2H); 8.02 (d, 1H); 8.18-8.22 (m, 2H); 9.53 (s, 1H); 12.5-13.2 (m, 1H).

### SYNTHESIS EXAMPLE 75

### 4-(1-Methyl-2,4-dioxo-6-(2-phenylethynyl)-1,4-dihydro-2H-quinazolin-3-ylmethyl)-benzoic acid

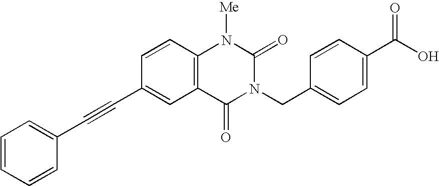

The compound was obtained according to the procedure described in Synthesis Example 70 using the compound of the Preparation A Step 4 (0.59 g, 1.35 mmol), 0.193 g (1.89 mmol) of 1-phenyleth-1-yne, 0.050 g of dichlorobis (triphenylphosphine)palladium, a catalytic amount of CuI and 0.700 g (5.4 mmol) of N-ethyl-N,N-diisopropylamine. The crude product was purified by crystallization in dichloromethane provided 0.55 g (yield: 100%) of an off-white solid pure in TLC.

Mp=260° C. N.M.R: DMSO ¹H δ (ppm): 3.55 (s, 3H); 5.21 (s, 2H); 7.36-7.50 (m, 5H); 7.50-7.65 (m, 3H); 7.82-7.99 (m, 3H); 8.16 (s, 1H); 12.7-13.1 (m, 1H).

1-45. (canceled).

46. A method of determining if a compound has potential to allosterically bind to S1′ and S1″ sites of MMP-13, the method comprising the steps of:
selecting a compound that comprises first and second hydrophobic groups and first and second hydrogen bond acceptors; and
determining whether the relative positions of centroids of the first and second hydrophobic groups and the first and second hydrogen bond acceptors are present at the following Cartesian coordinates in Å:
(i) first hydrogen bond acceptor, 0.00, 0.00, 0.00;
(ii) second hydrogen bond acceptor, 5.08, 2.23, 0.00;
(iii) first hydrophobic group, −1.52, −3.06, −0.23; and
(iv) second hydrophobic group, 9.07, 0.00, 0.00;
wherein tolerances in the positions of the hydrophobic groups and the hydrogen bond acceptors are ±1.0 Å and ±1.5 Å, respectively.

47. The method of claim 46, wherein the first hydrophobic group contains a bicyclic ring system containing between 8 and 10 atoms and which may contain one or several heteroatoms, or a 5- or 6-membered monocyclic aromatic group which may contain one or more heteroatoms and which may be 4-substituted or 3,4-disubstituted, but which is of width (including substituents) less than 4.0 Å.

48. The method of claim 47, wherein the pi-system of the aromatic ring is electron rich.

49. The method of claim 46, wherein first hydrophobic group, is linked by a first linker chain which is three atoms long to a first 5- or 6-membered ring of the scaffold, the first linker chain atom adjacent to said first scaffold ring forming part of the first hydrogen bond acceptor.

50. The method of claim 49, wherein the first linker chain has a methylene group located adjacent to the hydrophobic group.

51. The method of claim 49, wherein the scaffold further comprises a second scaffold ring fused to the first scaffold ring at locations two and three ring atoms distant from the junction between the first scaffold ring and the first linker chain, and the atom of the second scaffold ring adjacent to the atom of the first scaffold ring that is two positions distant from said junction forms part of the second hydrogen bond acceptor.

52. The method of claim 51, wherein the atom of the second scaffold ring adjacent to the atom of the first scaffold ring that is three positions distant from said junction has a substituent which is a single atom or is a methyl group.

53. The method of claim 46, wherein the second hydrophobic group is a 5- or 6-membered aromatic ring which may contain one or several heteroatoms, a bicyclic ring system containing between 8 and 10 atoms and which may contain one or several heteroatoms, or a planar saturated or unsaturated system.

54. A method of determining if a compound has potential to allosterically bind to S1′ and S1″ sites of MMP-13, the method comprising the steps of:
selecting a compound that comprises a first hydrophobic group and first, second and third hydrogen bond acceptors; and
determining whether the relative positions of centroids of the first hydrophobic group and the first, second and third hydrogen bond acceptors are present at the following Cartesian coordinates in Å:
(i) first hydrogen bond acceptor, 0.00, 0.00, 0.00;
(ii) second hydrogen bond acceptor, 5.08, 2.23, 0.0;
(iii) third hydrogen bond acceptor, 7.15, 0.80, 0.00; and
(iv) first hydrophobic group, −1.52, −3.06, −0.23;
wherein tolerances in the positions of the first hydrophobic group and the hydrogen bond acceptors are ±1.0 Å and ±1.5 Å, respectively.

55. The method of claim 54, wherein the first hydrophobic group contains a bicyclic ring system containing between 8 and 10 atoms and which may contain one or several heteroatoms, or a 5- or 6-membered monocyclic aromatic group which may contain one or more heteroatoms and which may be 4-substituted or 3,4-disubstituted, but which is of width (including substituents) less than 4.0 Å.

56. The method of claim 55, wherein the pi-system of the aromatic ring is electron rich.

57. The method of claim 55, wherein first hydrophobic group, is linked by a first linker chain which is three atoms long to a first 5- or 6-membered ring of the scaffold, the first linker chain atom adjacent to said first scaffold ring forming part of the first hydrogen bond acceptor.

58. The method of claim 57, wherein the chain has a methylene group located adjacent to the hydrophobic group.

59. The method of claim 57, wherein the scaffold further comprises a second ring fused to the first scaffold ring at locations two and three ring atoms distant from the junction between the first scaffold ring and the chain, and the atom of the second scaffold ring adjacent to the atom of the first scaffold ring that is two positions distant from said junction forms part of the second hydrogen bond acceptor.

60. The method of claim 59, wherein the atom of the second scaffold ring adjacent to the atom of the first scaffold ring that is three positions distant from said junction has a substituent which is a single atom or is a methyl group.

61. The method of claim 59, wherein the second scaffold ring is 6-membered and the atom of the second scaffold ring that is two positions distant from the atom that forms part of the second hydrogen bond acceptor forms part of the third hydrogen bond acceptor.

62. The method of claim 59, wherein the second scaffold ring is 6-membered and a third scaffold ring is fused to the second scaffold ring at those atoms of the second scaffold ring which are two and three positions distant from the atom that forms part of the second hydrogen bond acceptor, an atom of the third scaffold ring forming part of the third hydrogen bond acceptor.

63. A method of determining if a compound has potential to allosterically bind to S1′ and S1″ sites of MMP-13, the method comprising the steps of:
selecting a compound that comprises first and second hydrophobic groups and first, second and third hydrogen bond acceptors; and
determining whether the relative positions of centroids of the first and second hydrophobic groups and the first, second and third hydrogen bond acceptors are present at the following Cartesian coordinates in Å:
(i) first hydrogen bond acceptor, 0.00, 0.00, 0.00;
(ii) second hydrogen bond acceptor, 5.08, 2.23, 0.0;
(iii) third hydrogen bond acceptor, 7.15, 0.80, 0.00;
(iv) first hydrophobic group, −1.52, −3.06, −0.23; and
(v) second hydrophobic group, 9.07, 0.00, 0.00;
wherein tolerances in the positions of the hydrophobic groups and the hydrogen bond acceptors are ±1.0 Å and ±1.5 Å, respectively.

64. The method of claim 63, wherein the first hydrophobic group contains a bicyclic ring system containing between 8 and 10 atoms and which may contain one or several heteroatoms, or a 5- or 6-membered monocyclic aromatic group which may contain one or more heteroatoms and which may be 4-substituted or 3,4-disubstituted, but which is of width (including substituents) less than 4.0 Å.

65. The method of claim 64, wherein the pi-system of the aromatic ring is electron rich.

66. The method of claim 64, wherein first hydrophobic group, is linked by a first linker chain which is three atoms long to a first 5- or 6-membered ring of the scaffold, the first linker chain atom adjacent to said first scaffold ring forming part of the first hydrogen bond acceptor.

67. The method of claim 66, wherein the chain has a methylene group located adjacent to the hydrophobic group.

68. The method of claim 66, wherein the scaffold further comprises a second scaffold ring fused to the first scaffold ring at locations two and three ring atoms distant from the junction between the first scaffold ring and the first linker chain, and the atom of the second scaffold ring adjacent to the atom of the first scaffold ring that is two positions distant from said junction forms part of the second hydrogen bond acceptor.

69. The method of claim 68, wherein the atom of the second scaffold ring adjacent to the atom of the first scaffold ring that is three positions distant from said junction has a substituent which is a single atom or is a methyl group.

70. The method of claim 68, wherein the second scaffold ring is 6-membered and the atom of the second scaffold ring that is two positions distant from the atom that forms part of the second hydrogen bond acceptor forms part of the third hydrogen bond acceptor.

71. The method of claim 68, wherein the second scaffold ring is 6-membered and a third scaffold ring is fused to the second scaffold ring at those atoms of the second scaffold ring which are two and three positions distant from the atom that forms part of the second hydrogen bond acceptor, an atom of the third scaffold ring forming part of the third hydrogen bond acceptor.

72. The method of claim 63, wherein the second hydrophobic group is a 5- or 6-membered aromatic ring which may contain one or several heteroatoms, a bicyclic ring system containing between 8 and 10 atoms and which may contain one or several heteroatoms, or a planar saturated or unsaturated system.

73. A method of determining if a compound has potential to allosterically bind to S1′ and S1″ sites of MMP-13, the method comprising the steps of:
selecting a compound that comprises a scaffold, first and second hydrogen bond acceptors and first and second hydrophobic groups connected by side chains to the scaffold, a cyclic structure forming part of the scaffold being located between the first and second hydrogen bond acceptors; and
determining whether the first and second hydrogen bond acceptors and the first and second hydrophobic groups are arranged so that:
The first and second hydrogen bond acceptors can bind with Thr 245 and Thr 247, respectively, of the MMP-13;
The first hydrophobic group can locate within the S1′ site; and
The second hydrophobic group can be open to solvent.

74. A method of determining if a compound has potential to allosterically bind to S1′ and S1″ sites of MMP-13, the method comprising the steps of:
selecting a compound that comprises a scaffold, first, second and third hydrogen bond acceptors, and a hydrophobic group connected by a side chain to the scaffold, a cyclic structure forming part of the scaffold being located between the first and second hydrogen bond acceptors; and
determining whether the first, second and third hydrogen bond acceptors, and the hydrophobic group are arranged so that:
The first, second and third hydrogen bond acceptors can bind with Thr 245, Thr 247, and Met 253, respectively, of the MMP-13; and
The hydrophobic group can locate within the S1′ site.

75. A method of determining if a compound has potential to allosterically bind to S1′ and S1″ sites of MMP-13, the method comprising the steps of:
selecting a compound that comprises a scaffold, first, second and third hydrogen bond acceptors, and first and second hydrophobic groups connected by side chains to the scaffold, a cyclic structure forming part of the scaffold being located between the first and second hydrogen bond acceptors; and
determining whether the first, second and third hydrogen bond acceptors, and the first and second hydrophobic groups are arranged so that:
The first, second and third hydrogen bond acceptors can bind with Thr 245, Thr 247, and Met 253, respectively, of the MMP-13;
The first hydrophobic group can locate within the S1′ site; and
The second hydrophobic group can be open to solvent.

76. The method according to any one of the preceding claims, wherein the compound is a pyrido[3,4-d]pyrimidine-2,4(1H,3H)-dione.
